(19) United States
(12) Patent Application Publication
(10) Pub. No.: US 2006/0030016 A1
(43) Pub. Date: Feb. 9, 2006

(54) Crystal structure of interleukin-2 tyrosine kinase (ITK) and binding pockets thereof

(76) Inventors: Graham Cheetham, Abingdon (GB); Kieron Brown, Bicester (GB); Ronald Knegtel, Abingdon (GB); Suzanne Renwick, Middlesex (GB); Sarah Vial, Newbury (GB)

Correspondence Address: CHOATE, HALL & STEWART LLP, TWO INTERNATIONAL PLACE, BOSTON, MA, 02110, US

(21) Appl. No.: 11/003,787

(22) Filed: Dec. 3, 2004

Related U.S. Application Data
(60) Provisional application No. 60/527,372, filed on Dec. 5, 2003.

(51) Int. Cl. G06F 19/00; C12N 9/12; G01N 33/48; G01N 33/50

(52) U.S. Cl. 435/194; 702/19

(57) ABSTRACT

The invention relates to molecules or molecular complexes which comprise binding pockets of ITK or its structural homologues. The invention relates to crystallizable compositions and crystals comprising ITK. The present invention also relates to a data storage medium encoded with the structural coordinates of molecules and molecular complexes which comprise the ITK or ITK-like ATP-binding pockets. The present invention also relates to a computer comprising such data storage material. The computer may generate a three-dimensional structure or graphical three-dimensional representation of such molecules or molecular complexes. This invention also relates to methods of using the structure coordinates to solve the structure of homologous proteins or protein complexes. In addition, this invention relates to methods of using the structure coordinates to screen for and design compounds, including inhibitory compounds, that bind to ITK or homologues thereof.

### PRIORITY CLAIM

This application asserts priority to Provisional Application No. 60/527,372, filed Dec. 5, 2003; which is incorporated herein by reference in its entirety.

### TECHNICAL FIELD OF THE INVENTION

The present invention relates to expression, purification, characterization and X-ray analysis of crystalline molecules or molecular complexes of Interleukin-2 Tyrosine kinase (ITK). The present invention provides for the first time the crystal structure of ITK bound to staurosporine or 3-(8-Phenyl-5,6-dihydrothieno[2,3-h]quinazolin-2-ylamino)benzene sulfonamide. The present invention also provides crystalline molecules or molecular complexes that comprise binding pockets of ITK kinase (ITK) and/or its structural homologues, the structure of these molecules or molecular complexes. The present invention further provides crystals of ITK complexed with staurosporine or 3-(8-Phenyl-5,6-dihydrothieno[2,3-h]quinazolin-2-ylamino)benzenesulfonamide and methods for producing these crystals. This invention also relates to crystallizable compositions from which the protein-ligand complexes may be obtained. The present invention also relates to a data storage medium encoded with the structural coordinates of molecules and molecular complexes that comprise the ATP-binding pockets of ITK or their structural homologues. The present invention also relates to a computer comprising such data storage material. The computer may generate a three-dimensional structure or graphical three-dimensional representation of such molecules or molecular complexes. This invention also relates to methods of using the structure coordinates to solve the structure of homologous proteins or protein complexes. This invention also relates to computational methods of using structure coordinates of the ITK complex(es) to screen for and design compounds, including inhibitory compounds and antibodies, that interact with ITK or homologues thereof.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. (See, Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J., 9:576-596 (1995); Knighton et al., Science, 253:407-414 (1991); Hiles et al., Cell, 70:419-429 (1992); Kunz et al., Cell, 73:585-596 (1993); Garcia-Bustos et al., EMBO J., 13:2352-2361 (1994)).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there has been a substantial effort in medicinal chemistry to find protein kinase inhibitors that are effective as therapeutic agents.

Among medically important kinases are the tyrosine kinases. The tyrosine kinase family includes the Src-related tyrosine kinases (Sicheri F and Kuriyan J. Curr Opin Struct Biol., 6:77-85 (1997)). The activity of tyrosine kinases is modulated my phosphorylation of the catalytic kinase domain and also the adjacent SH2- and SH3-domains.

The TEC-family of protein kinases is another important subgroup of five closely related tyrosine protein kinases (amino acid residues located in the ATP-binding site are shown in Table 1). The Tec family of non-receptor tyrosine kinases plays a central role in signalling through antigen-receptors such as the TCR, BCR and Fcε receptors (reviewed in Miller A, et al. Current Opinion in Immunology 14:331-340 (2002)). Tec family kinases are essential for T cell activation. Three members of the Tec family, ITK, RLK and TEC, are activated downstream of antigen receptor engagement in T cells and transmit signals to downstream effectors, including PLC-γ. Deletion of ITK in mice results in reduced T cell receptor (TCR)-induced proliferation and secretion of the cytokines IL-2, IL-4, IL-5, IL-10 and IFN-γ (Schaeffer et al, Science 284; 638-641 (1999)), Fowell et al, Immunity 11; 399-409 (1999), Schaeffer et al, Nature Immunology 2(12):1183-1188 (2001))). The immunological symptoms of allergic asthma are attenuated in ITK−/− mice. Lung inflammation, eosinophil infiltration and mucous production are drastically reduced in ITK−/− mice in response to challenge with the allergen OVA (Mueller et al, Journal of Immunology 170: 5056-5063 (2003)). ITK has also been implicated in atopic dermatitis. This gene has been reported to be more highly expressed in peripheral blood T cells from patients with moderate and/or severe atopic dermatitis than in controls or patients with mild atopic dermatitis (Matsumoto et al, International archives of Allergy and Immunology 129:327-340 (2002)).

Splenocytes from RLK−/− mice secrete half the IL-2 produced by wild type animals in response to TCR engagement (Schaeffer et al, Science 284:638-641 (1999)), while combined deletion of ITK and RLK in mice leads to a profound inhibition of TCR-induced responses including proliferation and production of the cytokines IL-2, IL-4, IL-5 and IFN-γ (Schaeffer et al, Nature Immunology 2(12):1183-1188 (2001)), Schaeffer et al, Science 284:638-641 (1999)). Intracellular signalling following TCR engagement is effected in ITK/RLK deficient T cells; inositol triphosphate production, calcium mobilization, MAP kinase activation, and activation of the transcription factors NFAT and AP-1 are all reduced (Schaeffer et al, Science 284:638-641 (1999), Schaeffer et al, Nature Immunology 2(12):1183-1188 (2001)).

Tec family kinases are also essential for B cell development and activation. Patients with mutations in BTK have a profound block in B cell development, resulting in the almost complete absence of B lymphocytes and plasma cells, severely reduced Ig levels and a profound inhibition of humoral response to recall antigens (reviewed in Vihinen et al, Frontiers in Bioscience 5:d917-928). Mice deficient in BTK also have a reduced number of peripheral B cells and greatly decreased levels of IgM and IgG3. BTK deletion in mice has a profound effect on B cell proliferation induced by anti-IgM, and inhibits immune responses to thymus-independent type II antigens (Ellmeier et al, J Exp Med 192:1611-1623 (2000)).

Tec kinases also play a role in mast cell activation through the high-affinity IgE receptor (FcεRI). ITK and BTK are expressed in mast cells and are activated by FcεRI cross-linking (Kawakami et al, Journal of Immunology; 3556-3562 (1995)). BTK deficient murine mast cells have reduced degranulation and decreased production of proinflammatory cytokines following FcεRI cross-linking (Kawakami et al., Journal of leukocyte biology 65:286-290). BTK deficiency also results in a decrease of macrophage effector functions (Mukhopadhyay et al, Journal of Immunology; 168:2914-2921 (2002)).

Together these studies have defined an important role for ITK in TCR signaling leading to thymic development, cytokine gene expression, and activation-induced cell death

Accordingly, there has been an interest in finding selective inhibitors of ITK or selective inhibitors of the TEC-family of kinases that are effective as therapeutic agents. A challenge has been to find protein kinase inhibitors that act in a selective manner, targeting only ITK or the Tec family kinases. Since there are numerous protein kinases that are involved in a variety of cellular responses, non-selective inhibitors may lead to unwanted side effects. In this regard, the three-dimensional structure of the kinase would assist in the rational design of inhibitors. The determination of the amino acid residues in ITK binding pockets and the determination of the shape of those binding pockets would allow one to design selective inhibitors that bind favorably to this class of enzymes. The determination of the amino acid residues in ITK binding pockets and the determination of the shape of those binding pockets (collected in Table 1) would also allow one to design inhibitors that can bind to ITK, or any combination of the TEC-family kinases thereof.

For example, a general approach to designing inhibitors that are selective for an enzyme target is to determine how a putative inhibitor interacts with the three dimensional structure of the enzyme. For this reason it is useful to obtain the enzyme protein in crystal form and perform X-ray diffraction techniques to determine its three dimensional structure coordinates. If the enzyme is crystallized as a complex with a ligand, one can determine both the shape of the enzyme binding pocket when bound to the ligand, as well as the amino acid residues that are capable of close contact with the ligand. By knowing the shape and amino acid residues in the binding pocket, one may design new ligands that will interact favorably with the enzyme. With such structural information, available computational methods may be used to predict how strong the ligand binding interaction will be. Such methods thus enable the design of inhibitors that bind strongly, as well as selectively to the target enzyme.

Despite the fact that the genes for various Tec family members have been isolated and the amino acid sequences of ITK, BTK, BMX, RLK and TEC proteins are known, no one has described X-ray crystal structural coordinate information of ITK protein. As discussed above, such information would be extremely useful in identifying and designing potential inhibitors of the ITK kinase or homologues thereof, which, in turn, could have therapeutic utility.

The structures of several Tyrosine kinases have been solved by X-ray diffraction and analyzed (reviewed in al-Obeidi F A et al., Biopolymers, 3:197-223 (1998)). Specifically, the crystal structures of Src-family Tyrosine kinases have been studied in detail (Sicheri F and Kuriyan J., Curr Opin Struct Biol., 6:777-785 (1997); Yamaguchi H., Hendrickson W. A., Nature, 384:484-489 (1996)).

Recently the crystal structure of BTK kinase domain, another member of the TEC-family, has been determined (Mao, C et al, J. Biol. Chem., 276:41435-41443 (2001)). This revealed that the un-complexed BTK enzyme adopts an inactive kinase conformation that is not commensurate with binding inhibitors or ATP. X-ray solution scattering has also been used to study the conformation of the full-length BTK enzyme and association of the SH and Tec-homology domains with the catalytic kinase domain (Marquez J A et al., EMBO J, 22:4616-4624 (2003)). Thus the crystal structure of unphosphorylated and phosphorylated ITK kinase domain complexes with inhibitors are of great importance for defining the active conformation of ITK and also the TEC-family kinases. This information is essential for the rational design of selective and potent inhibitors of ITK.

TABLE 1: Sequence comparison of active site residues in the Tec family kinases. Residues in and around the bound inhibitor have been classified according to binding of the adenosine, ribose (Rib) and first (TP1) and second (TP2) phosphate groups of ATP. Residue Phe435 in ITK is of great importance as it holds the key to specificity within the TEC-family of kinases and is the gatekeeper to a hydrophobic pocket (see FIG. 5). Numbering corresponds to ITK.

| | Adenine | | | | | | | | | | FP | | | | | Rib | | |
|---|---|---|---|---|---|---|---|---|---|---|---|---|---|---|---|---|---|---|
| Num | 369 | 377 | 389 | 419 | 436 | 437 | 438 | 439 | 489 | 499 | 391 | 410 | 421 | 433 | 435 | 442 | 445 | 486 |
| ITK | I | V | A | V | E | F | M | E | L | S | K | M | L | L | F | C | D | R |
| BTK | L | V | A | V | E | F | M | A | L | S | K | M | L | I | T | C | D | R |
| RLK | I | V | A | V | E | F | M | E | L | S | K | M | L | I | T | C | N | R |
| BMX | L | V | A | V | E | Y | I | S | L | S | K | M | F | I | T | C | N | R |
| TEC | L | V | A | V | E | F | M | E | L | S | K | M | L | I | T | C | N | R |

| | TP1 | | | | TP2 | | | | | | | | | |
|---|---|---|---|---|---|---|---|---|---|---|---|---|---|---|
| Num | 492 | 484 | 487 | 500 | 371 | 372 | 373 | 374 | 375 | 376 | 399 | 402 | 403 | 406 |
| ITK | D | A | N | D | S | G | Q | F | G | L | S | D | F | E |
| BTK | D | A | N | D | T | G | Q | F | G | V | S | E | F | E |
| RLK | D | A | N | D | S | G | Q | F | G | V | S | D | F | E |
| BMX | D | A | N | D | S | G | Q | F | G | V | S | E | F | E |
| TEC | D | A | N | D | S | G | L | F | G | V | C | D | F | E |

### SUMMARY OF THE INVENTION

The present invention provides for the first time, crystallizable compositions, crystals, and the crystal structures of ITK-inhibitor complexes. The ITK protein used in these studies corresponds to a single polypeptide chain, which encompasses the complete catalytic kinase domain, amino acids 357 to 620. Solving these crystal structures have allowed applicants to determine the key structural features of ITK, particularly the shape of its substrate and ATP-binding pockets.

Thus, in one aspect, the present invention provides molecules or molecular complexes comprising all or parts of these binding pockets, or homologues of these binding pockets that have similar three-dimensional shapes.

In another aspect, the present invention further provides crystal structures of ITK complexed with inhibitors thereof, and methods for producing these crystals. In another embodiment, the present invention provides crystals of ITK complexed with staurosporine and methods for producing these crystals. In another embodiment, the present invention provides crystals of ITK complexed with 3-(8-Phenyl-5,6-dihydrothieno[2,3-h]quinazolin-2-ylamino)benzenesulfonamide and methods for producing these crystals. In certain embodiments, ITK is unphosphorylated. In certain other embodiments, ITK is phosphorylated.

In a further aspect, the present invention provides crystallizable compositions from which ITK-ligand complexes may be obtained.

In another aspect, the invention provides a data storage medium that comprises the structure coordinates of molecules and molecular complexes that comprise all or part of the ITK binding pockets. Such storage medium encoded with these data when read and utilized by a computer programmed with appropriate software displays, on a computer screen or similar viewing device, a three-dimensional graphical representation of a molecule or molecular complex comprising such binding pockets or similarly shaped homologous binding pockets.

In yet another aspect, the invention provides computational methods of using structure coordinates of the ITK complex(es) to screen for and design compounds, including inhibitory compounds and antibodies, that interact with ITK or homologues thereof. In certain embodiments, the invention provides methods for designing, evaluating and identifying compounds, which bind to the aforementioned binding pockets. In certain embodiments, such compounds are potential inhibitors of ITK or their homologues.

In a further aspect, the invention provides a method for determining at least a portion of the three-dimensional structure of molecules or molecular complexes which contain at least some structurally similar features to ITK, particularly RLK, BTK, TEC and BMX and their homologues. In certain embodiments, this is achieved by using at least some of the structural coordinates obtained from the ITK complexes.

### BRIEF DESCRIPTION OF THE DRAWING

FIG. 1 lists the atomic structure coordinates for the unphosphorylated ITK-3-(8-Phenyl-5,6-dihydrothieno[2,3-h]quinazolin-2-ylamino)benzenesulfonamide inhibitor complex as derived by X-ray diffraction from the crystal. The crystallographic asymmetric unit contains two molecular complexes. The first complex is defined as PDB chain A and C. The second is chains B and D.

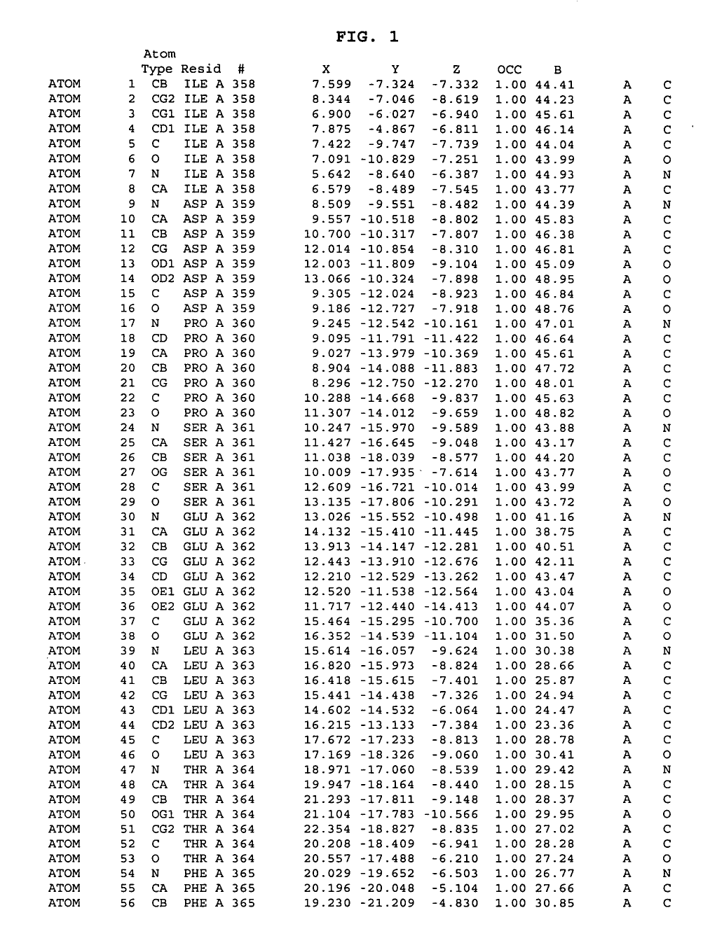

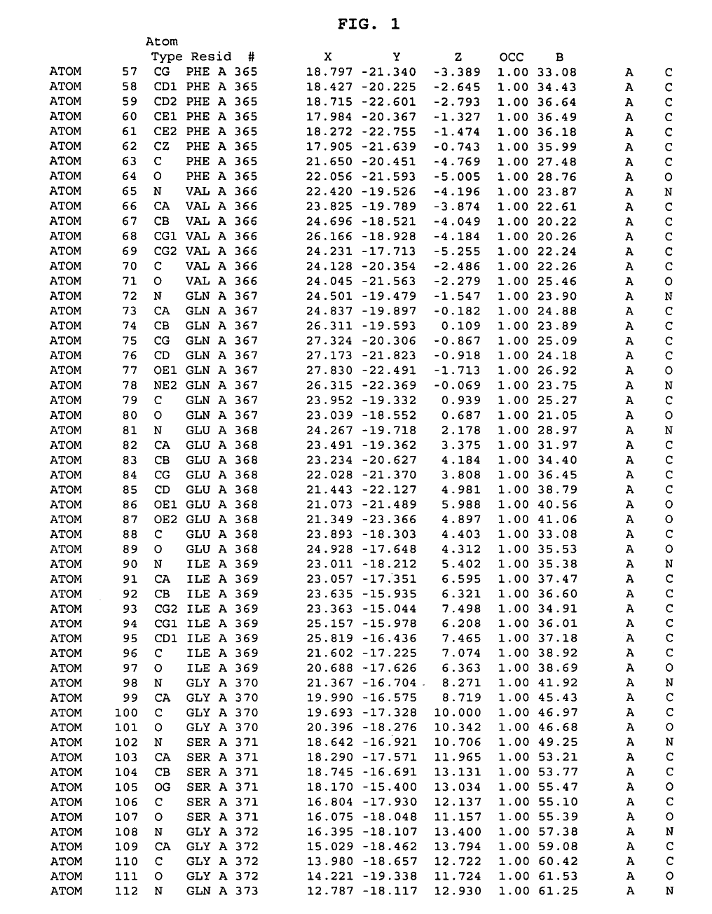

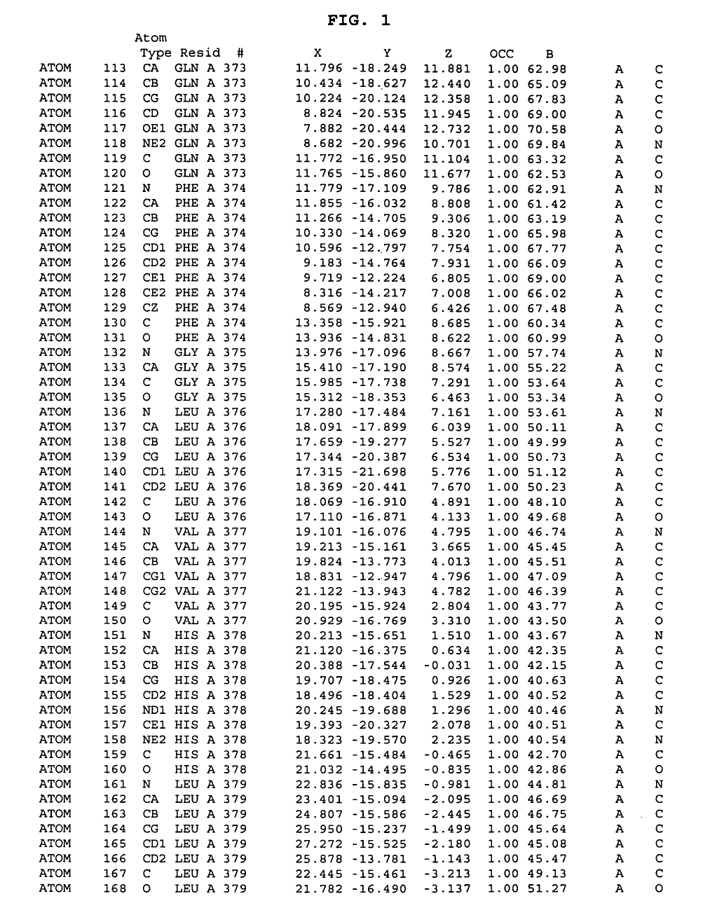

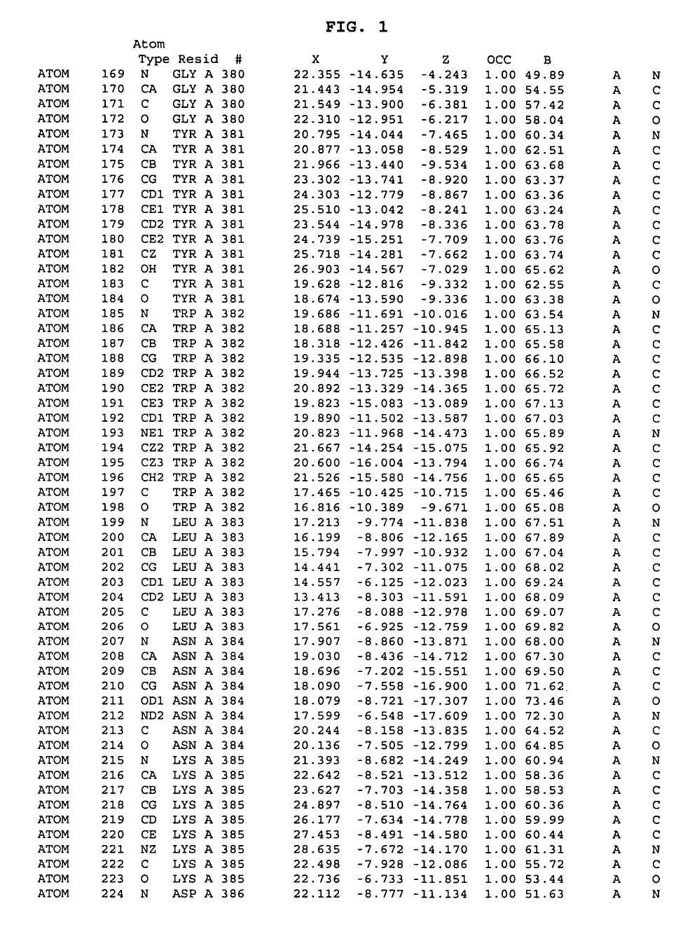

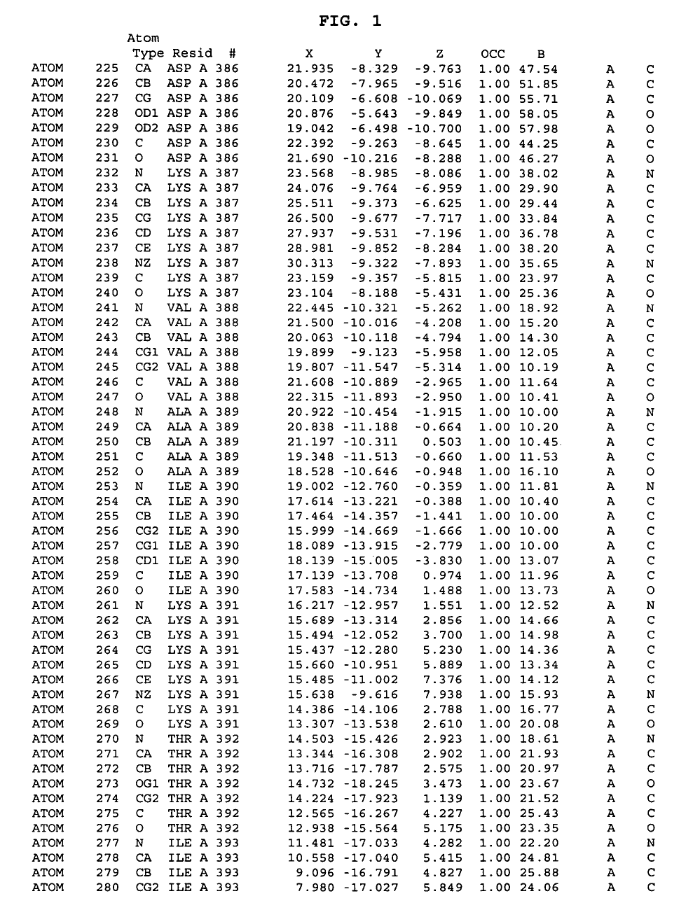

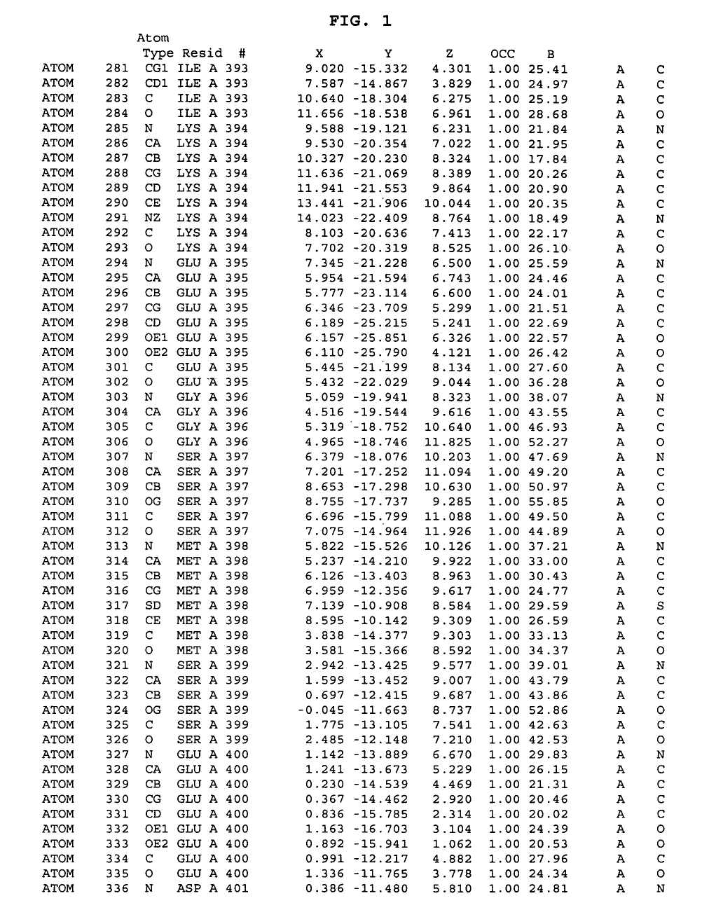

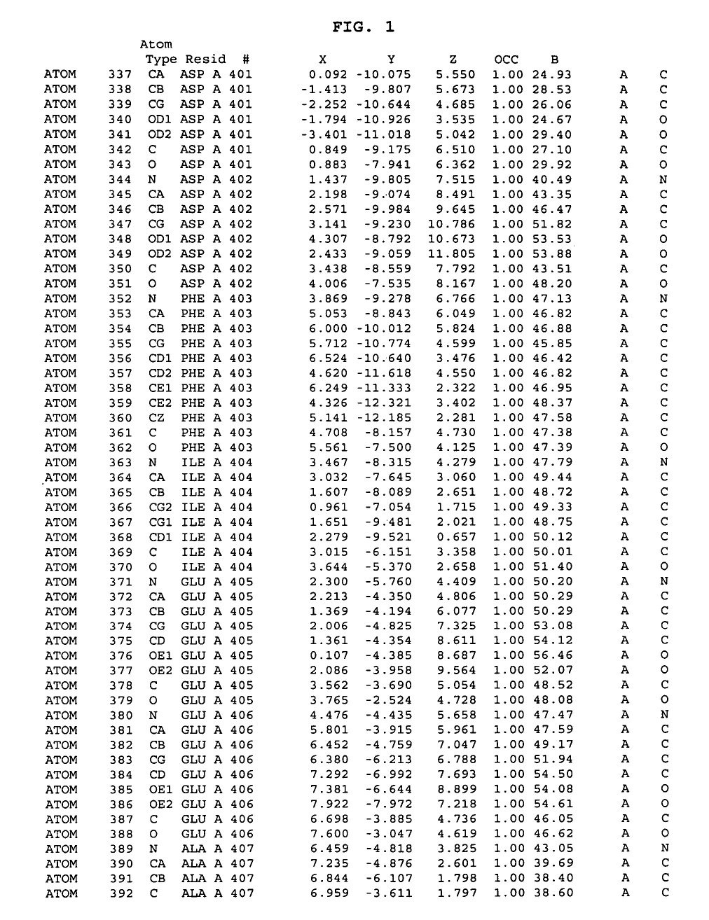

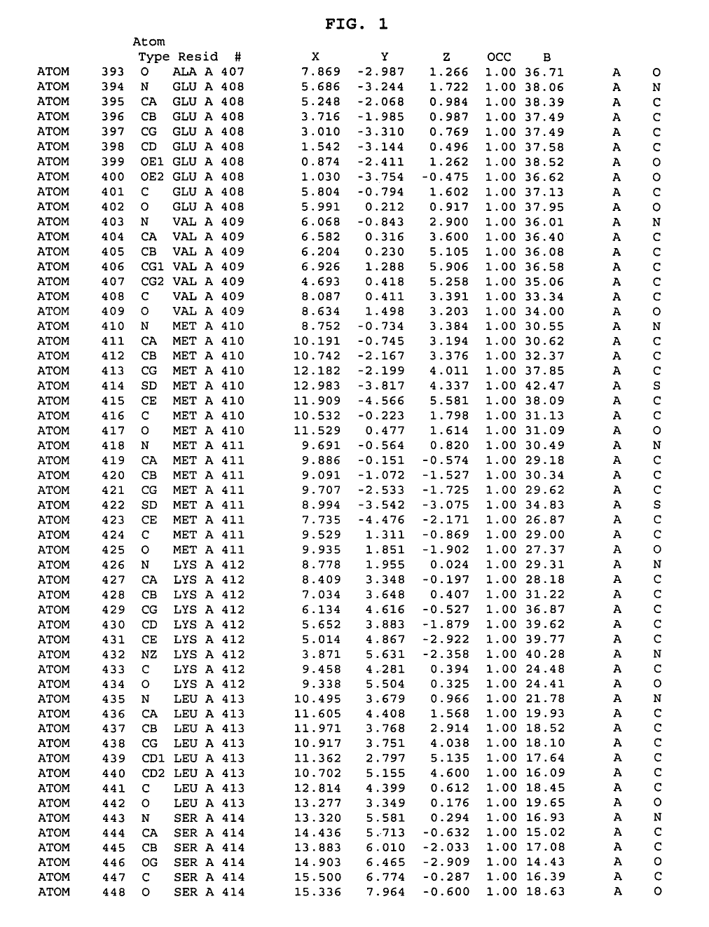

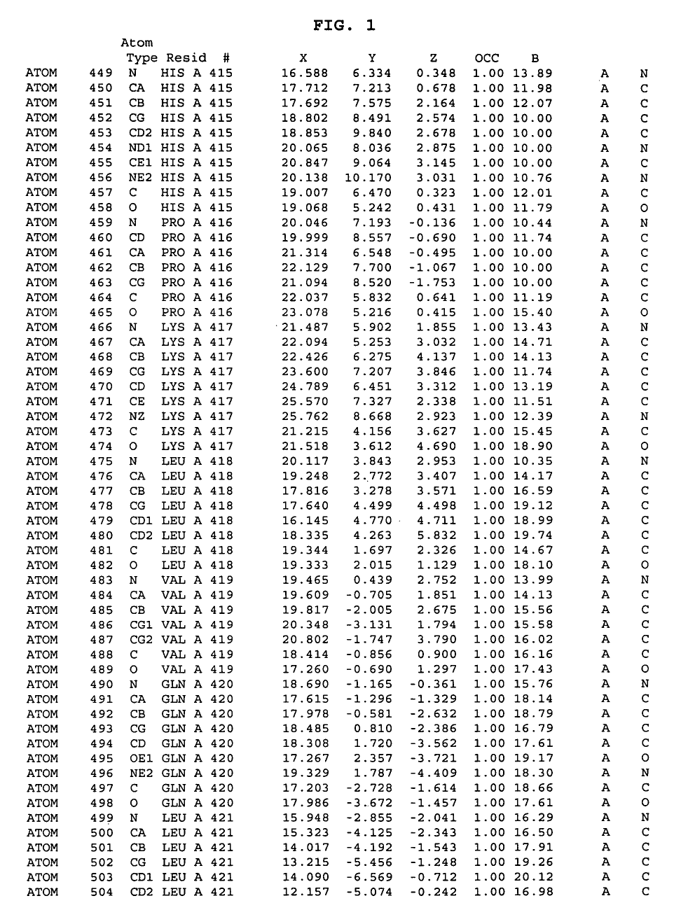

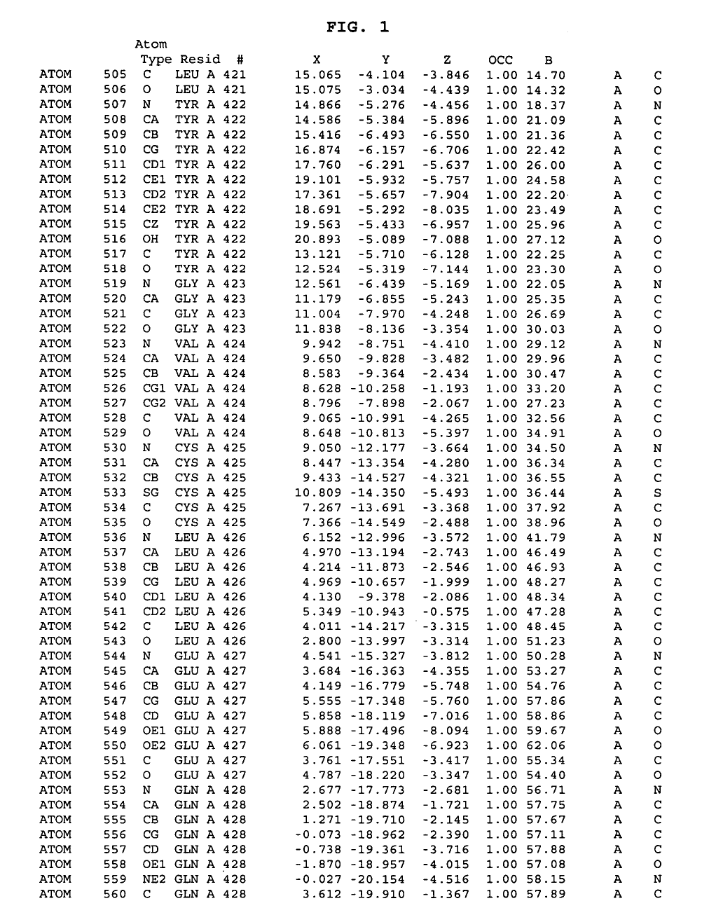

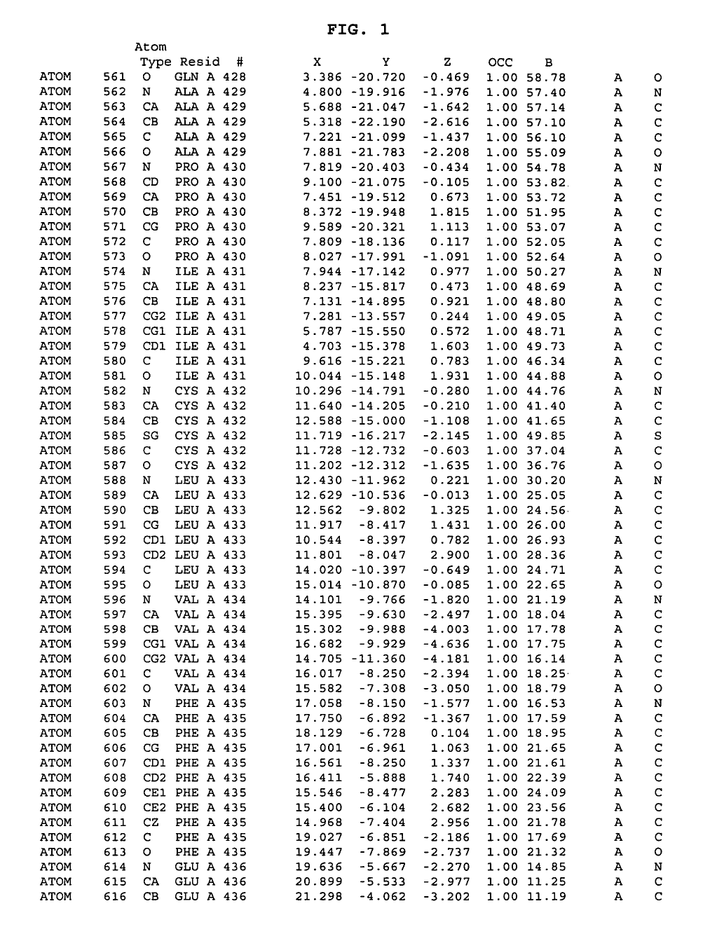

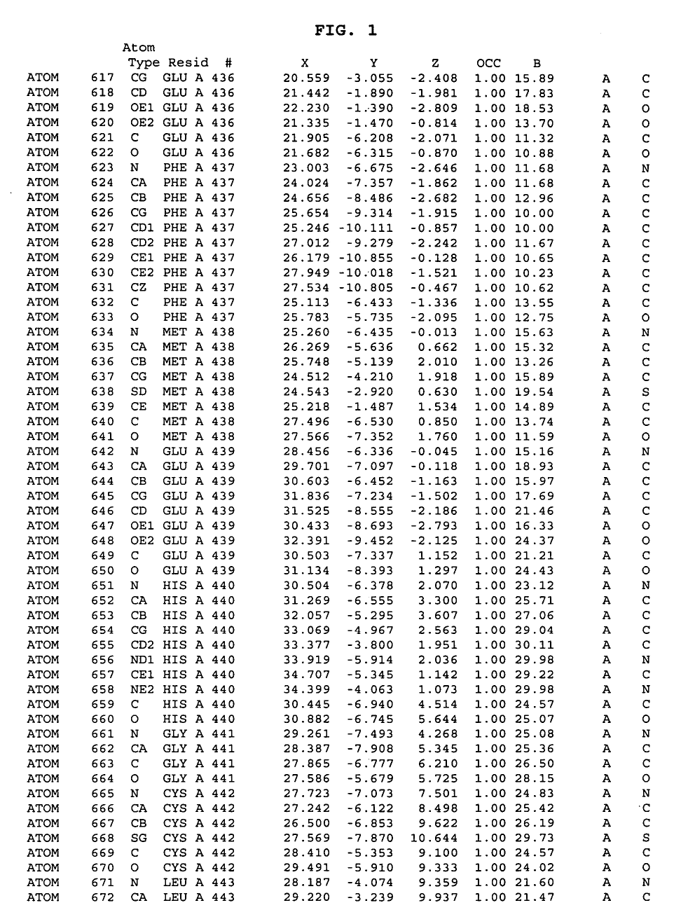

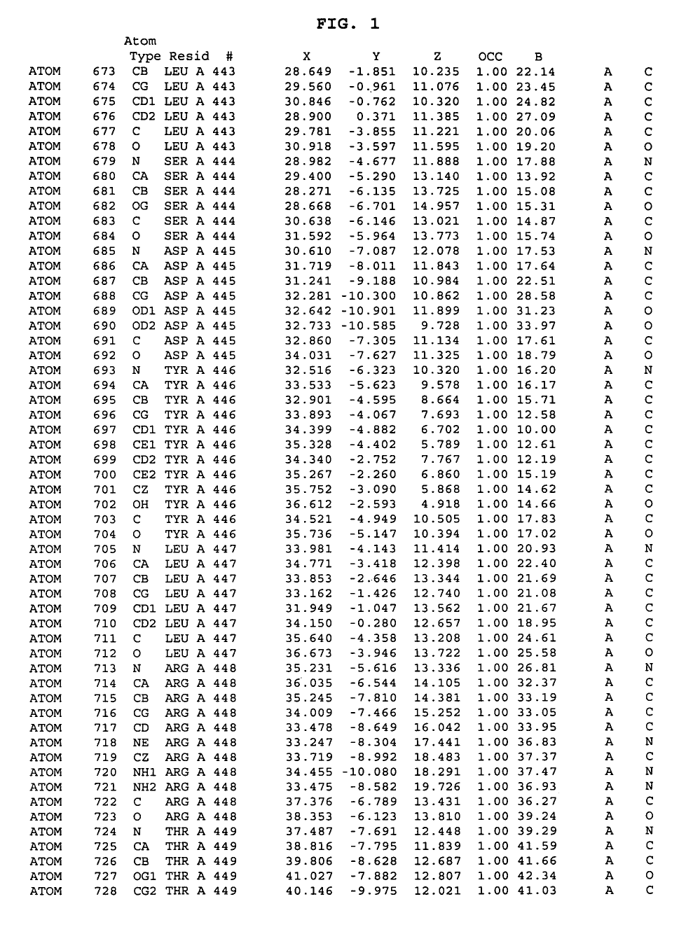

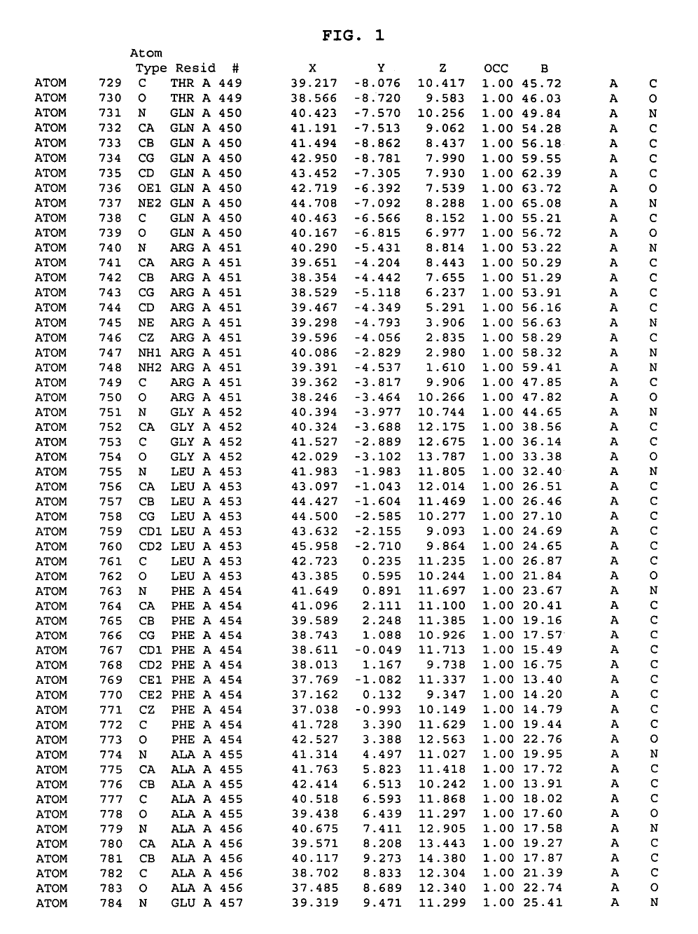

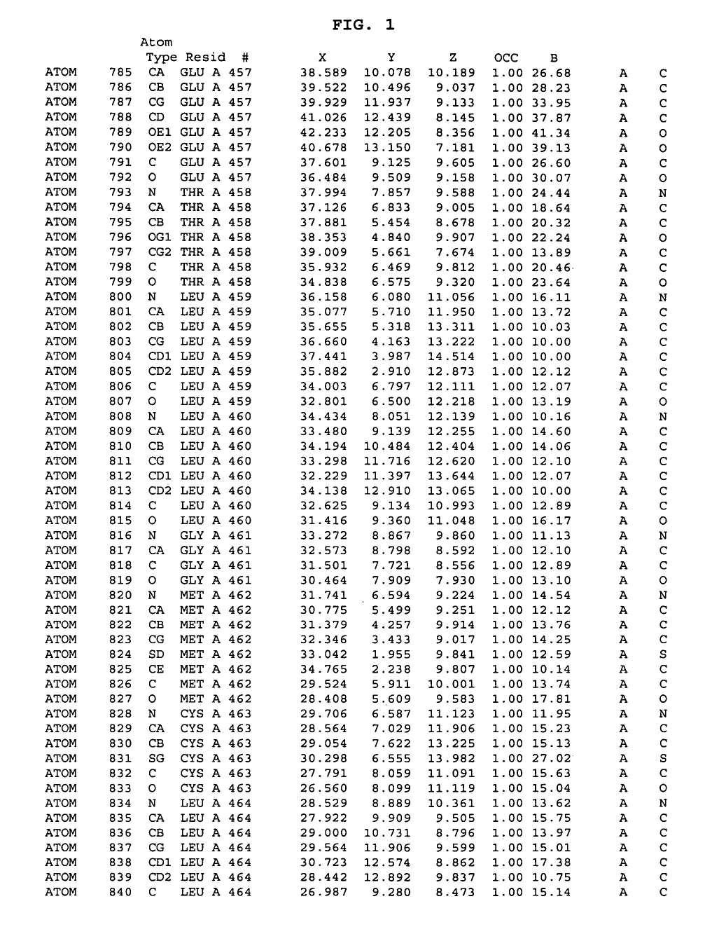

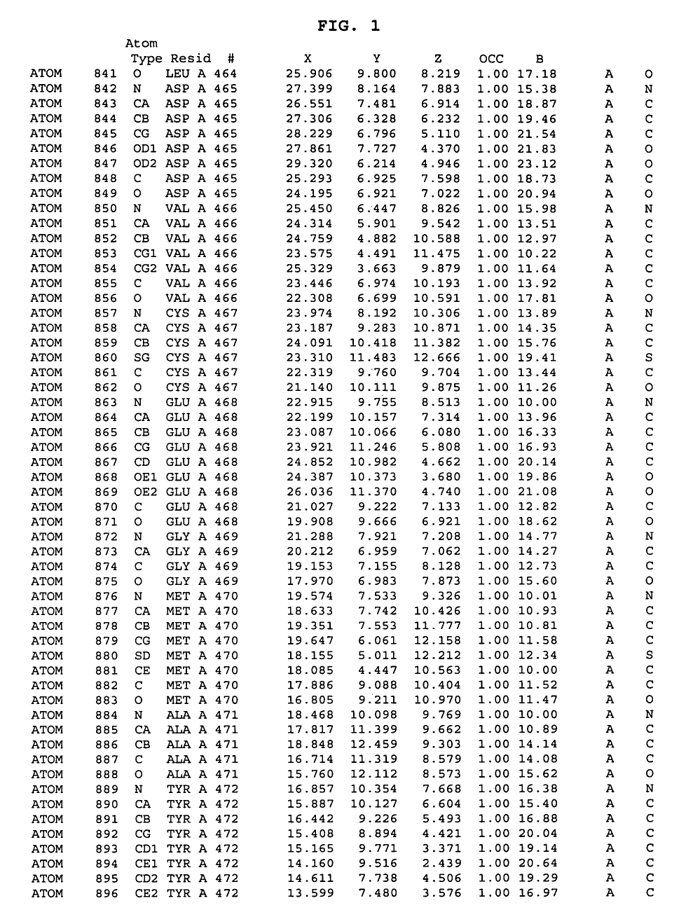

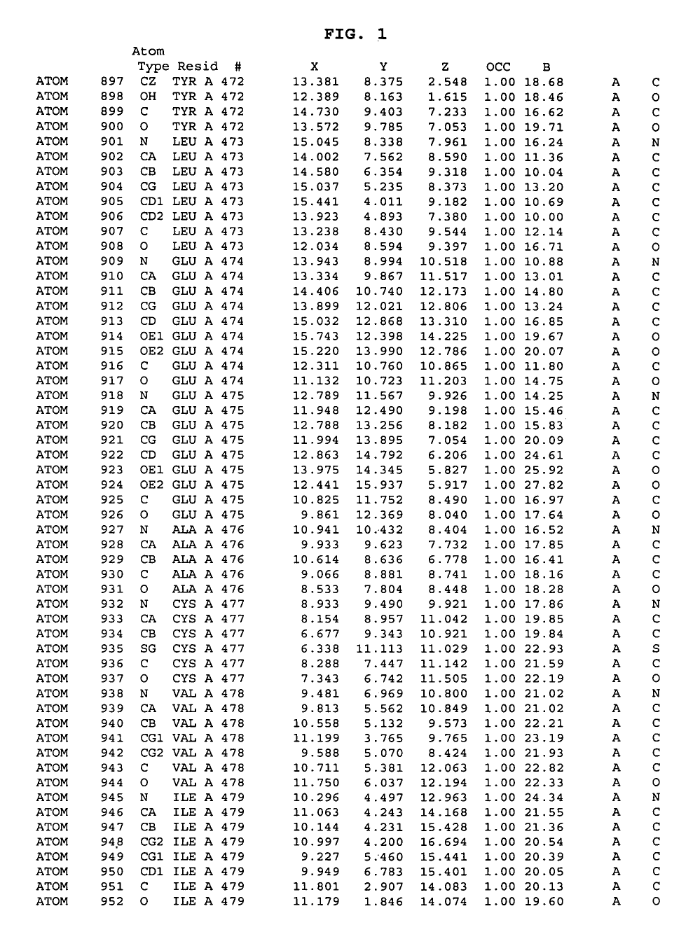

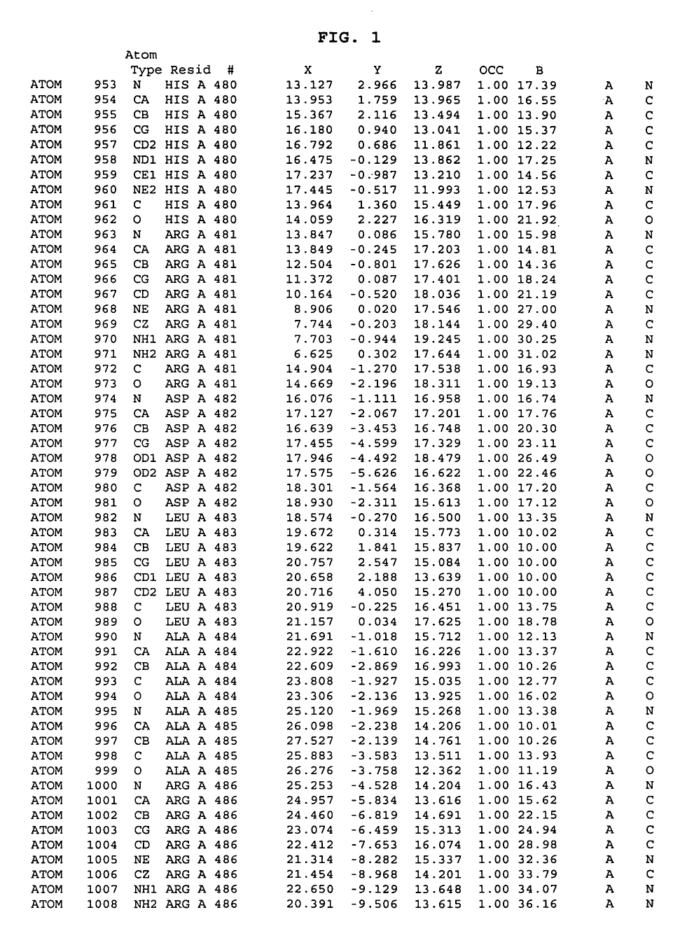

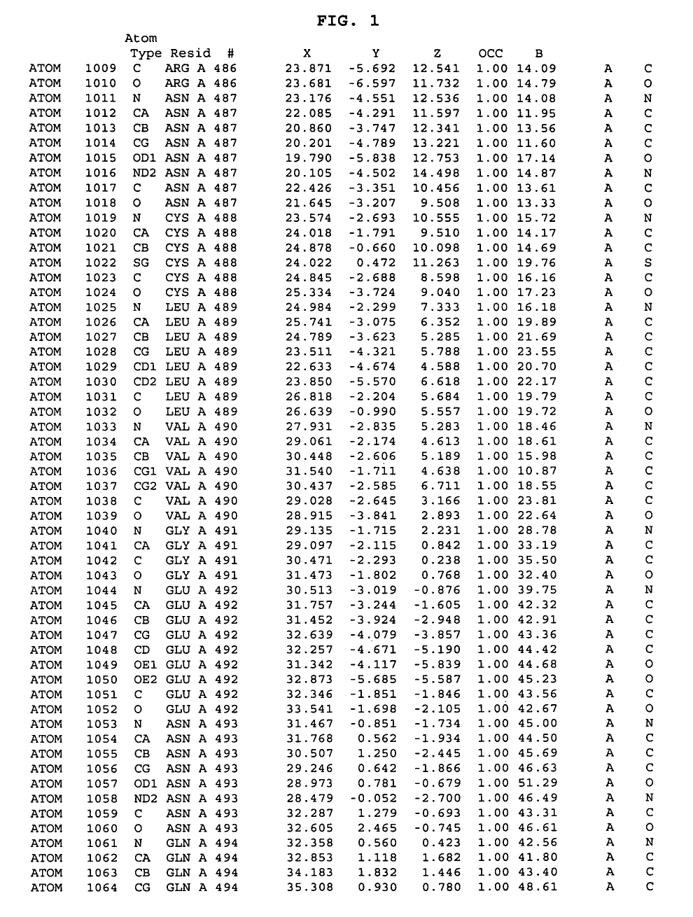

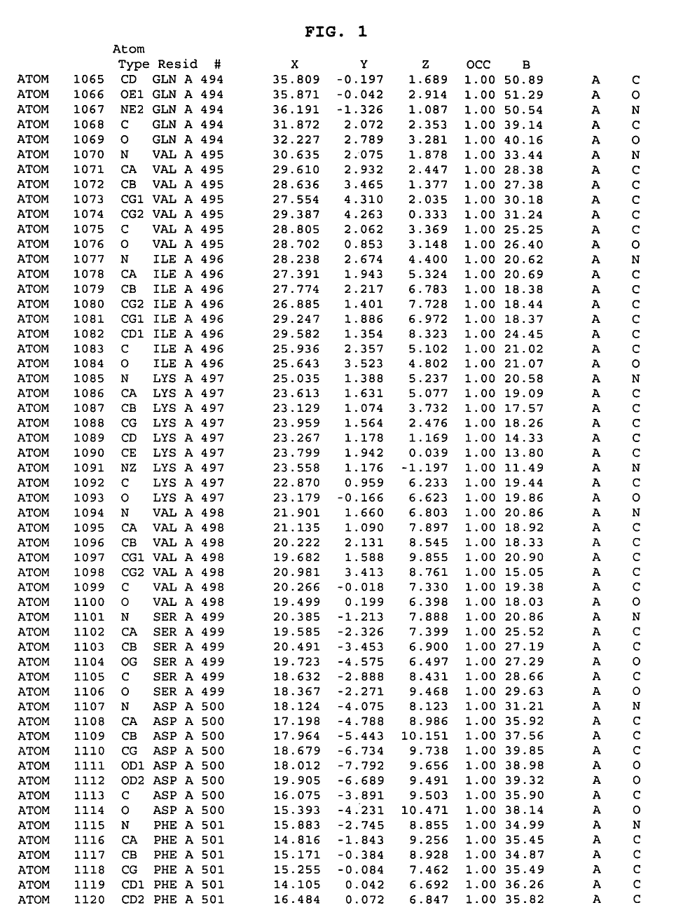

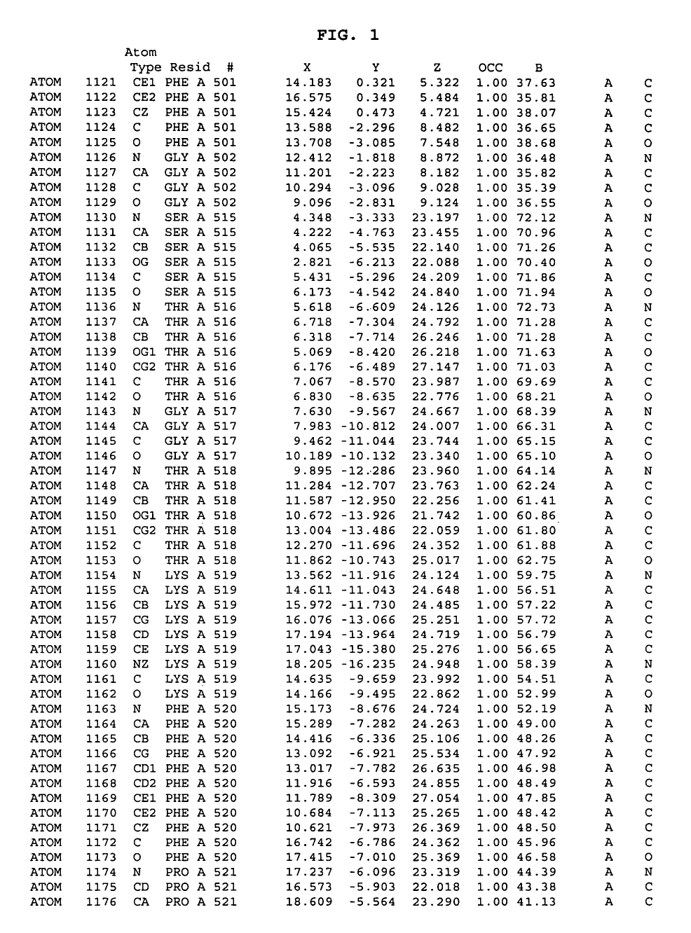

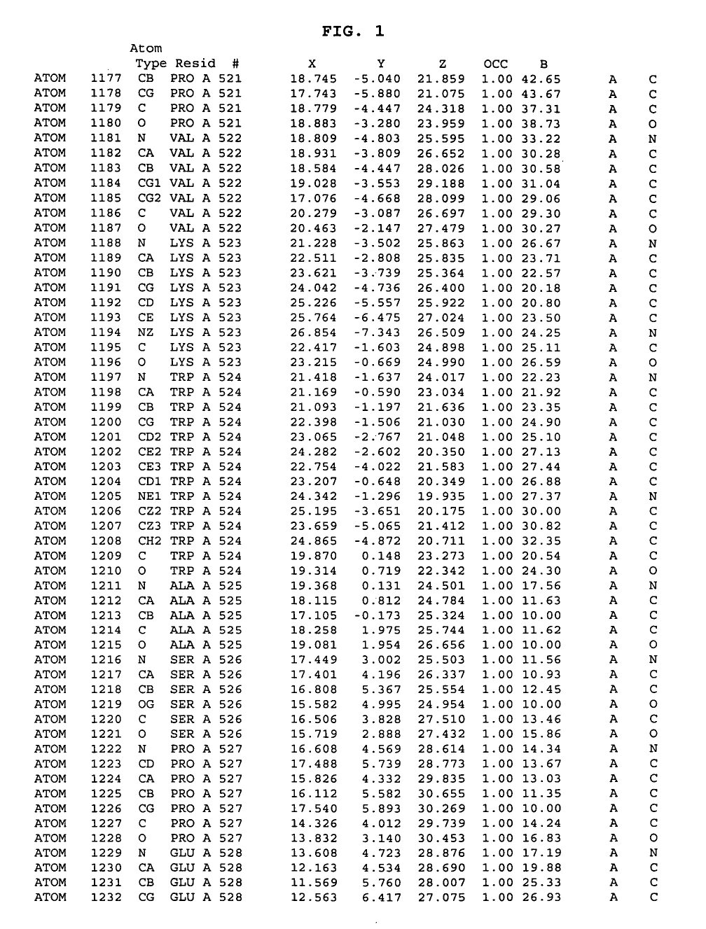

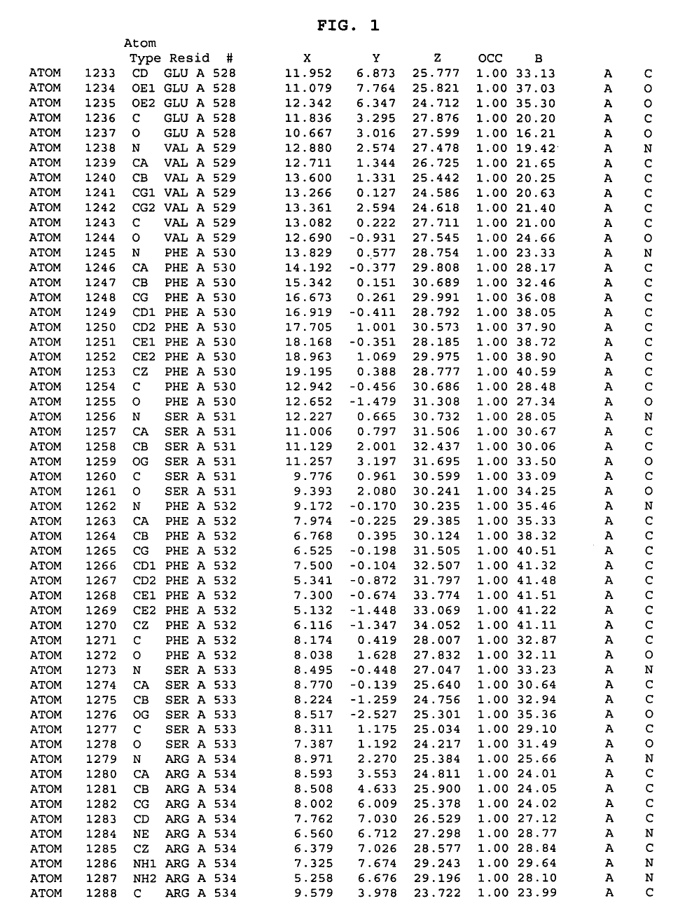

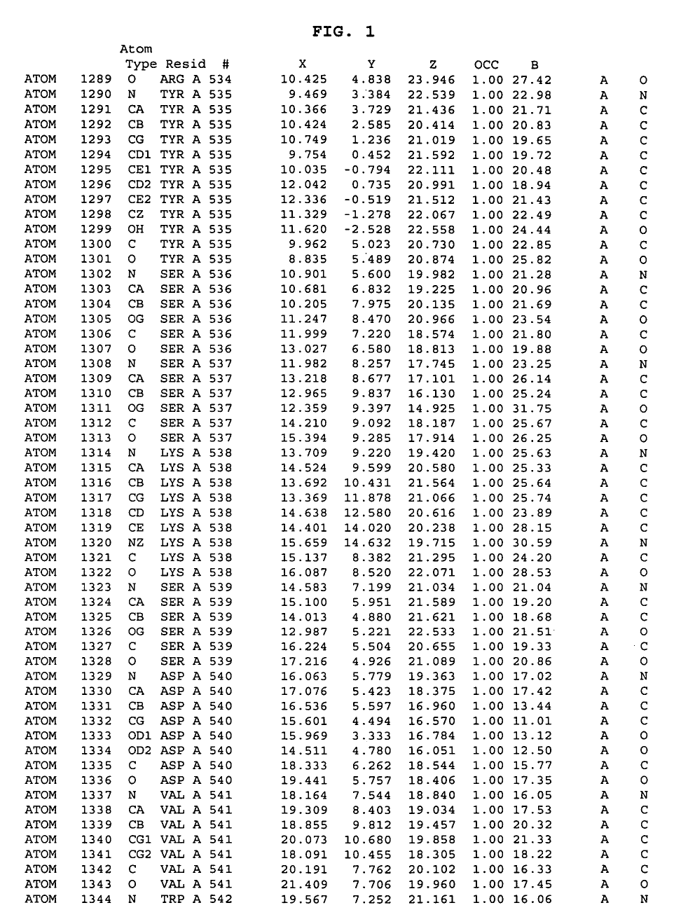

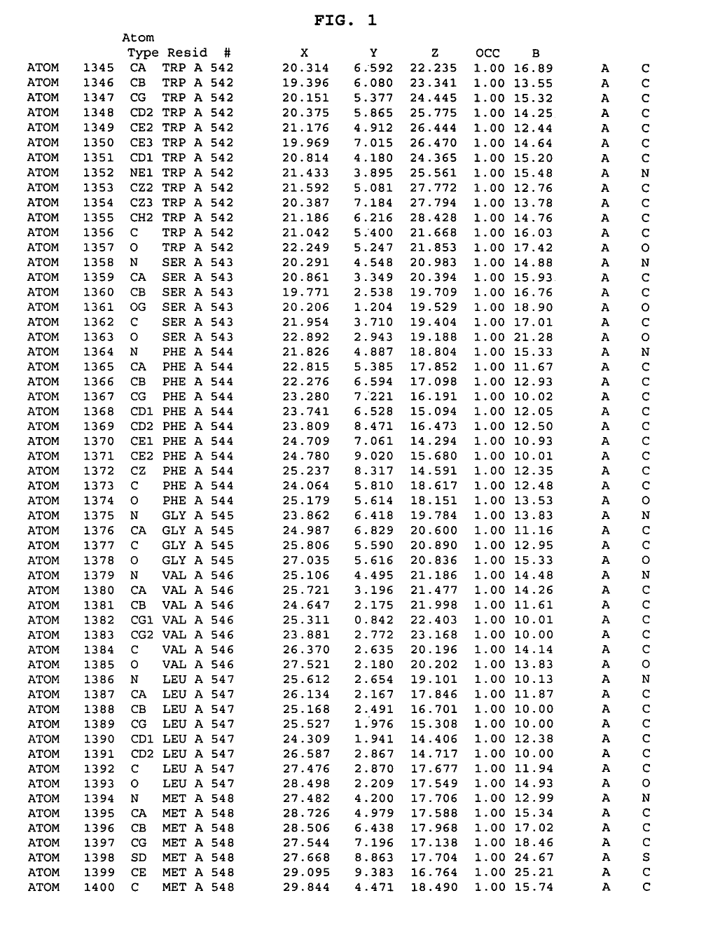

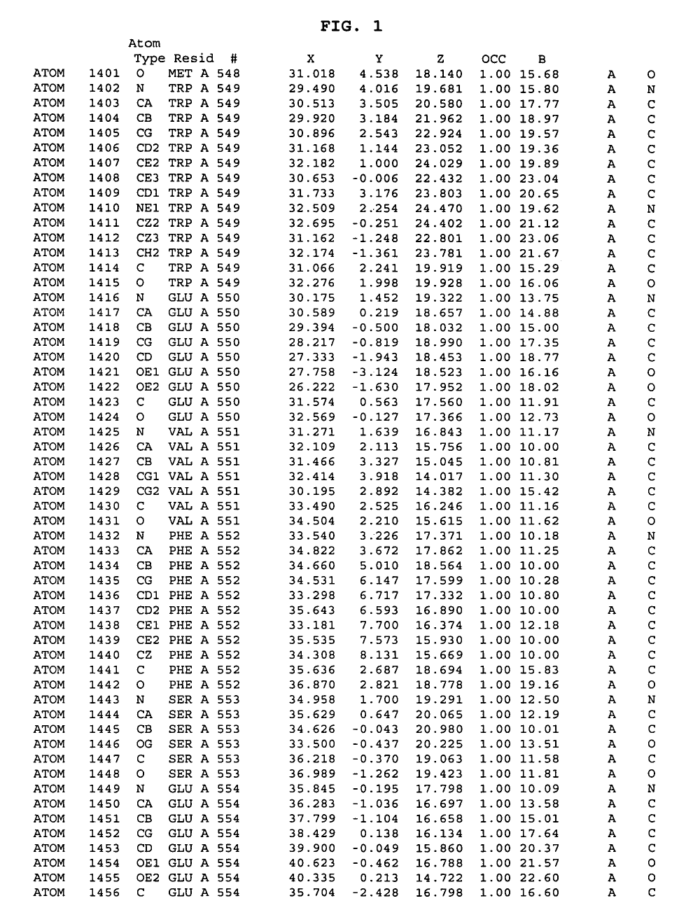

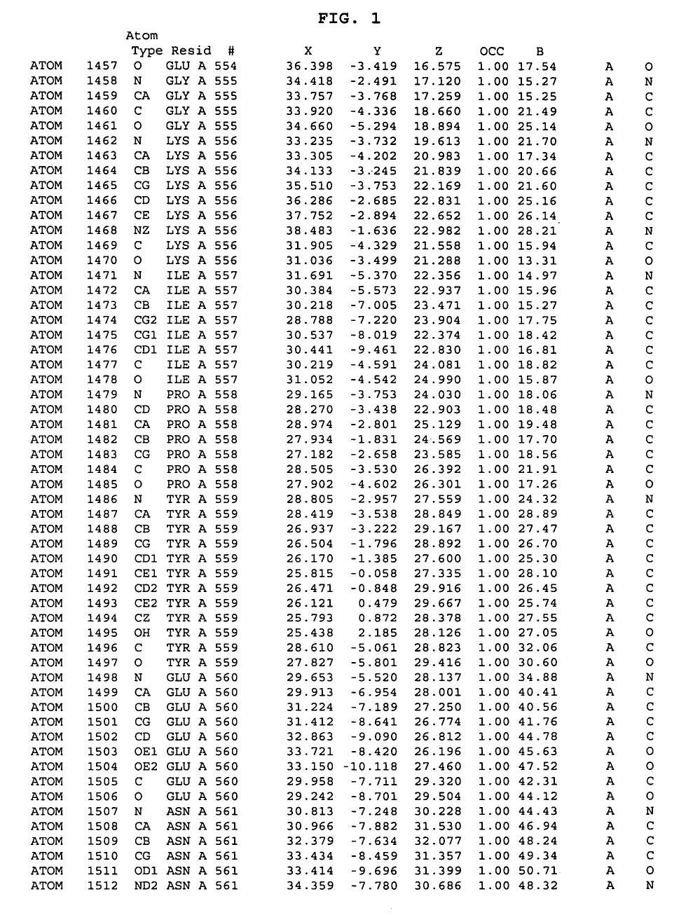

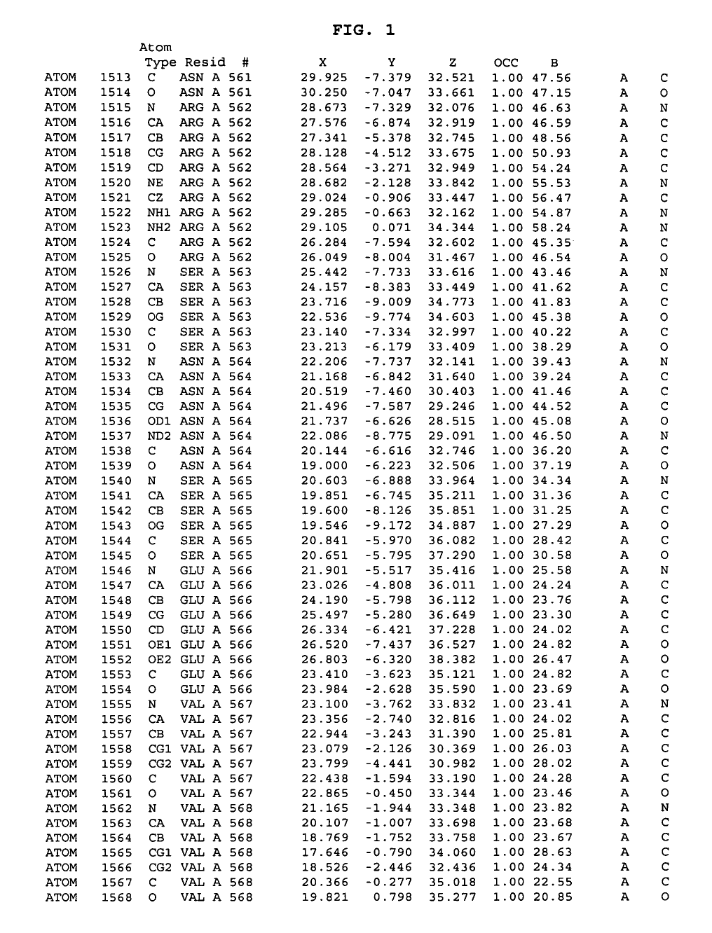

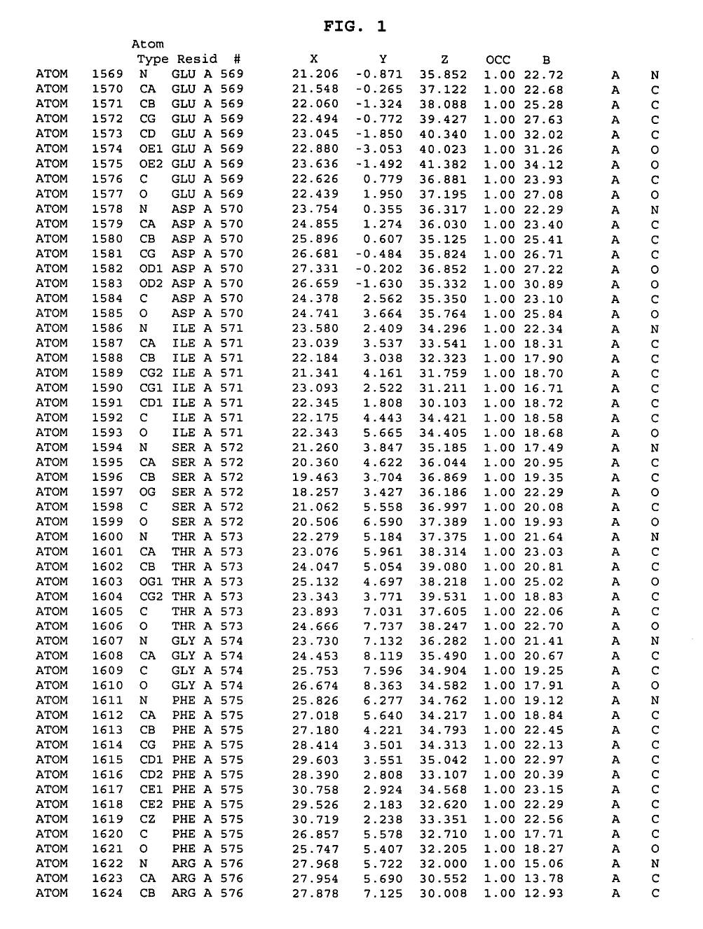

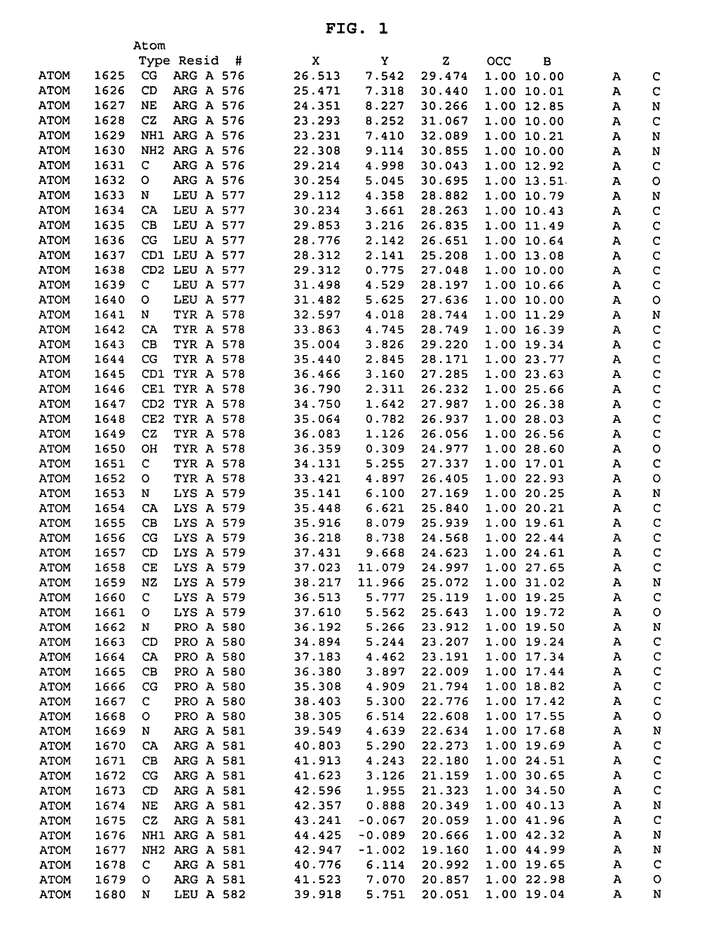

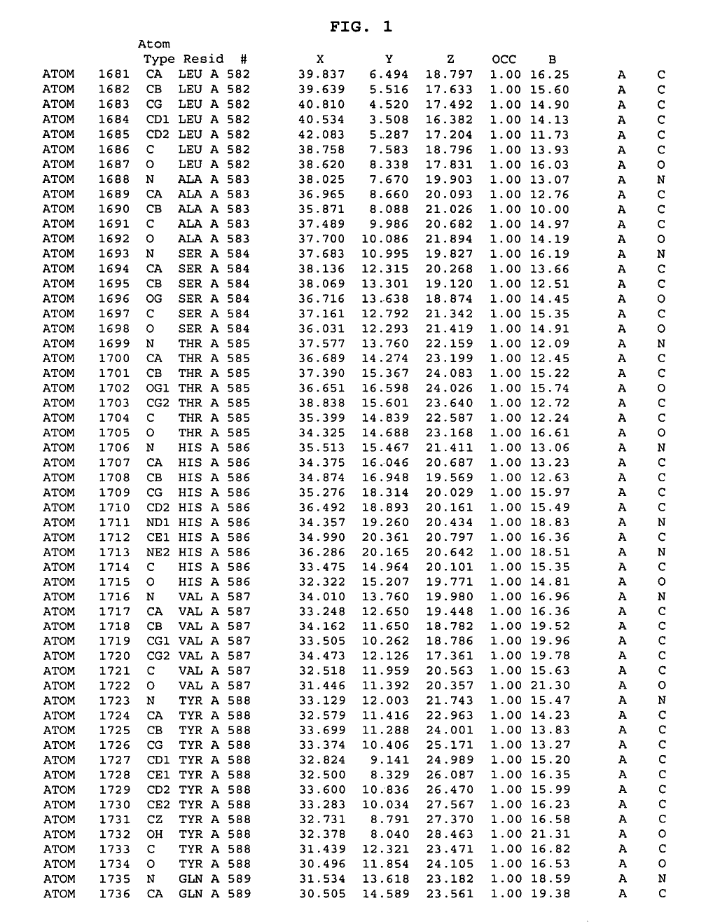

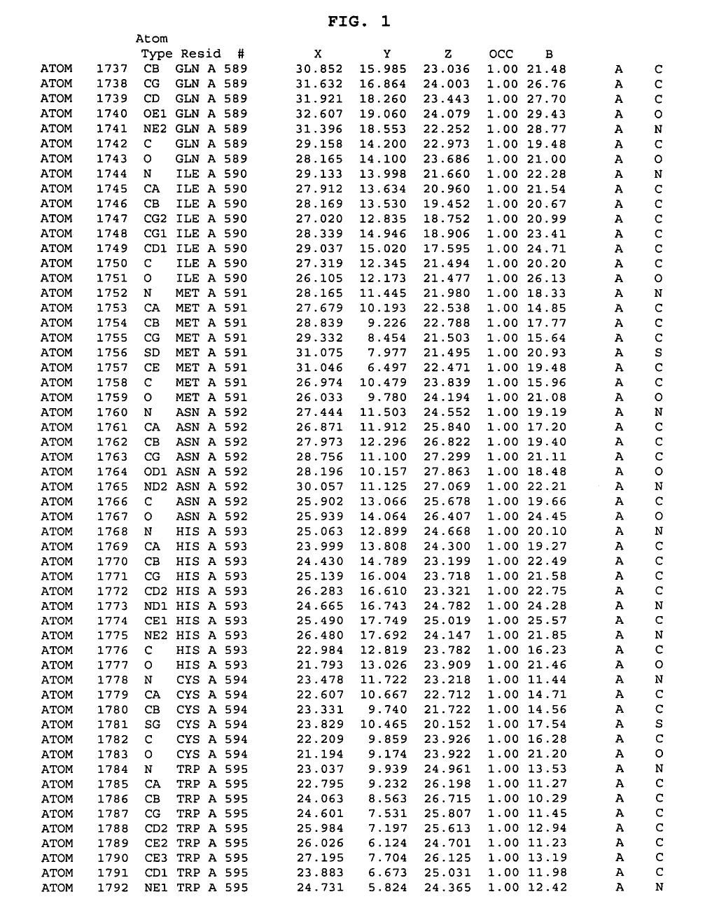

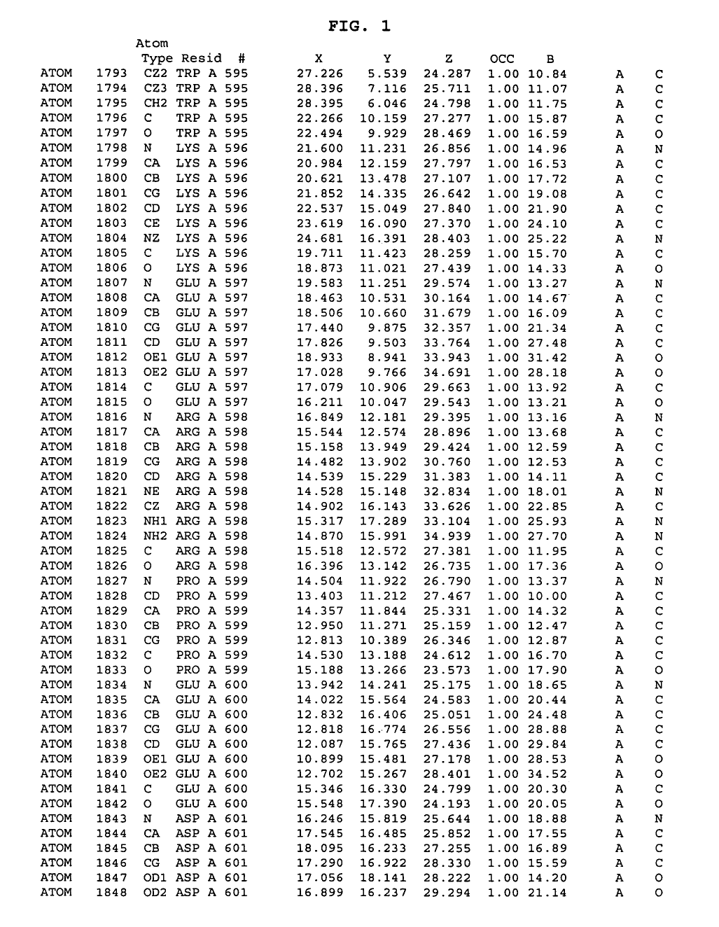

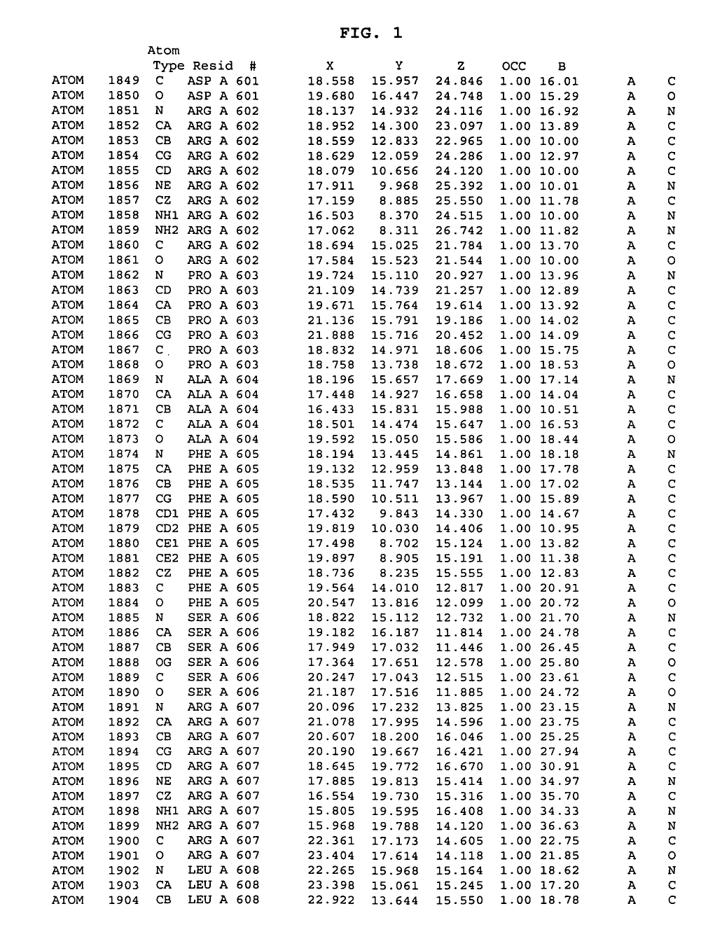

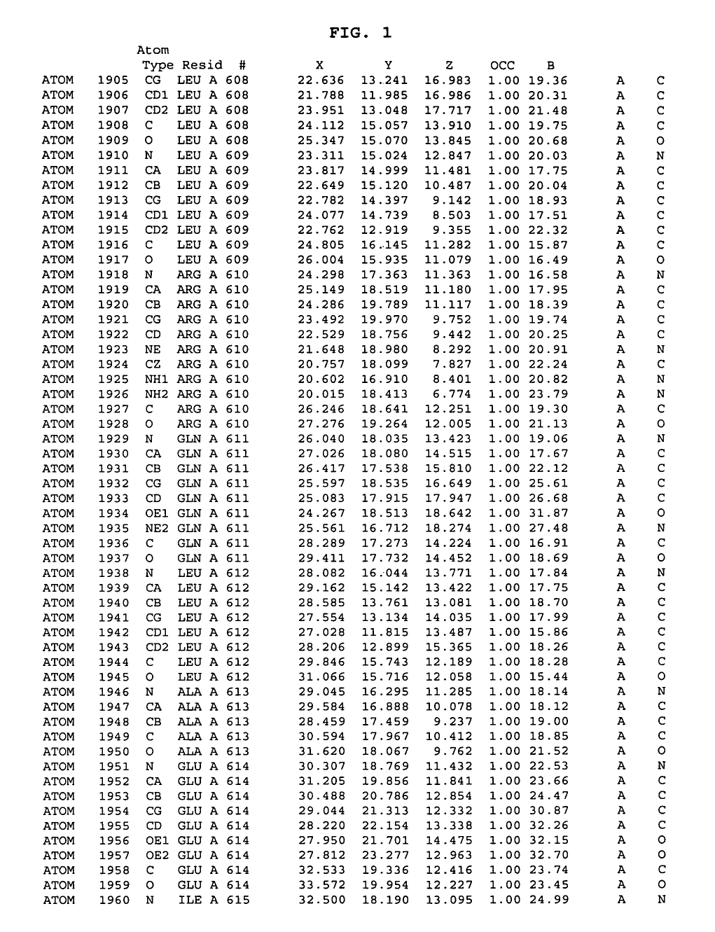

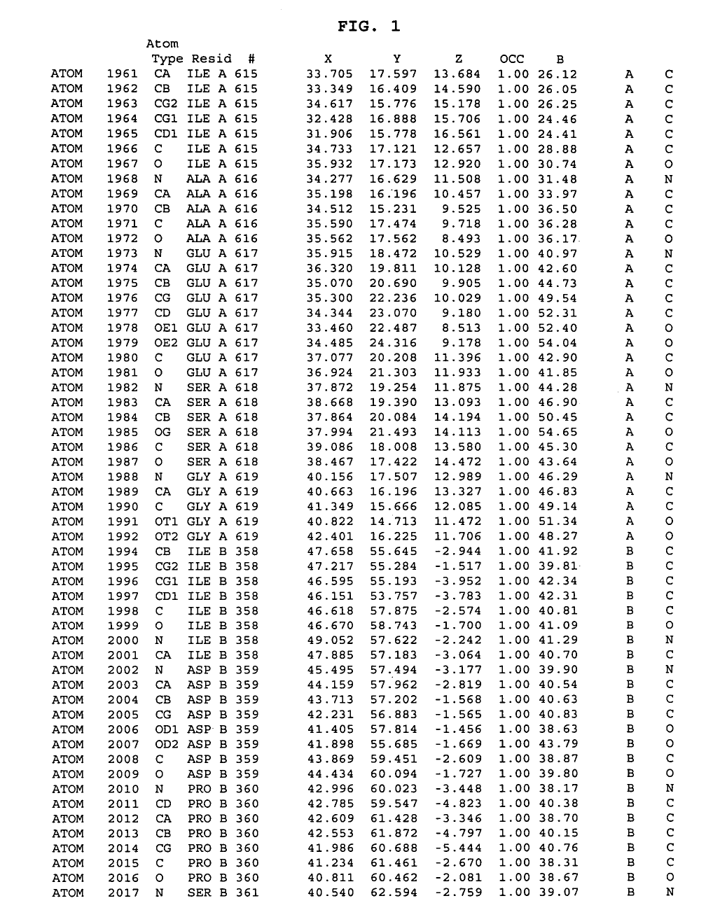

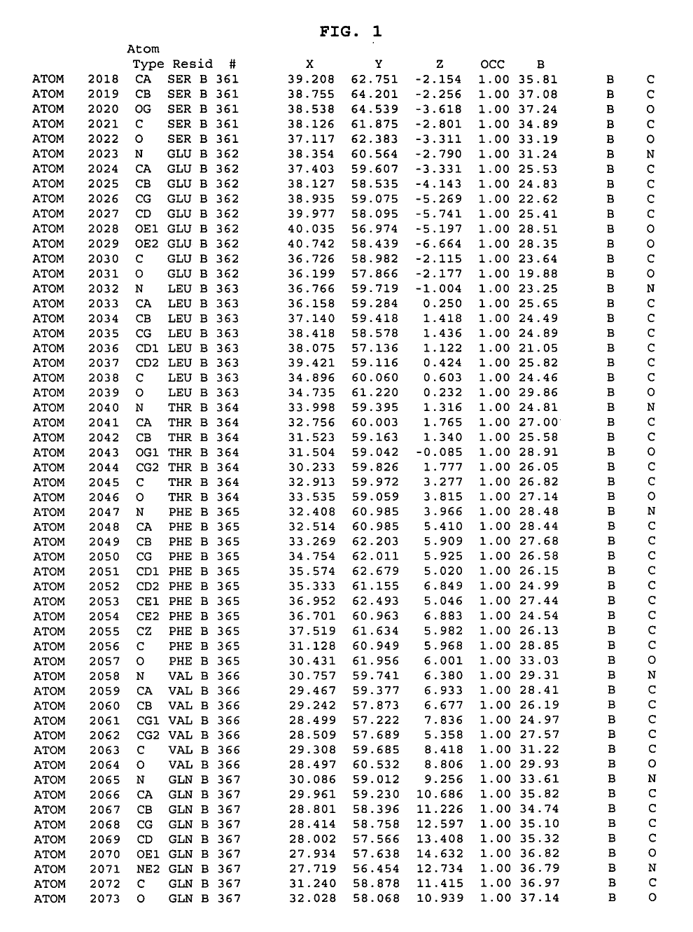

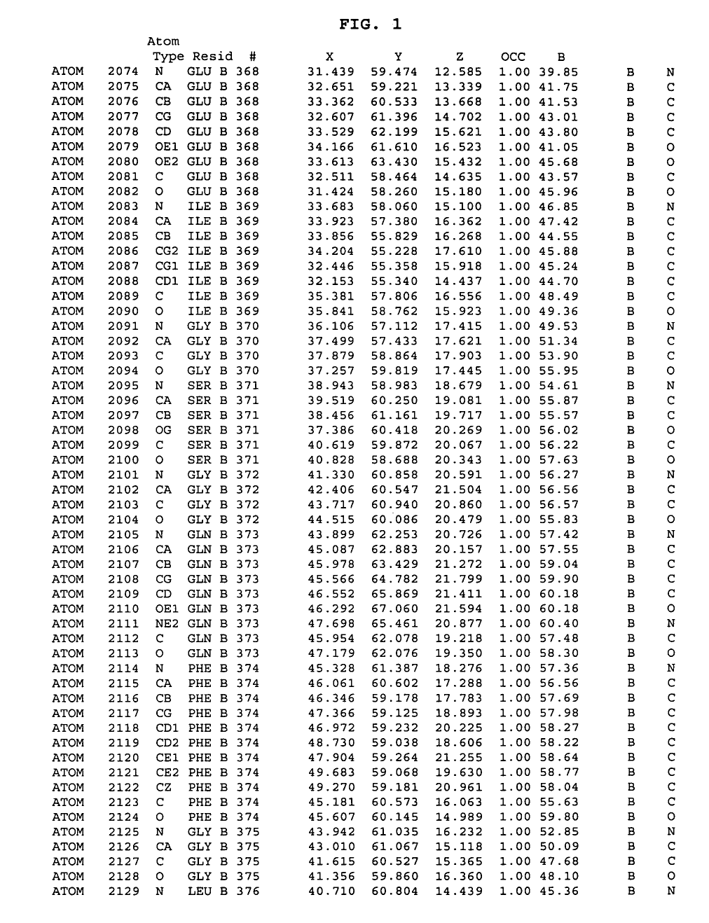

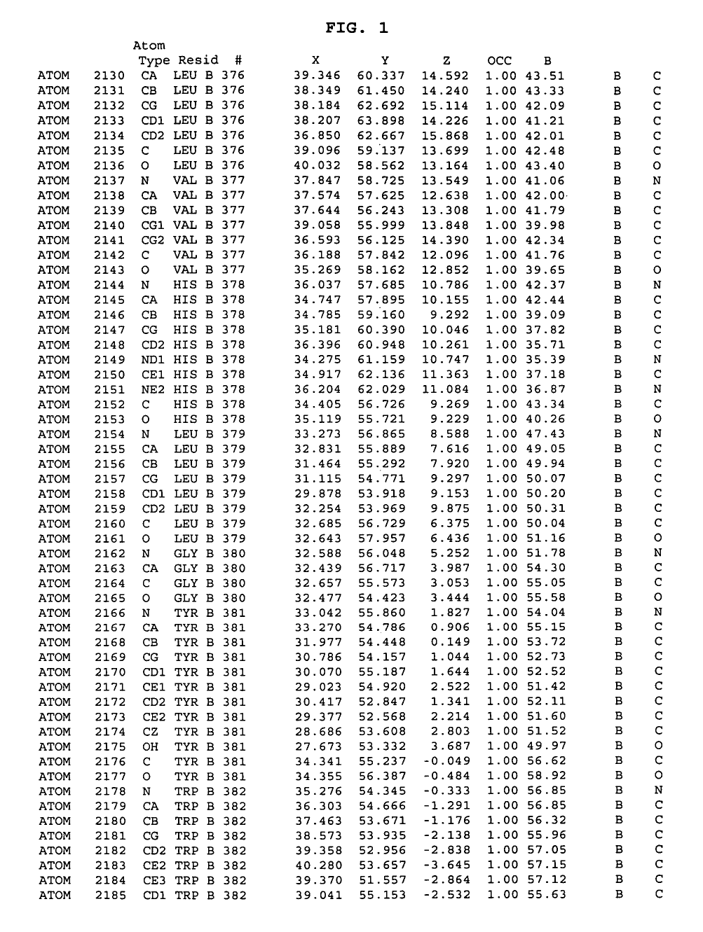

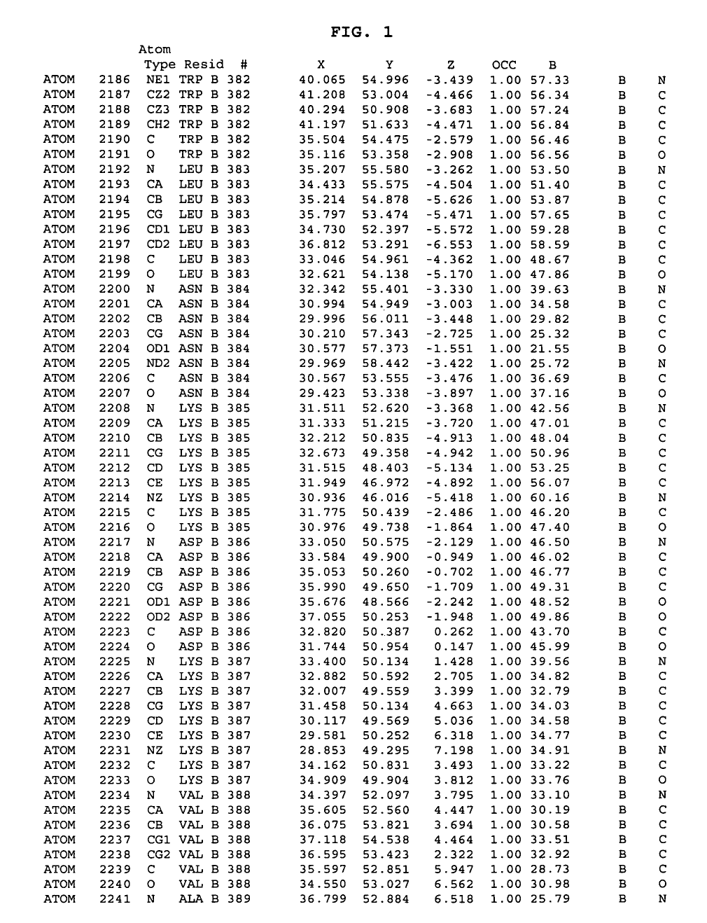

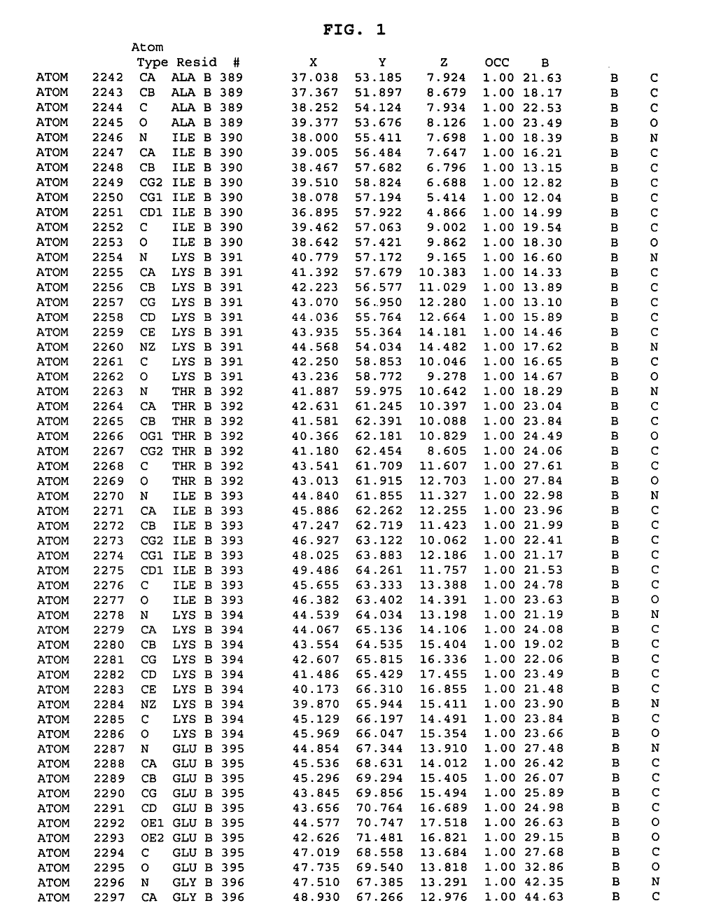

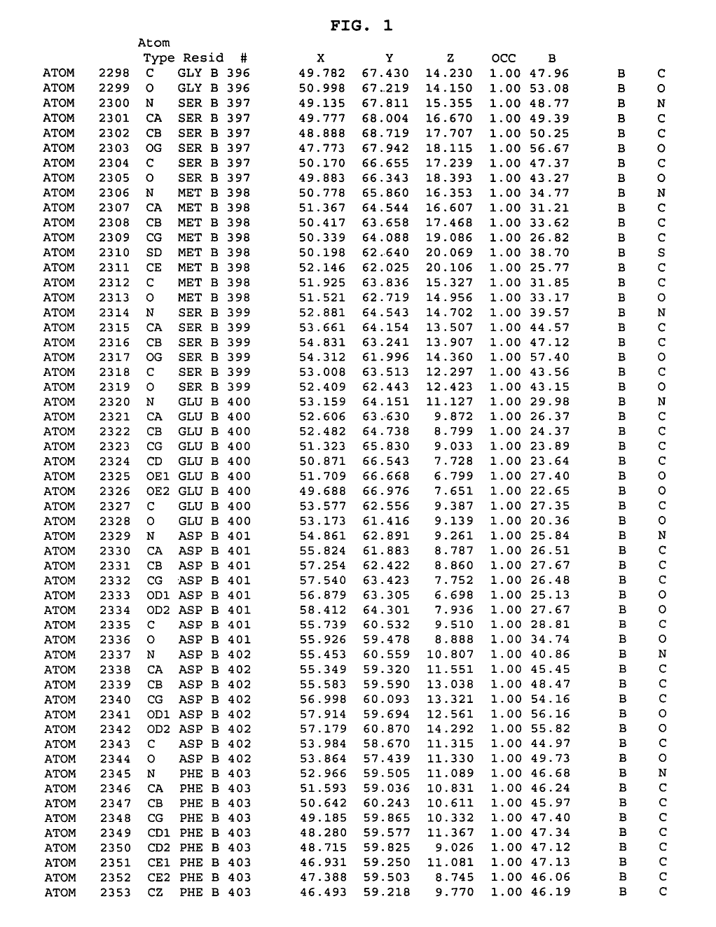

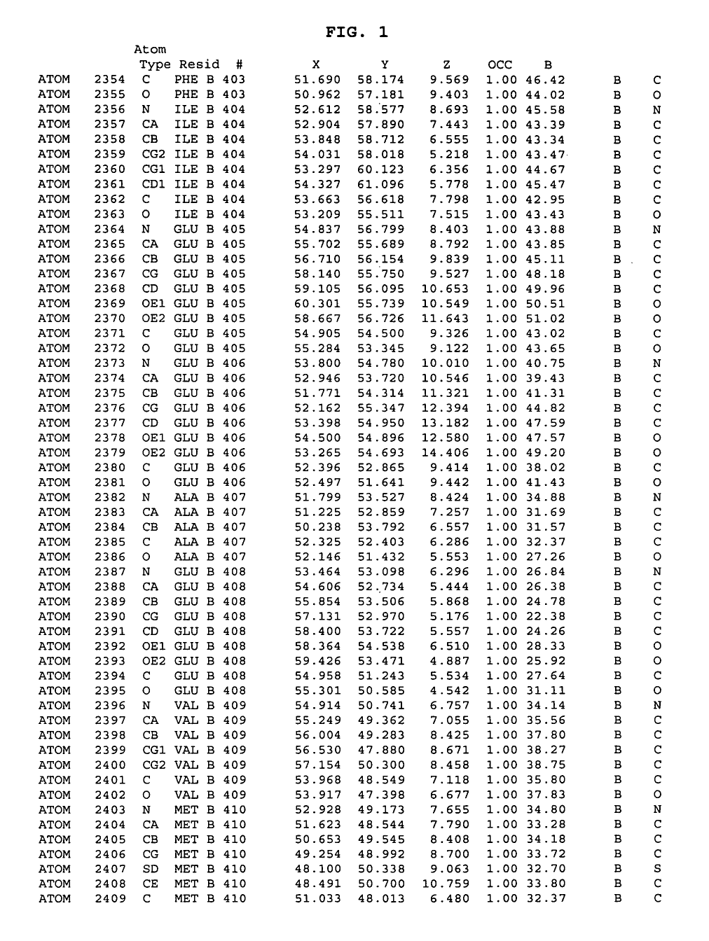

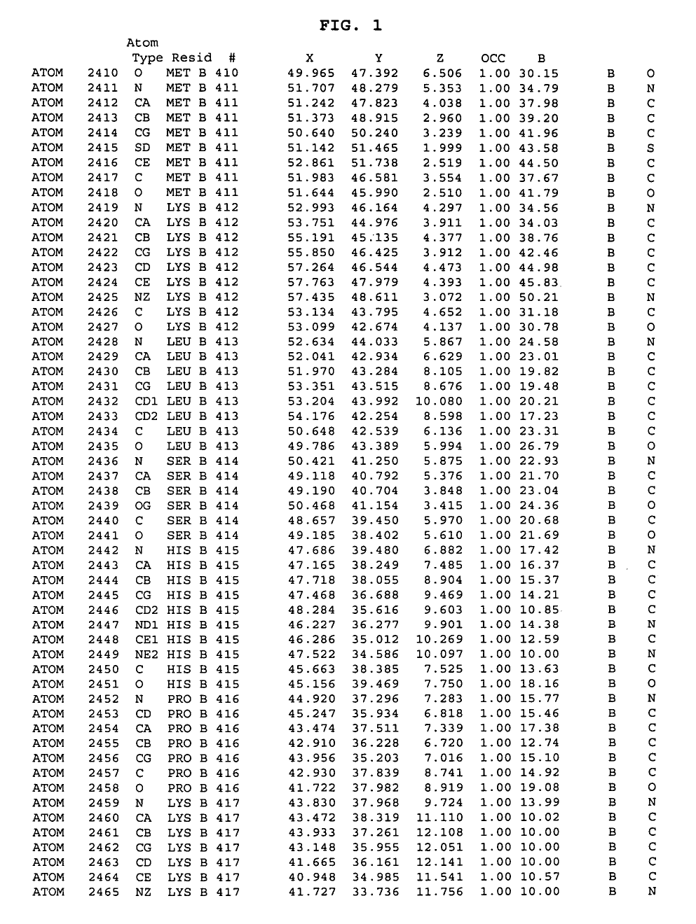

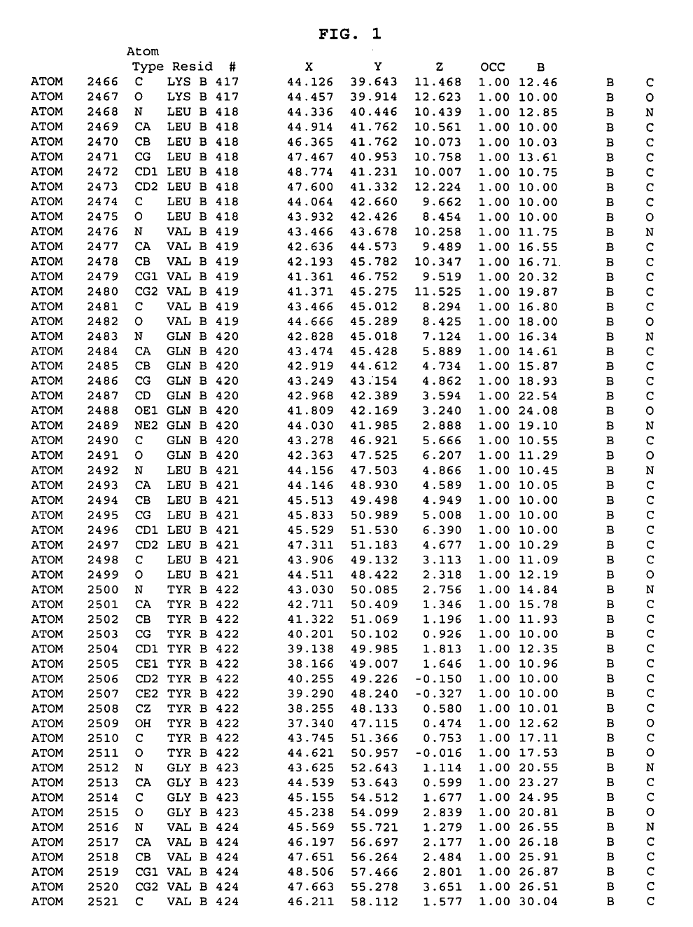

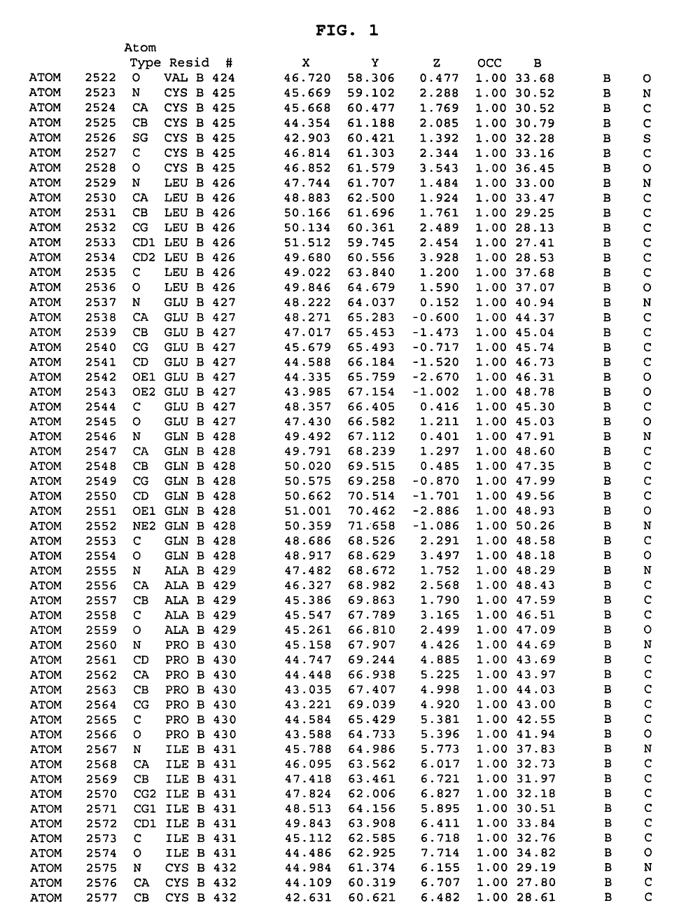

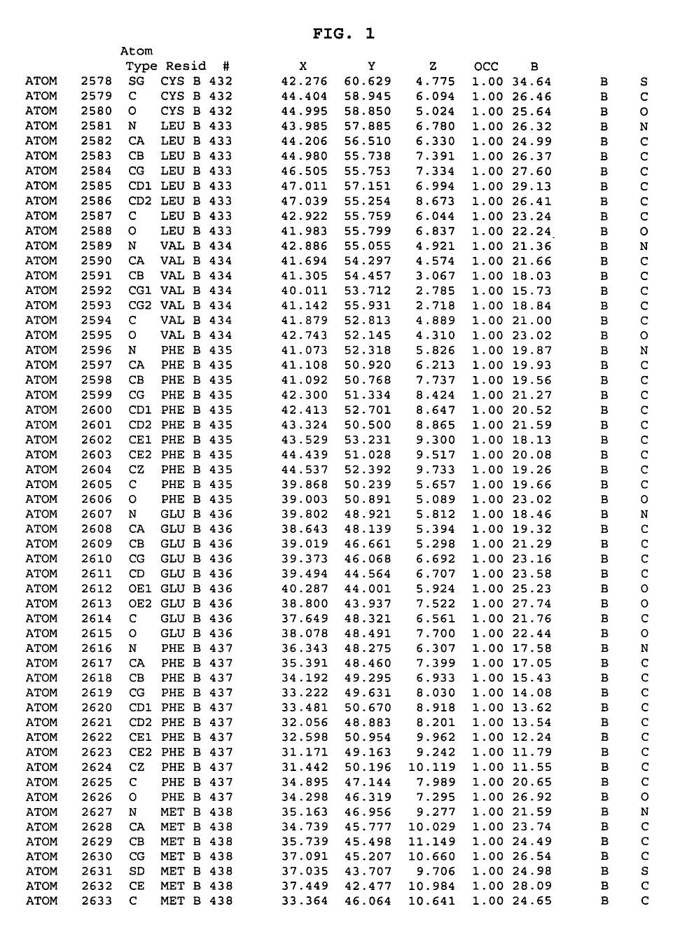

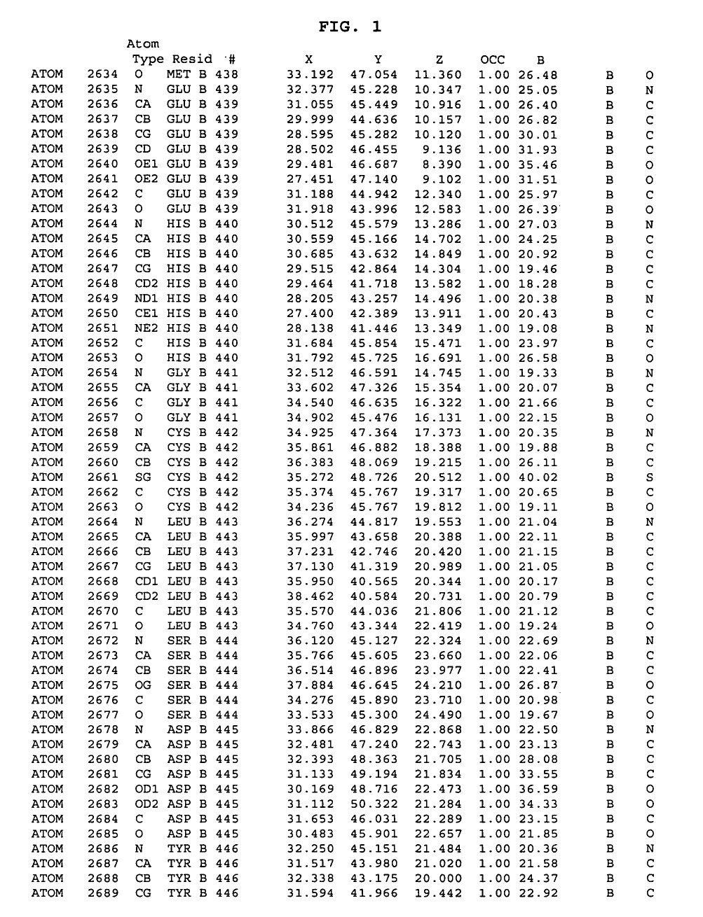

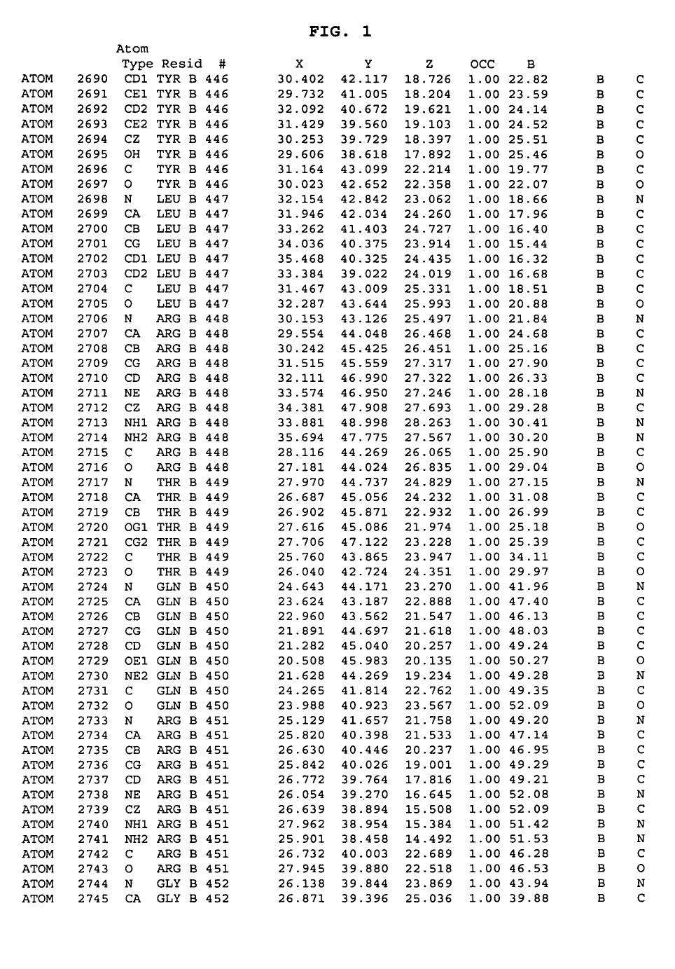

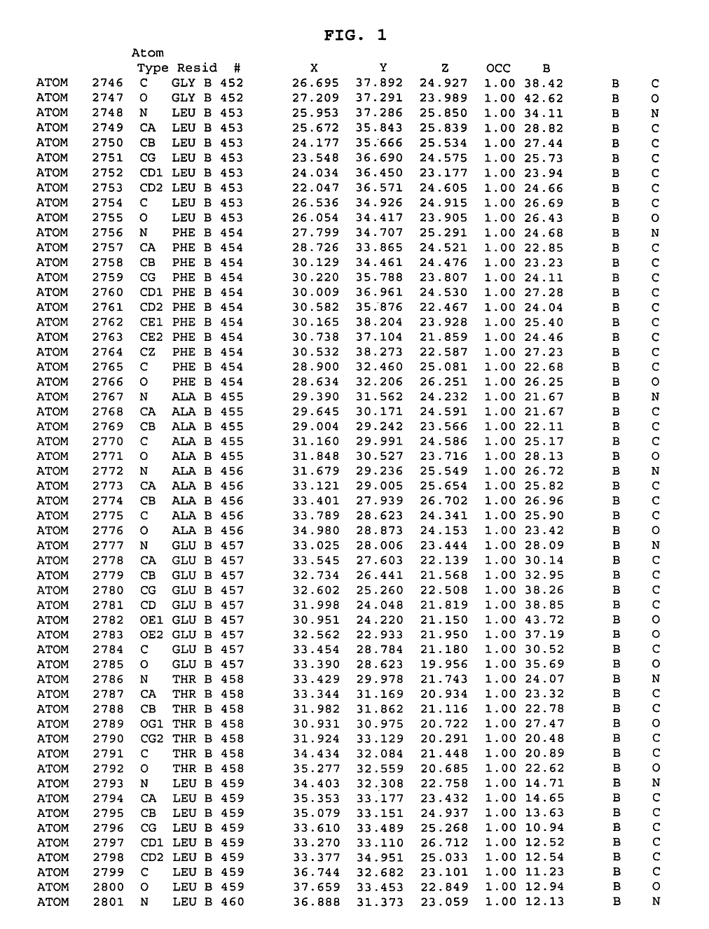

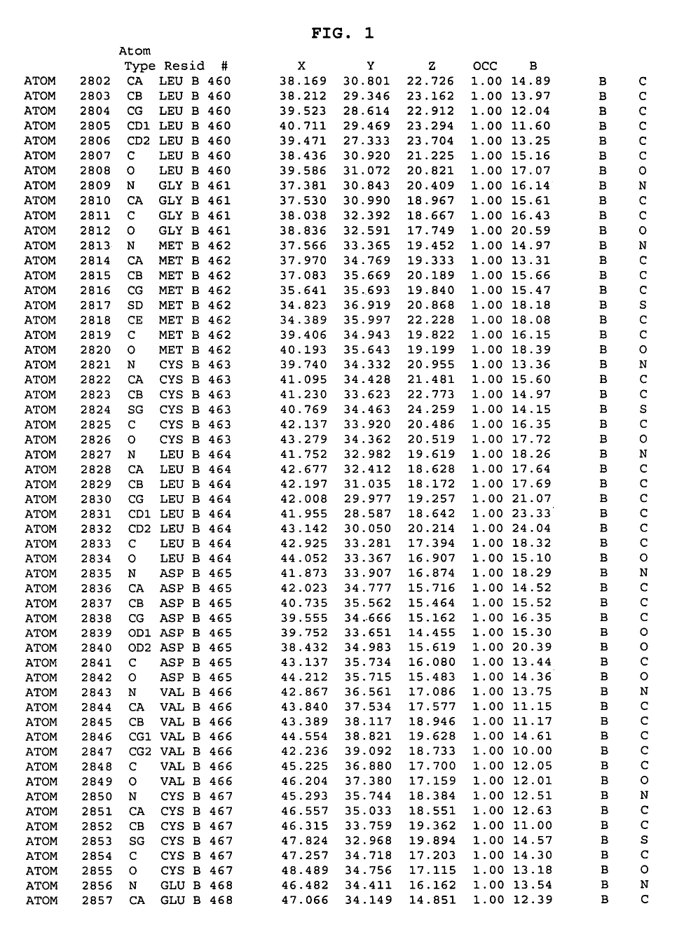

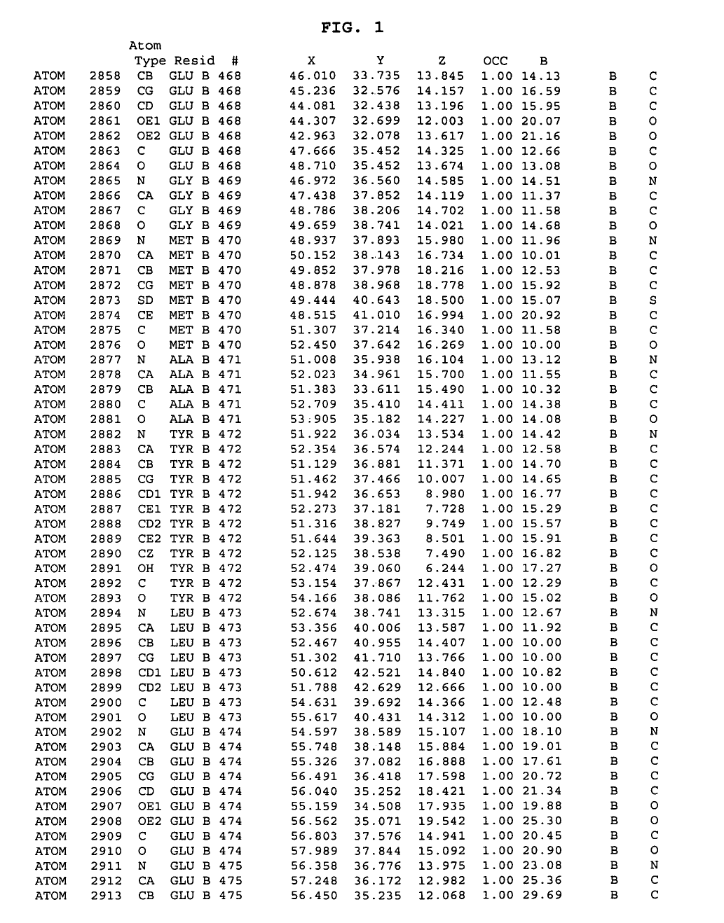

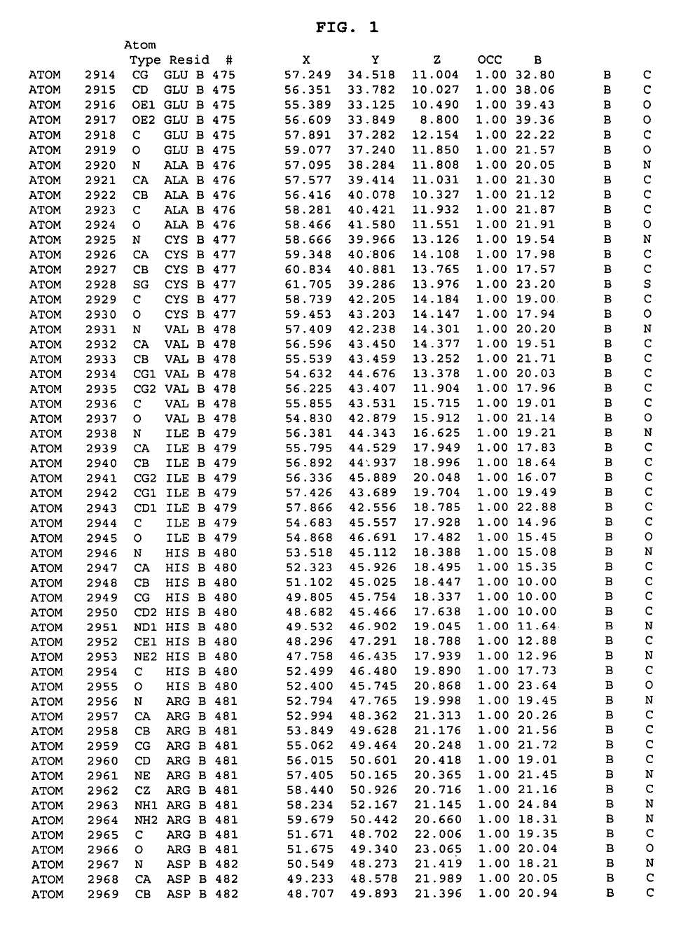

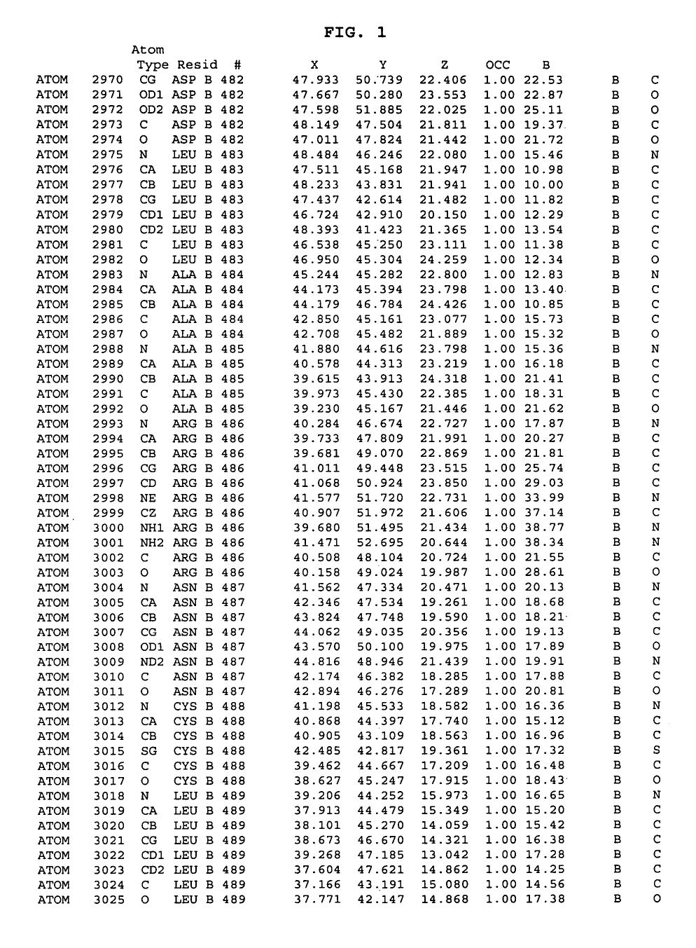

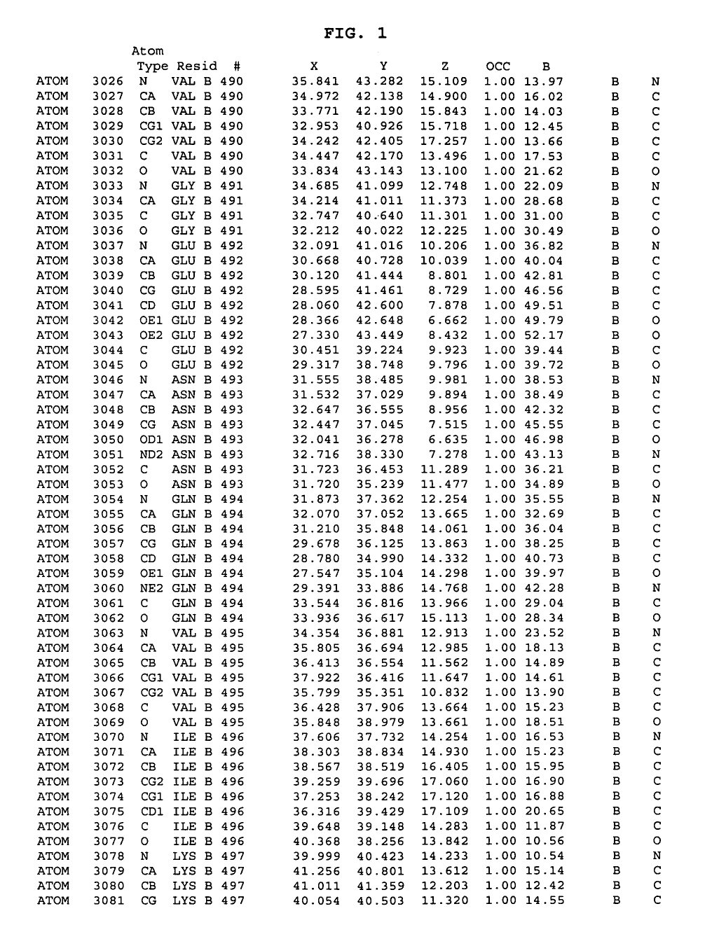

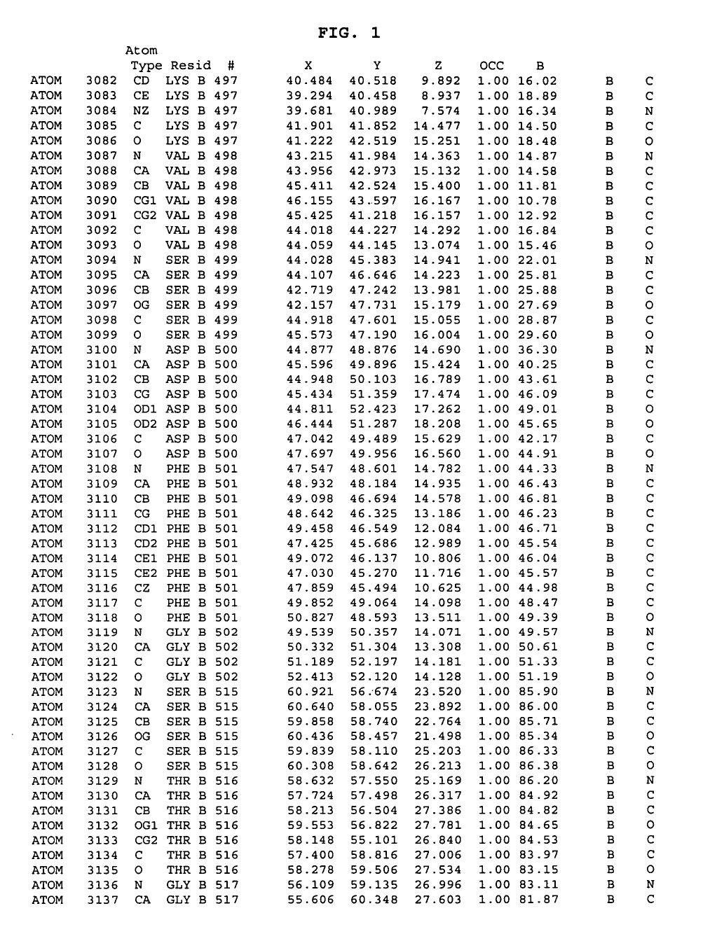

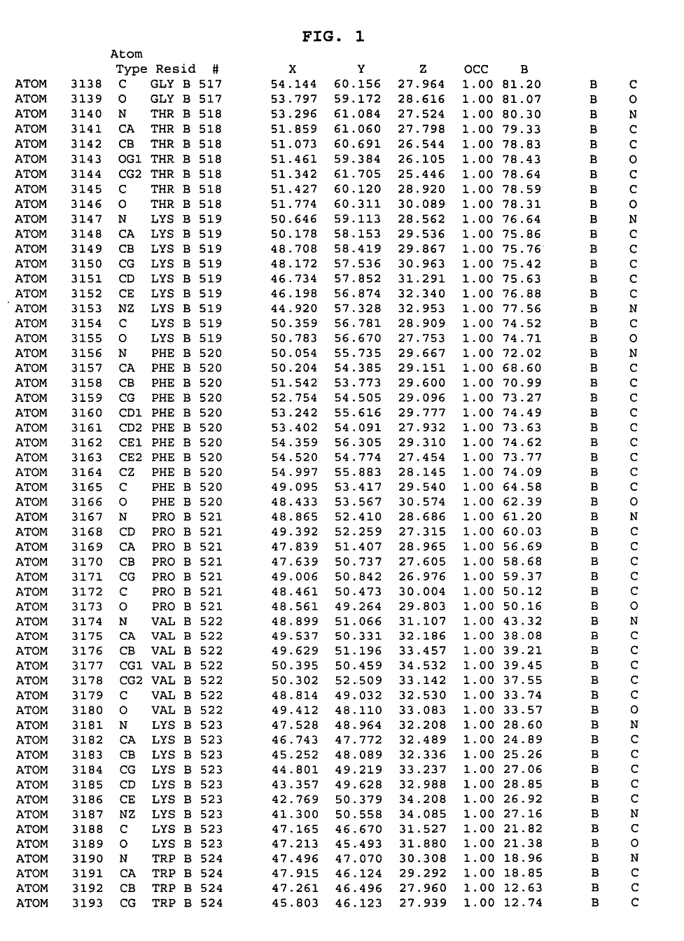

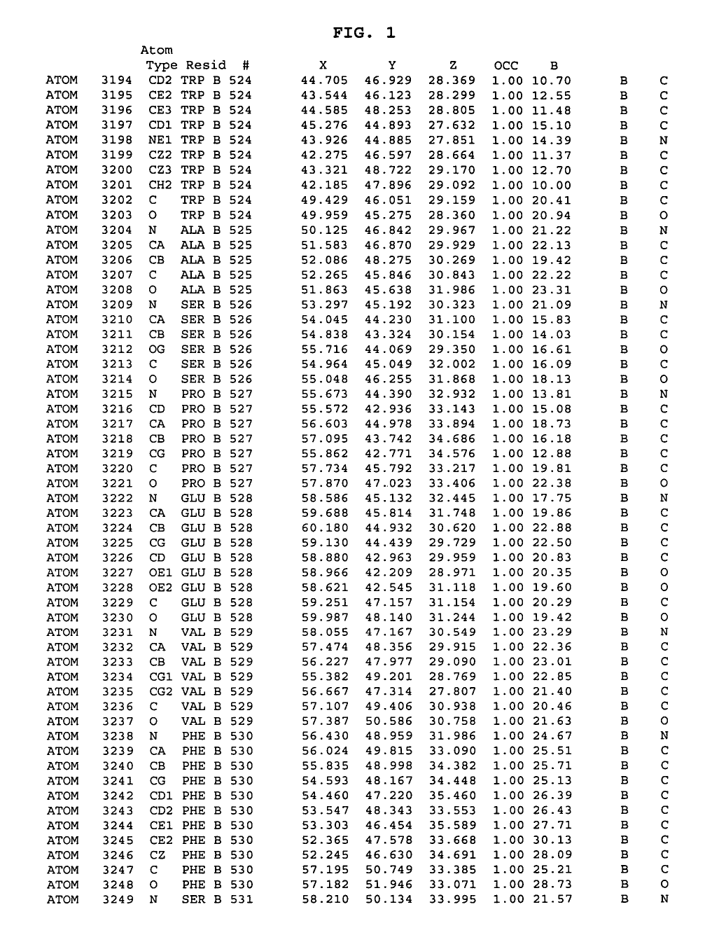

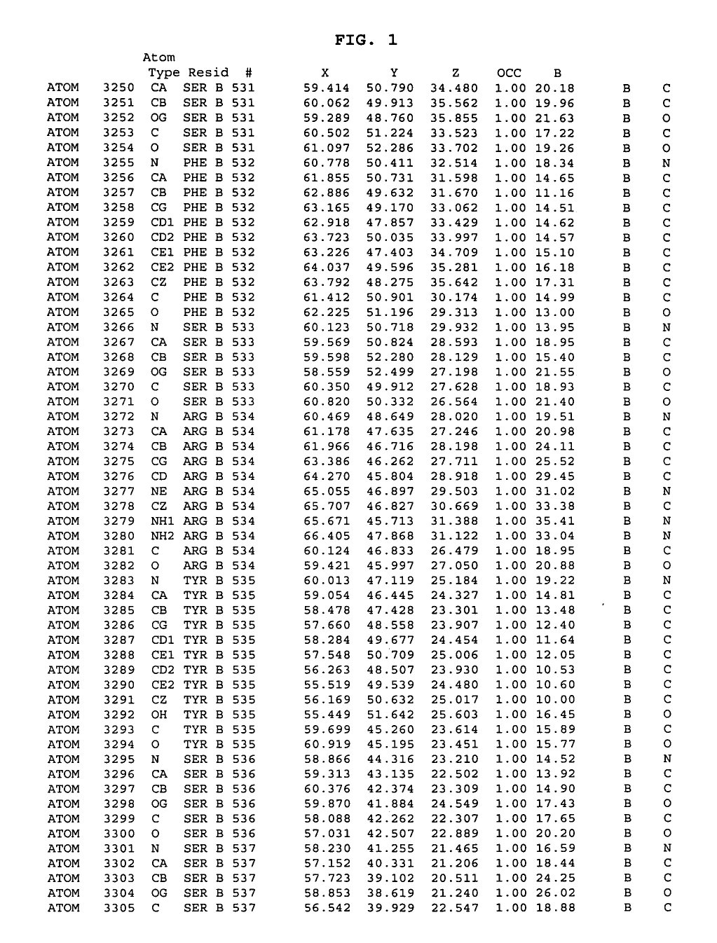

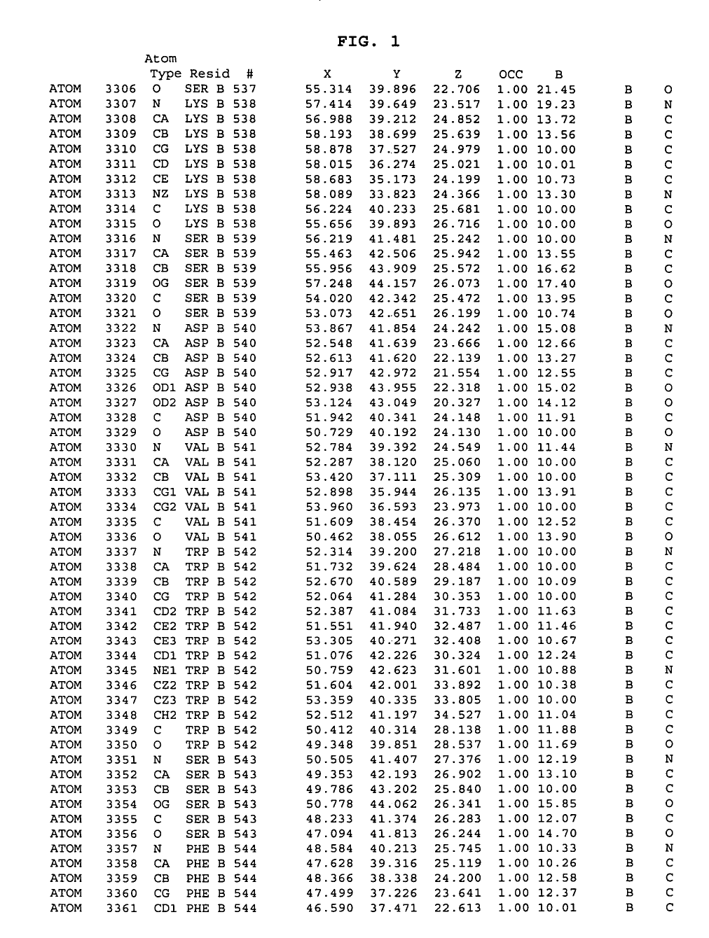

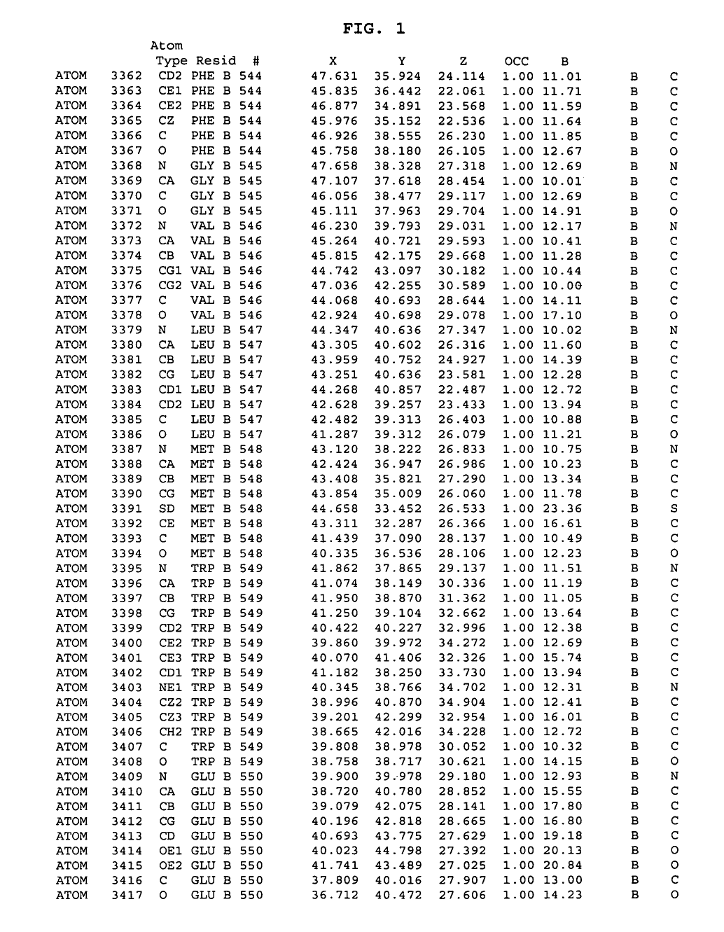

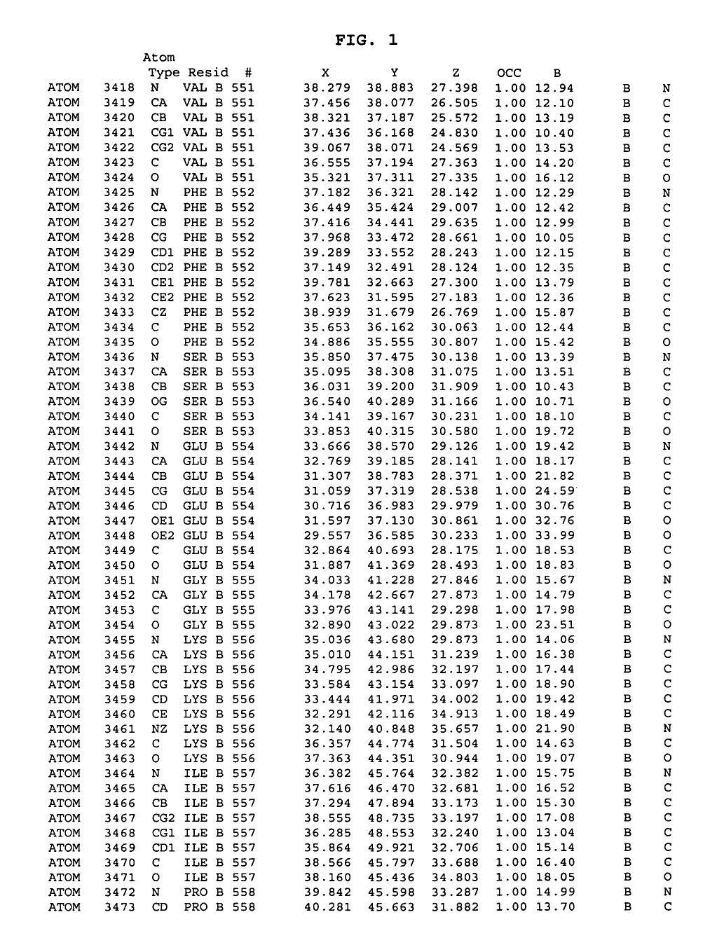

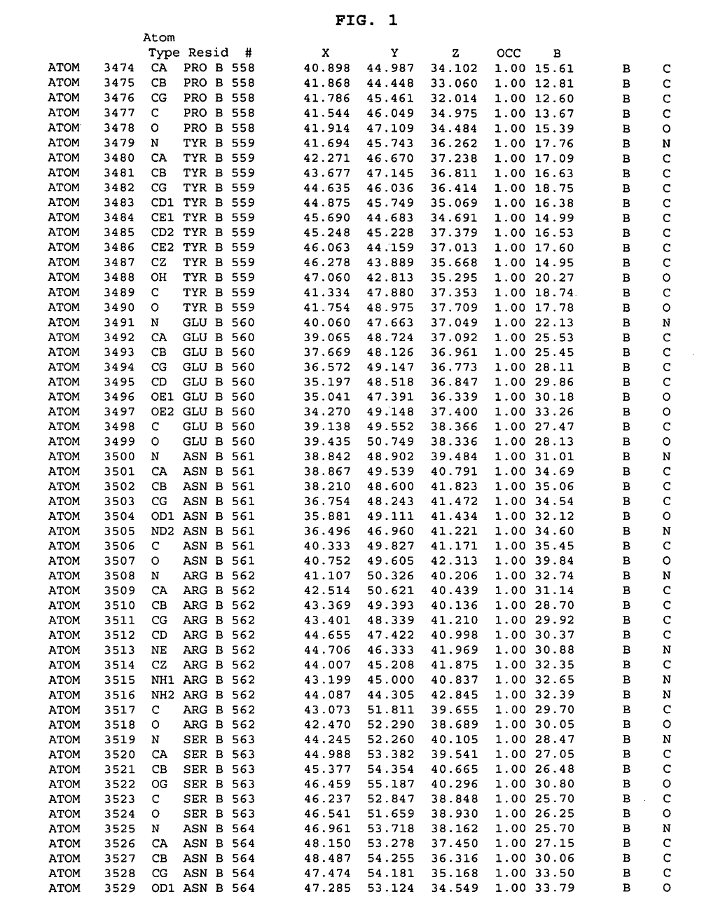

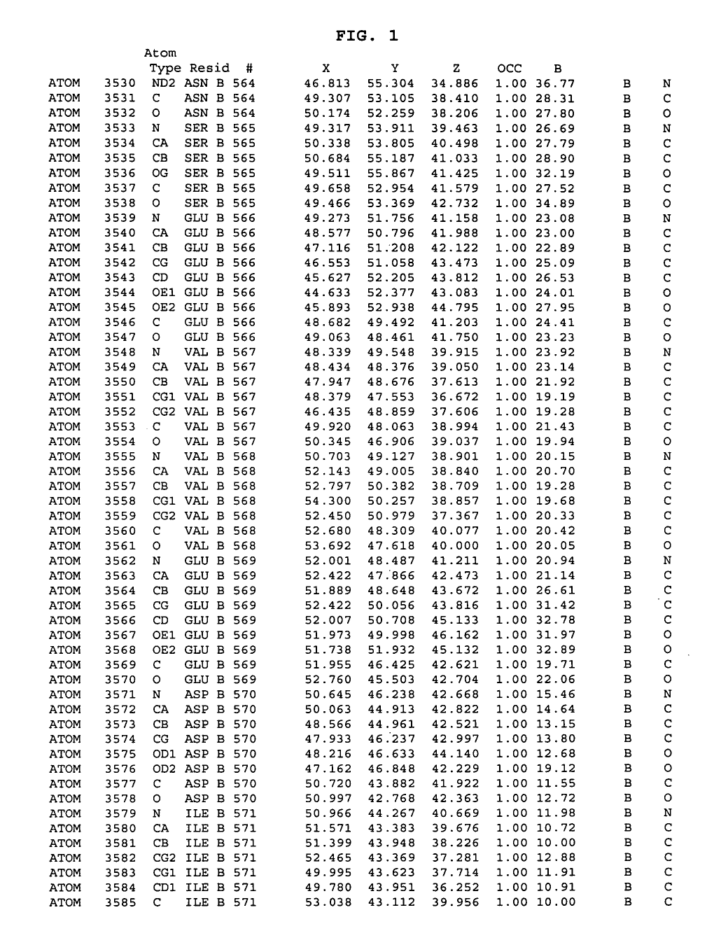

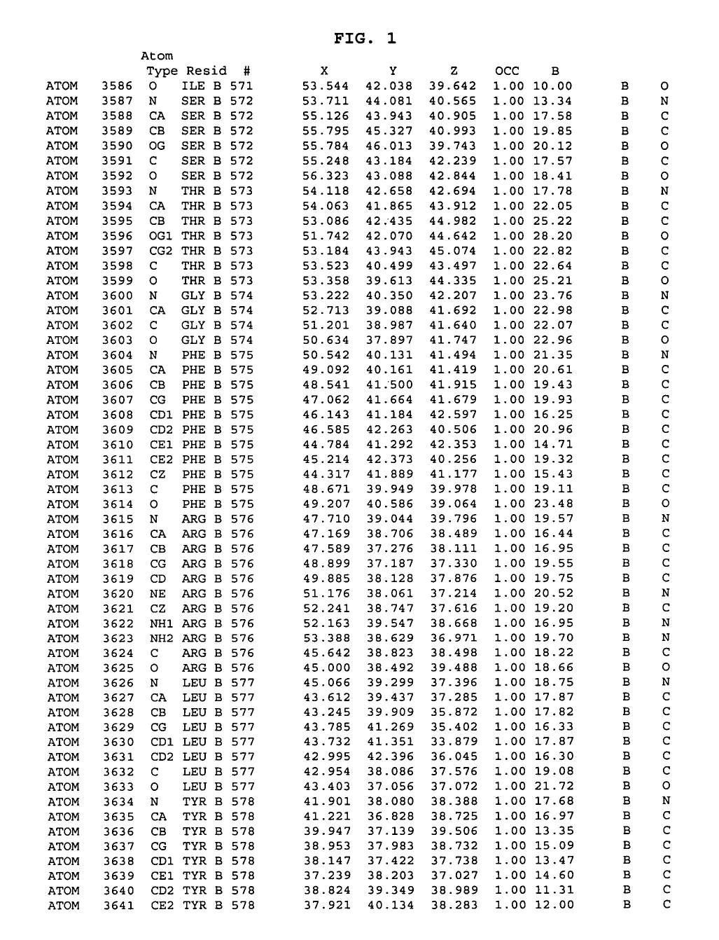

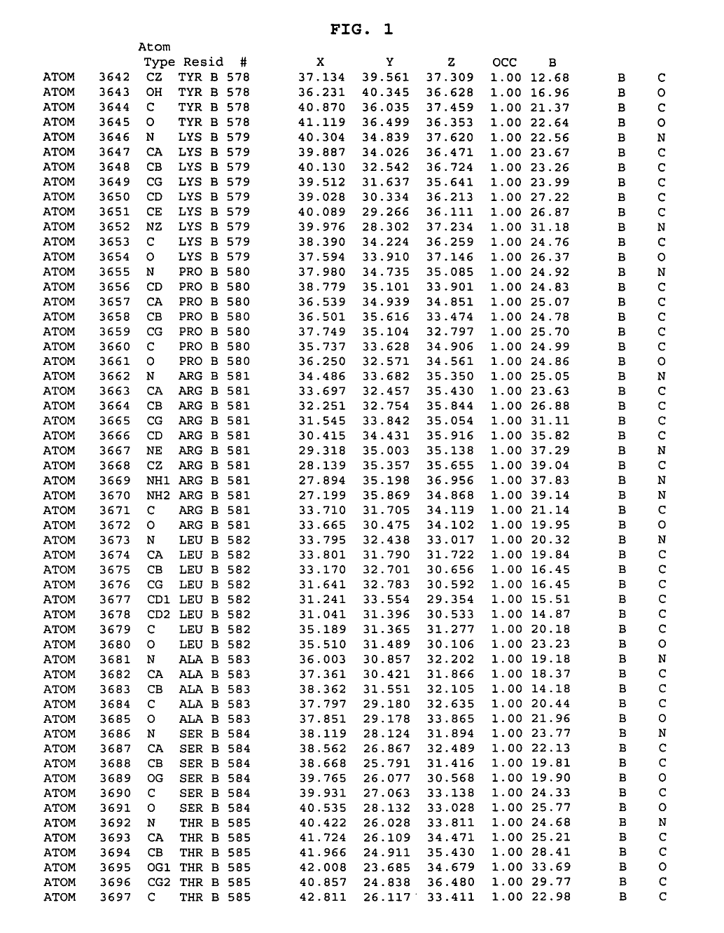

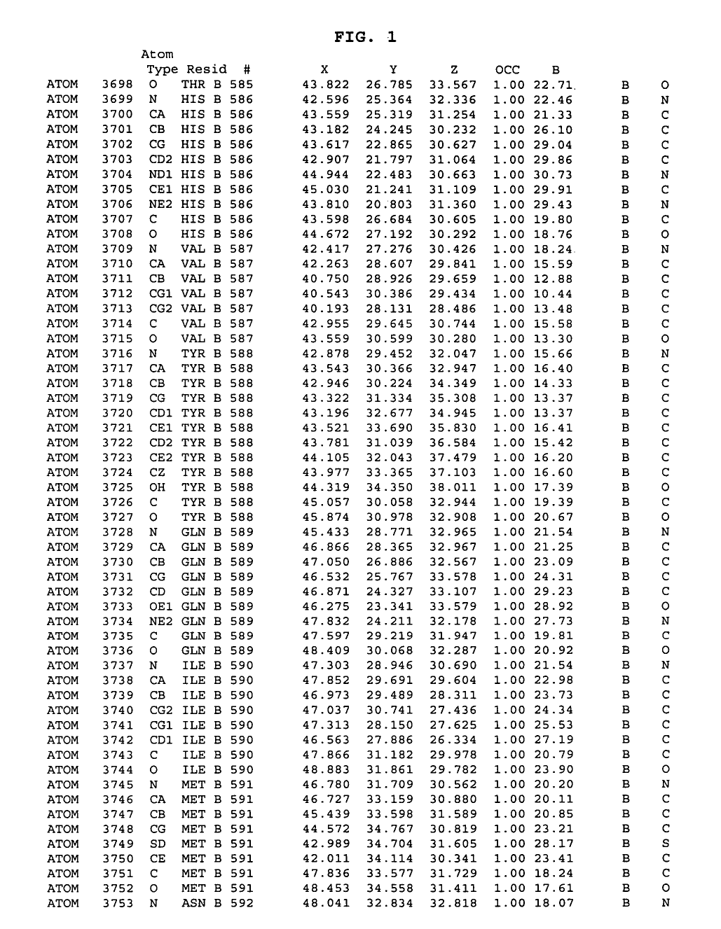

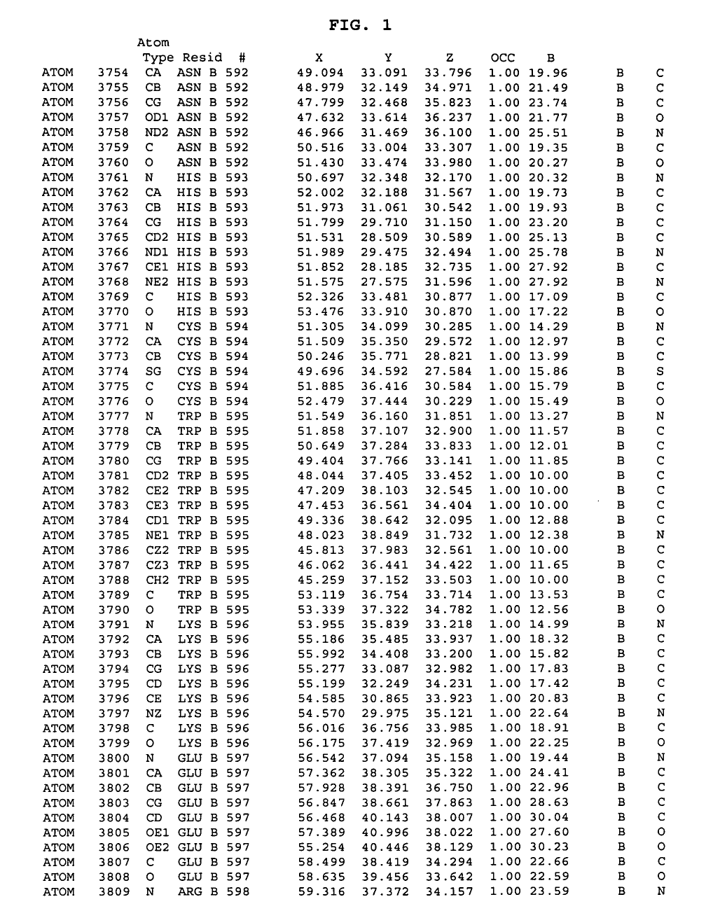

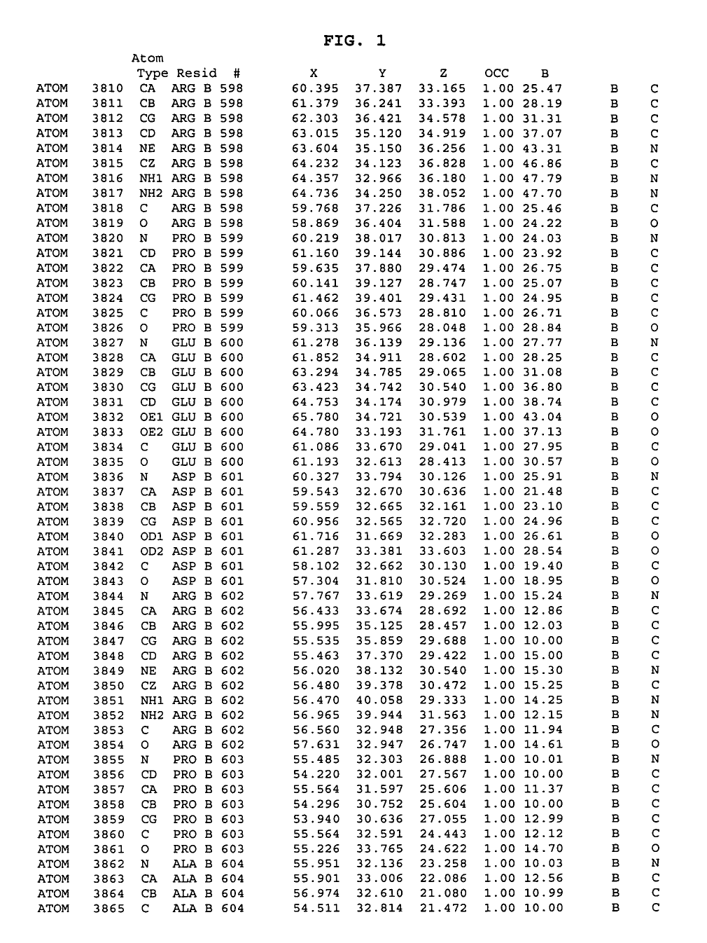

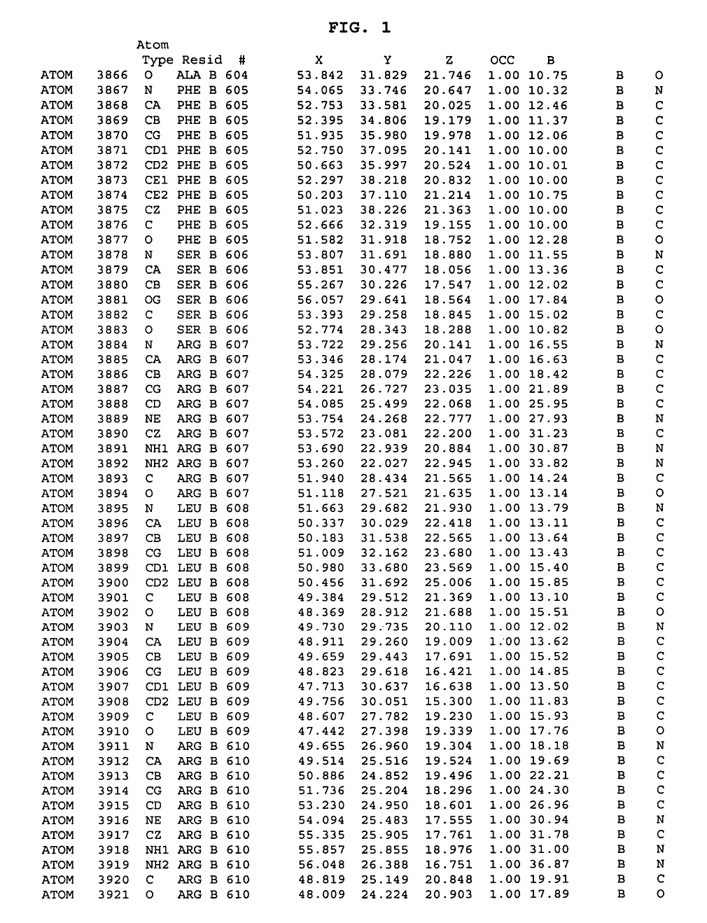

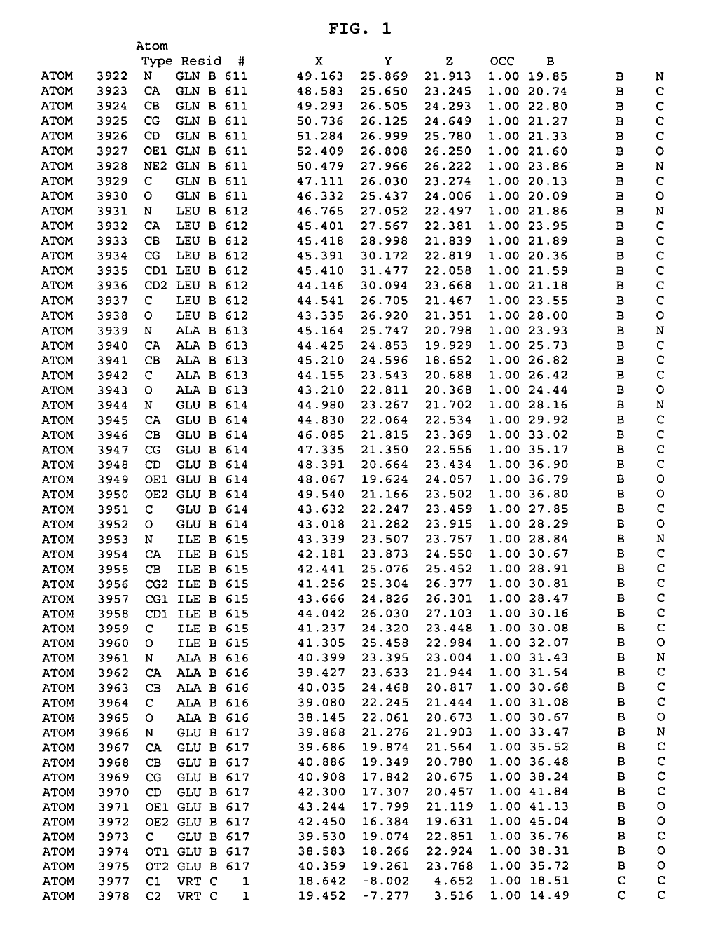

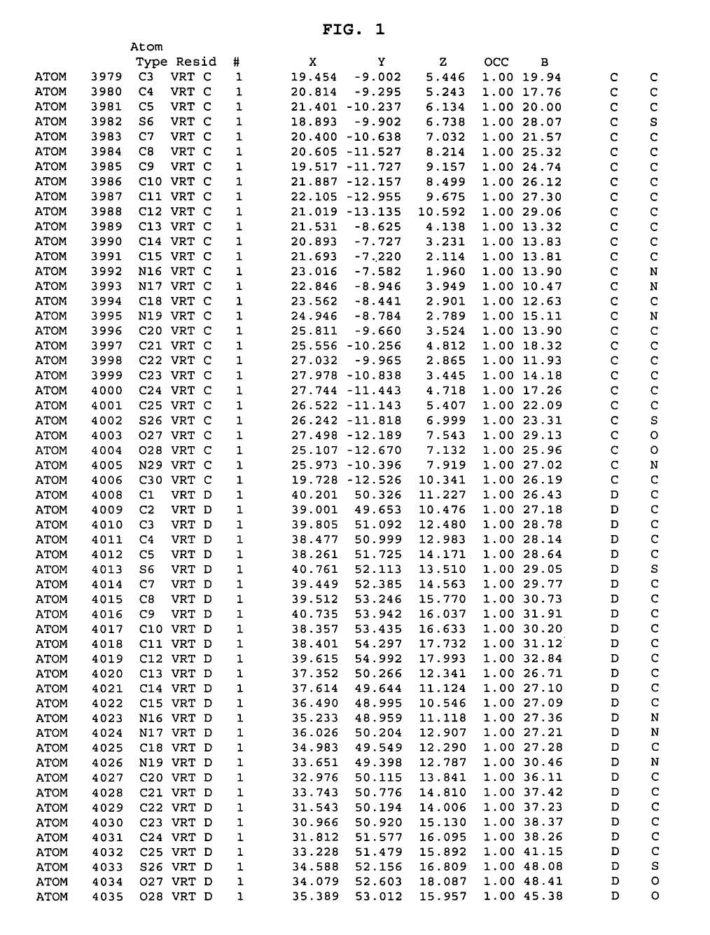

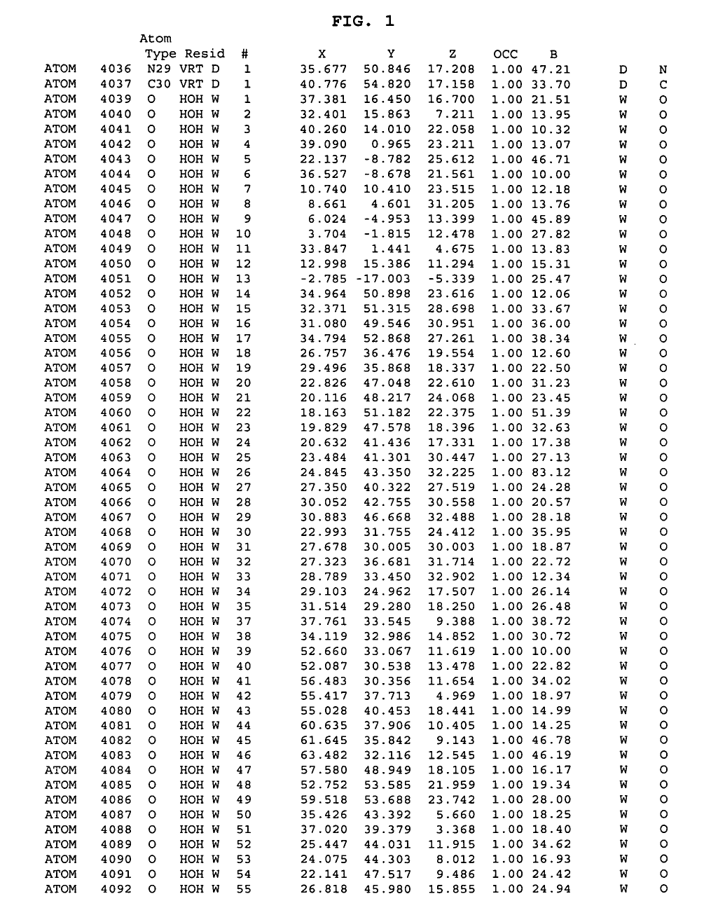

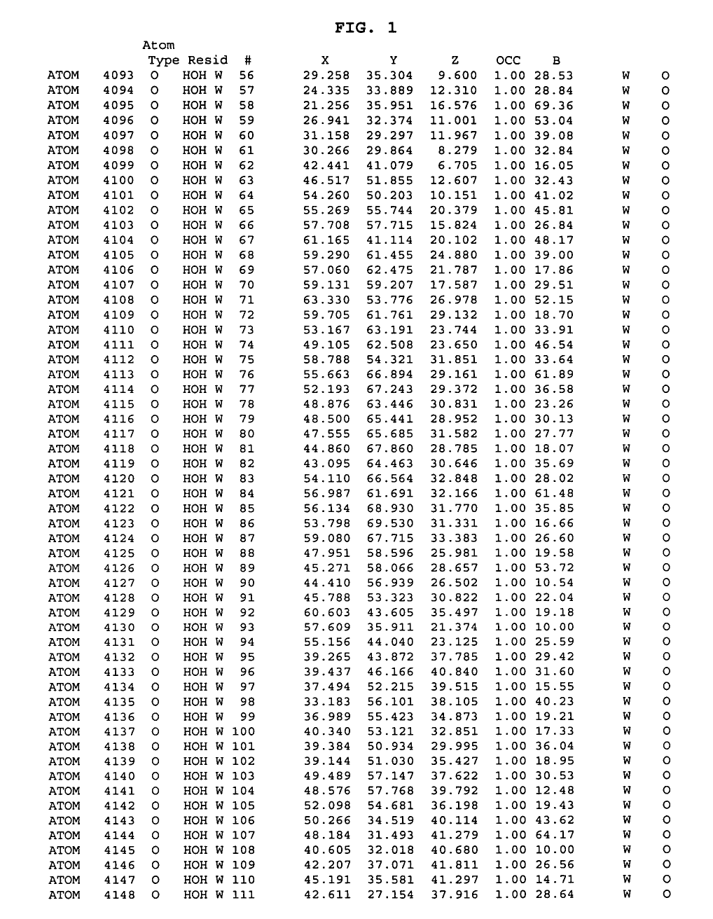

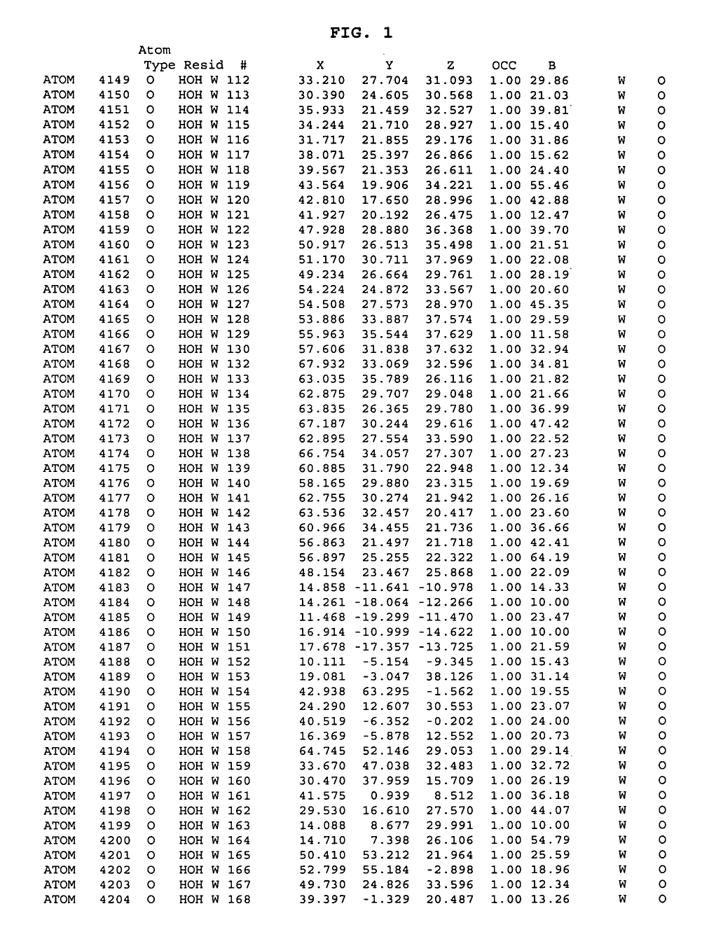

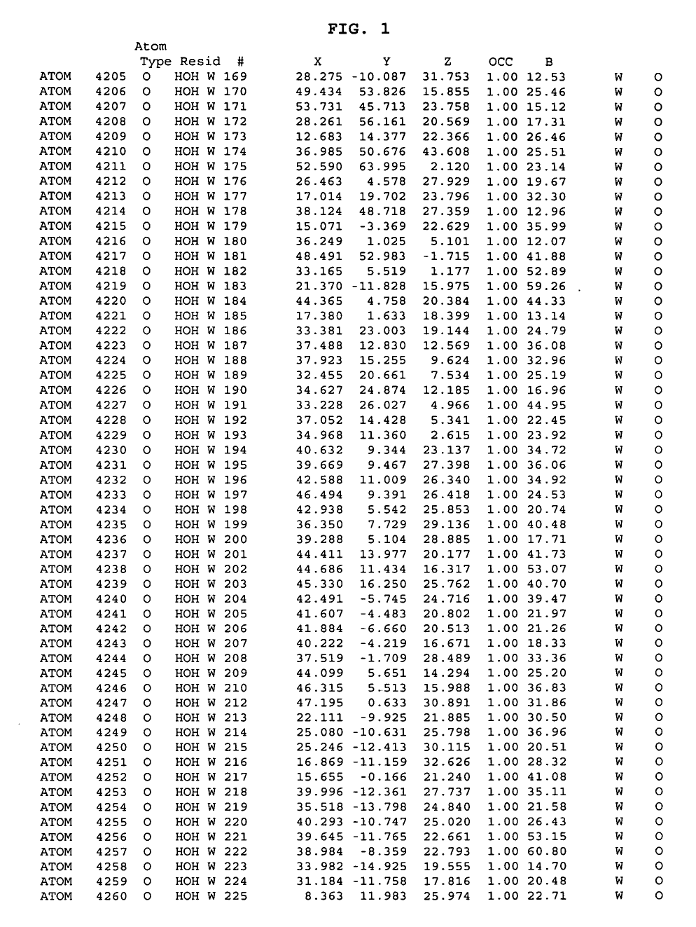

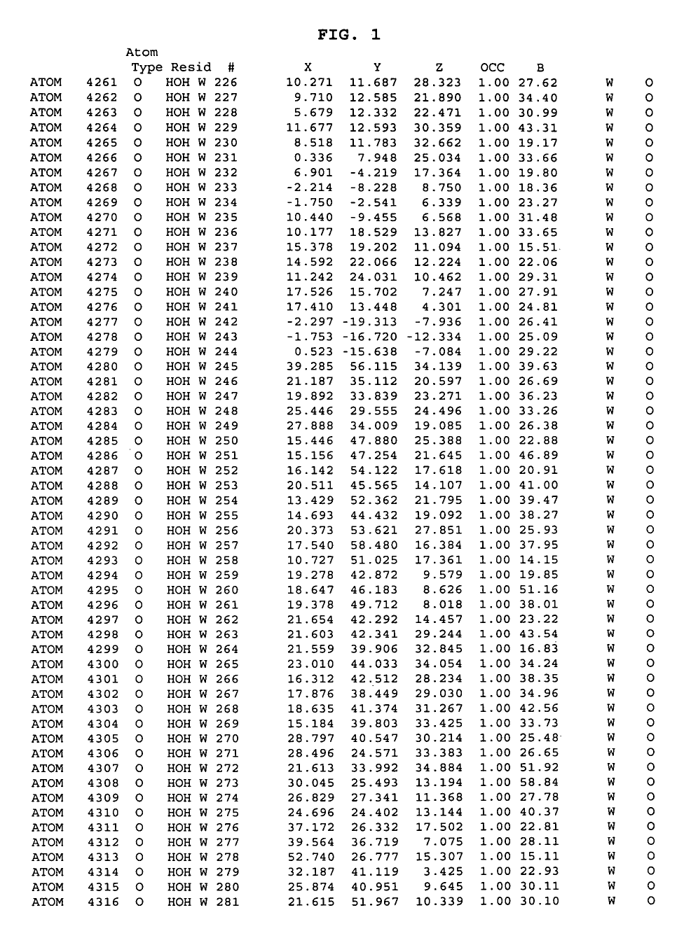

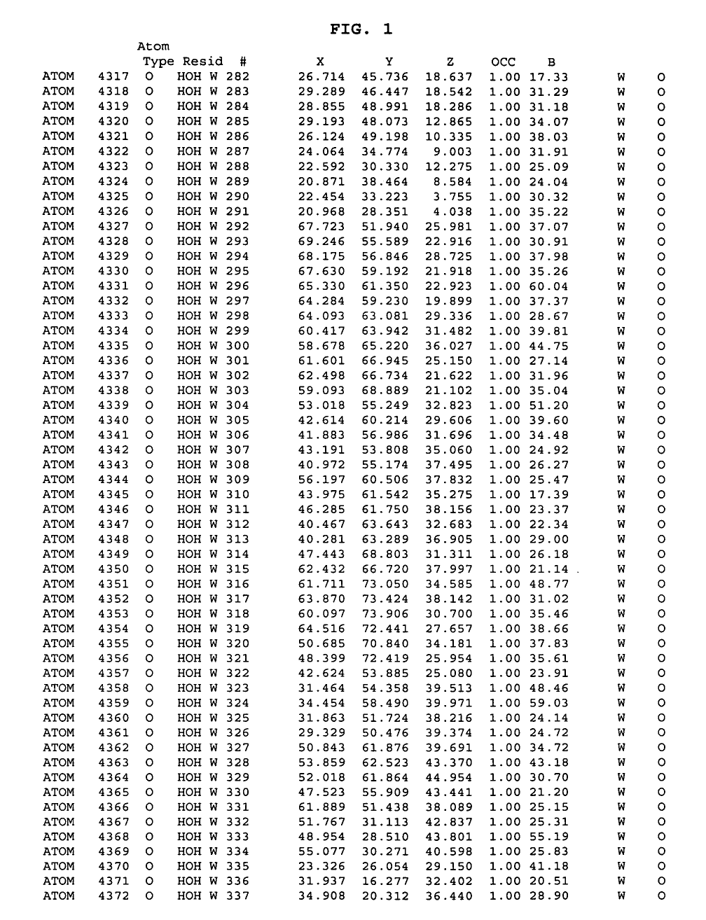

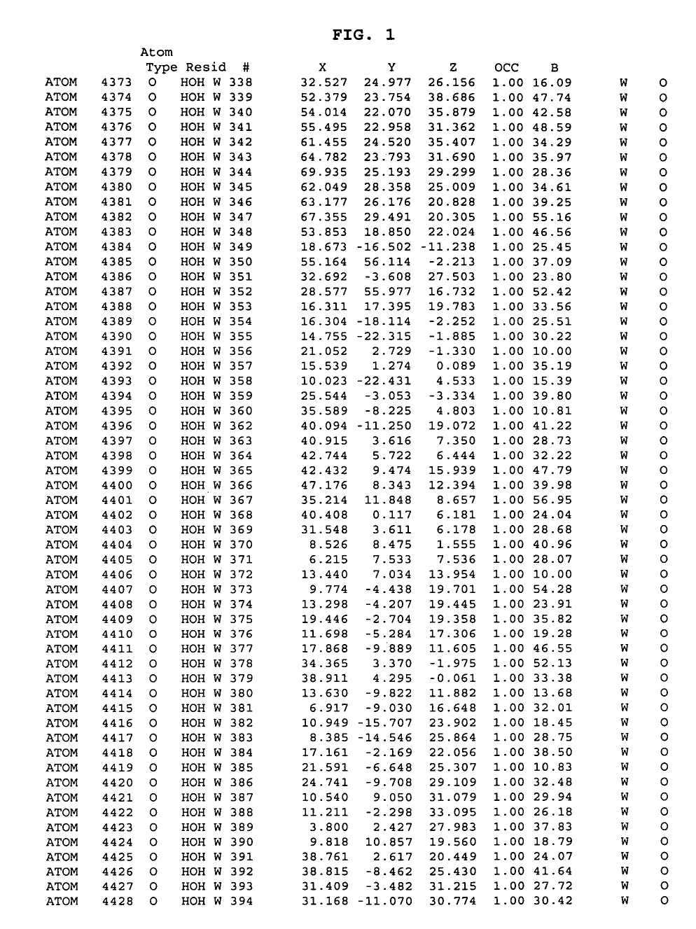

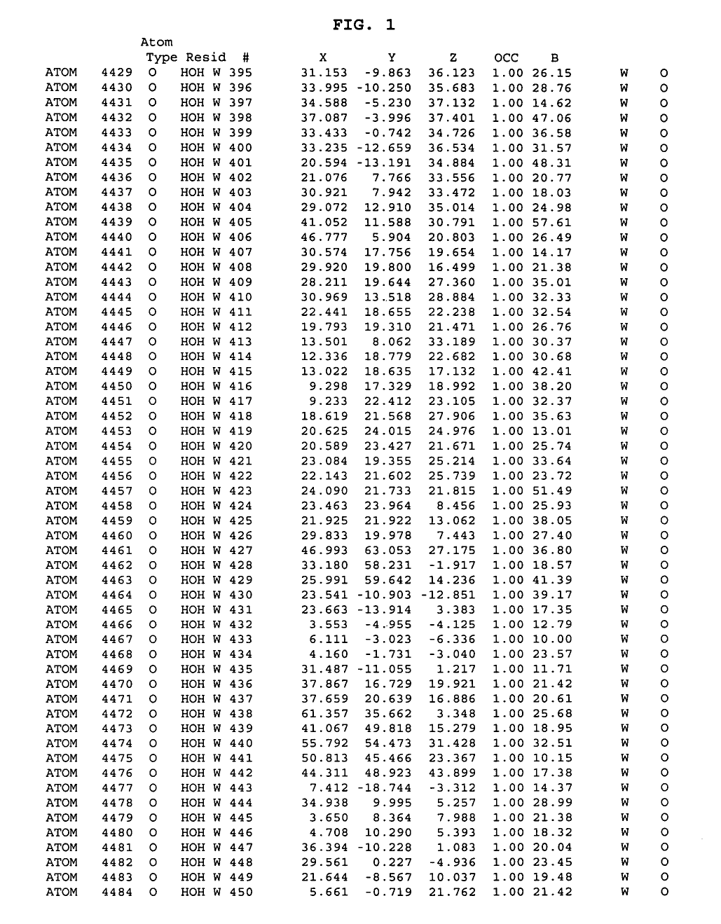

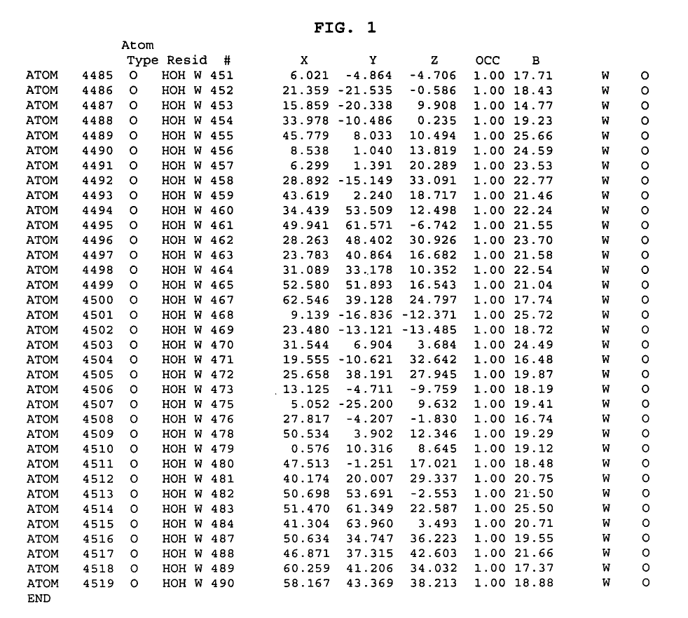

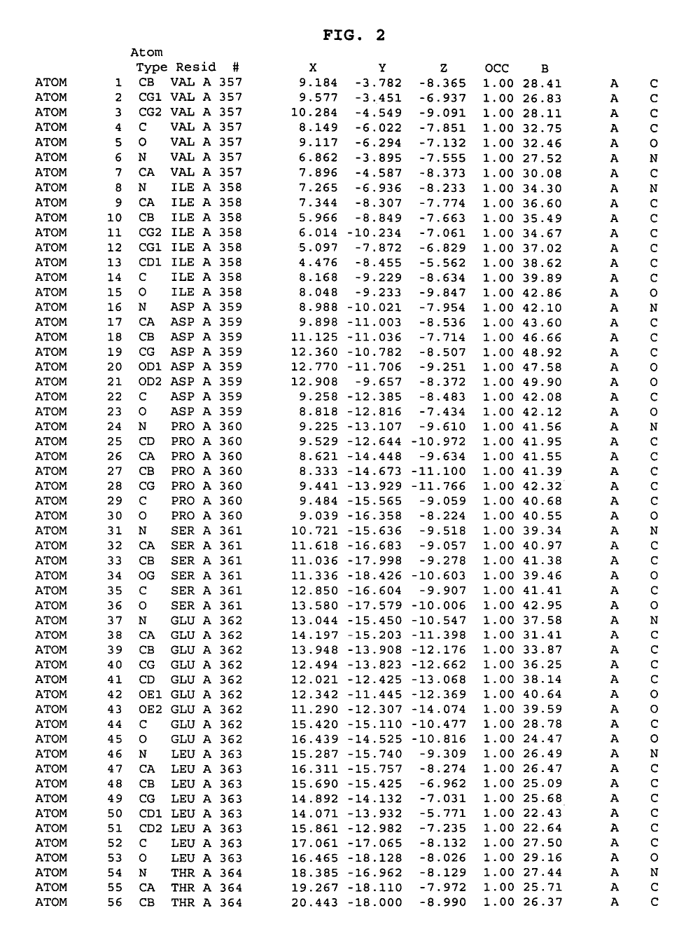

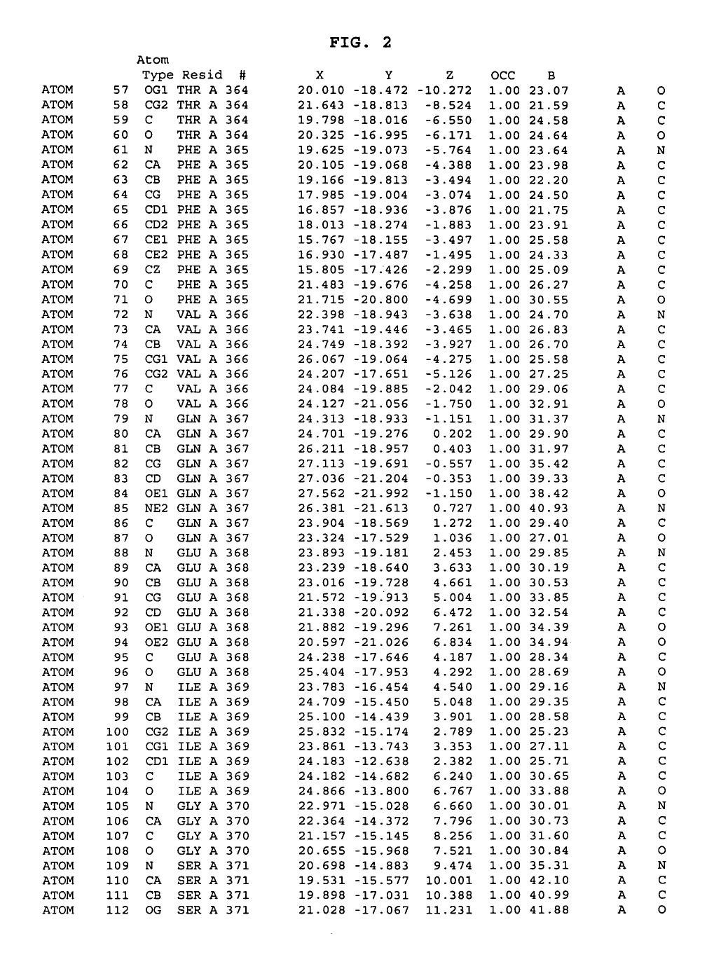

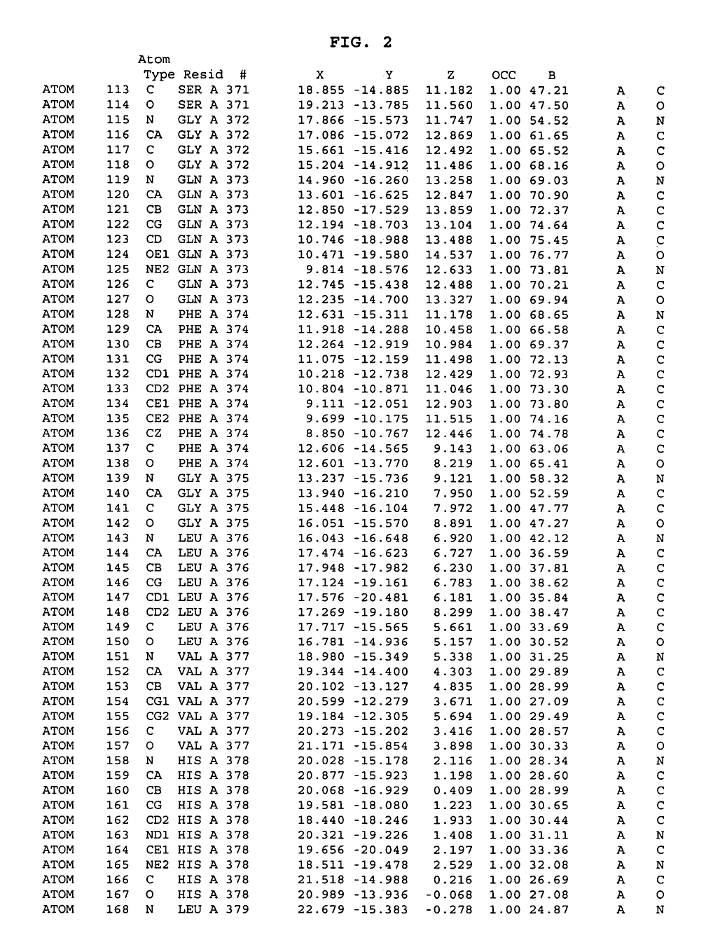

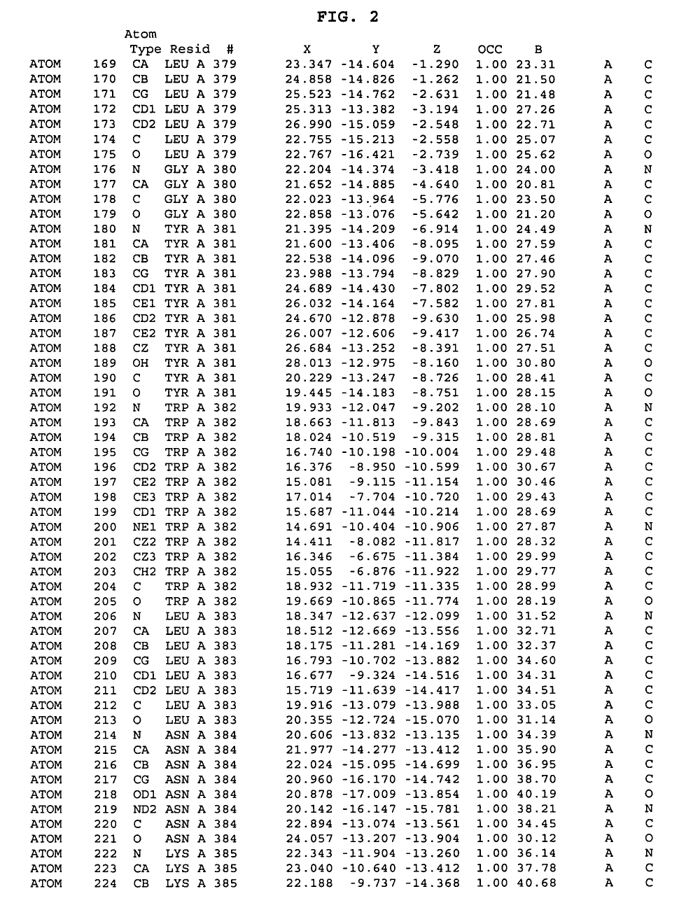

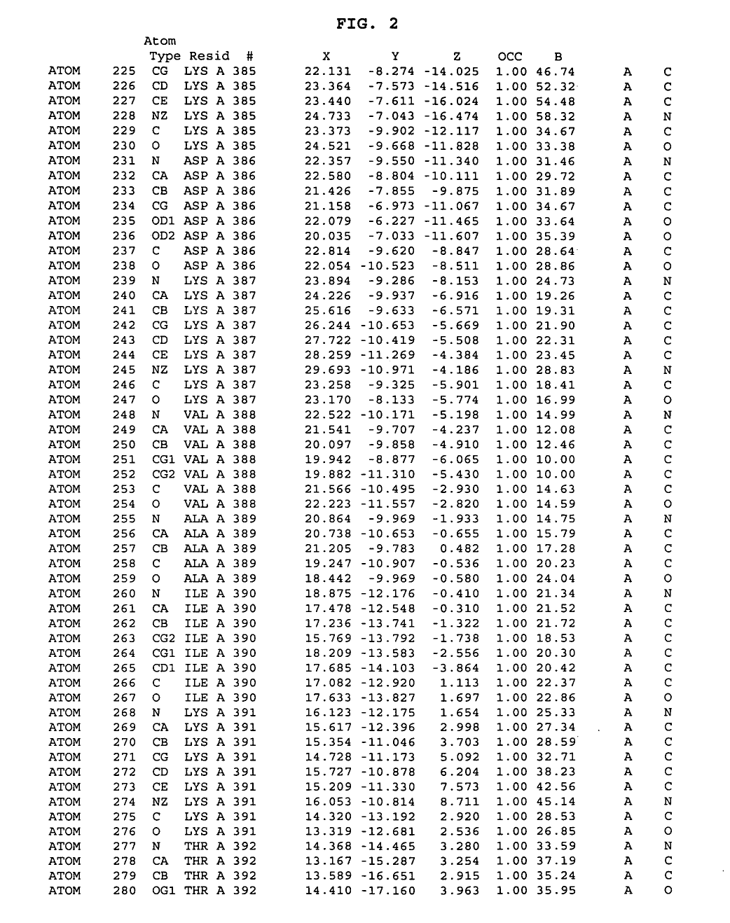

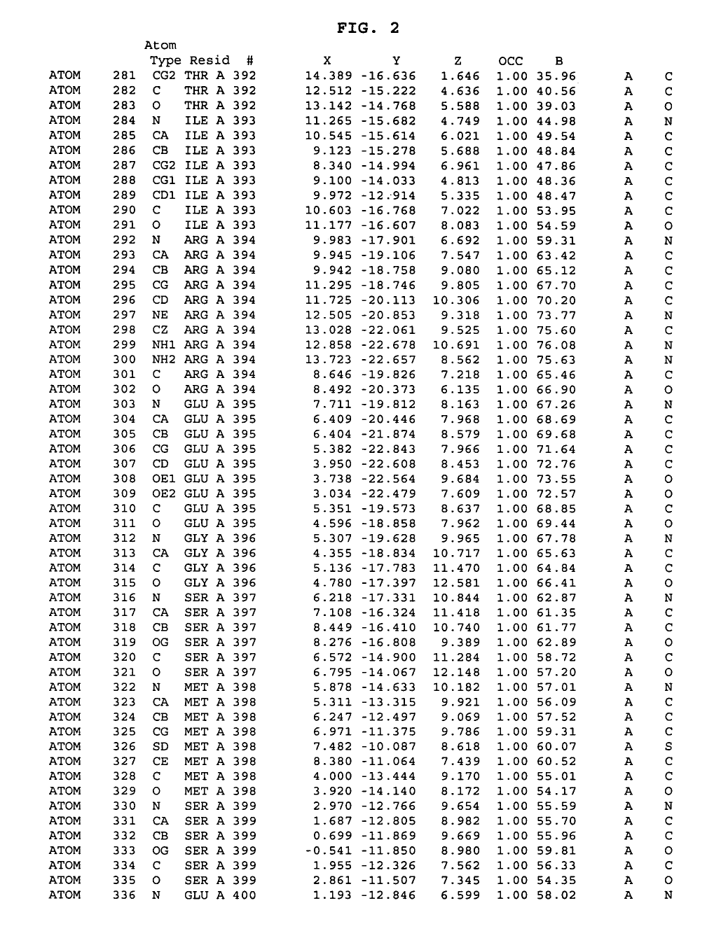

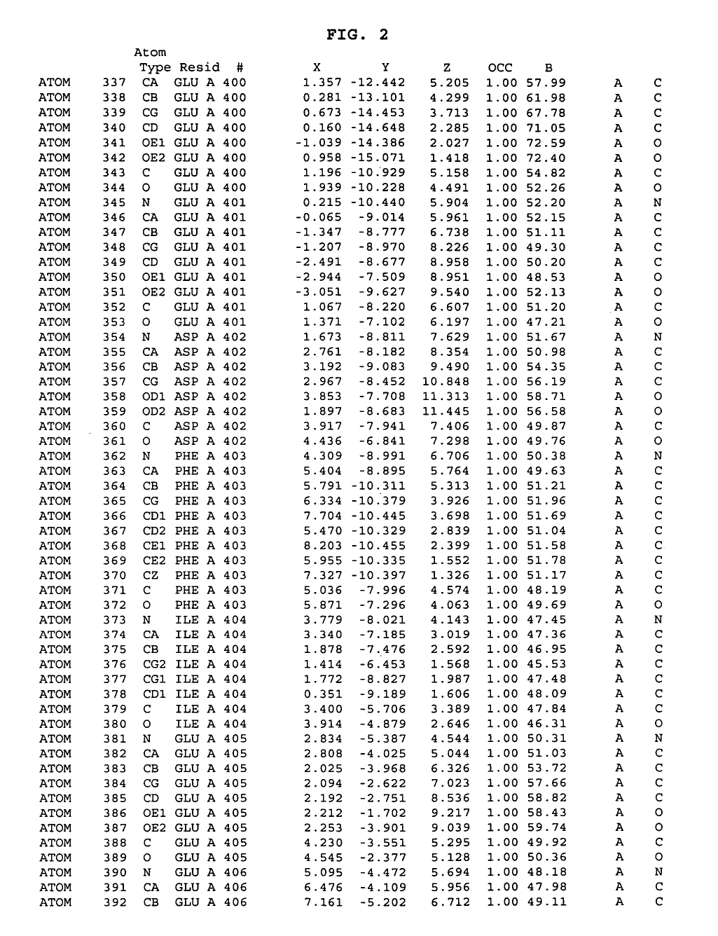

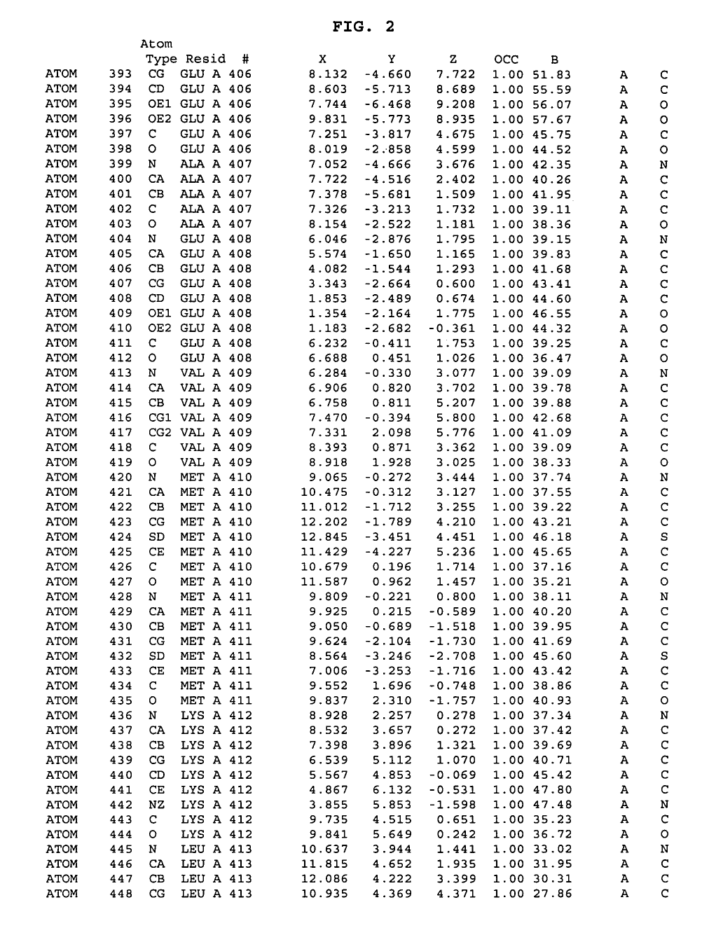

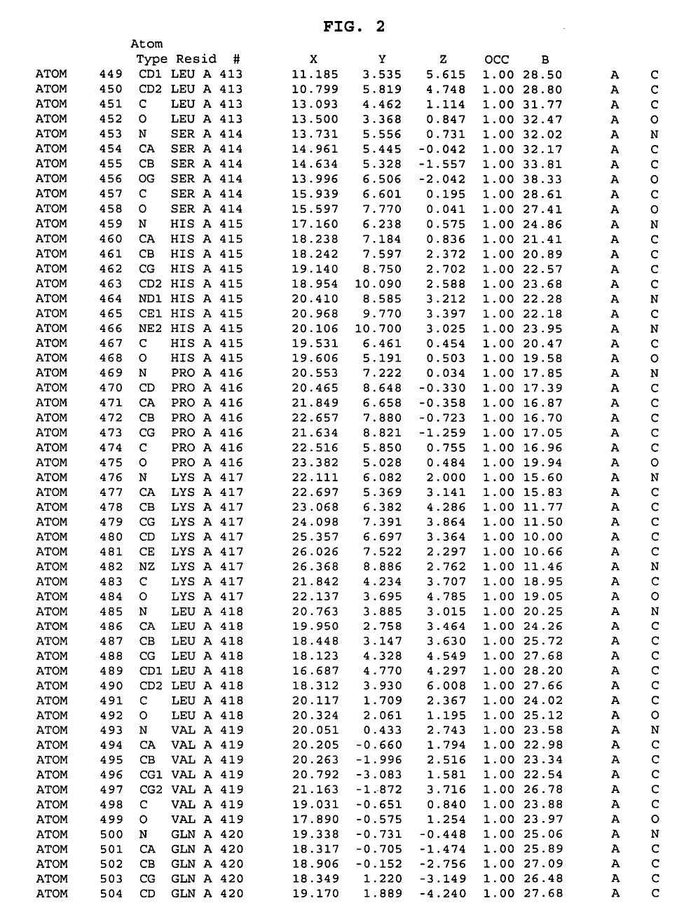

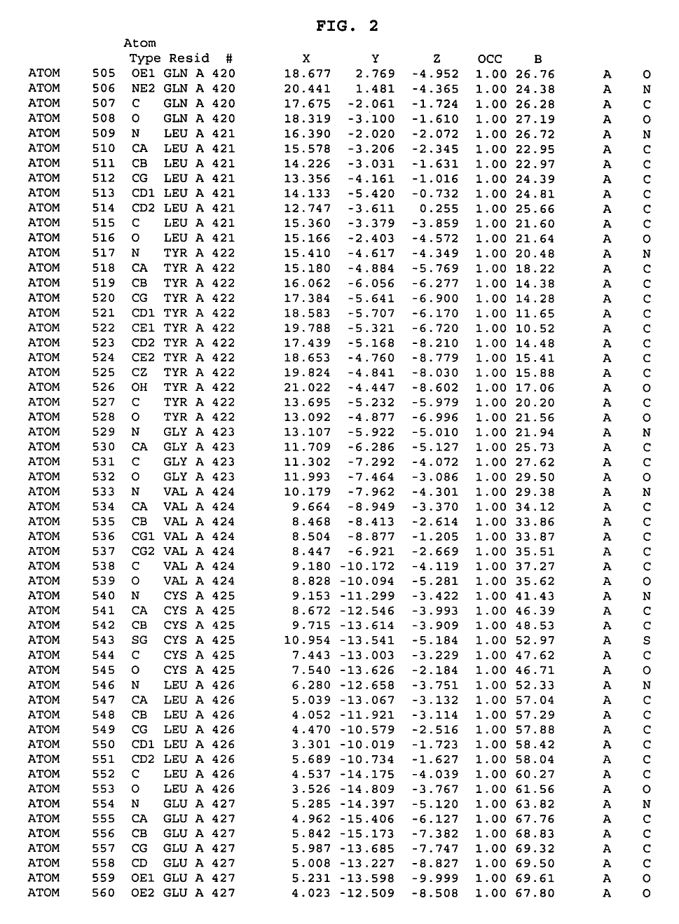

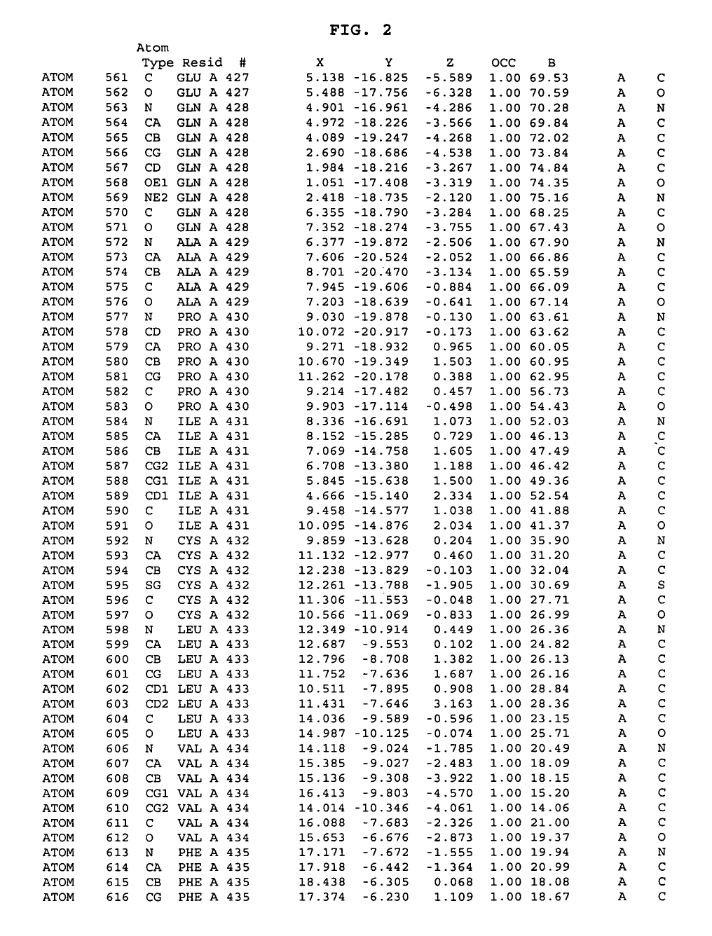

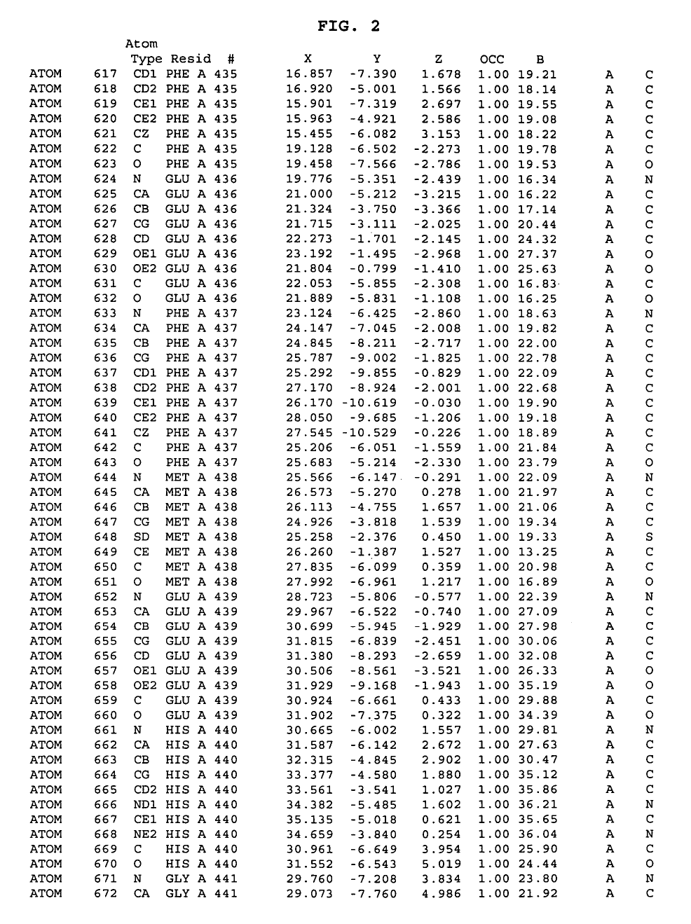

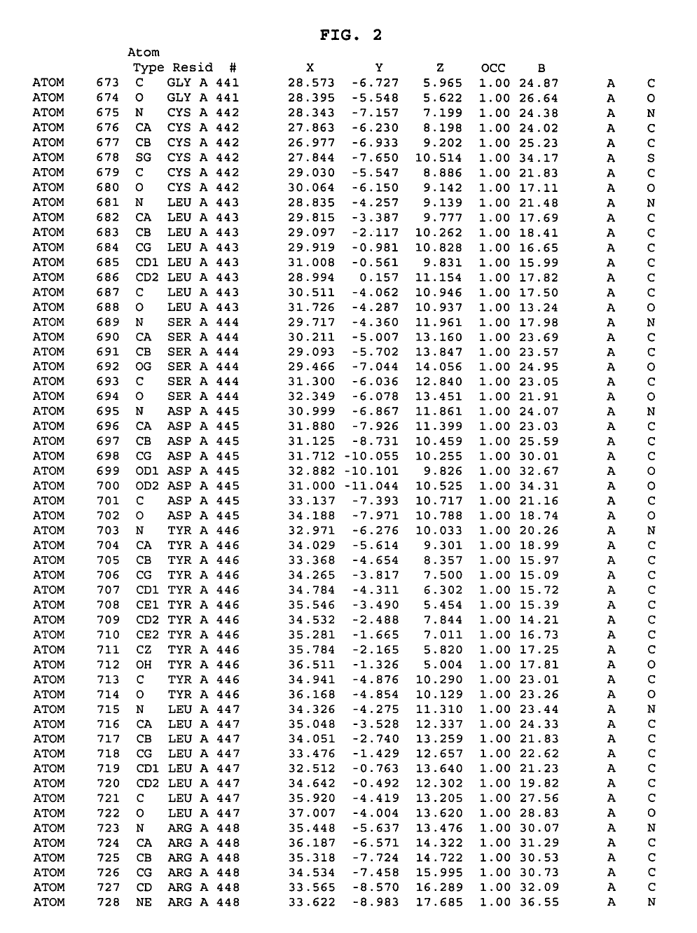

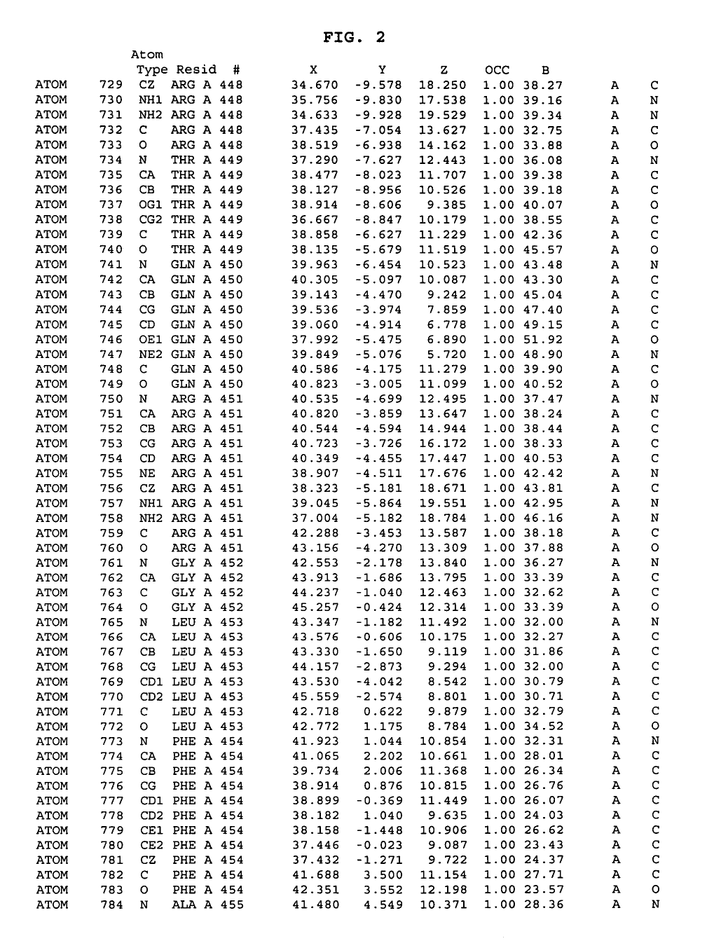

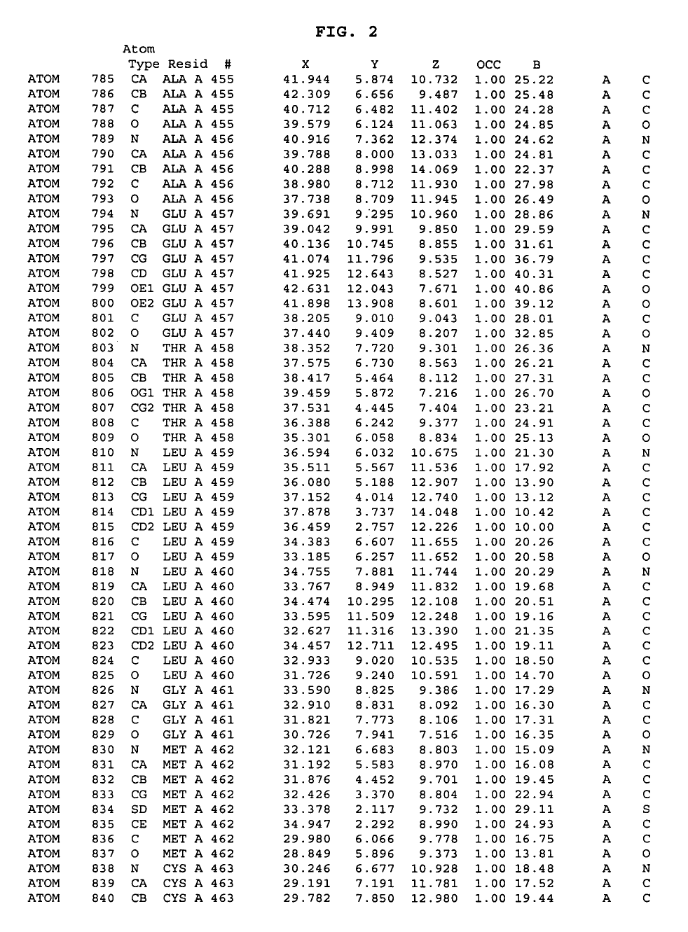

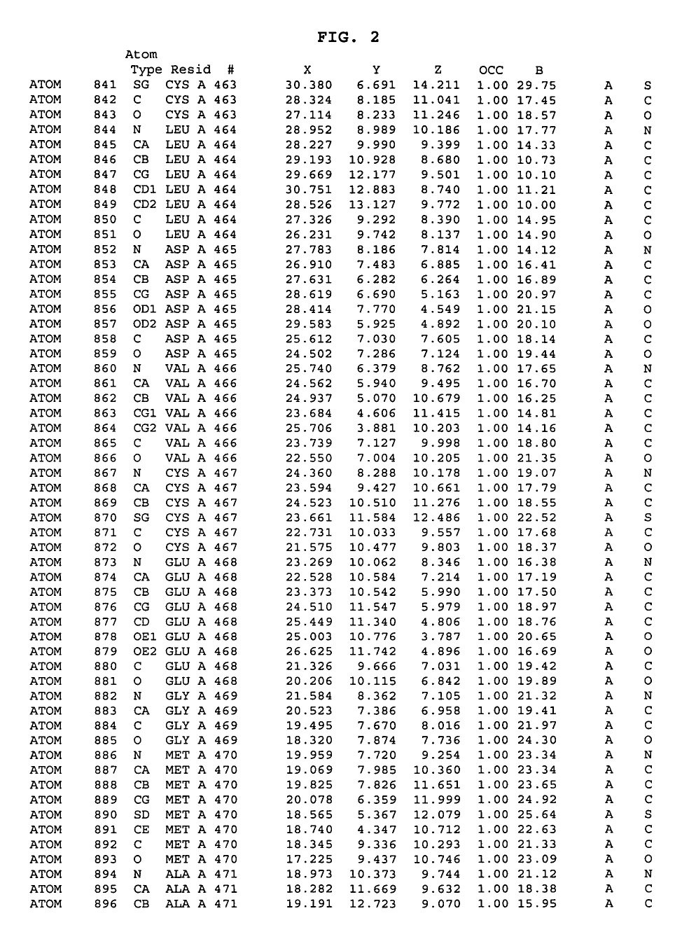

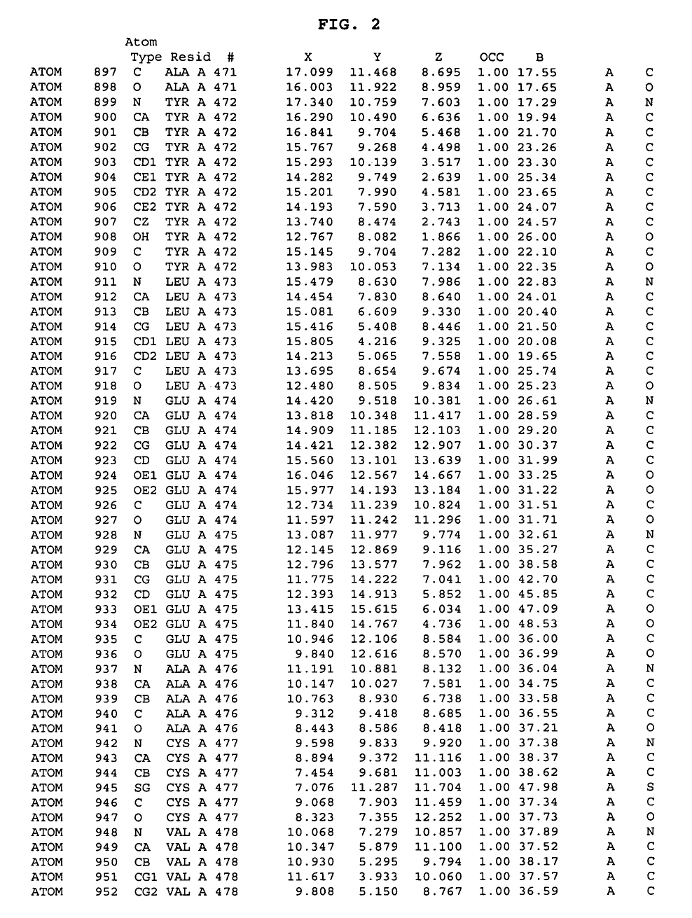

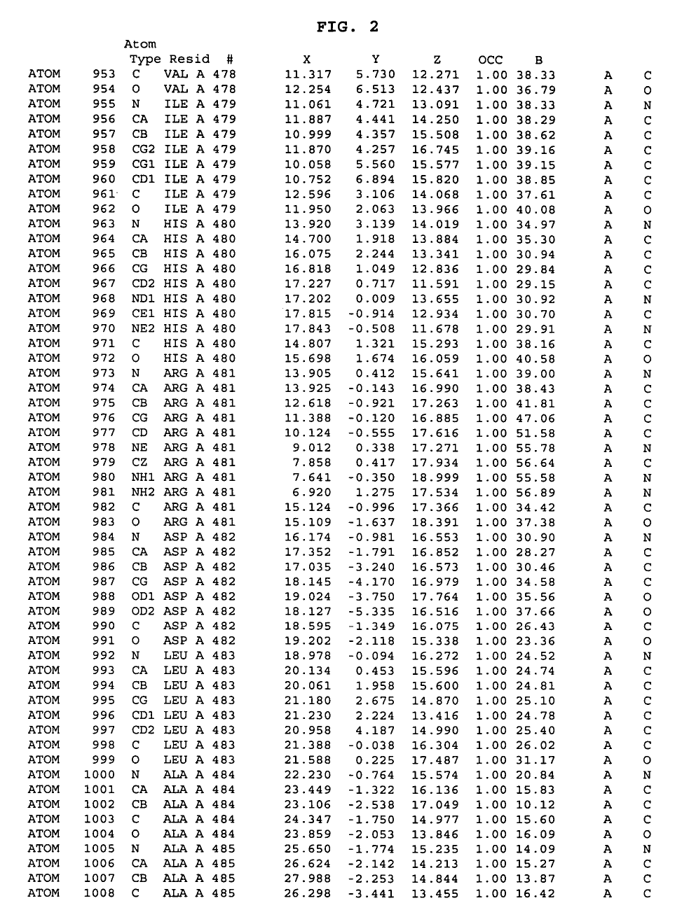

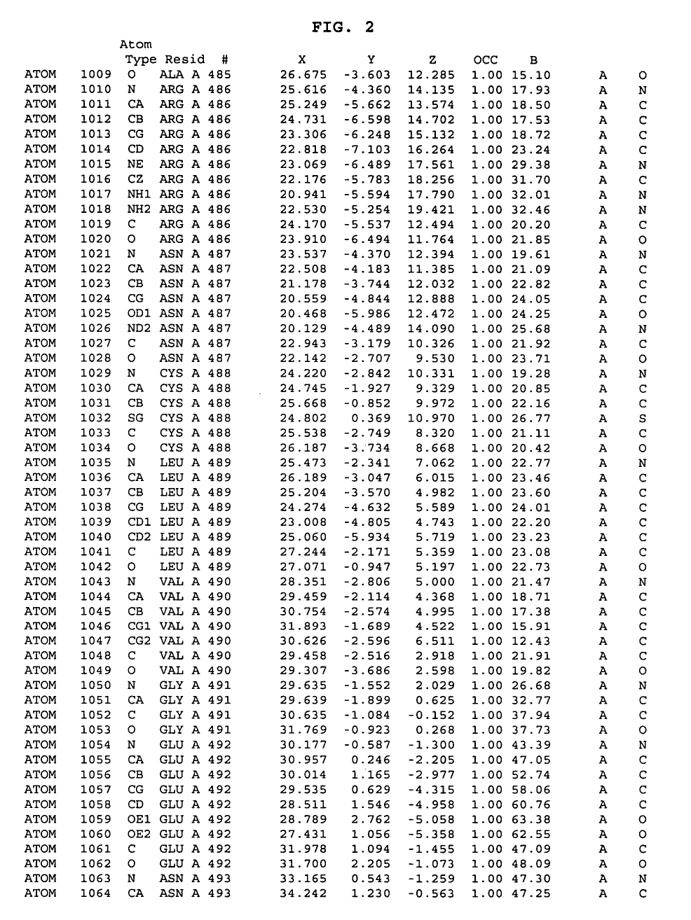

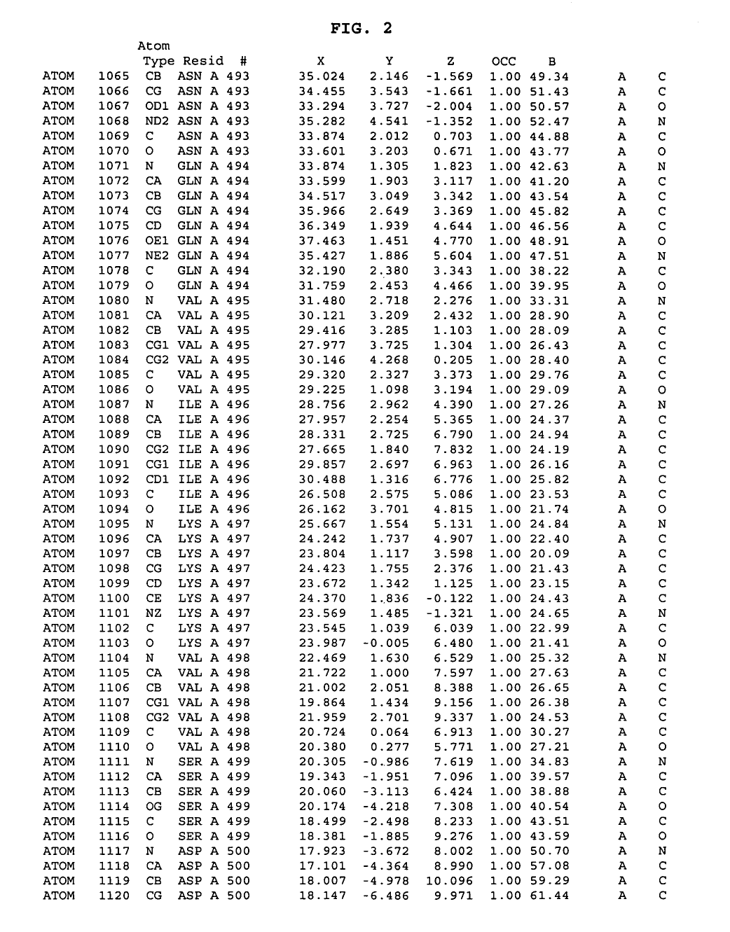

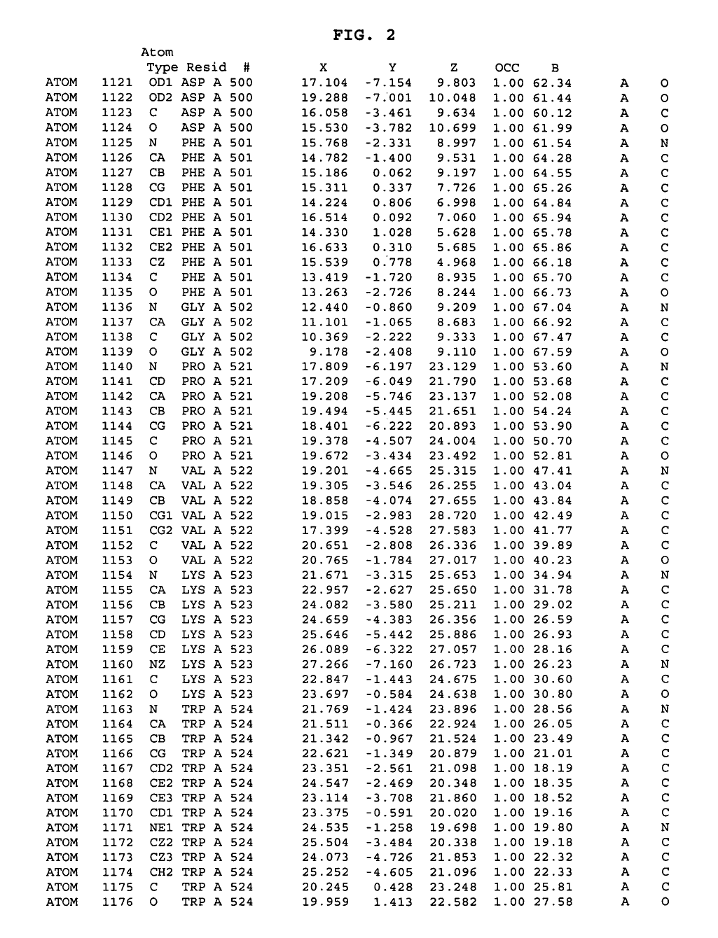

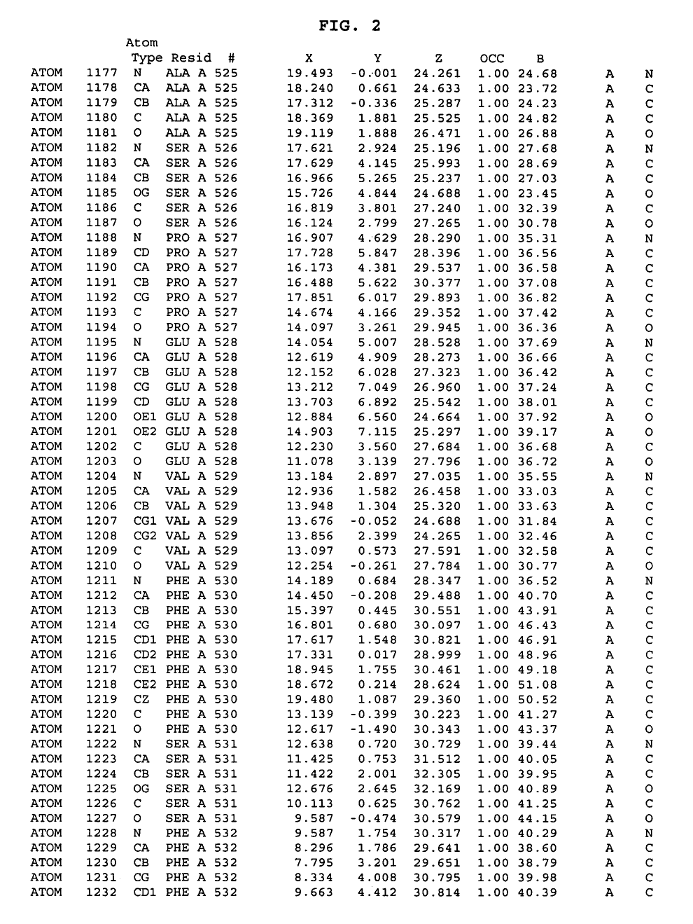

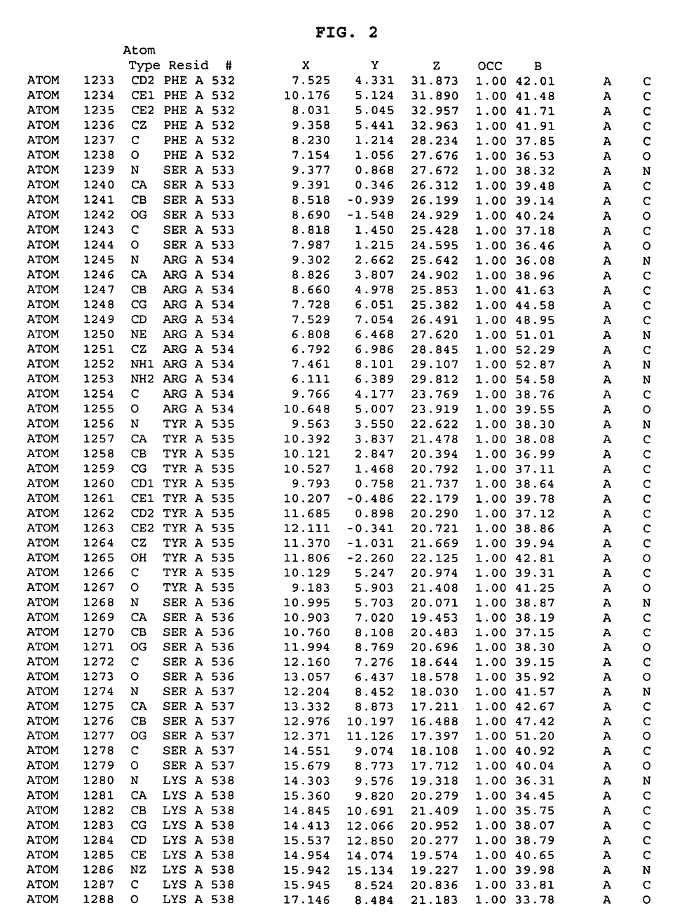

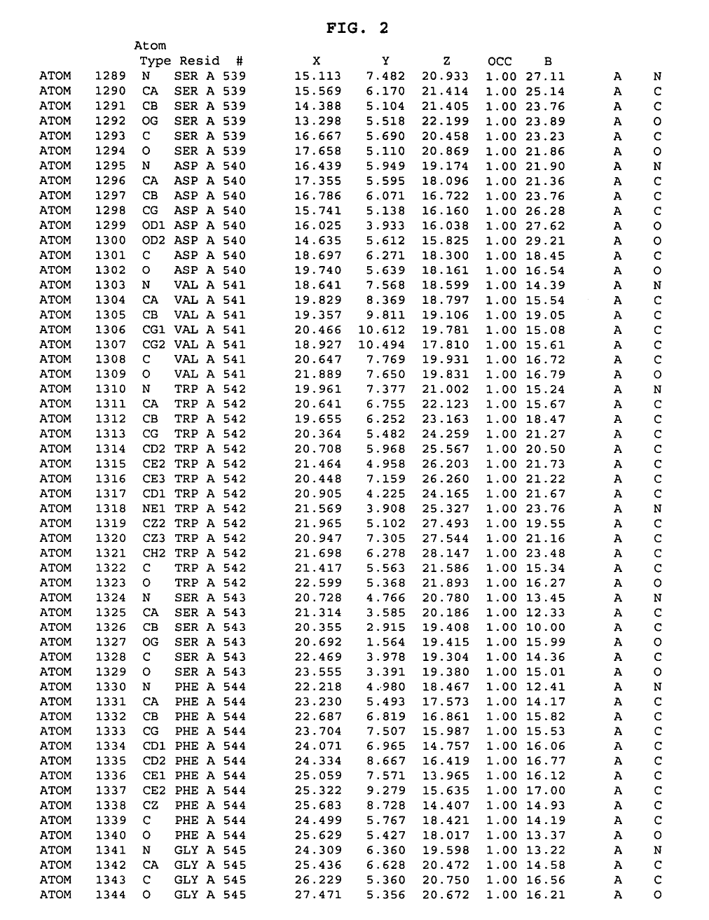

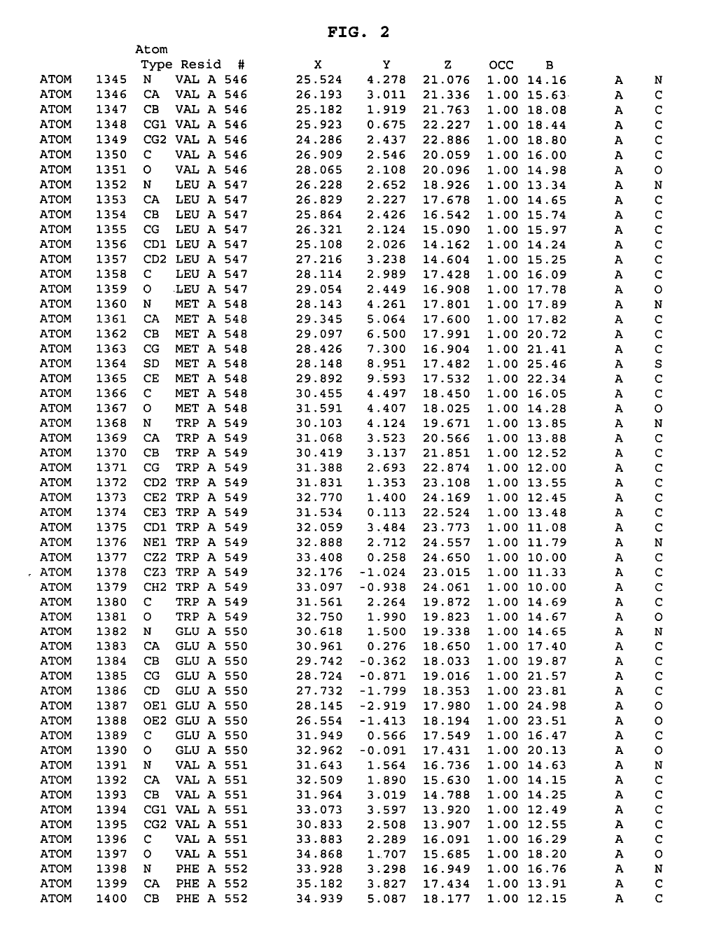

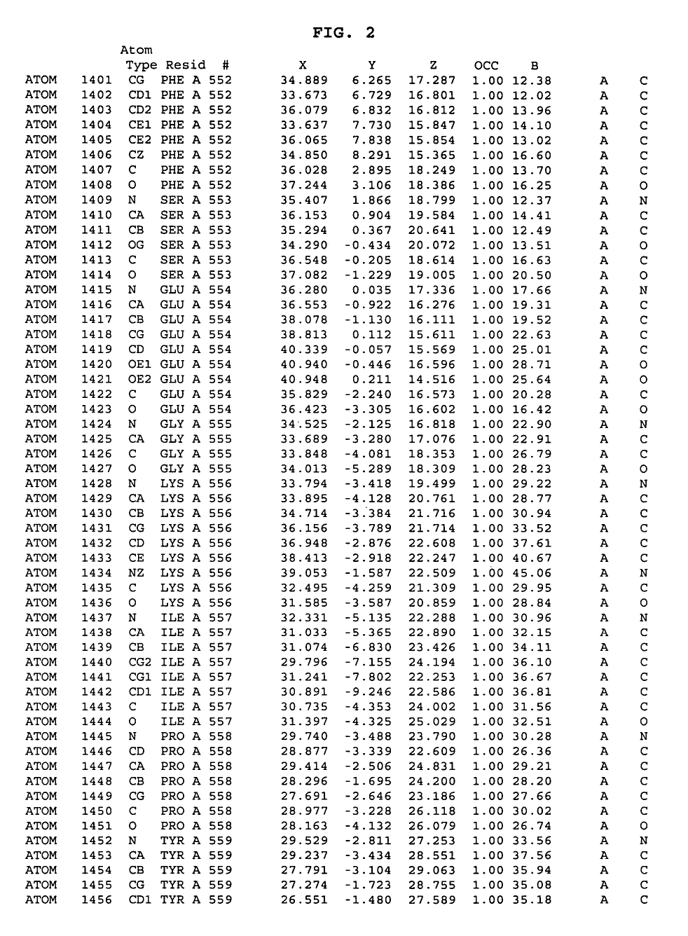

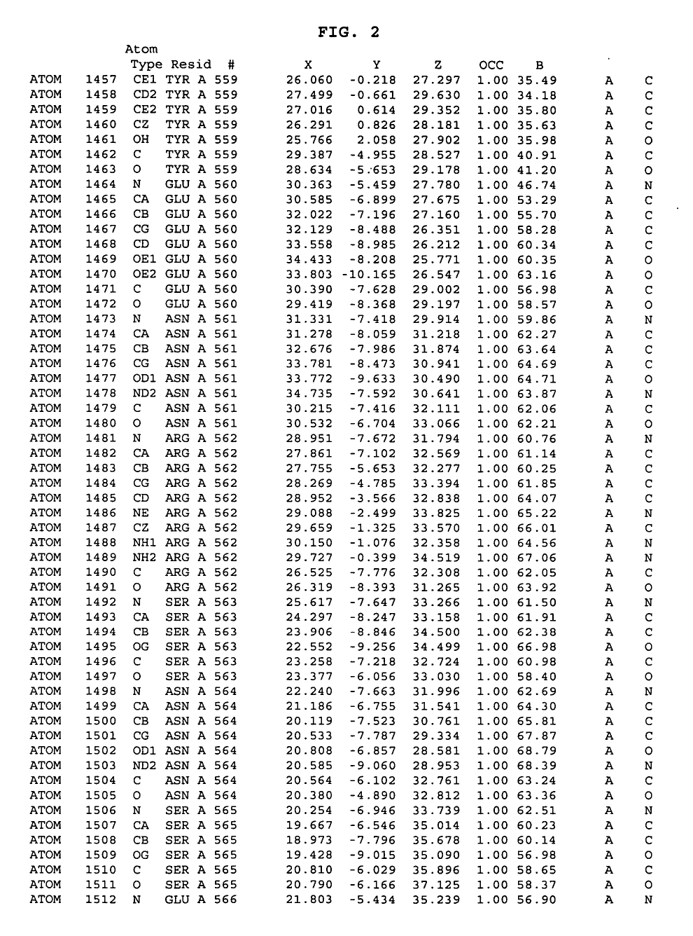

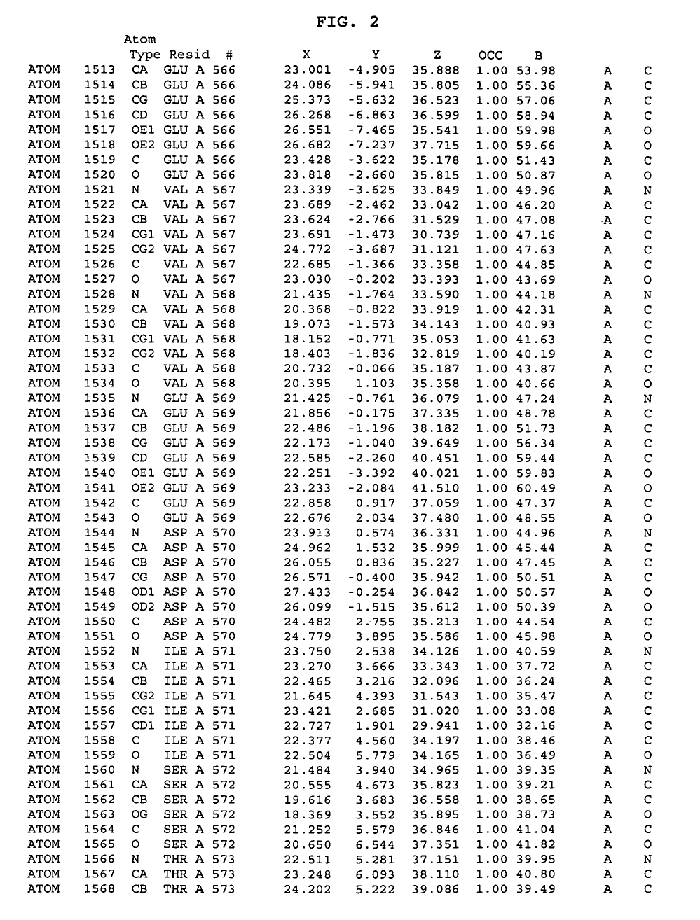

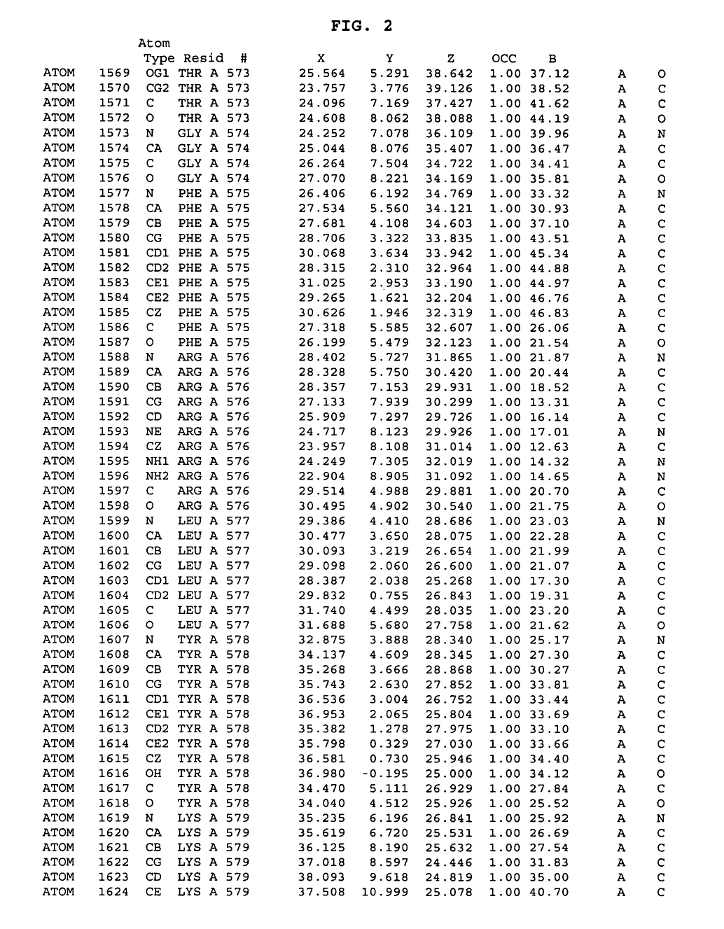

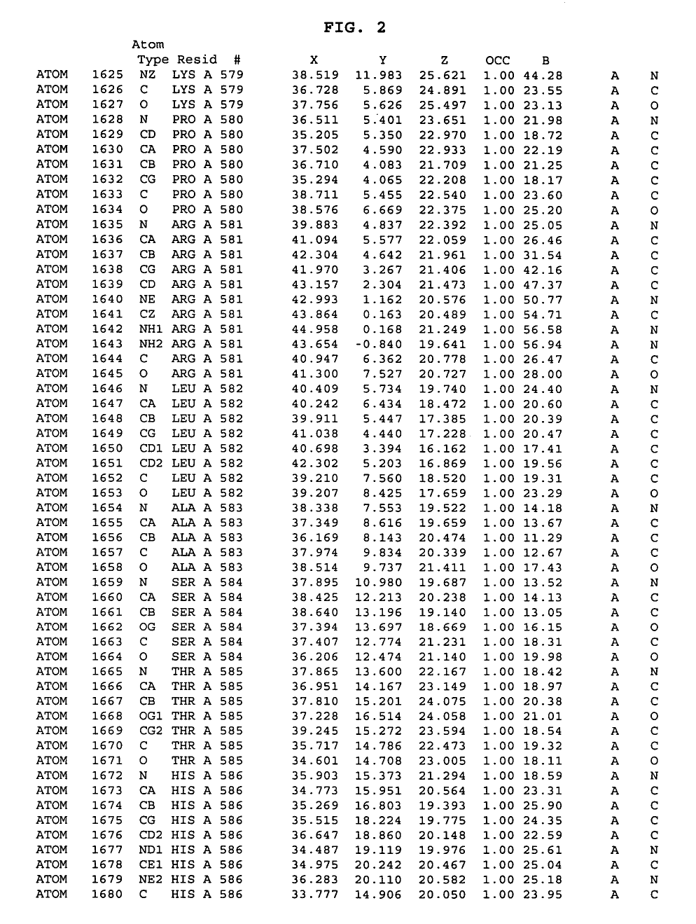

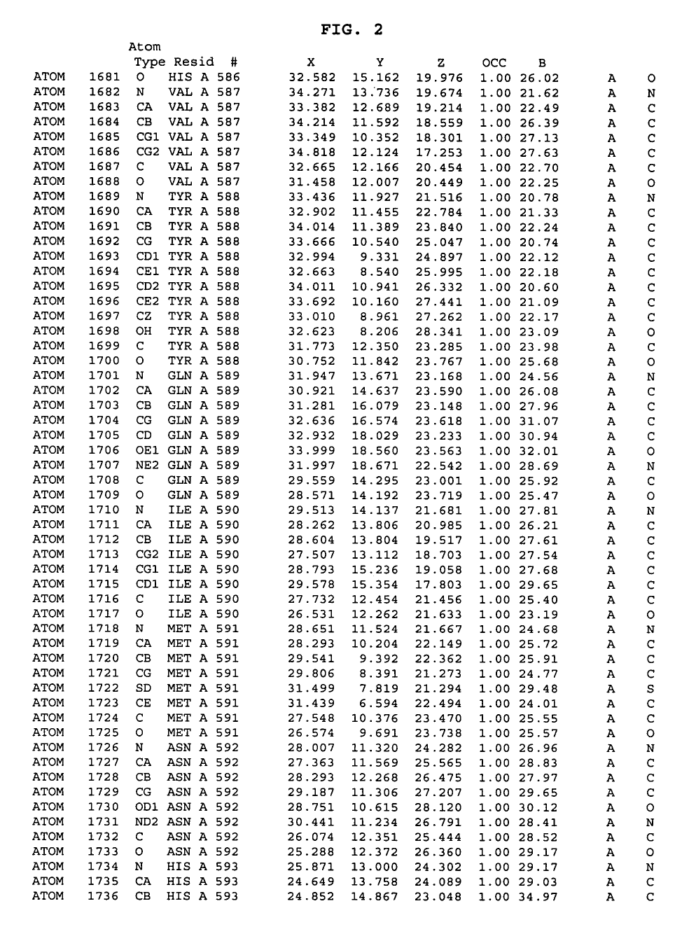

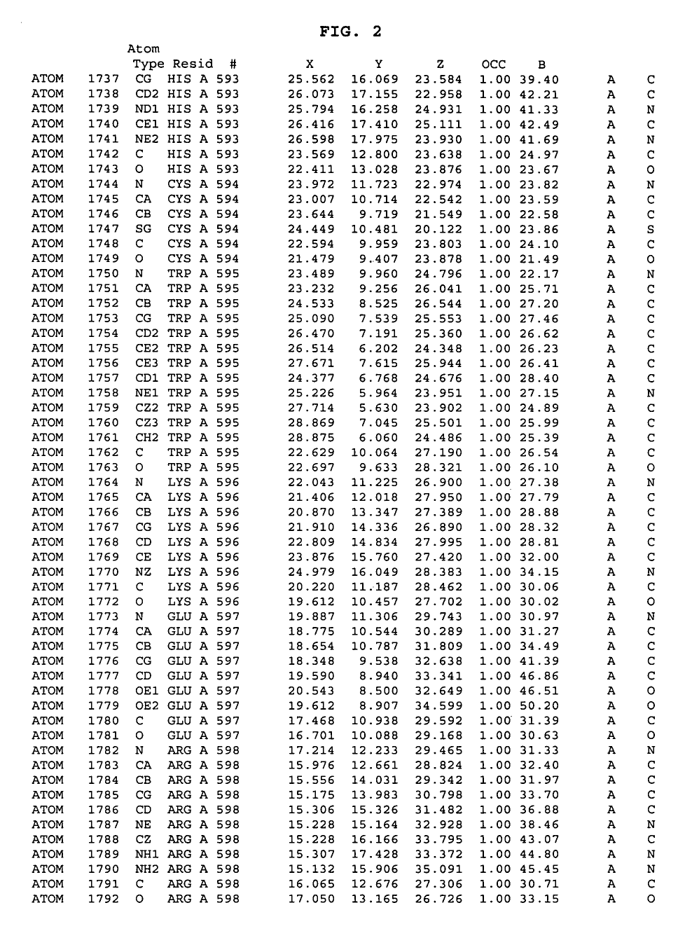

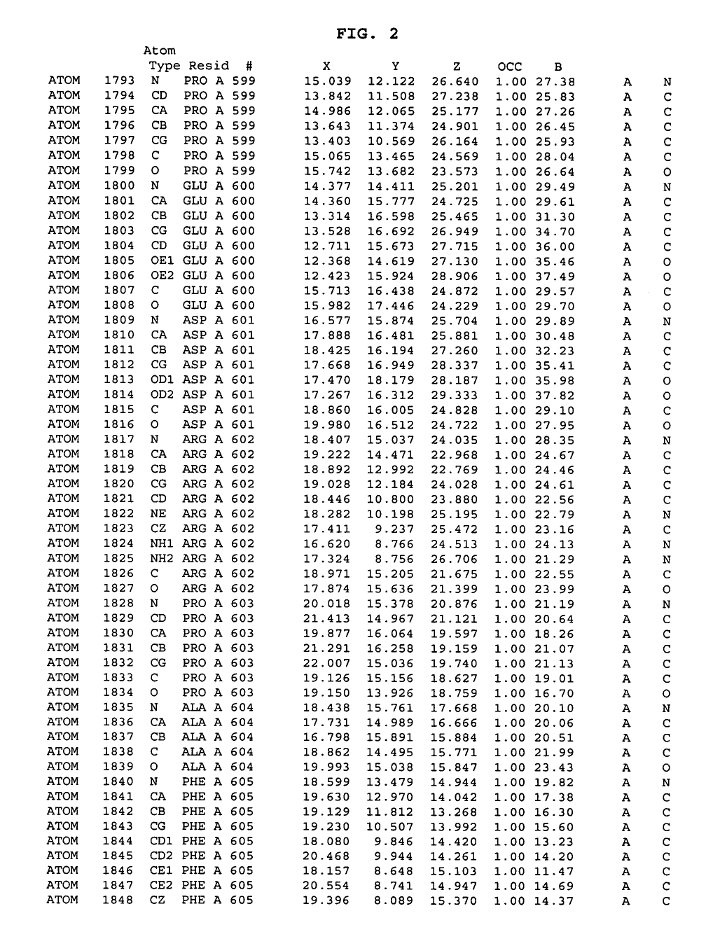

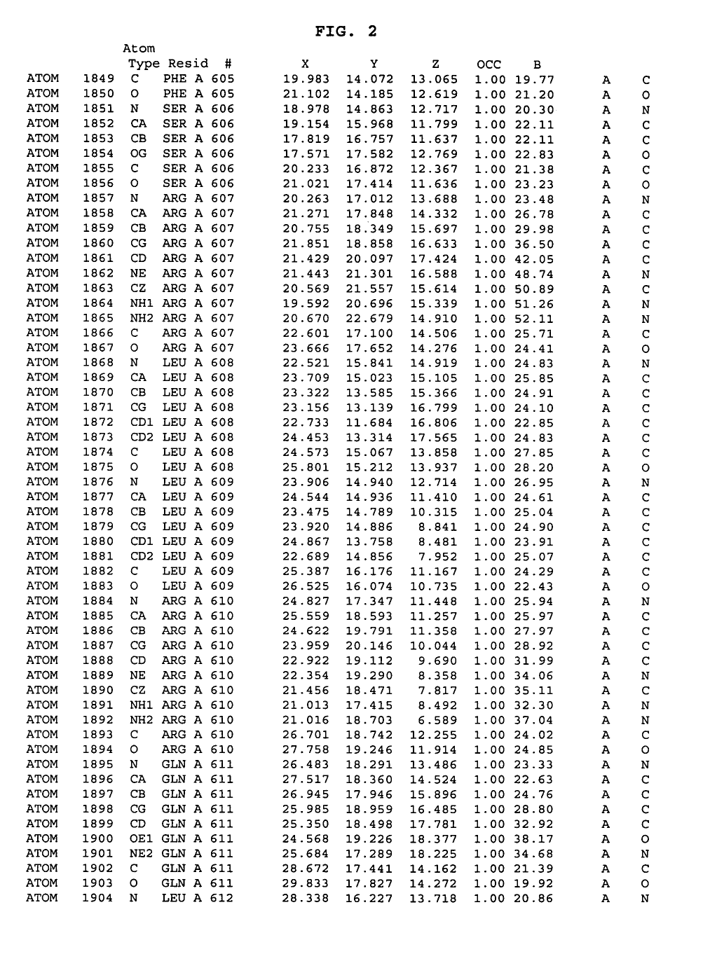

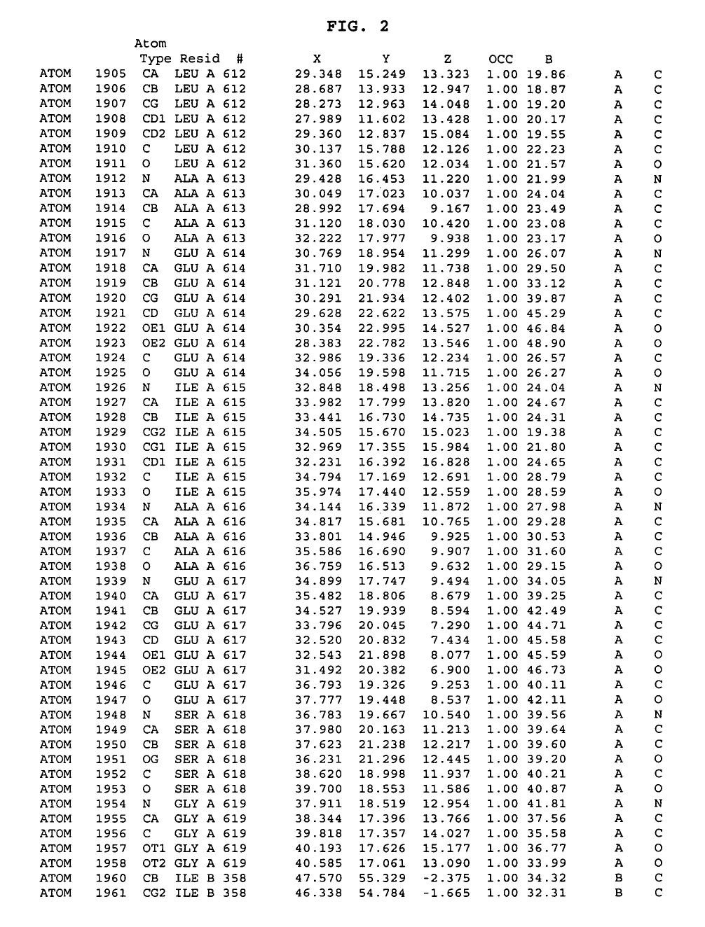

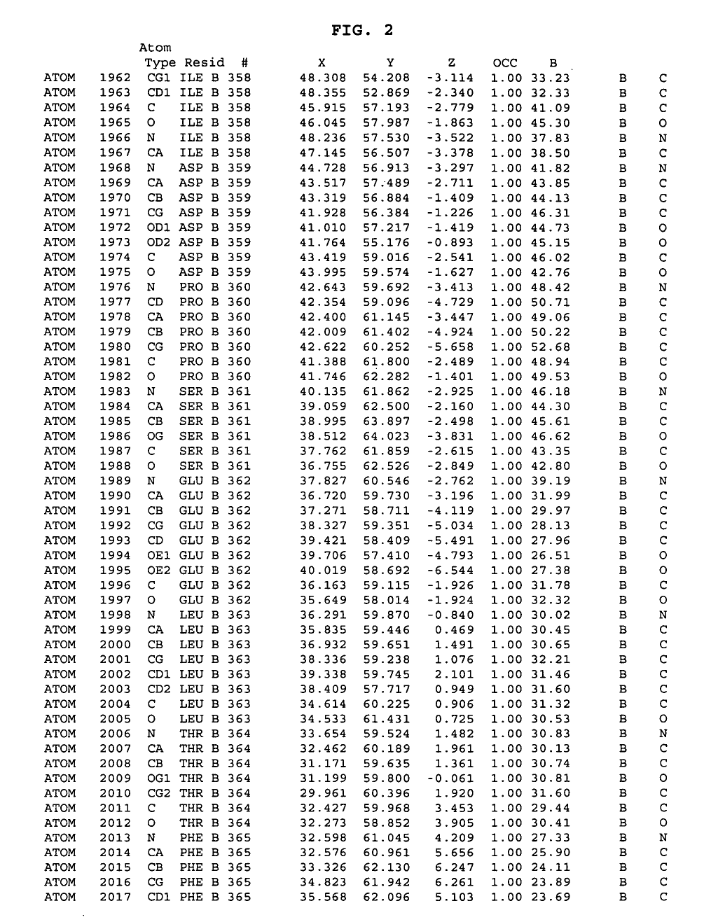

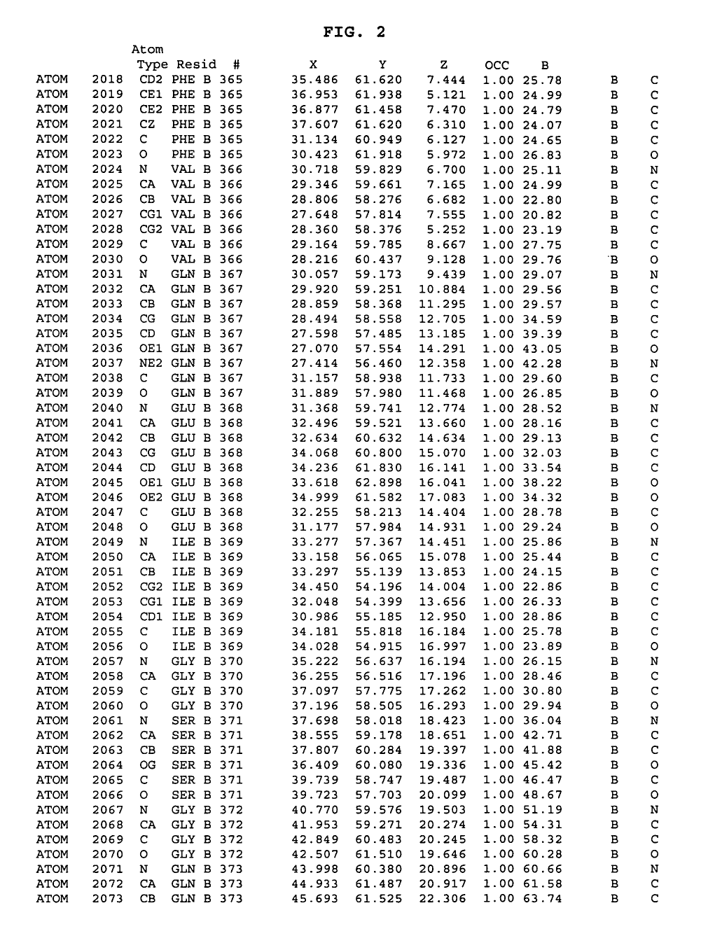

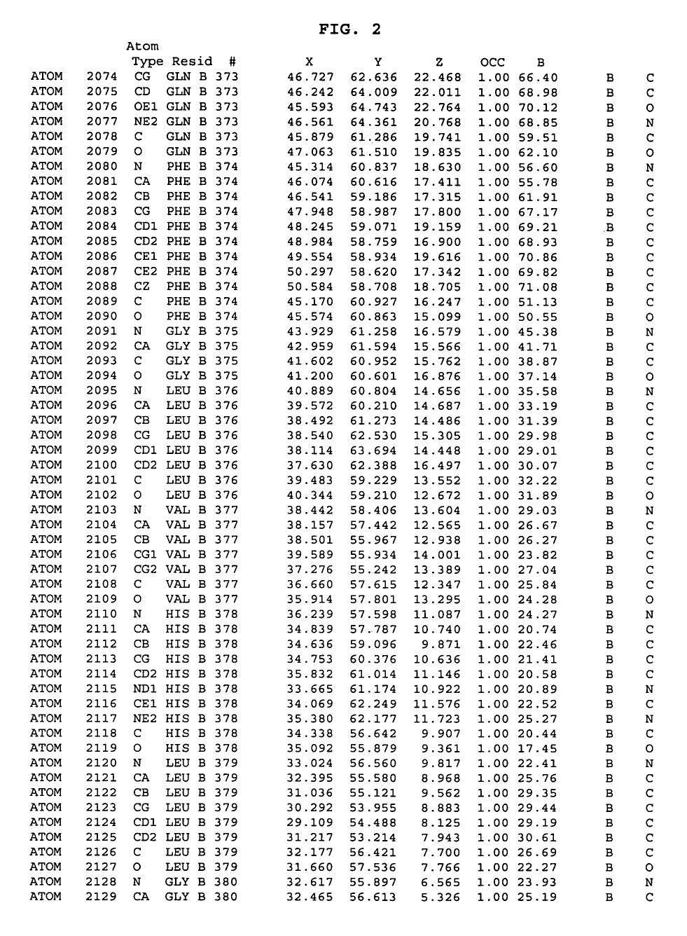

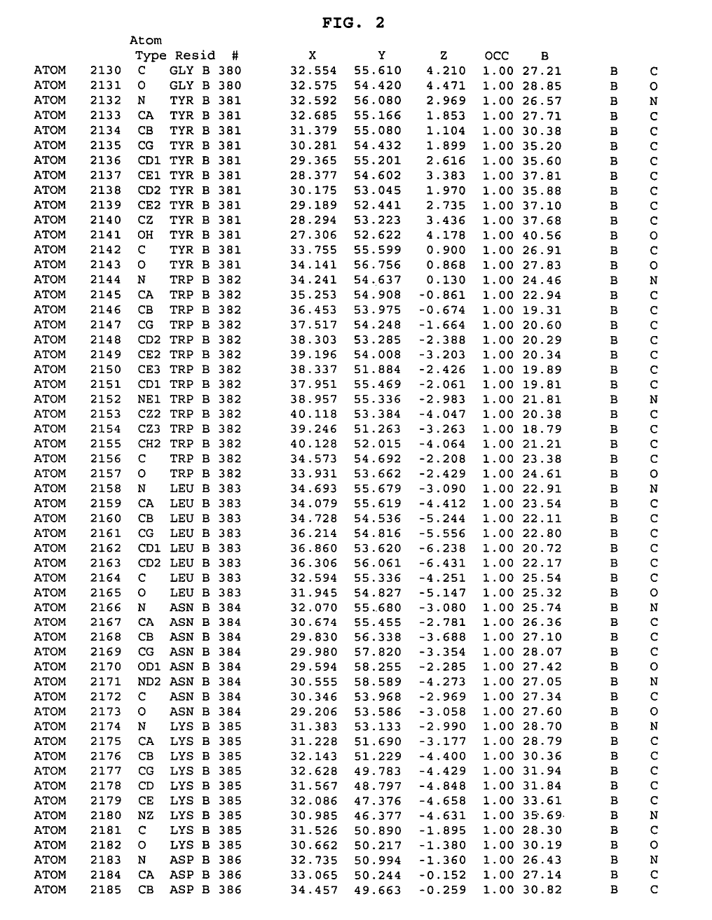

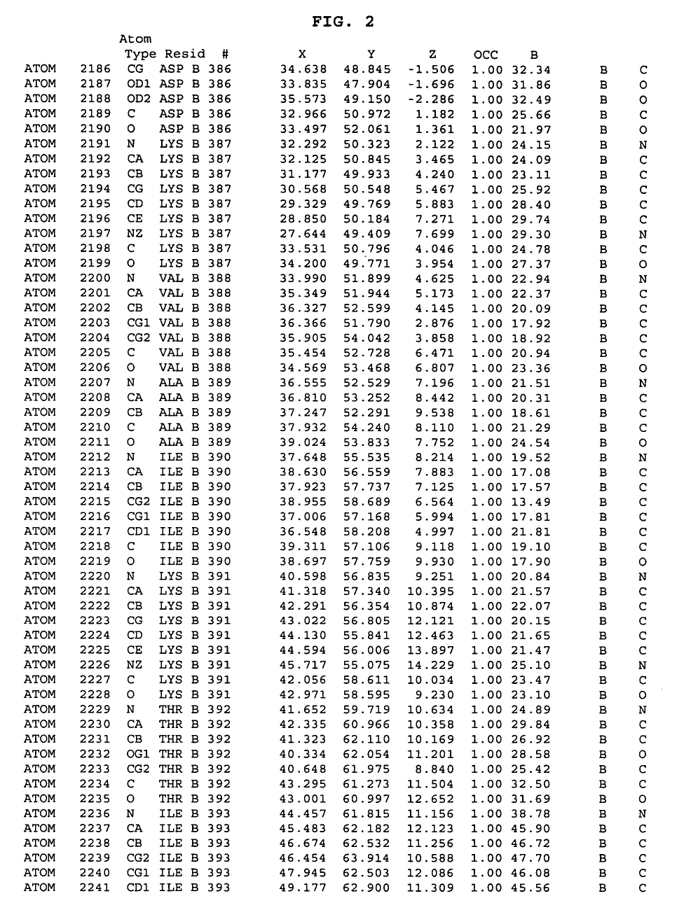

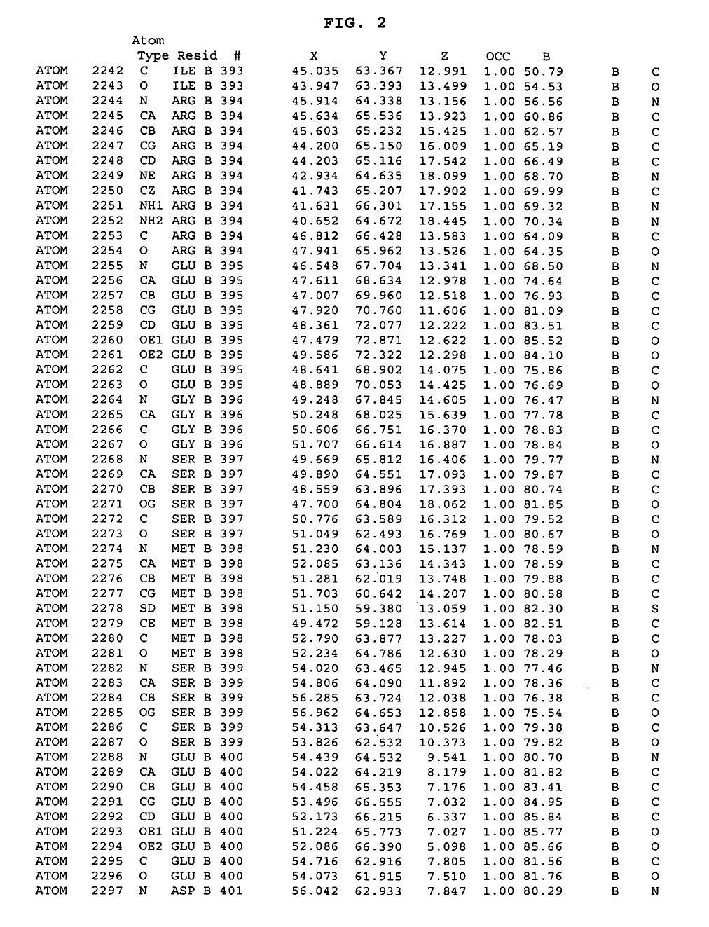

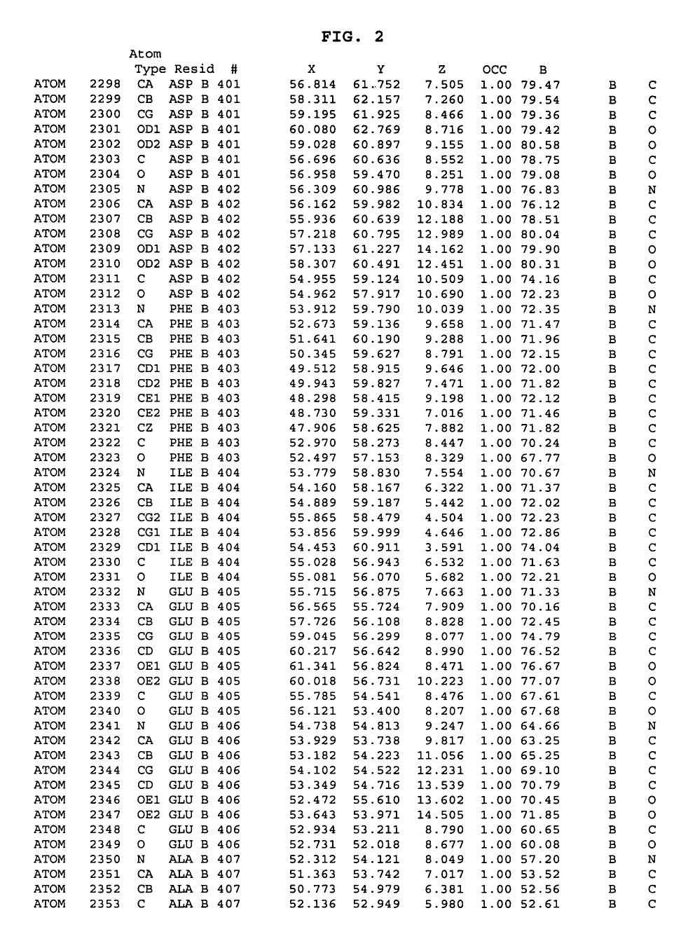

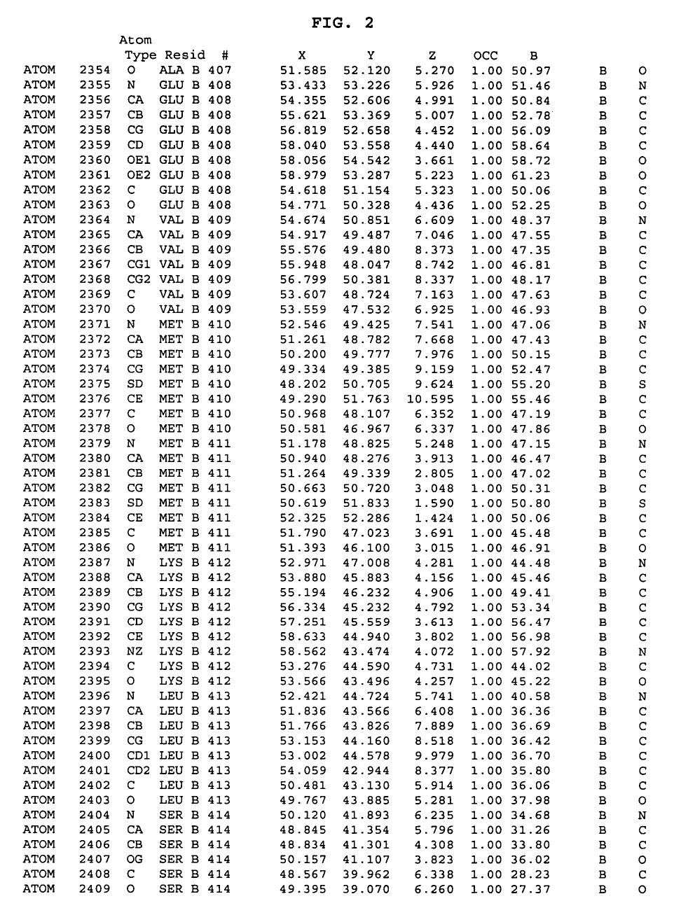

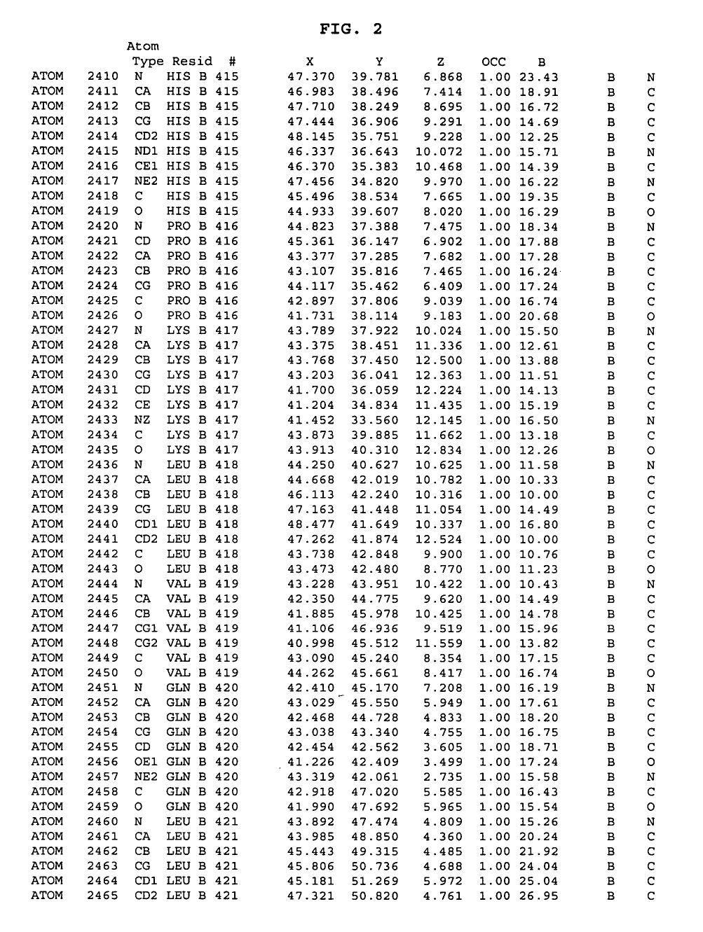

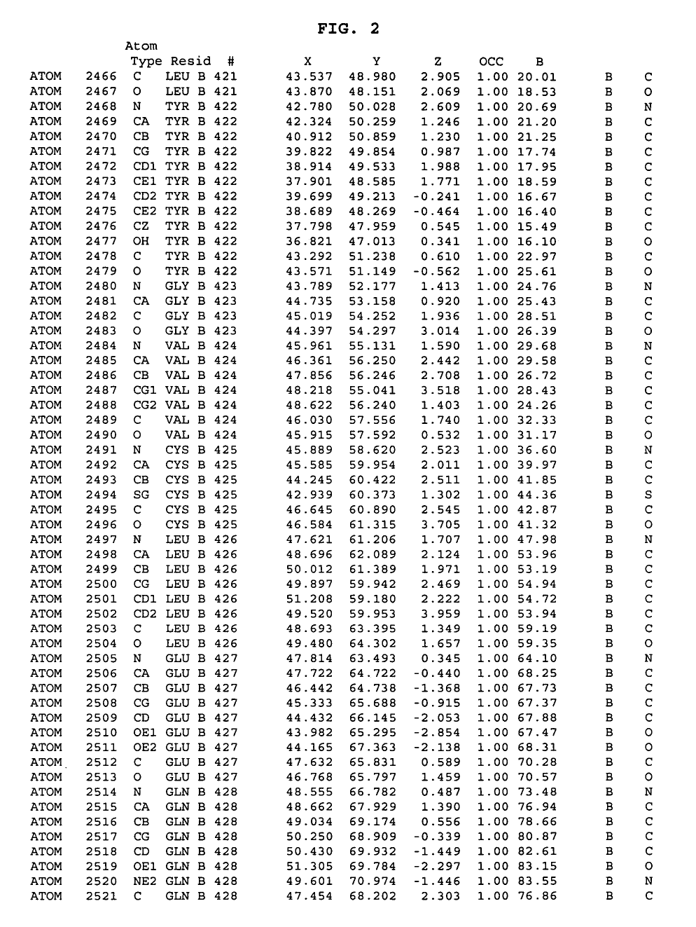

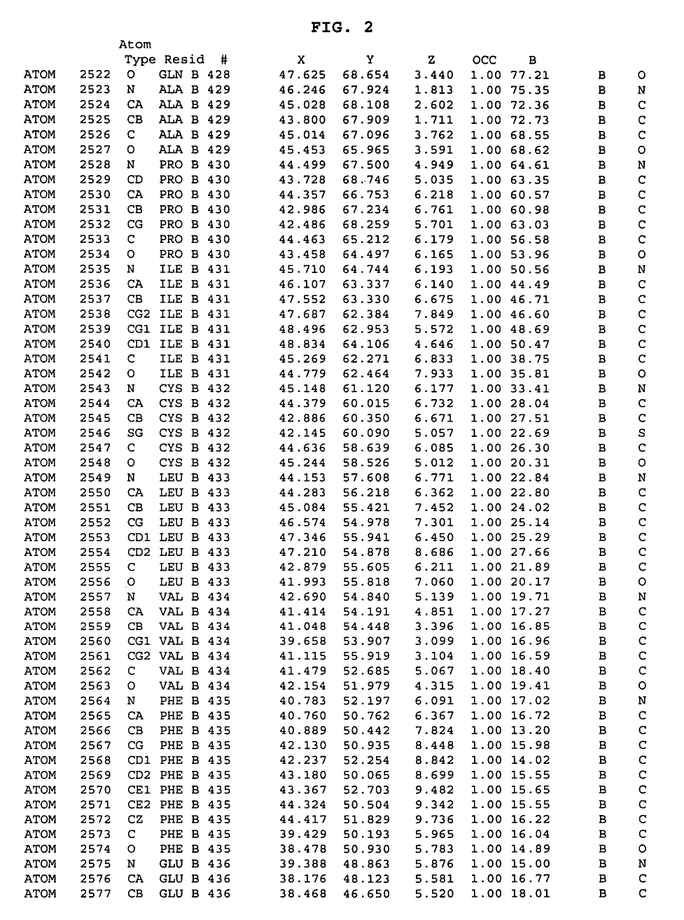

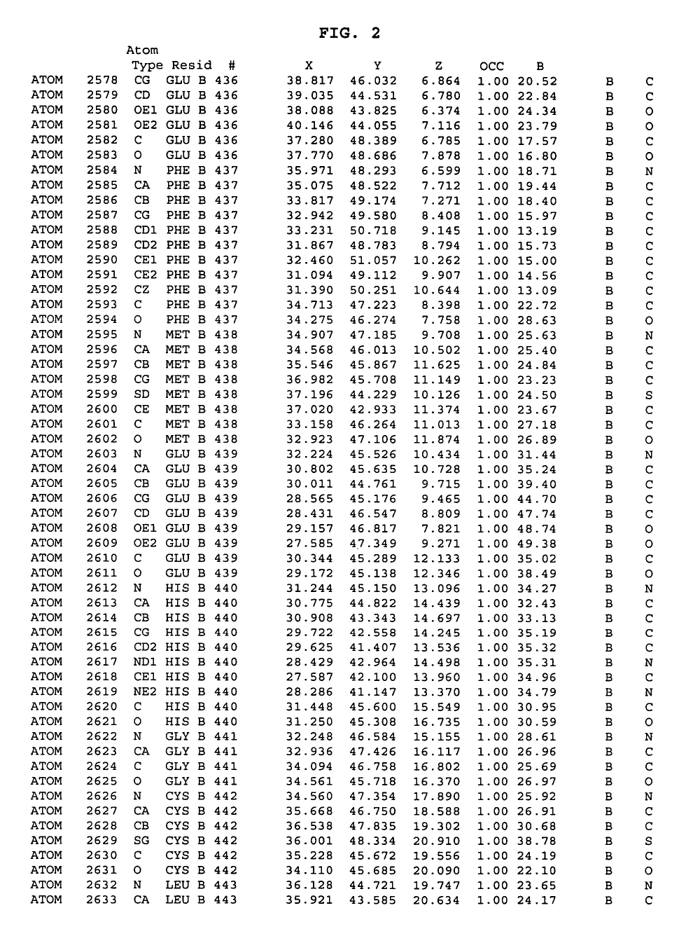

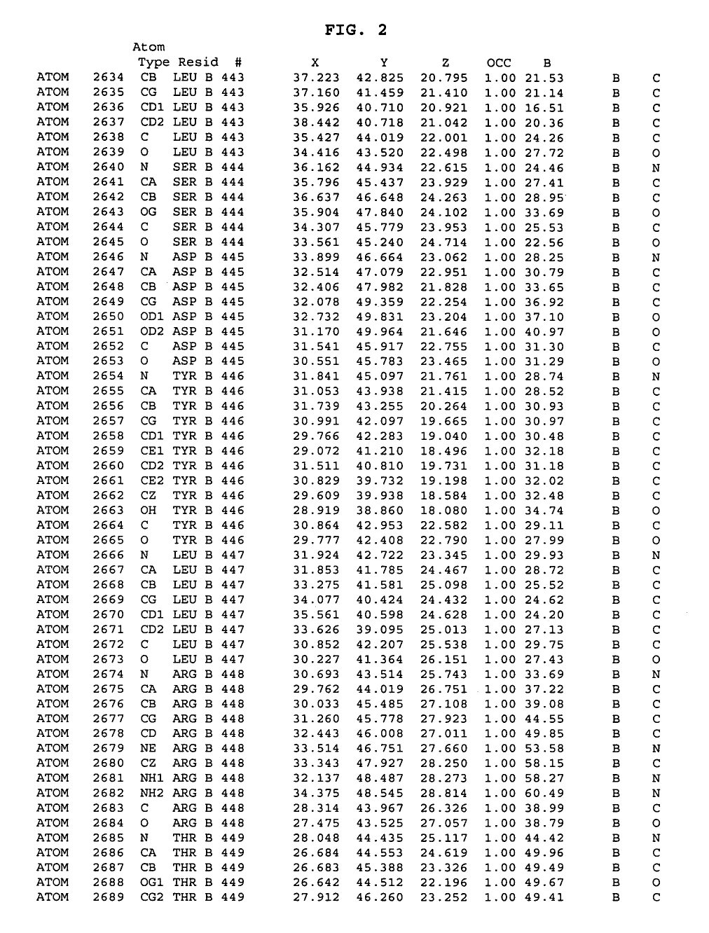

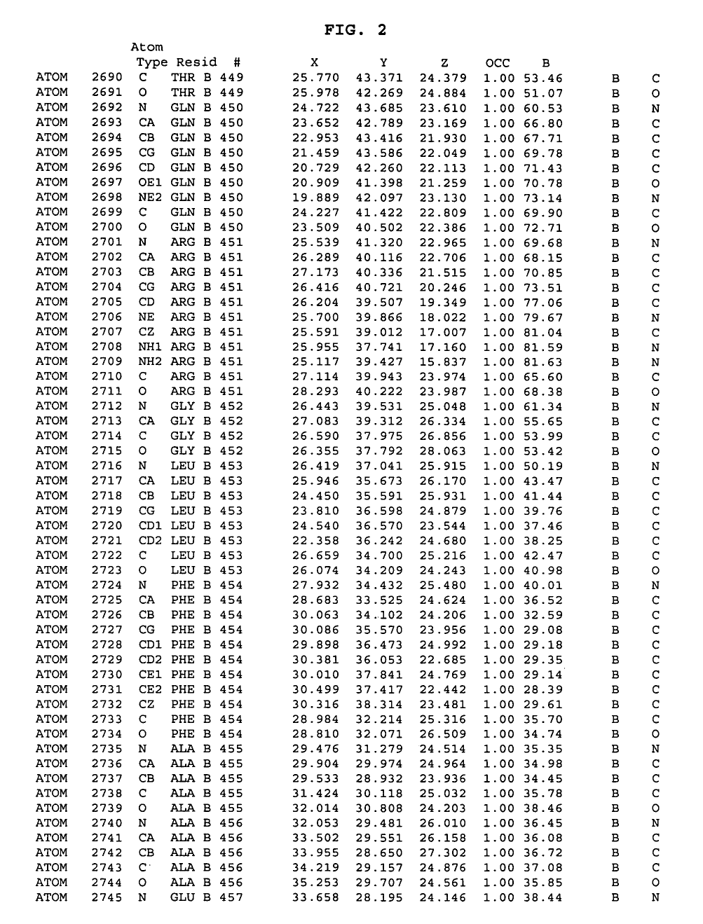

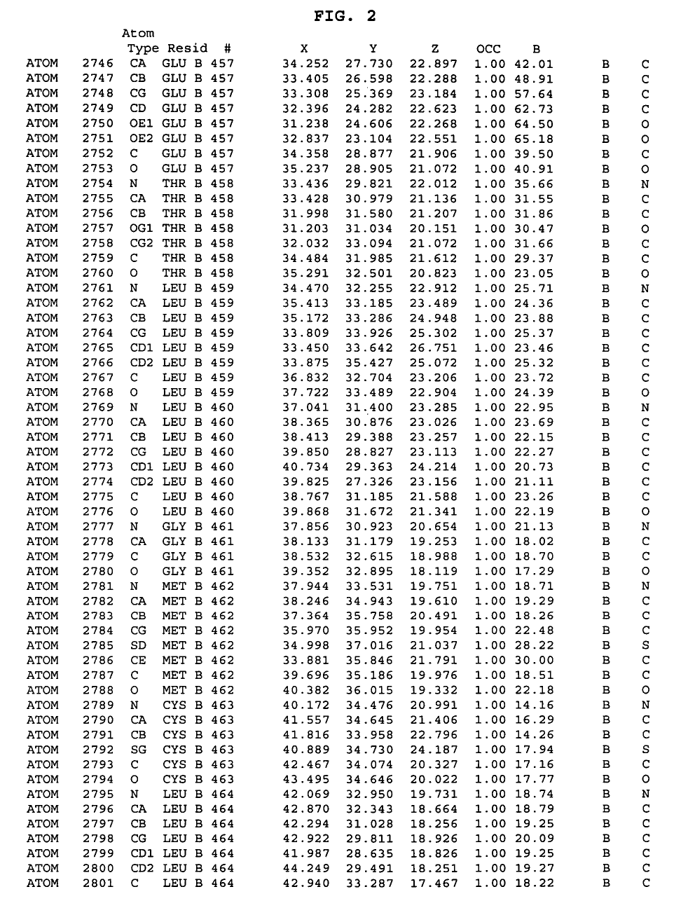

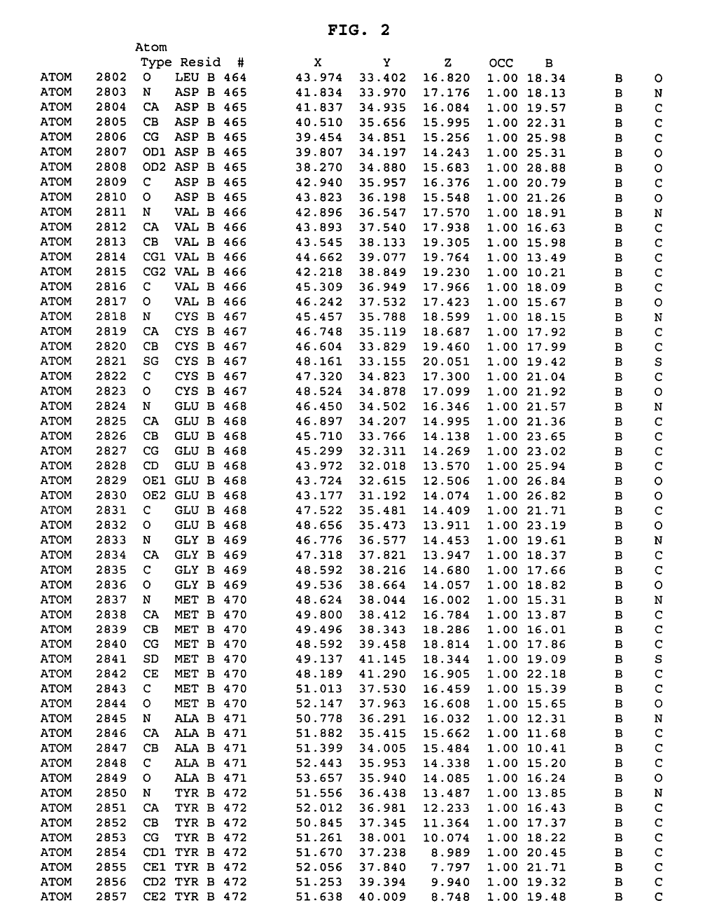

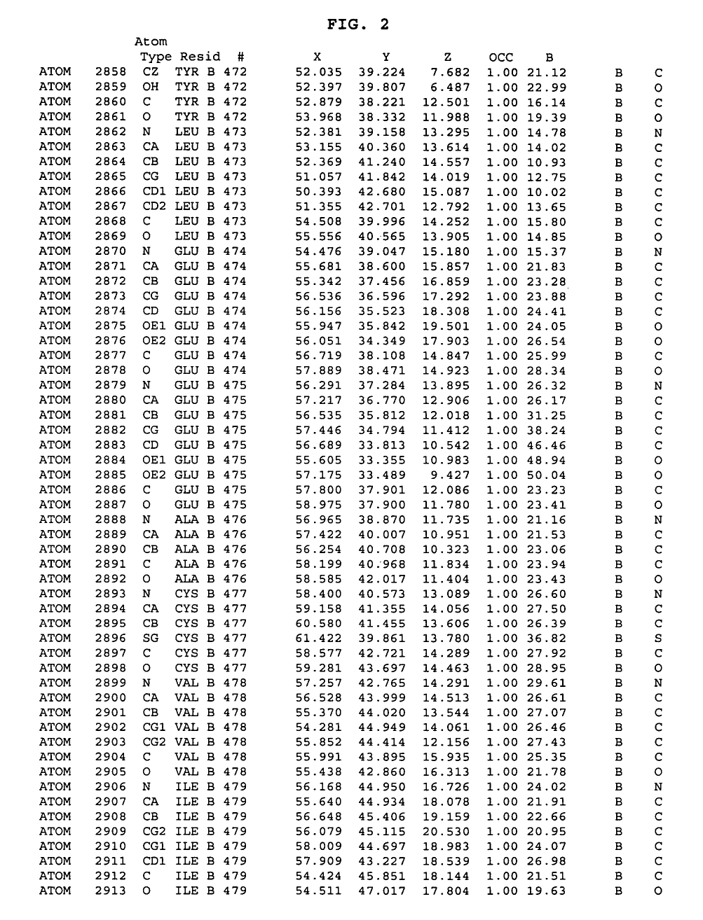

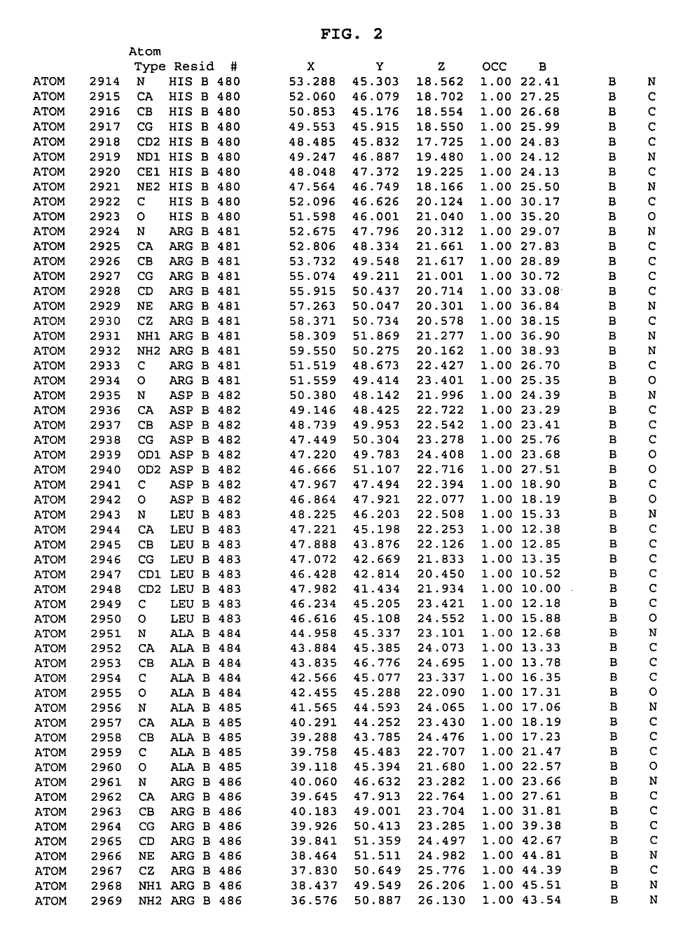

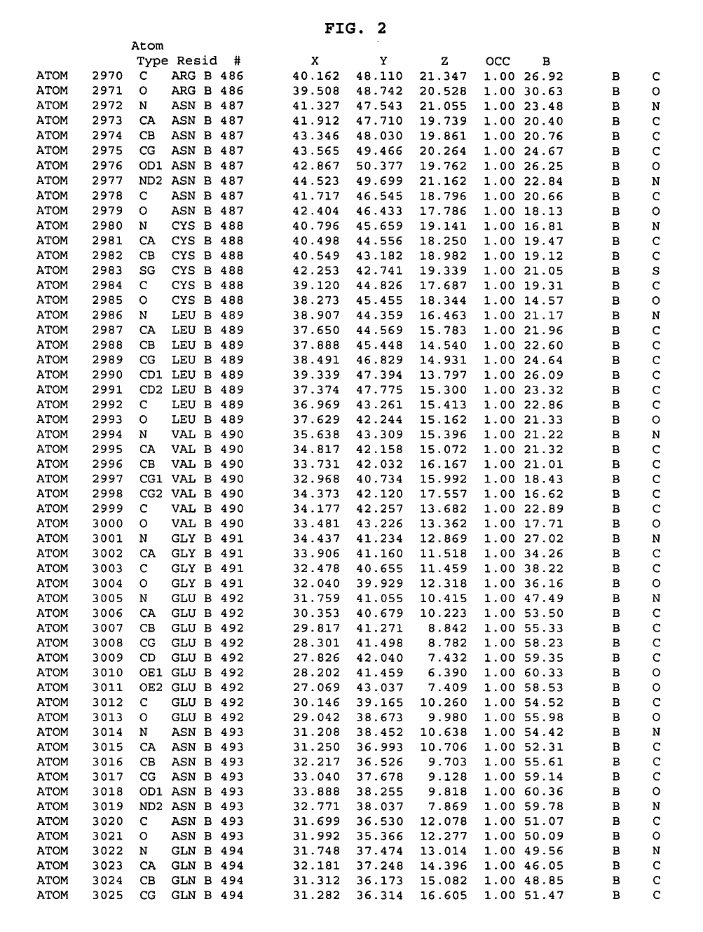

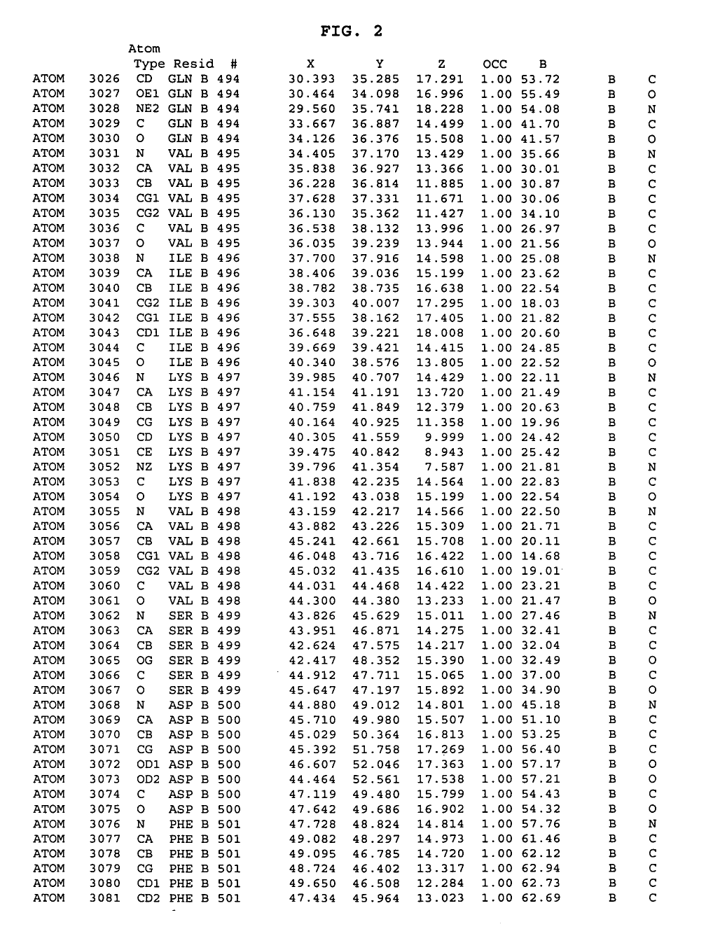

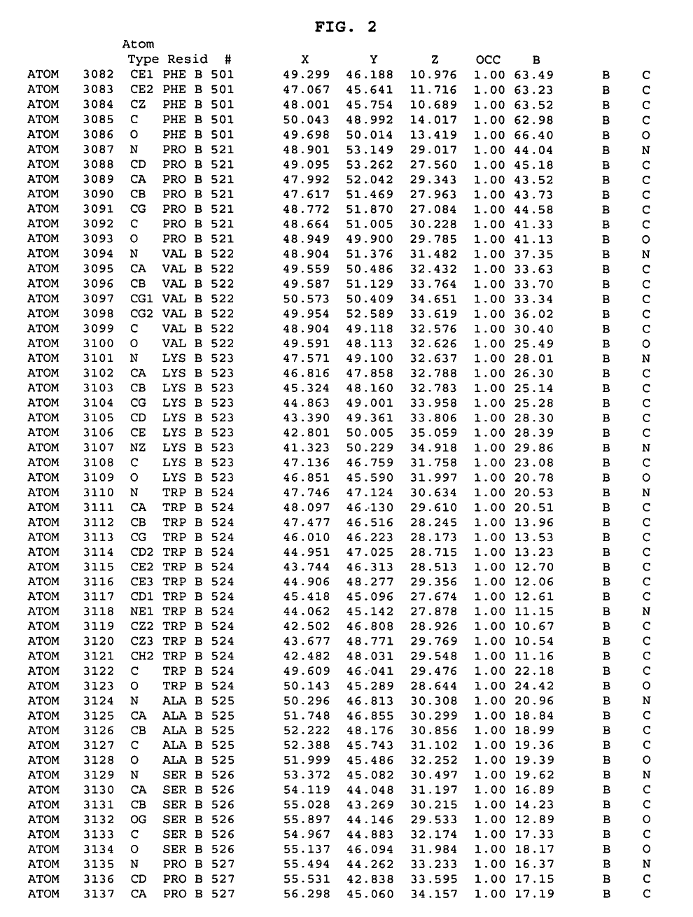

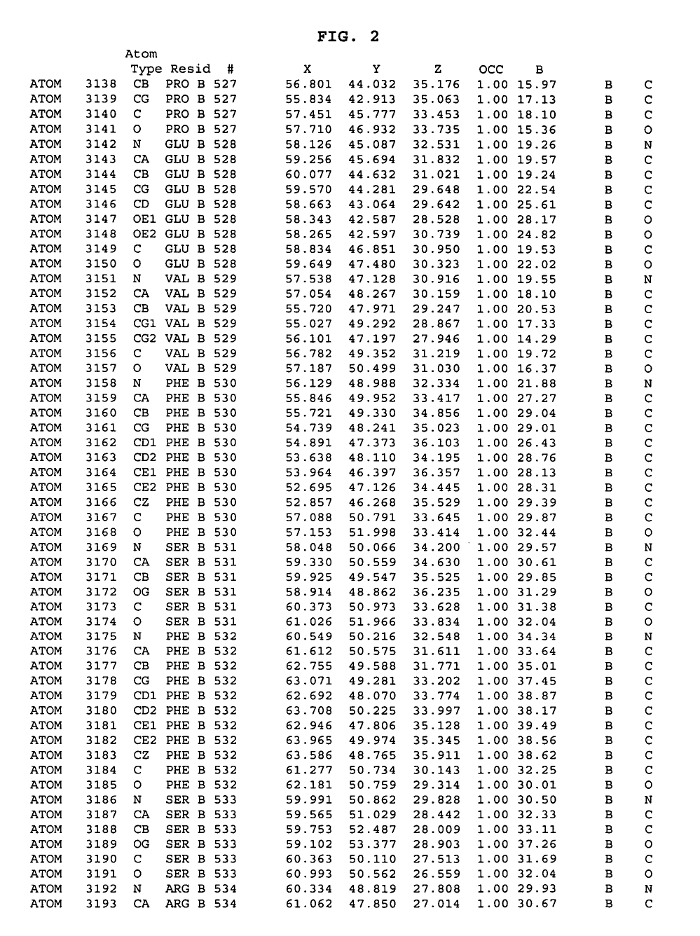

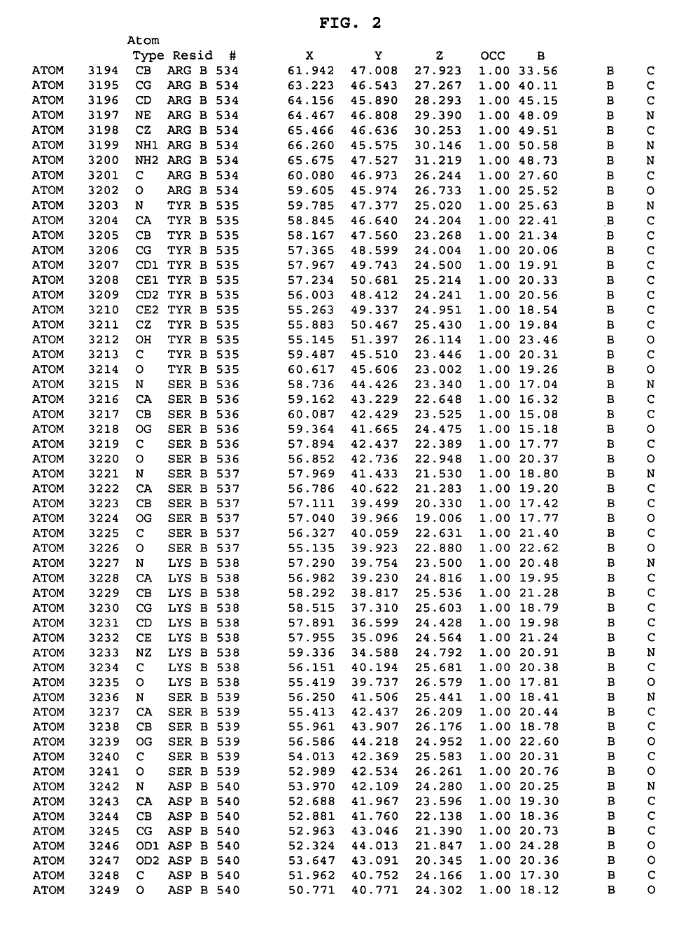

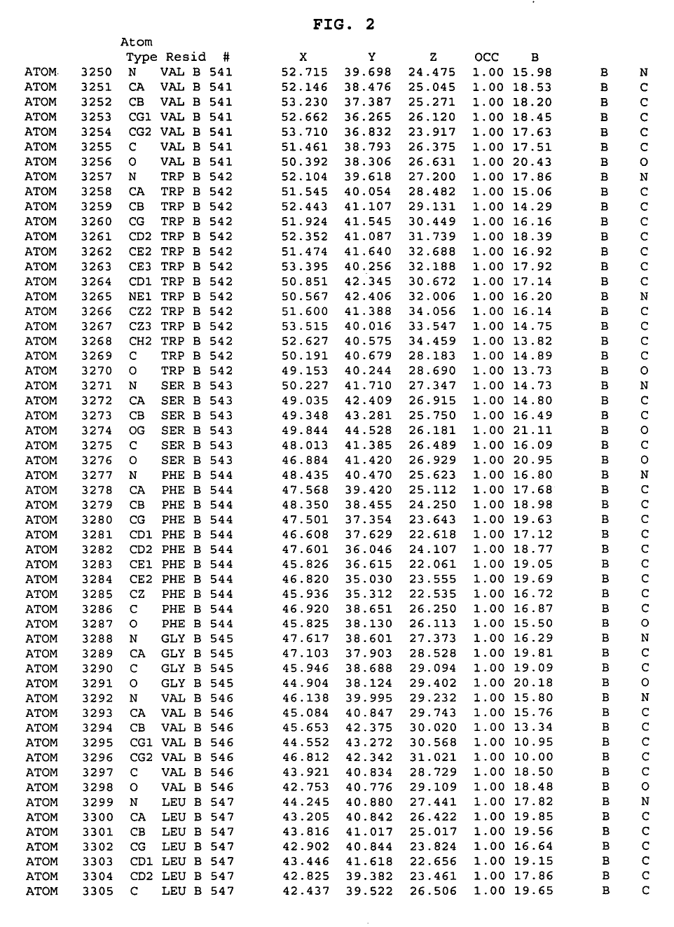

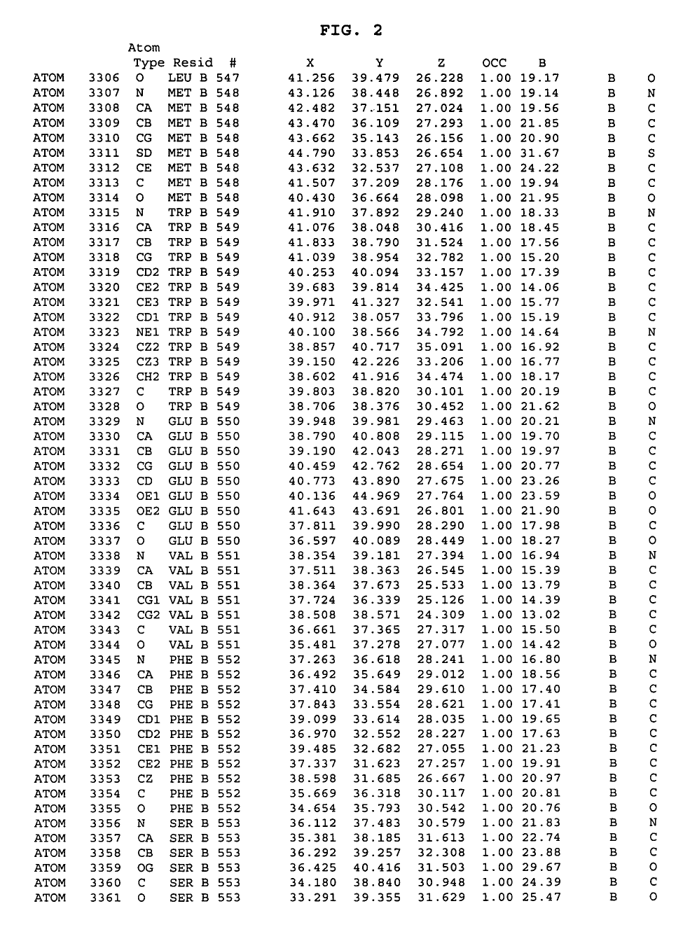

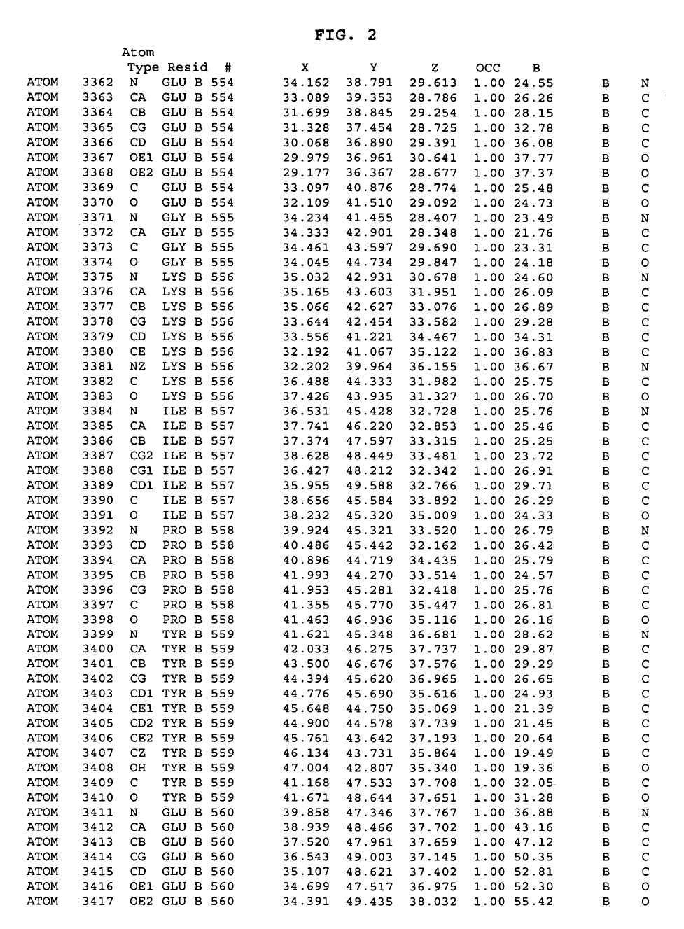

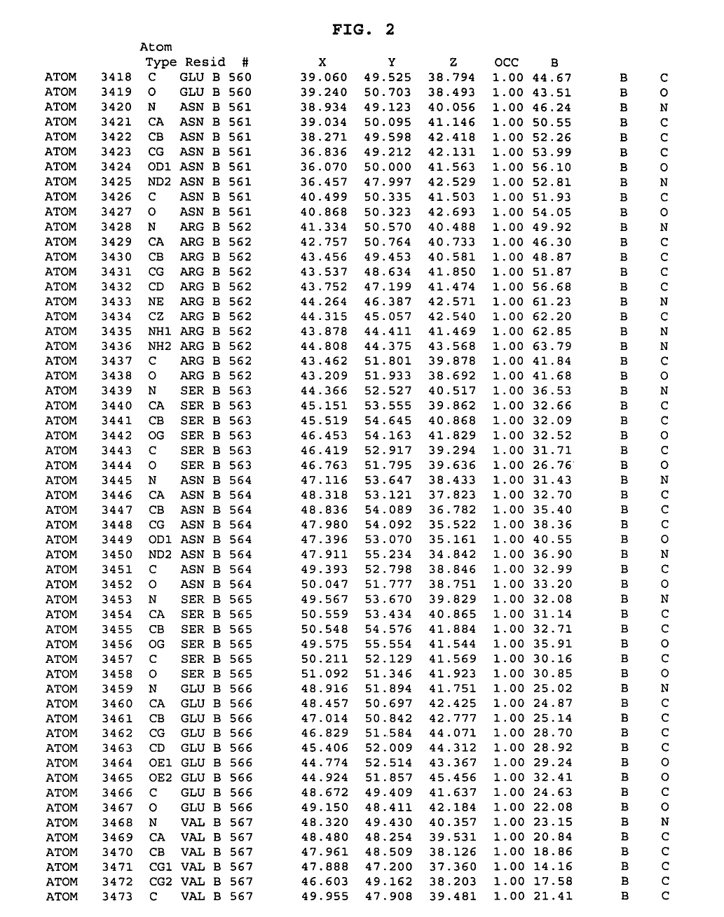

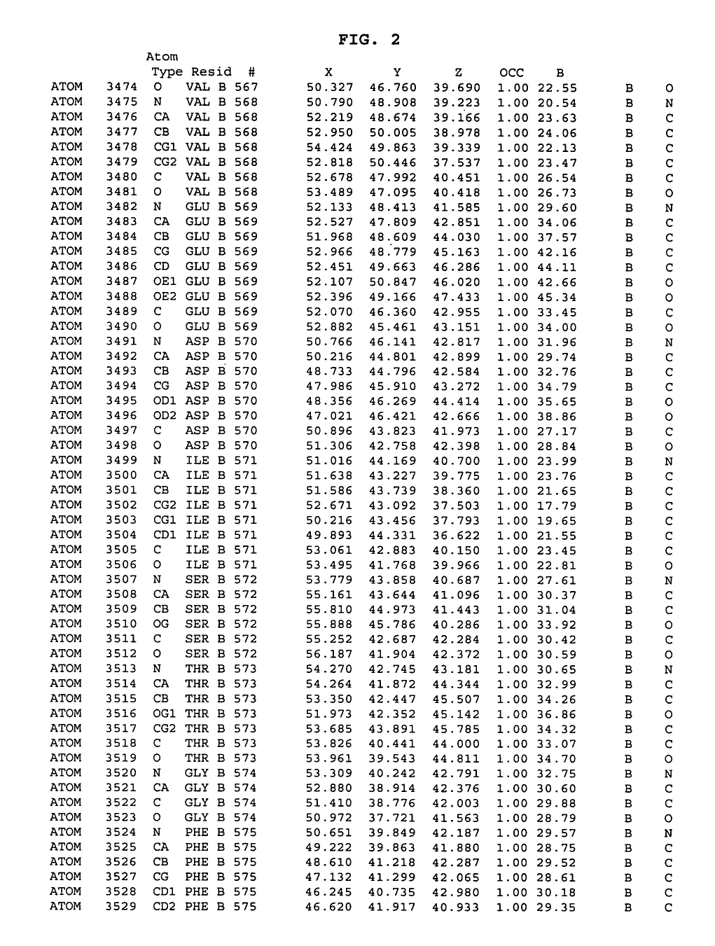

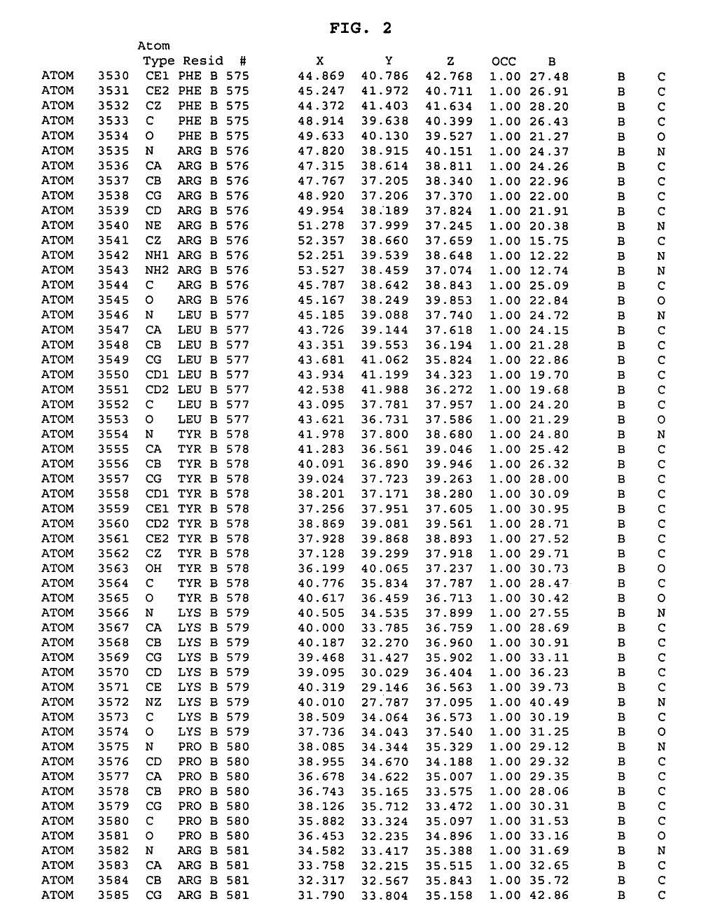

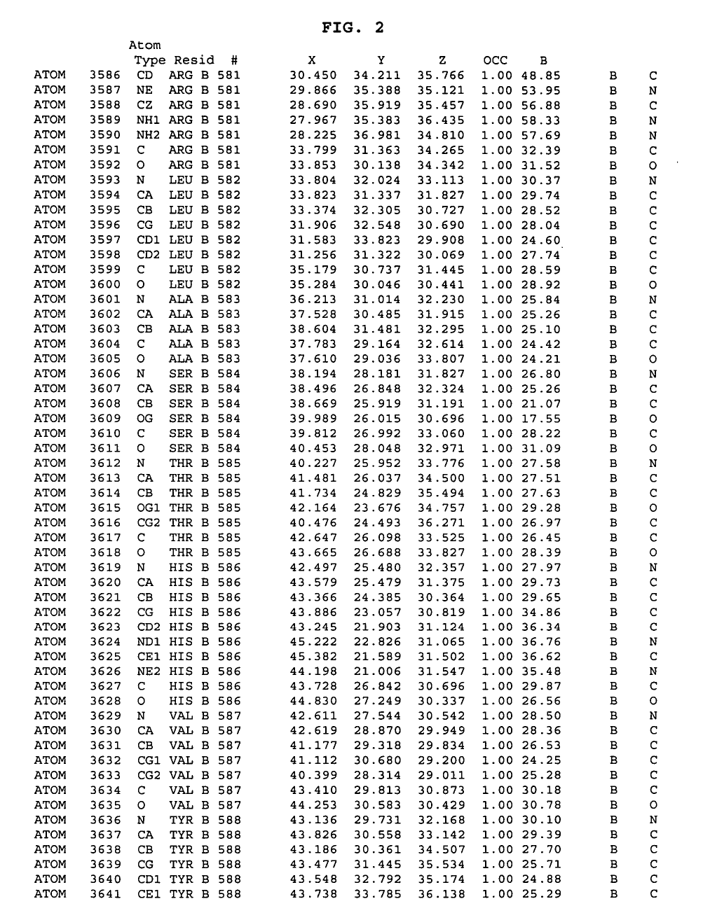

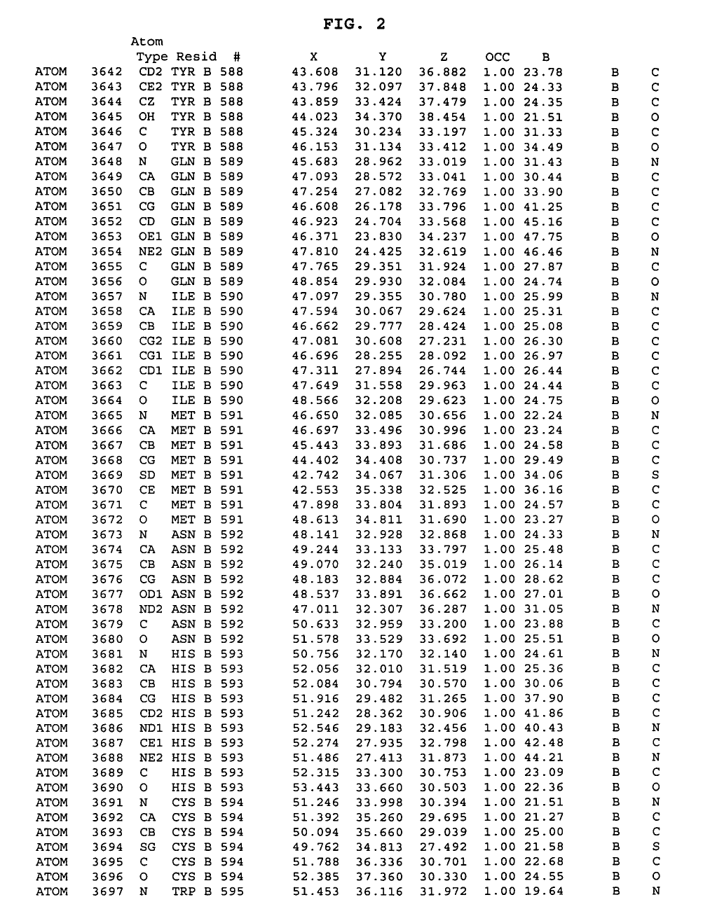

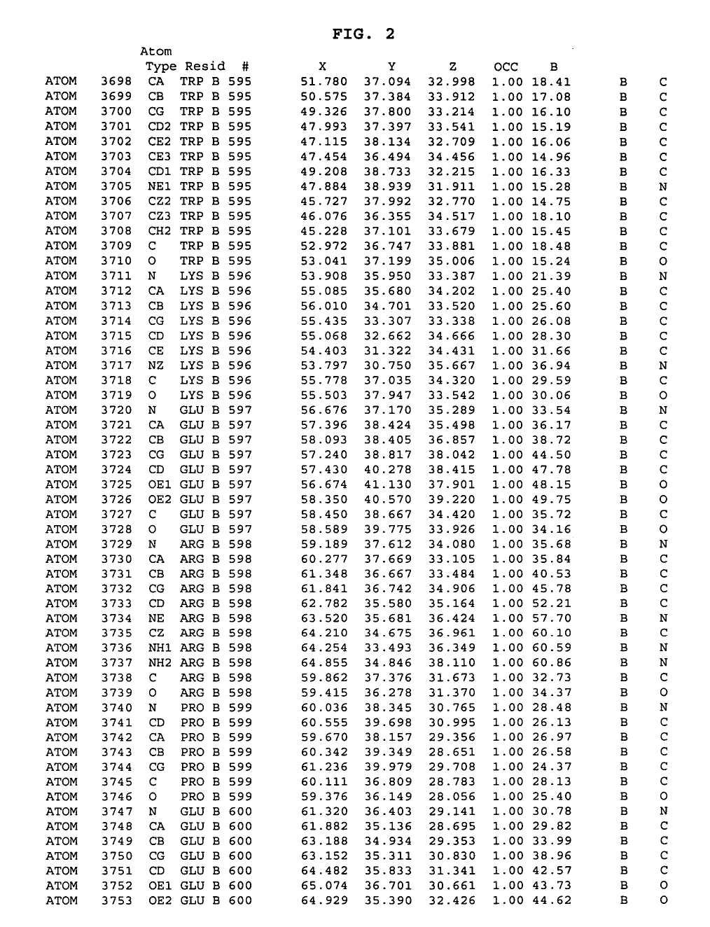

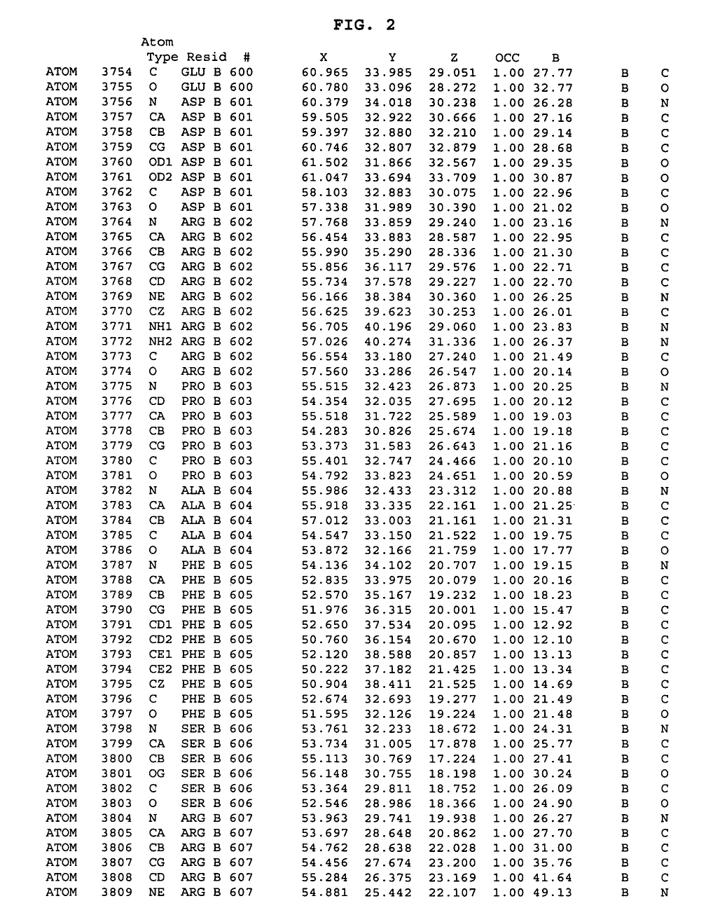

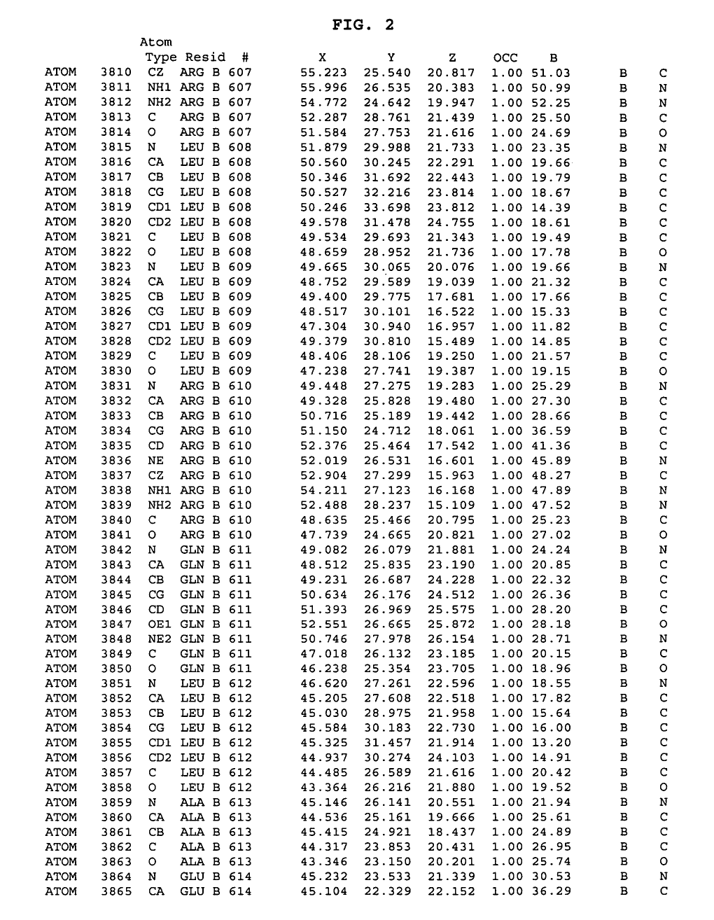

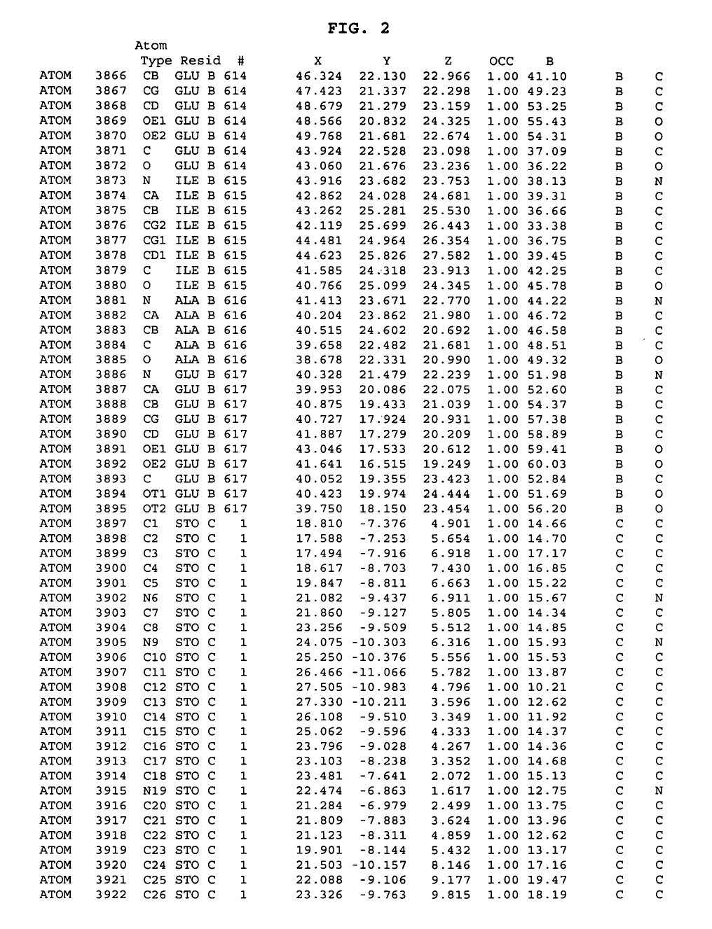

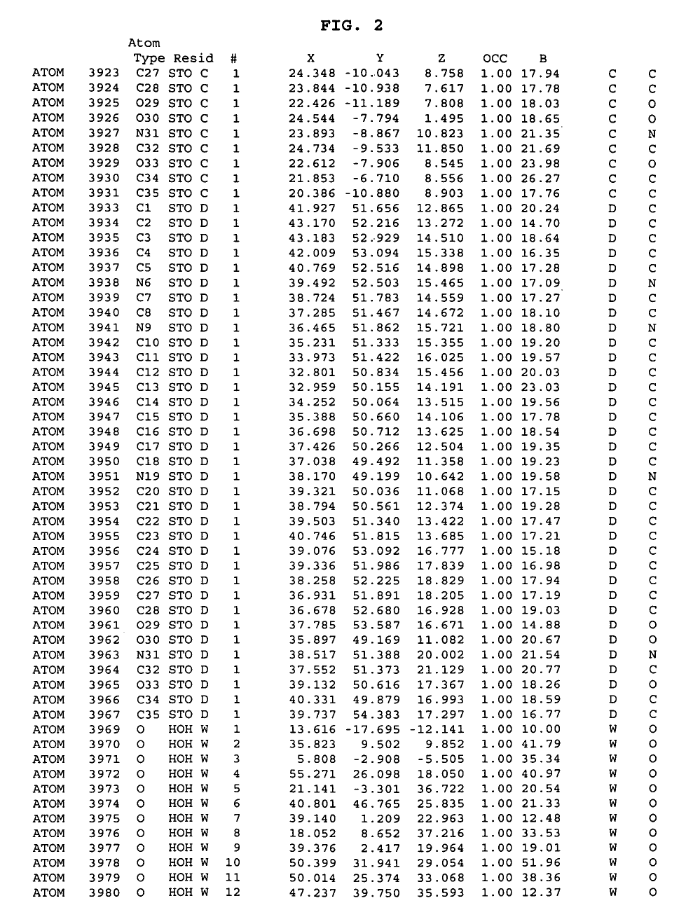

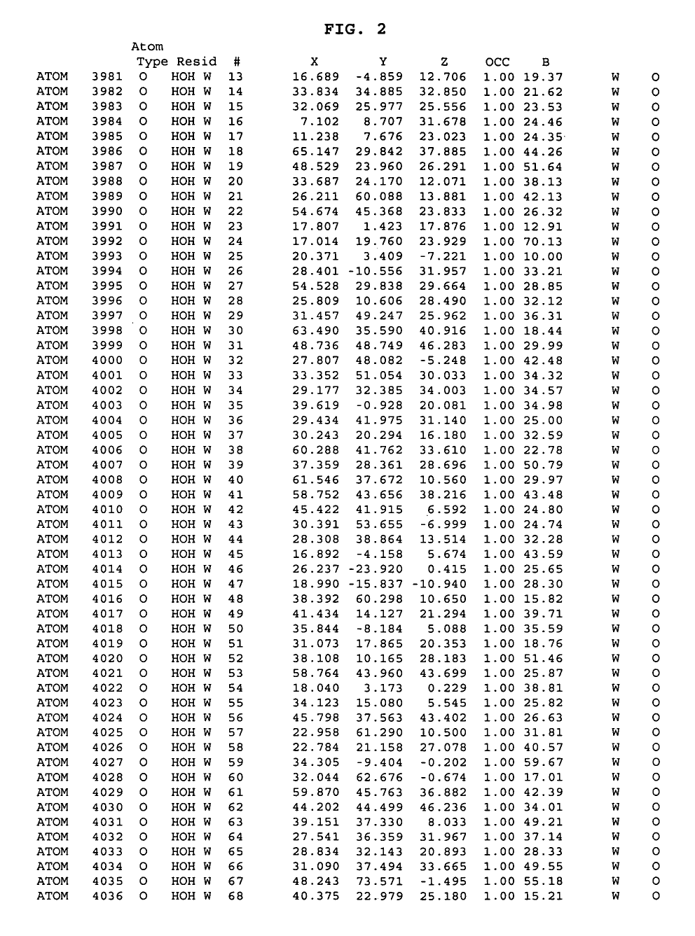

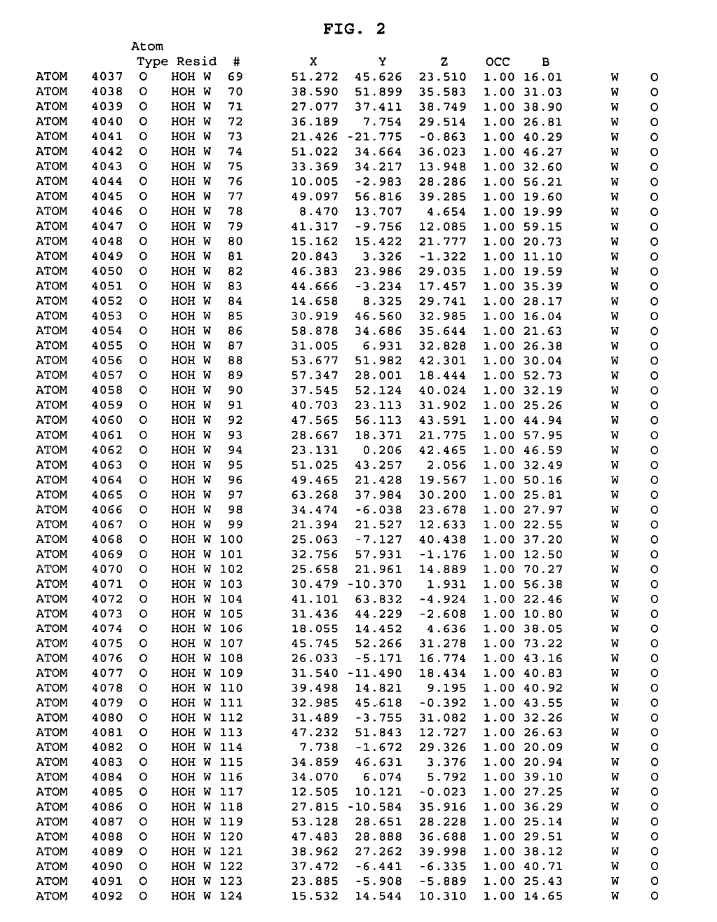

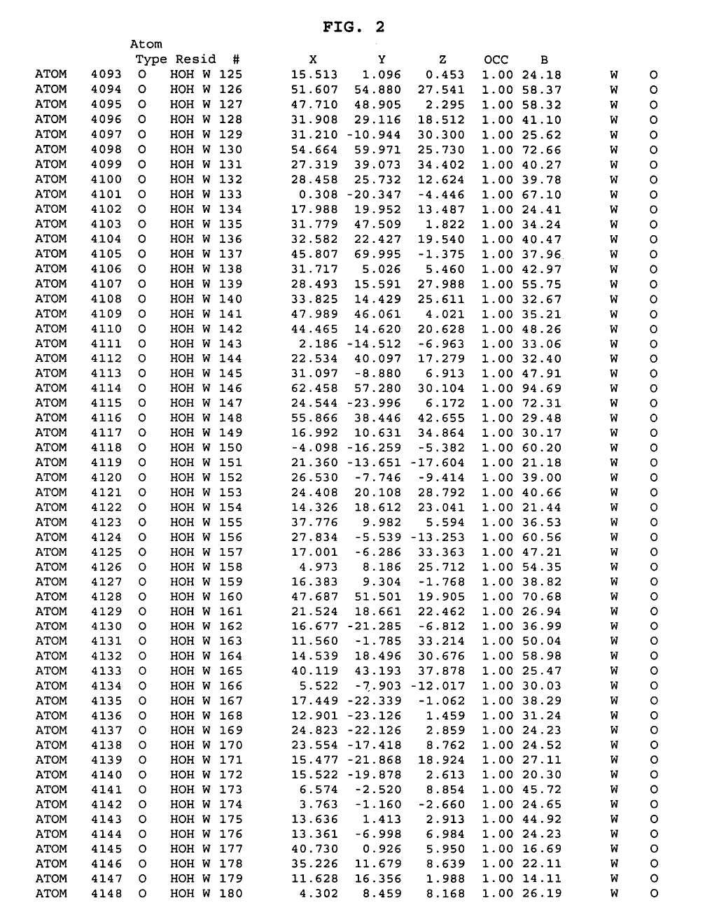

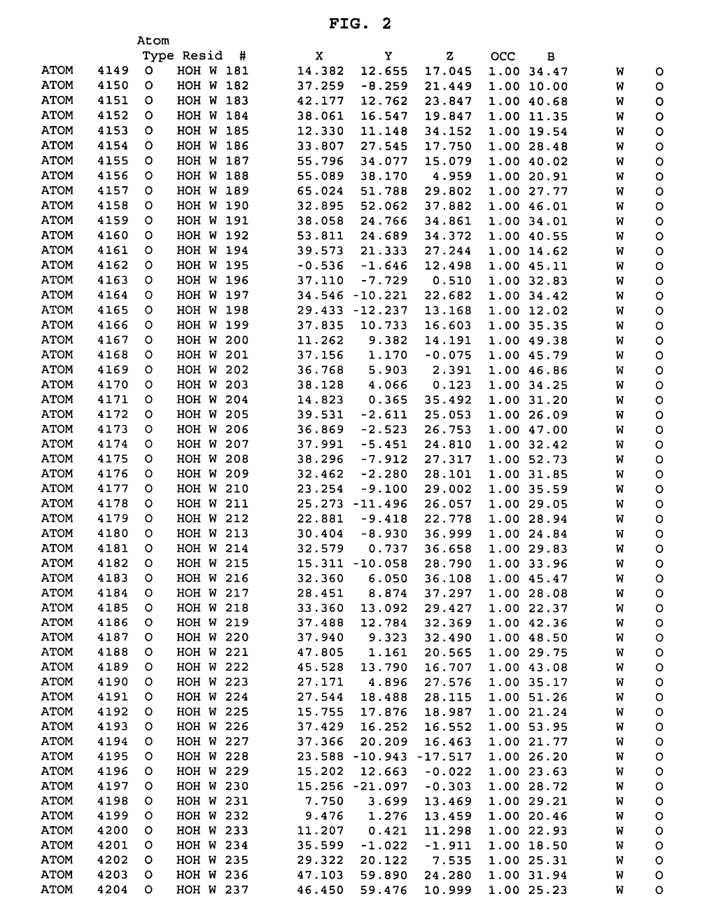

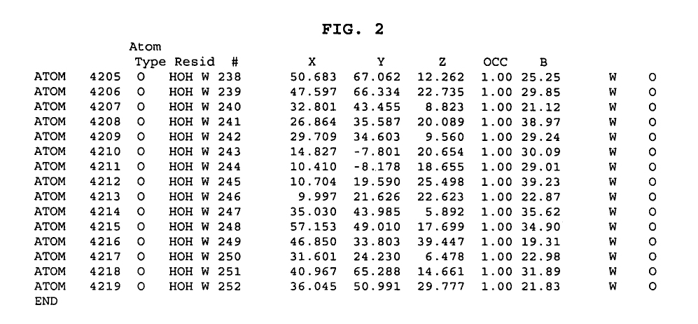

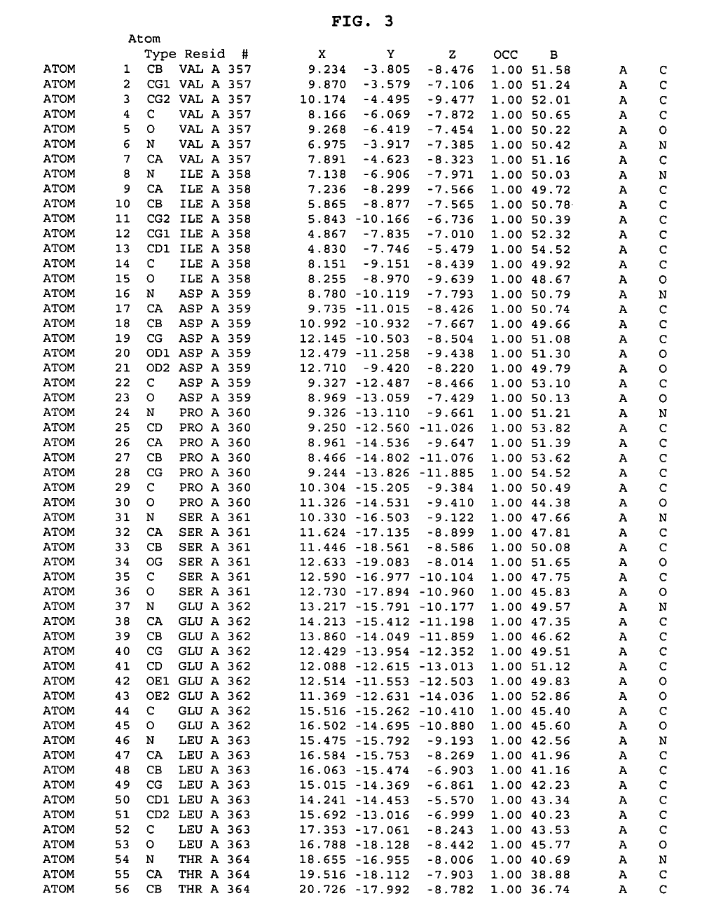

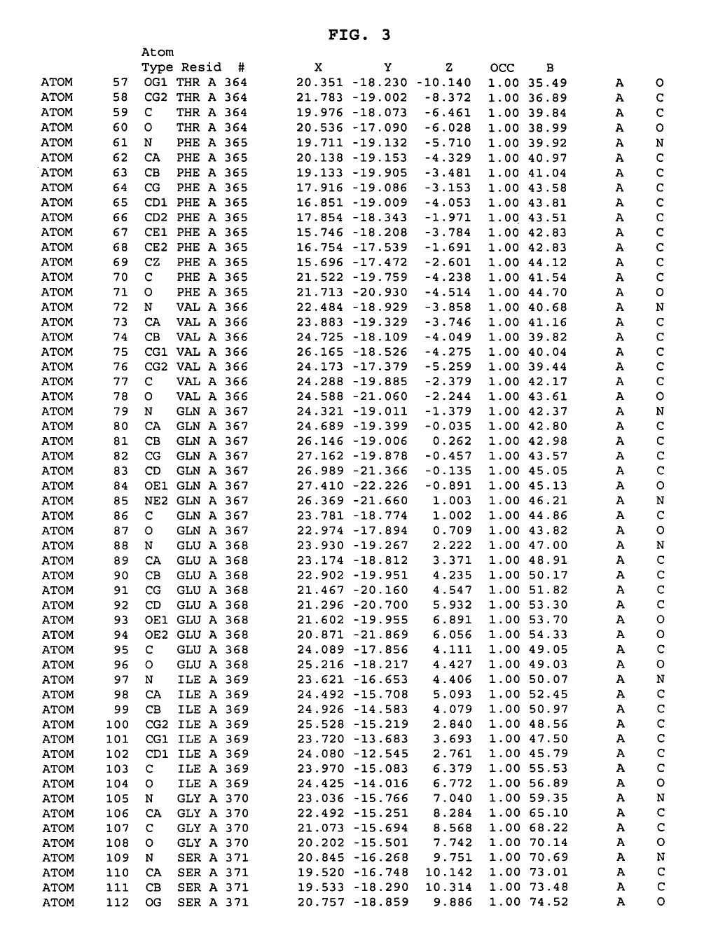

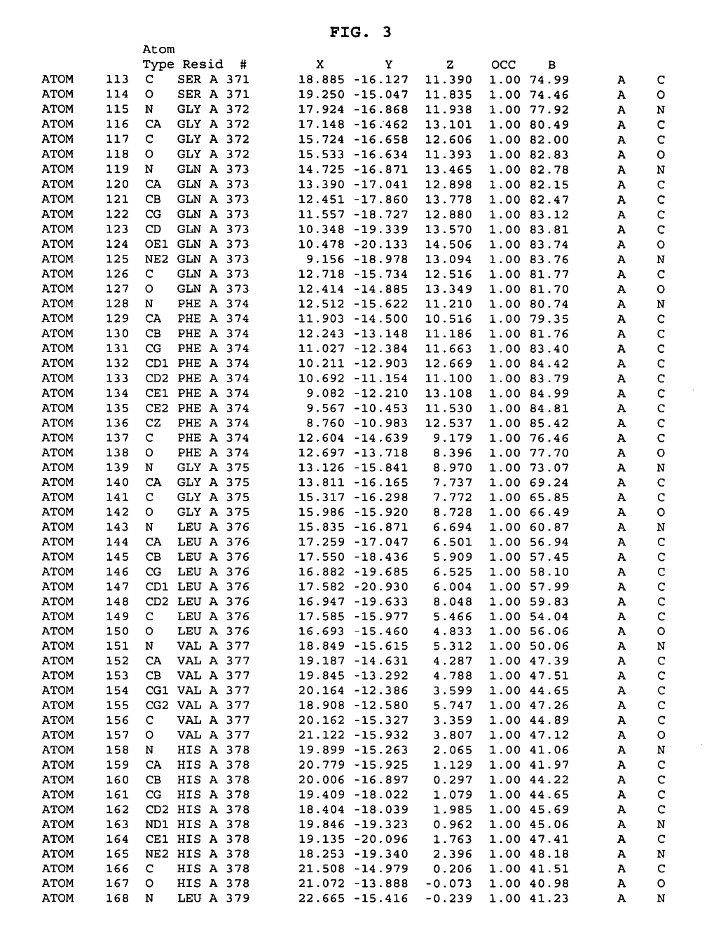

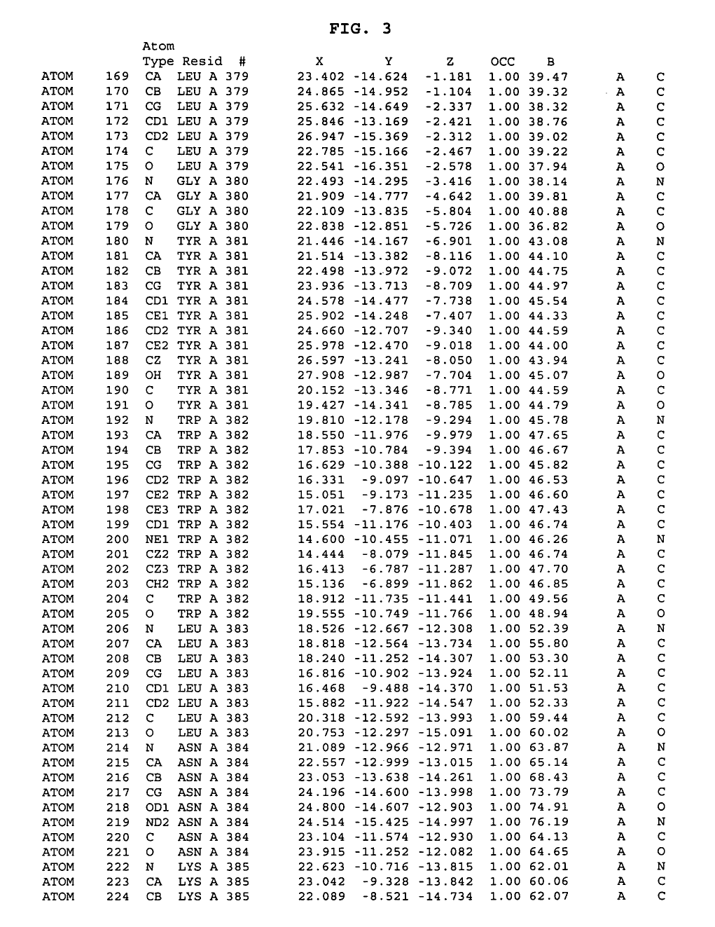

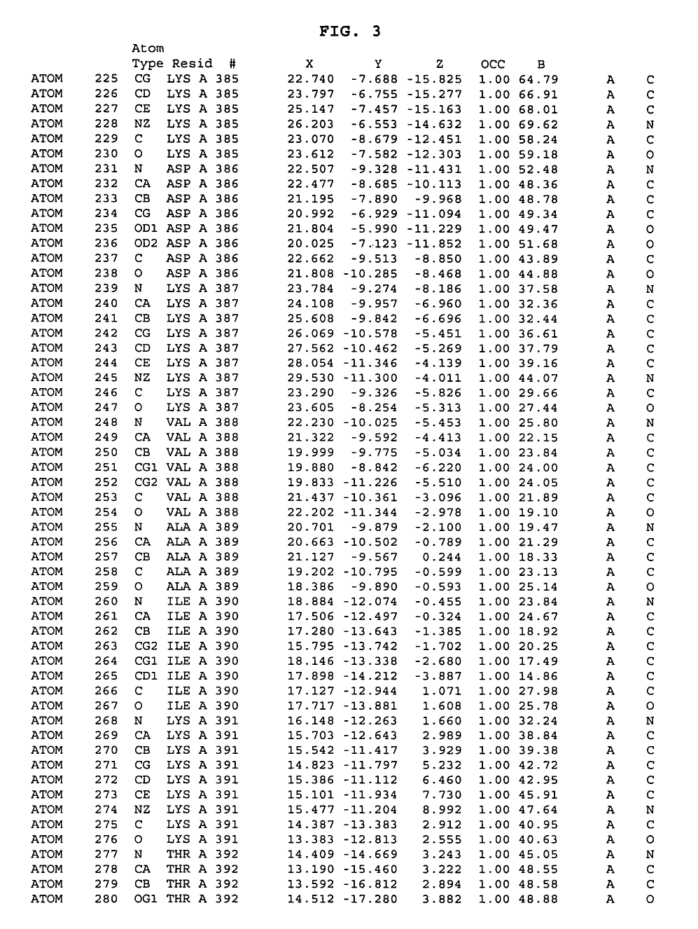

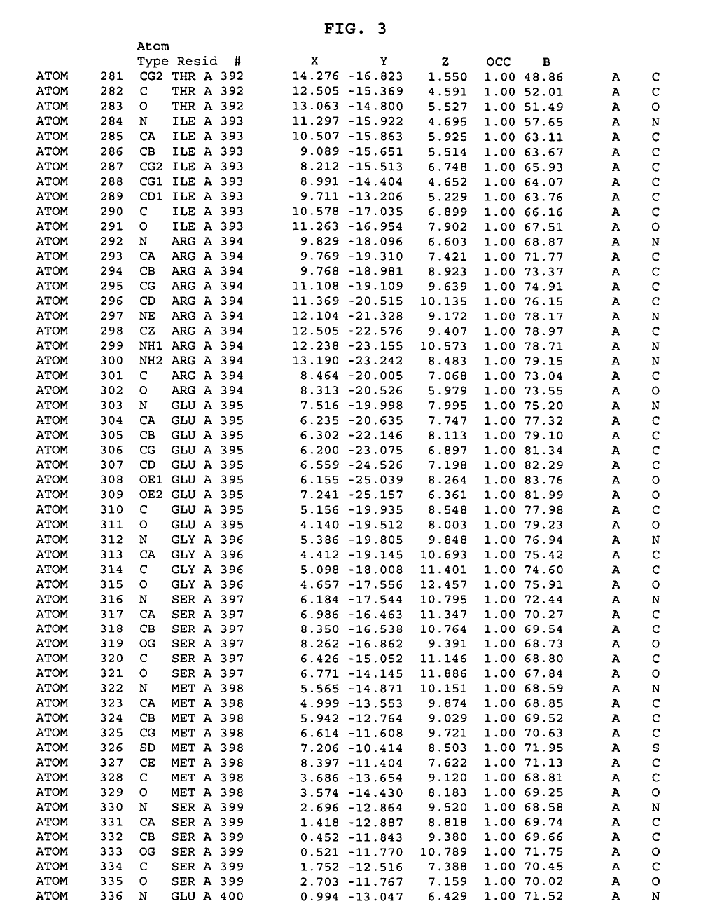

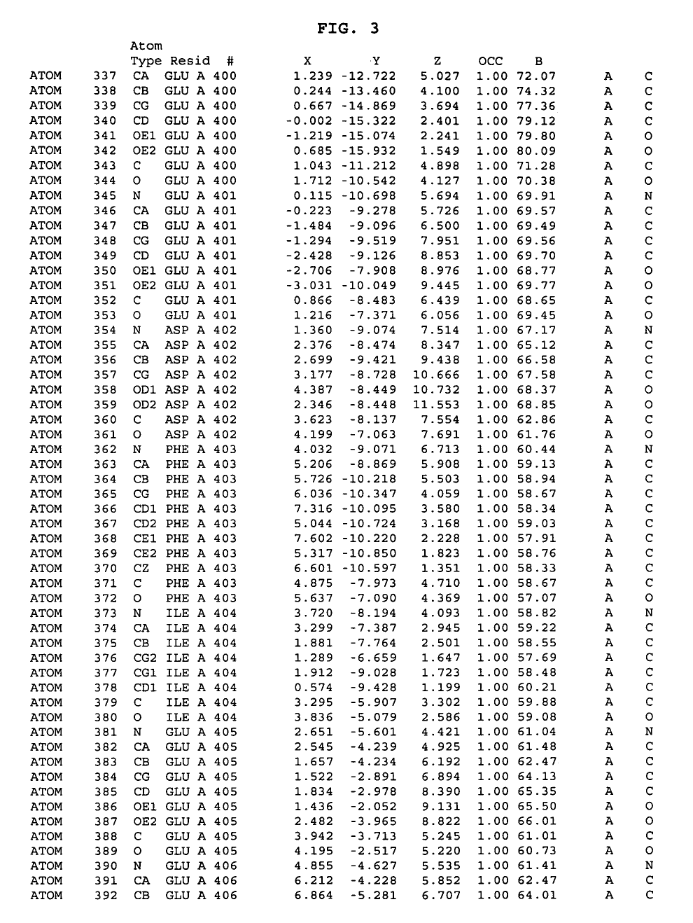

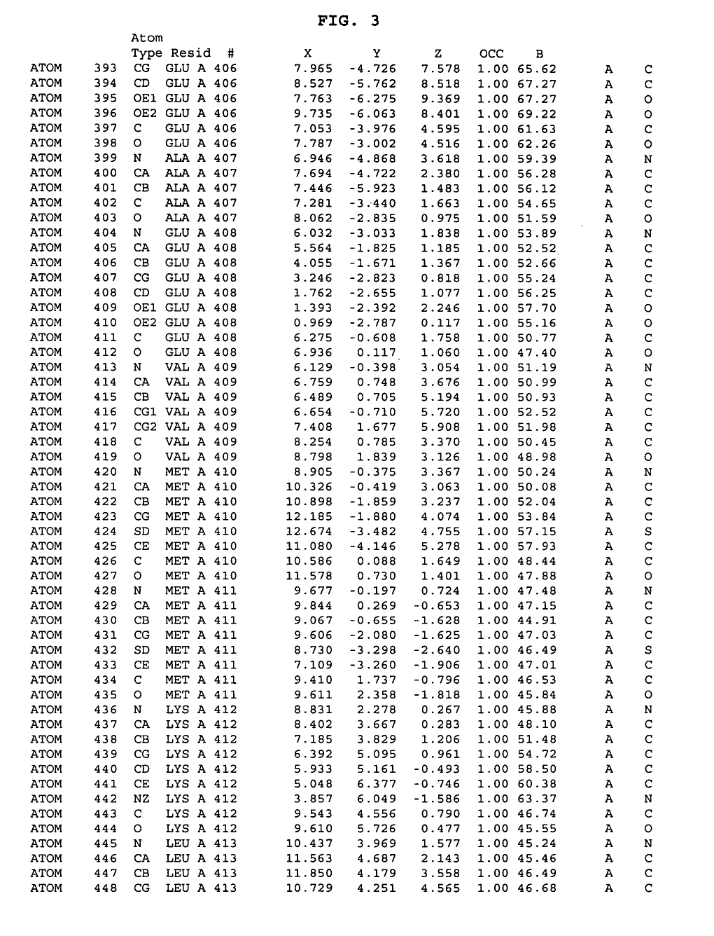

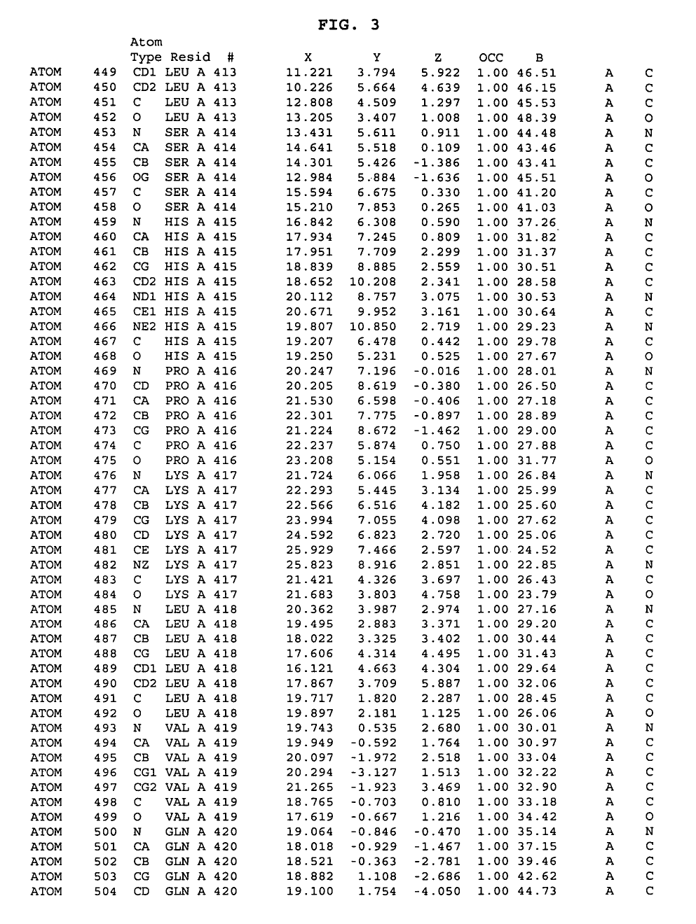

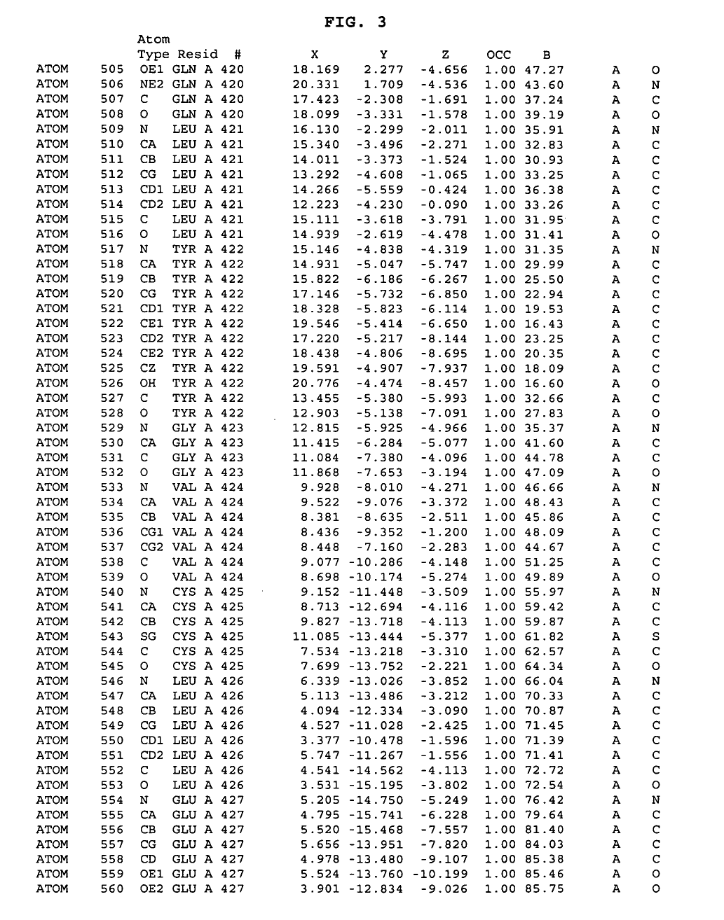

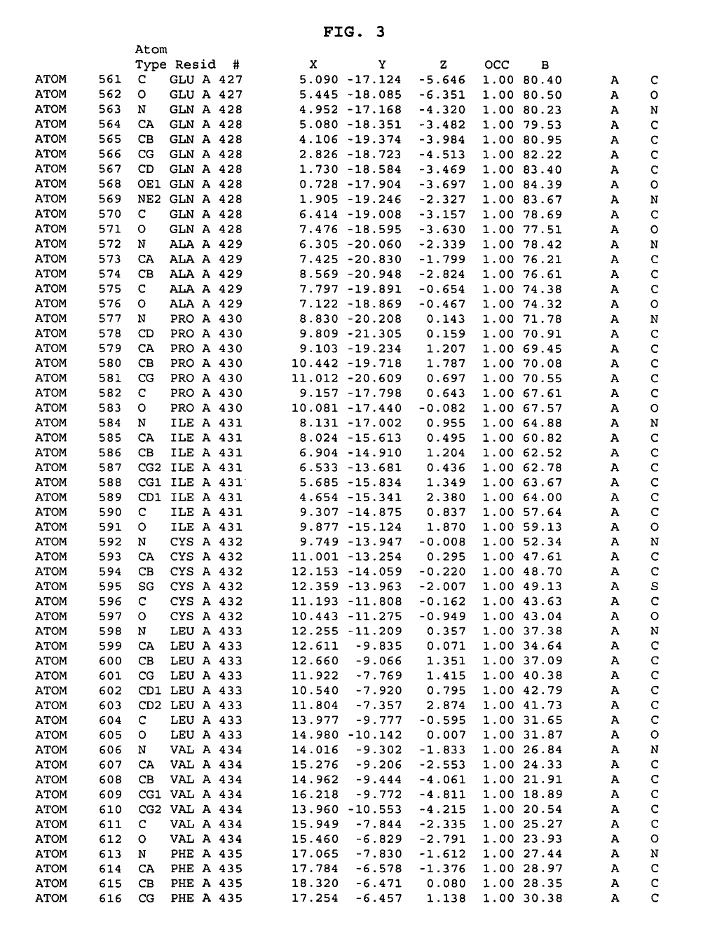

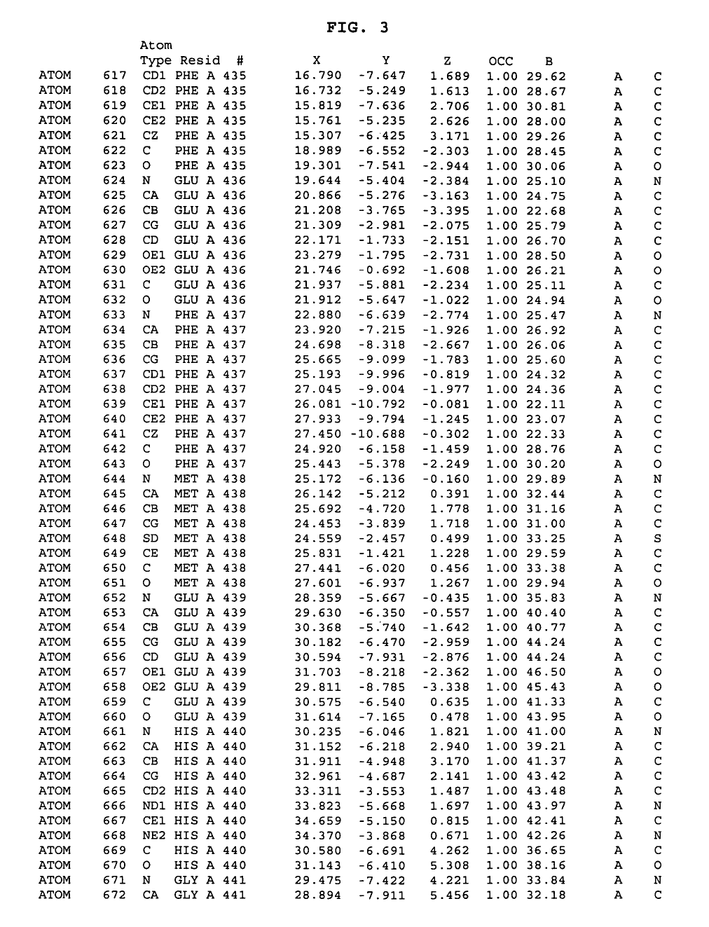

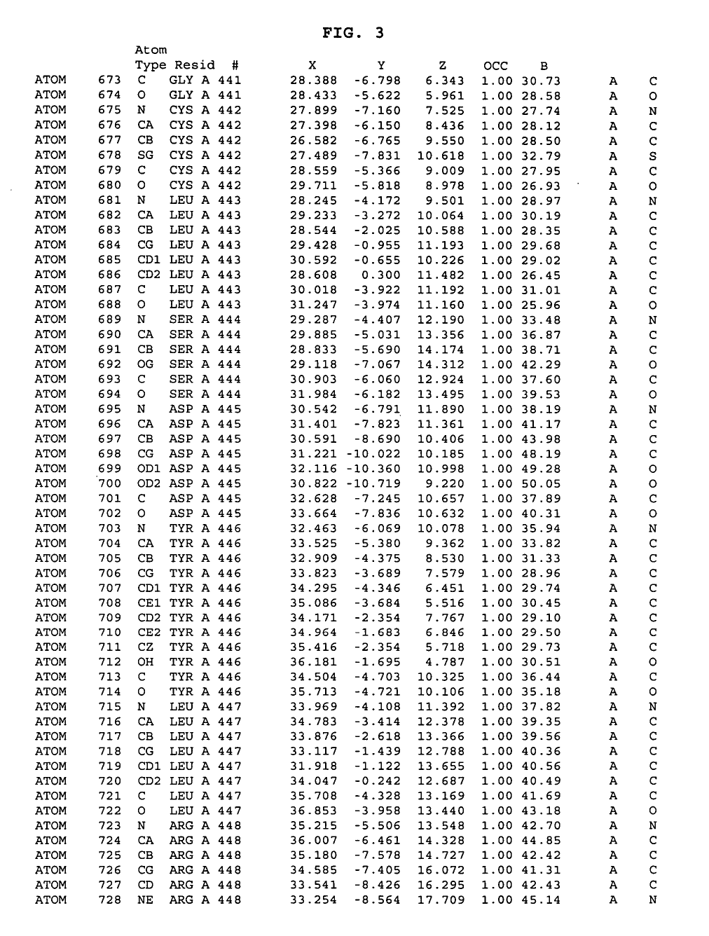

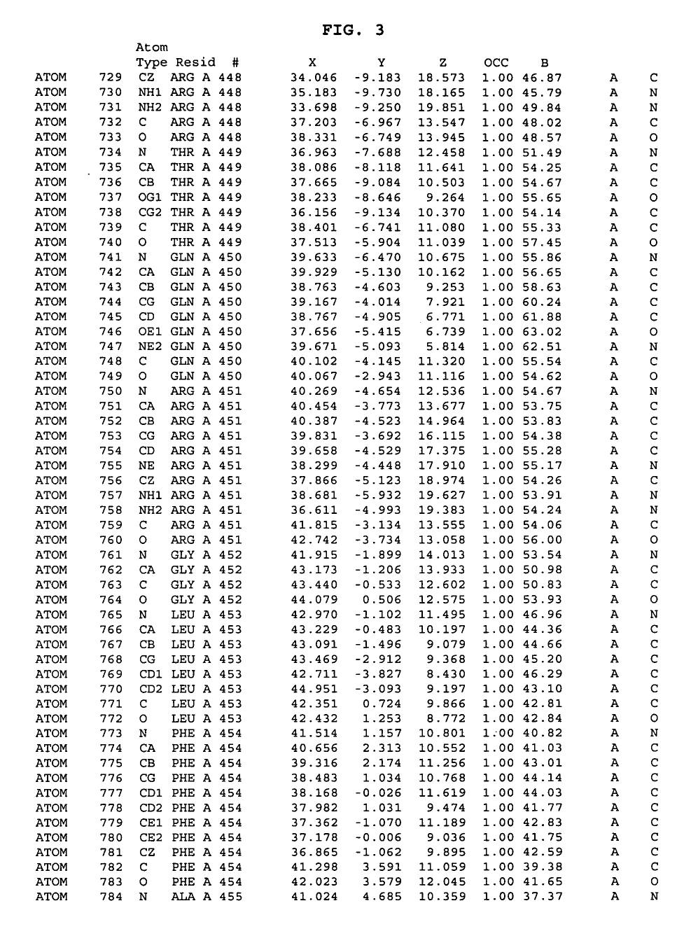

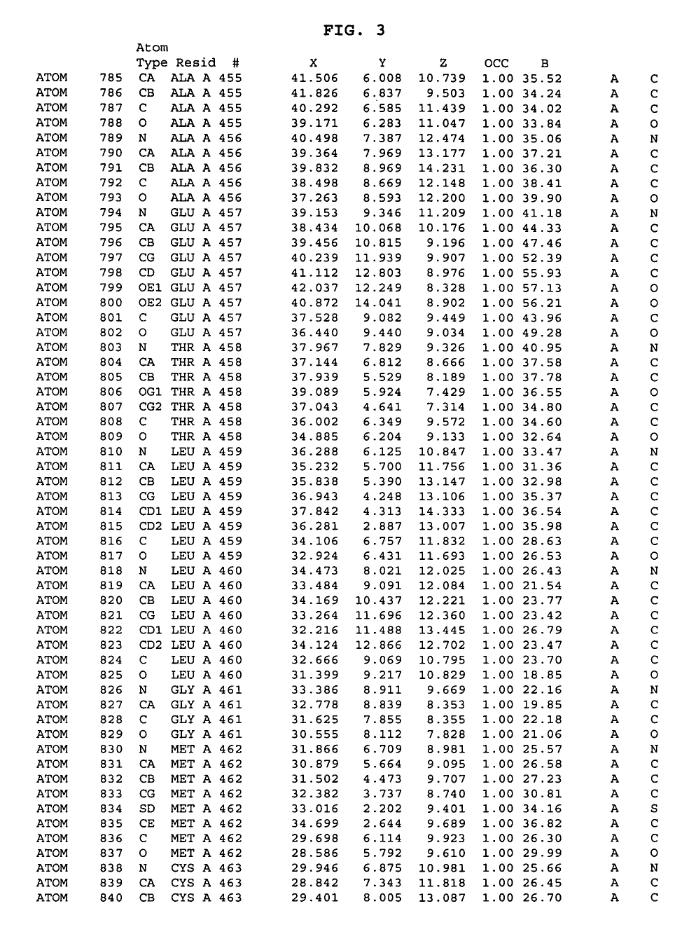

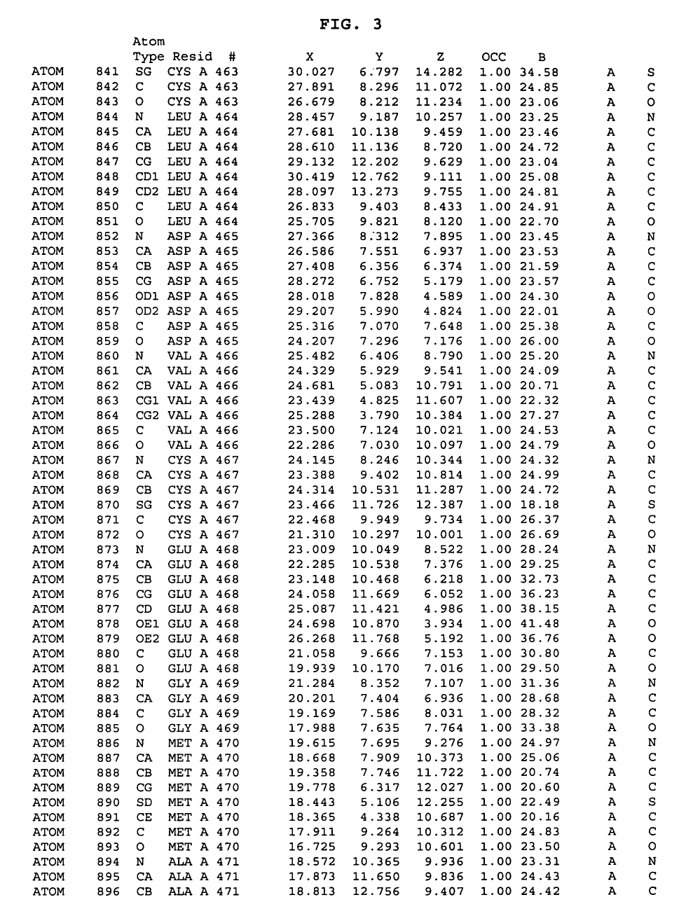

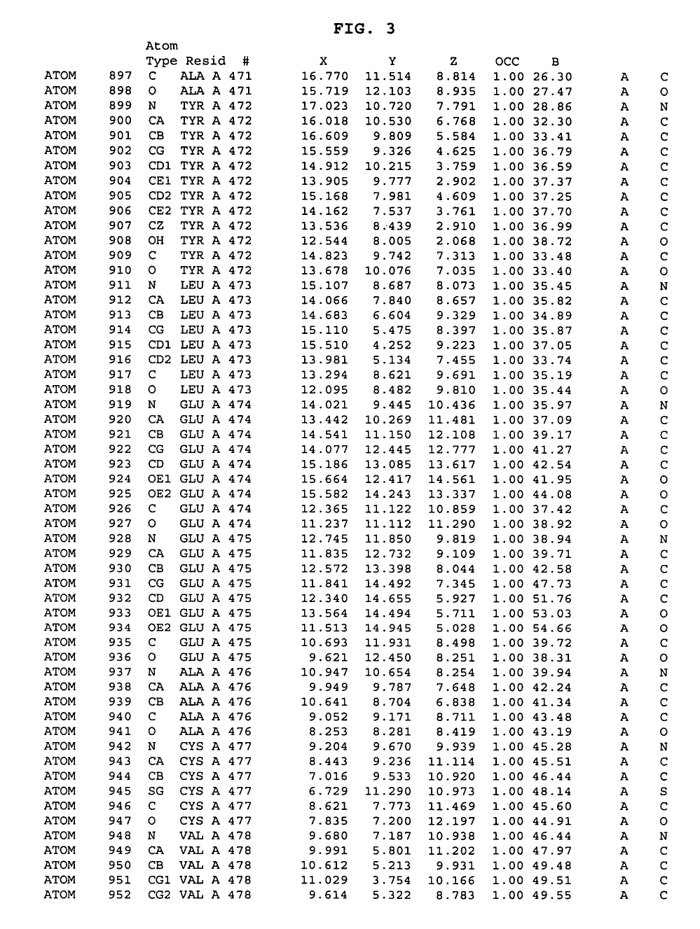

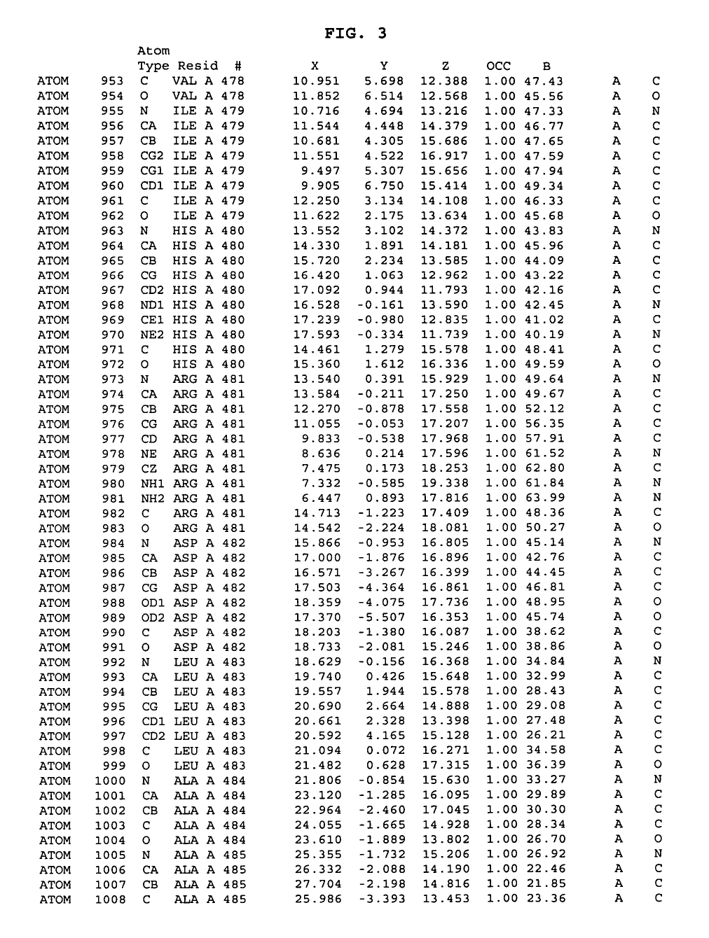

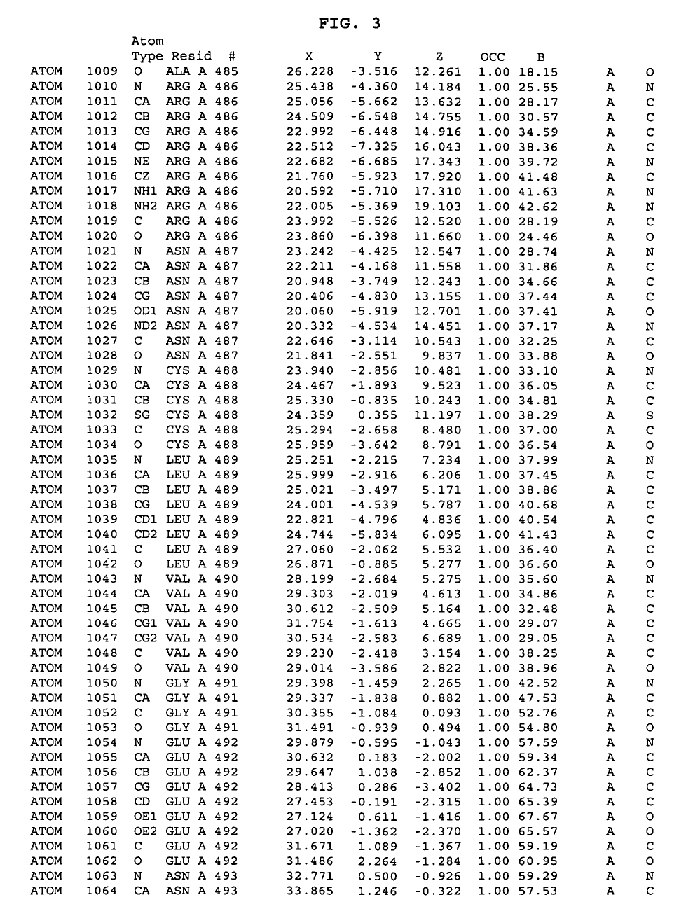

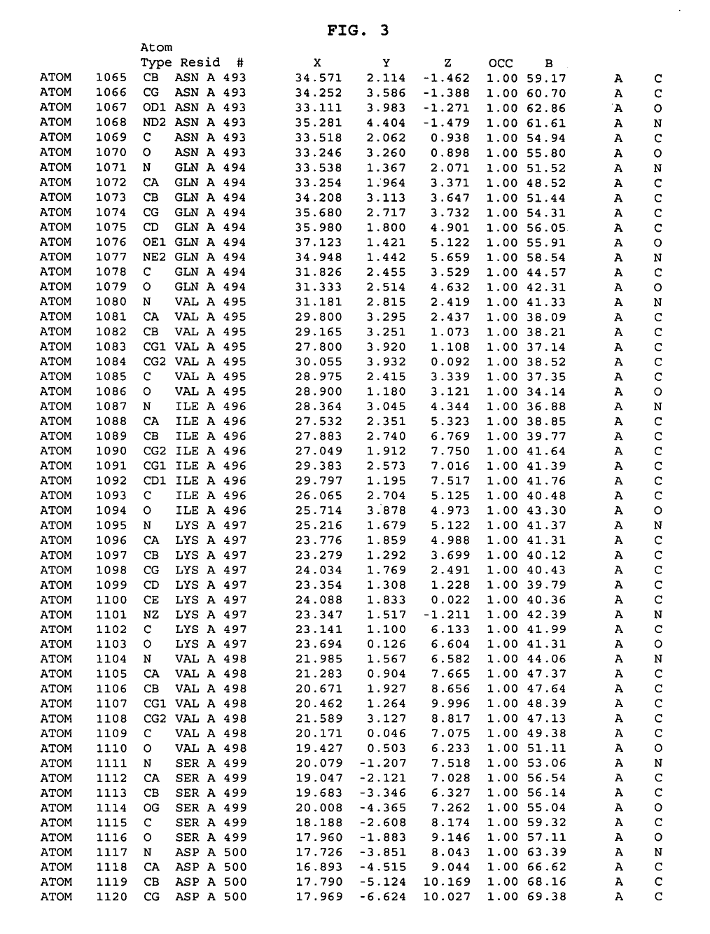

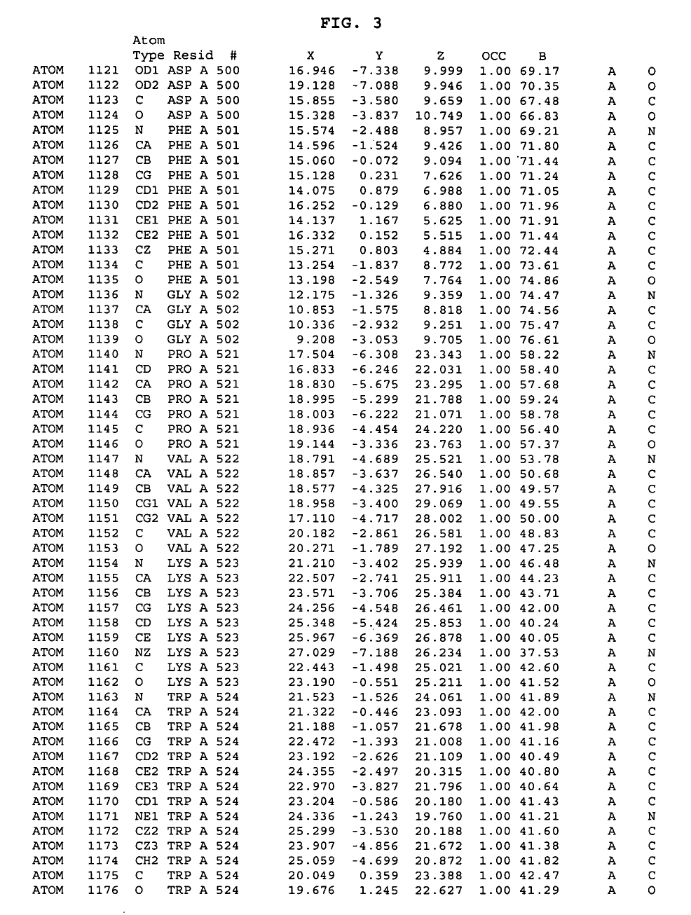

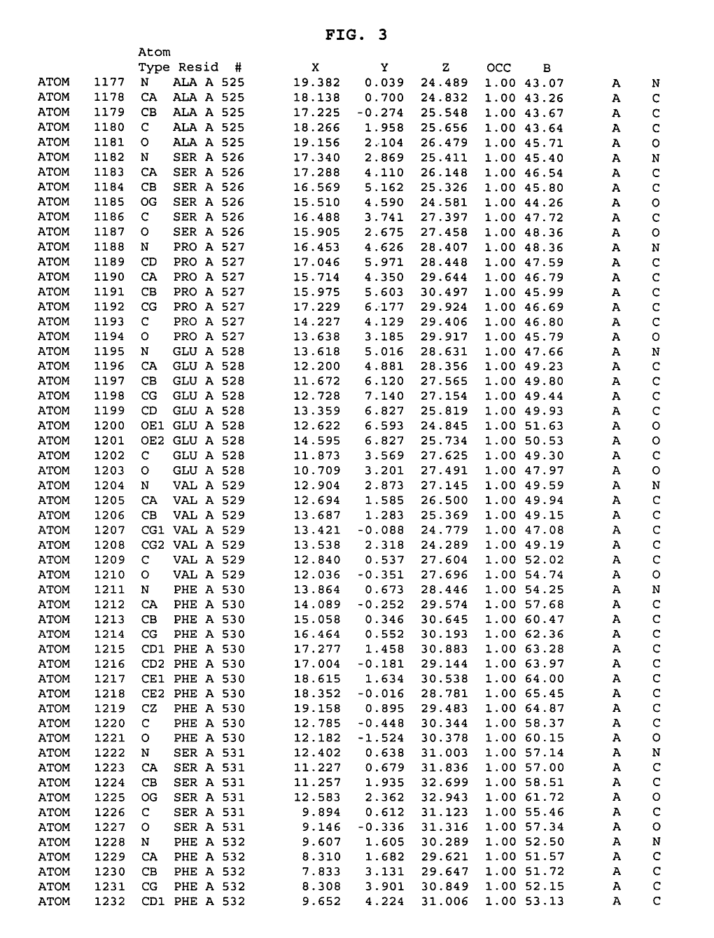

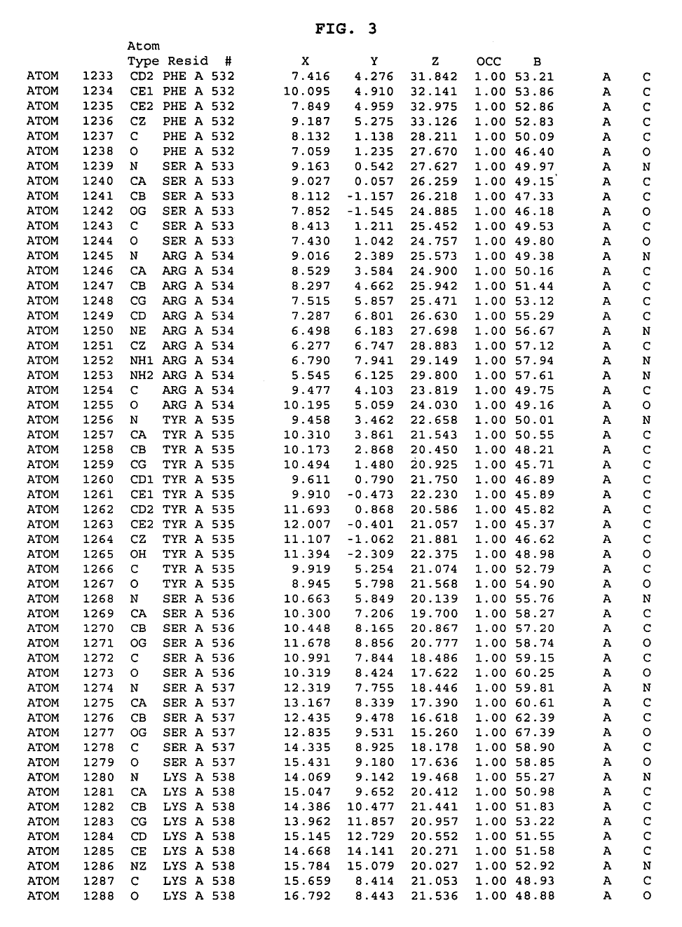

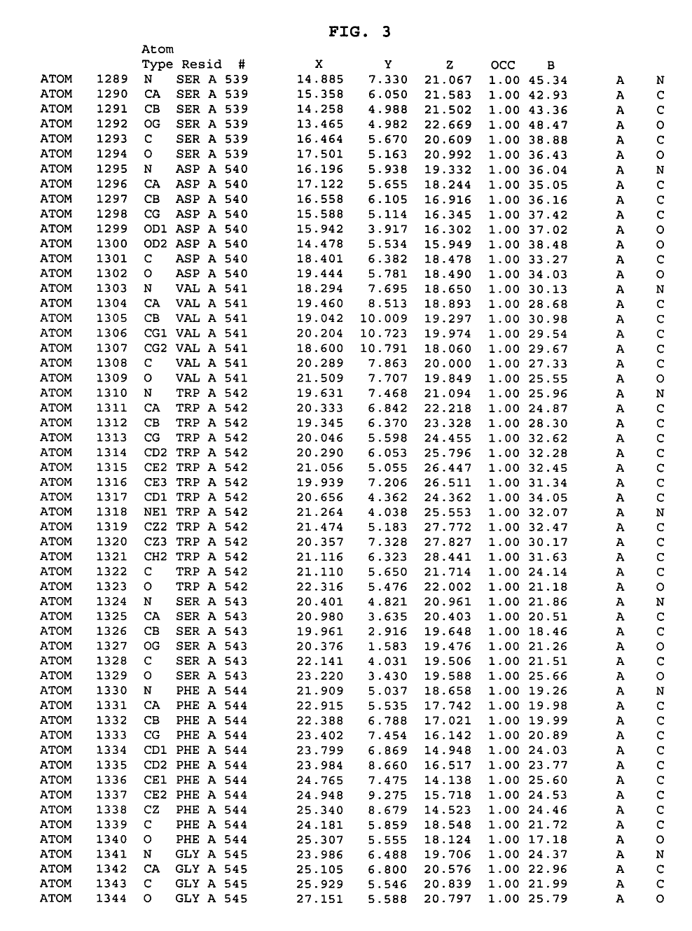

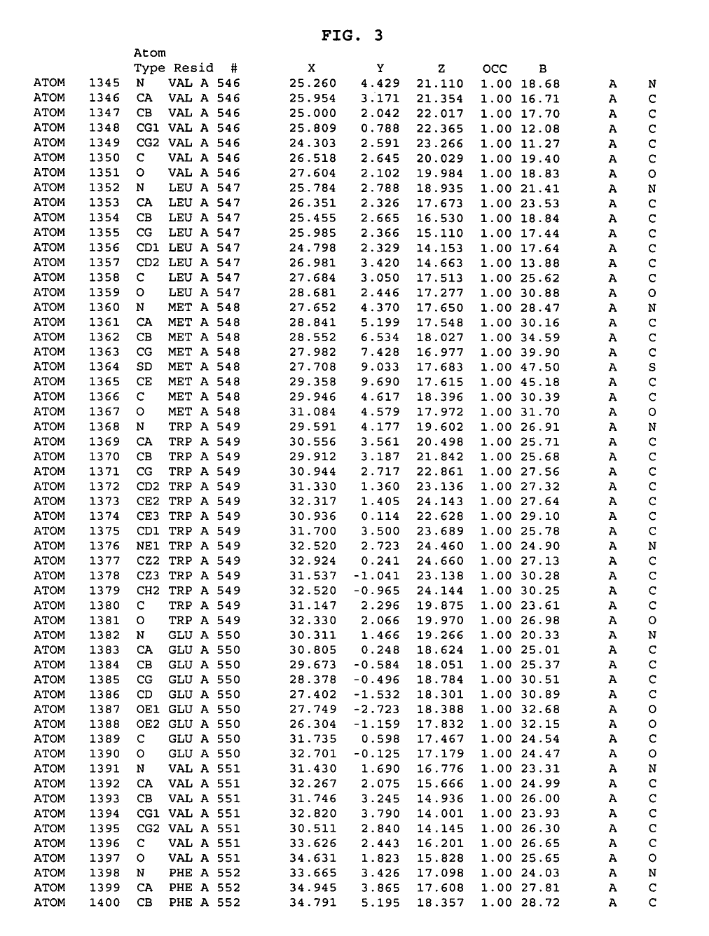

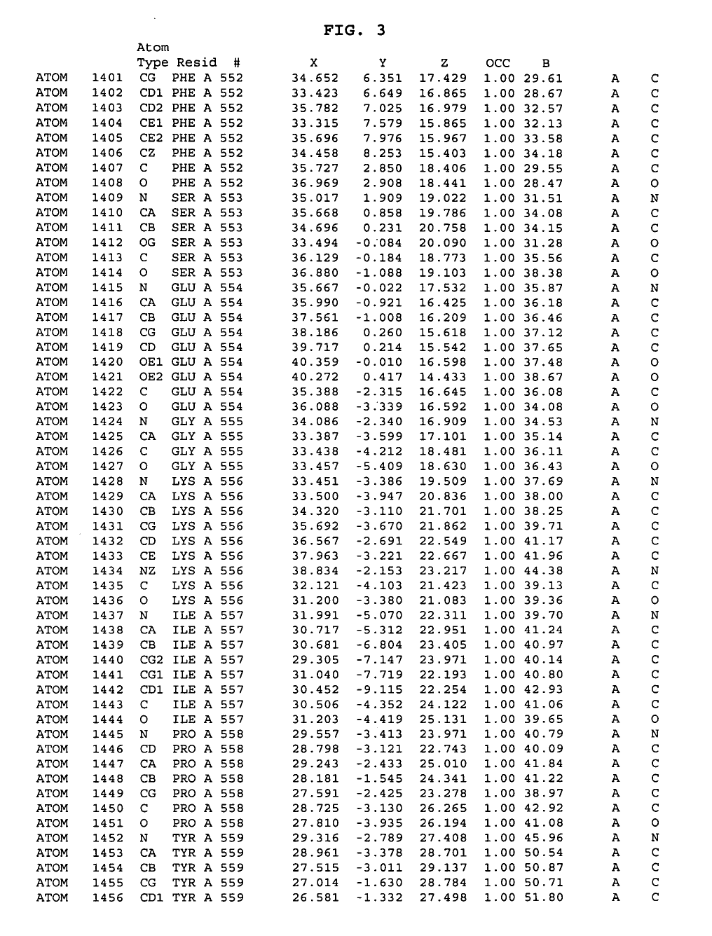

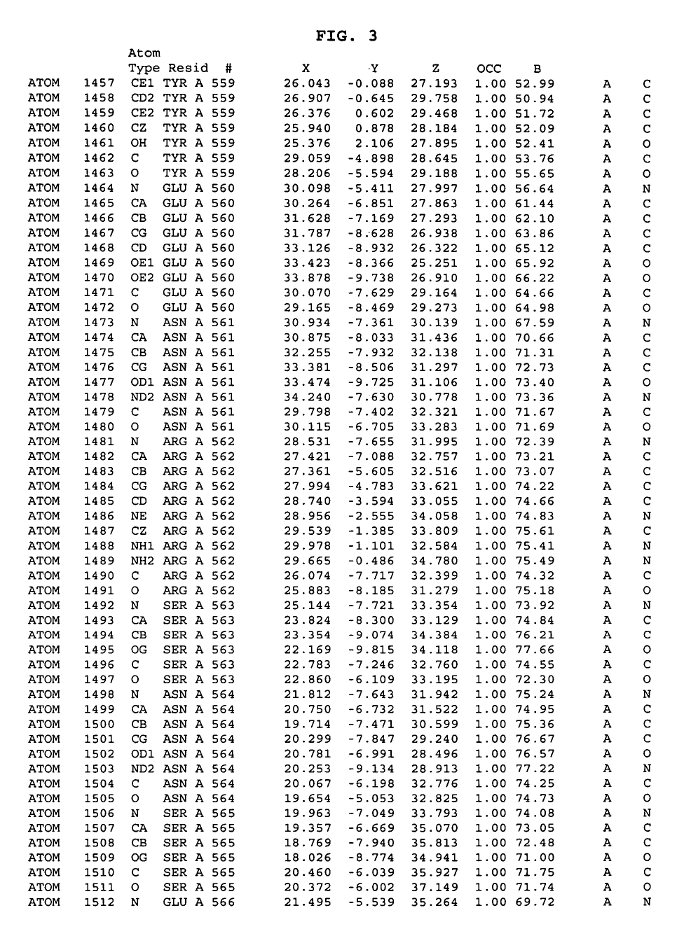

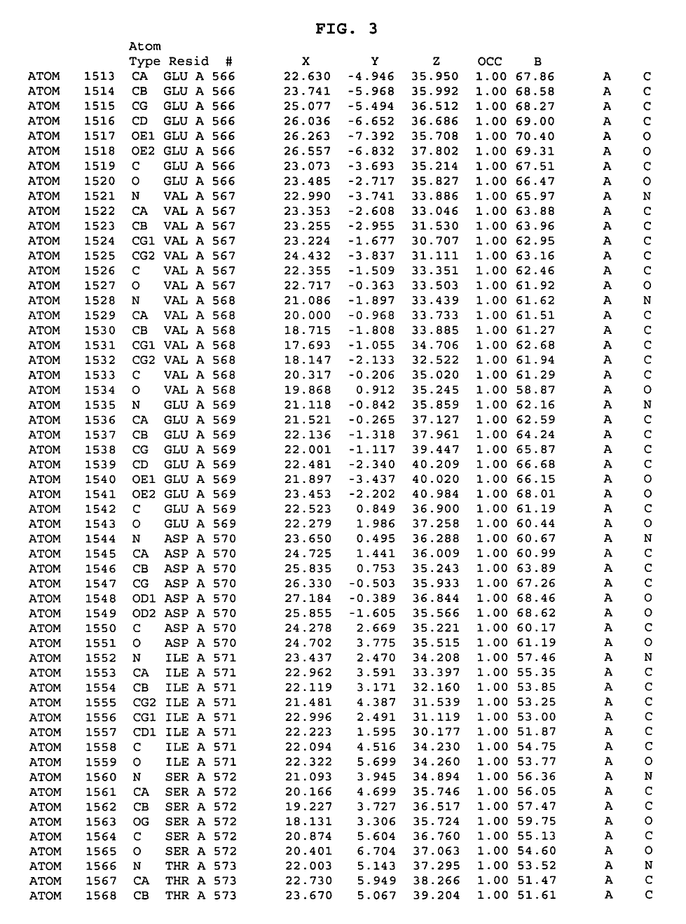

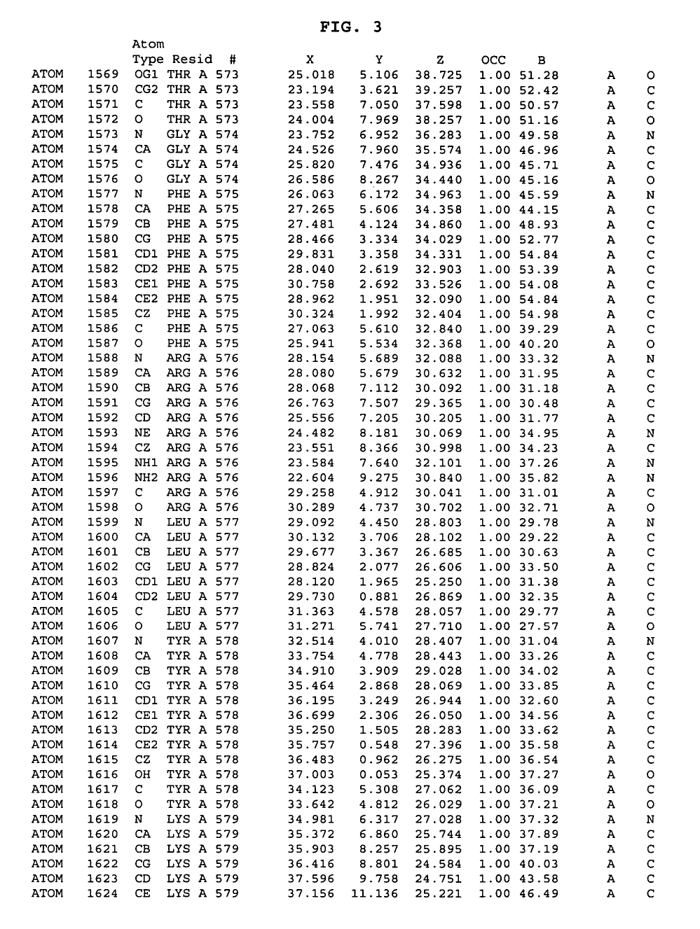

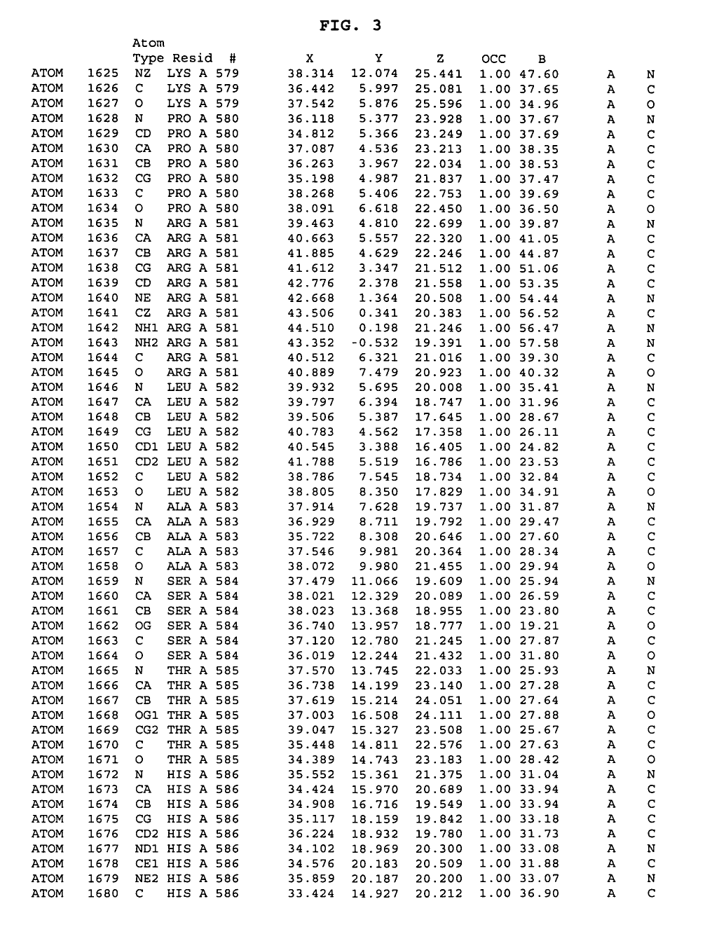

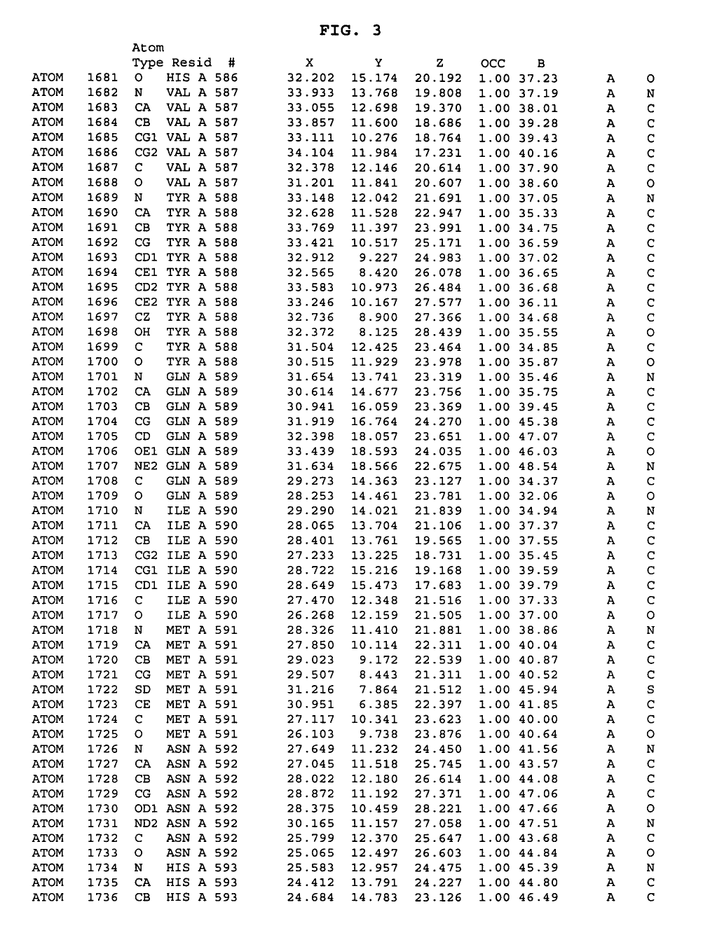

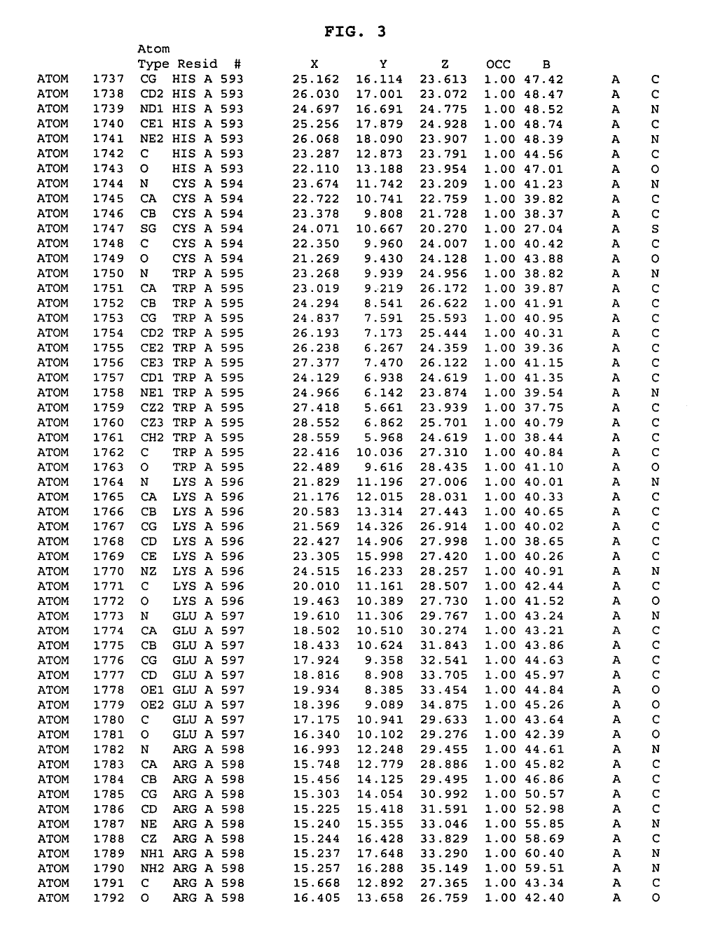

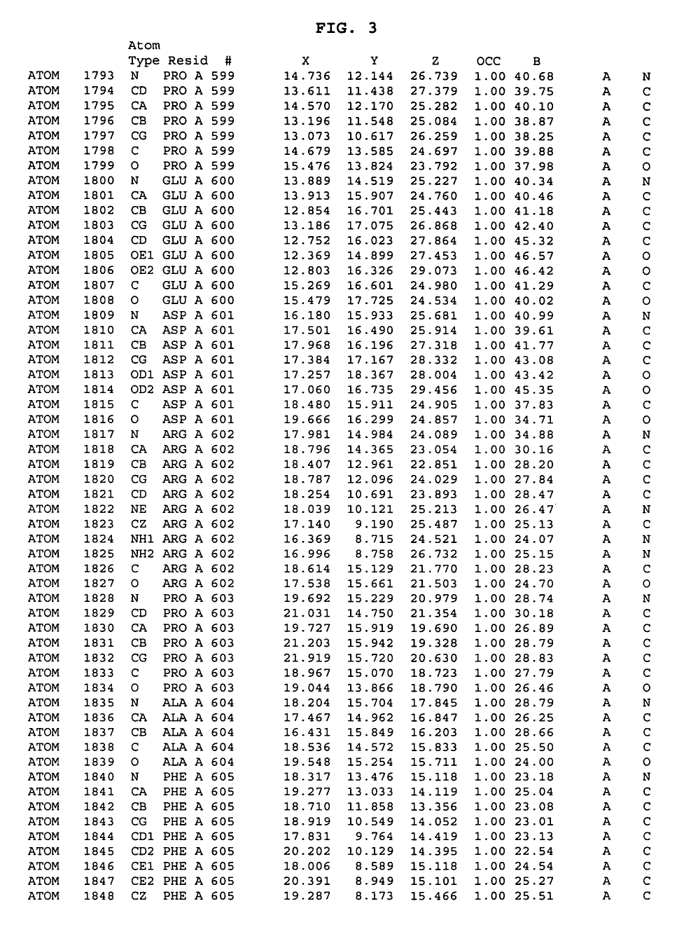

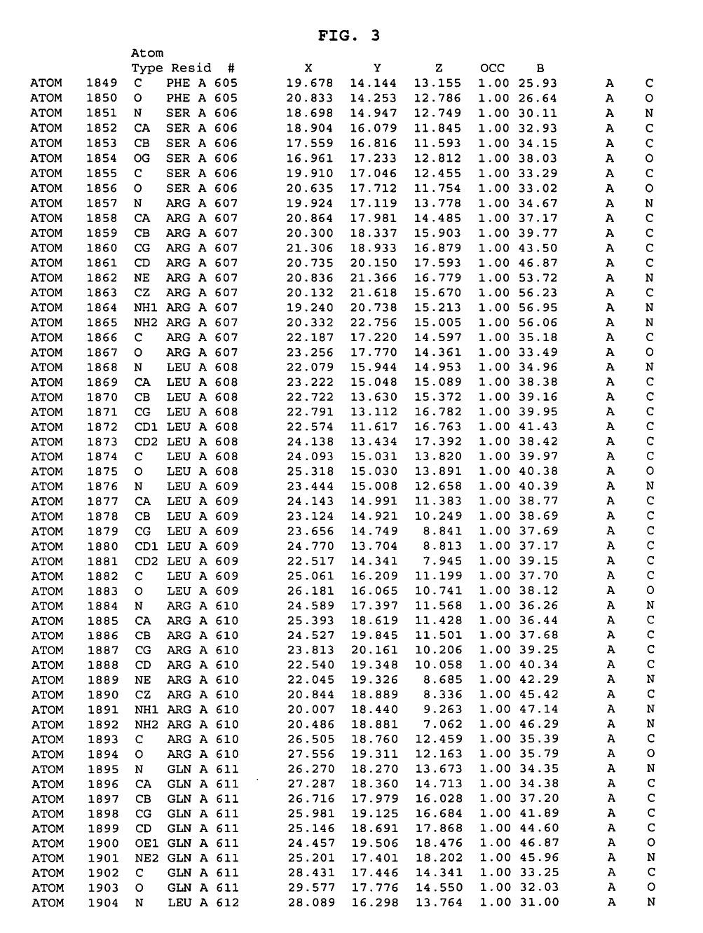

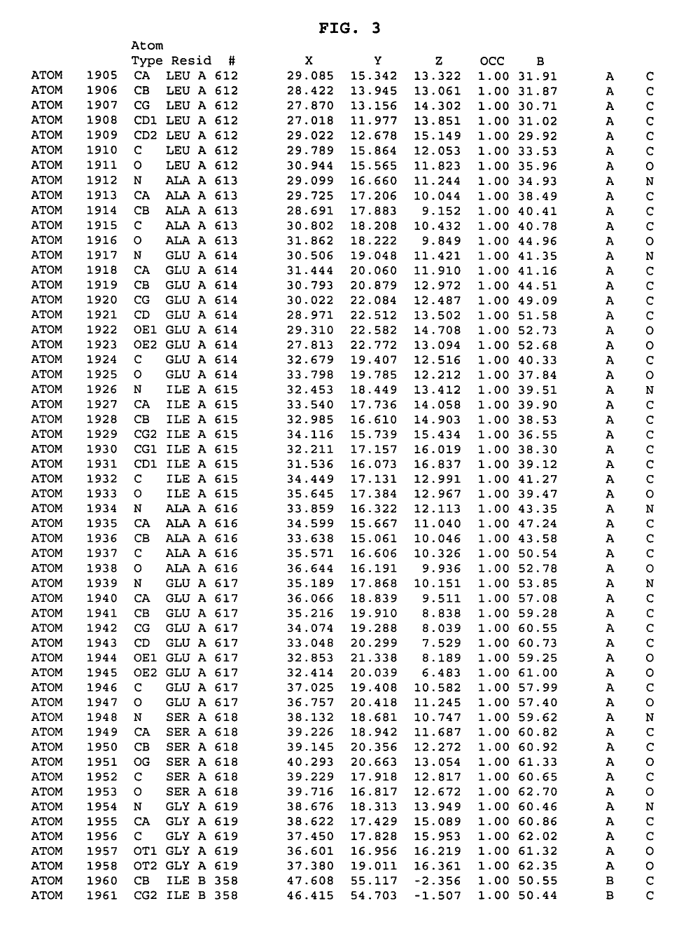

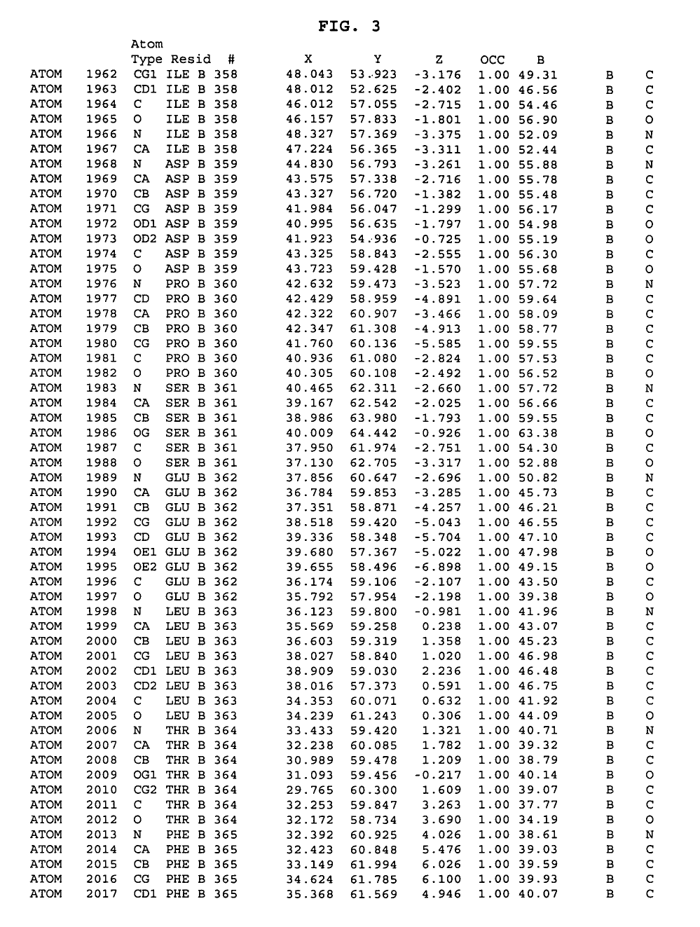

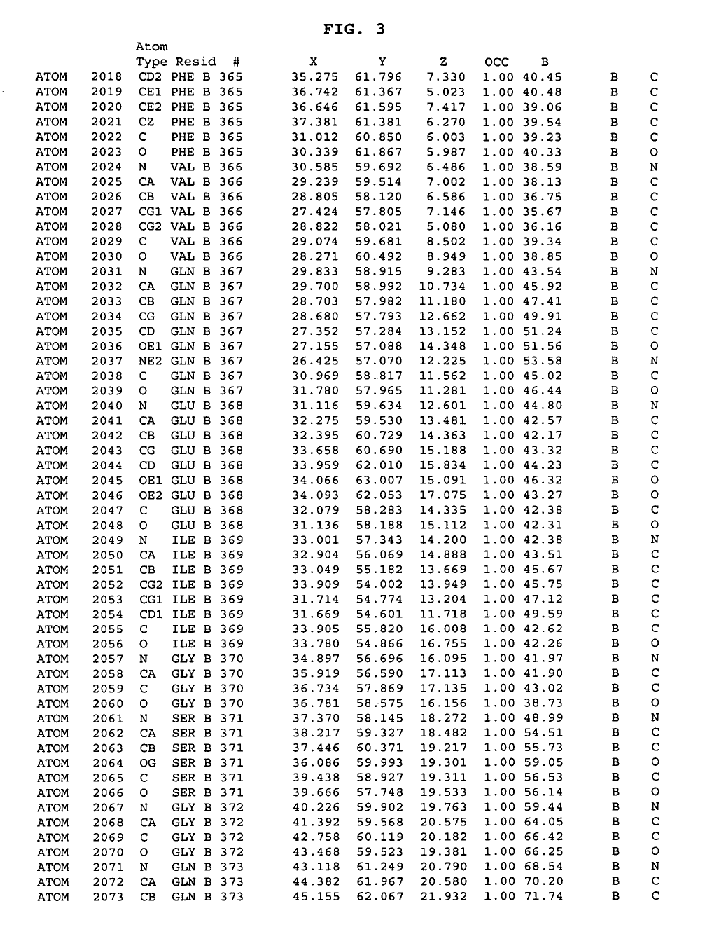

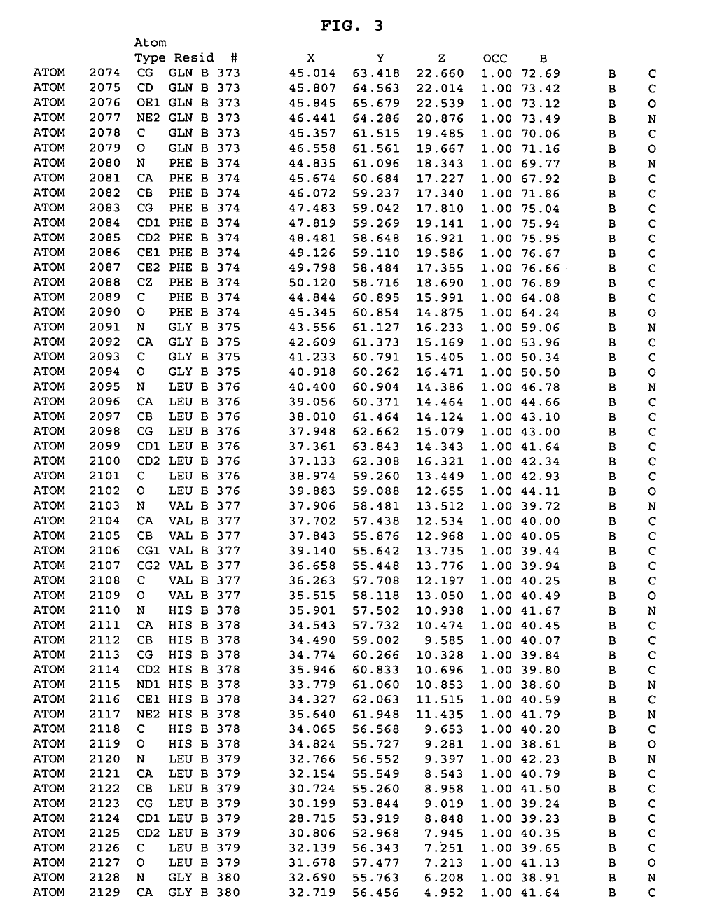

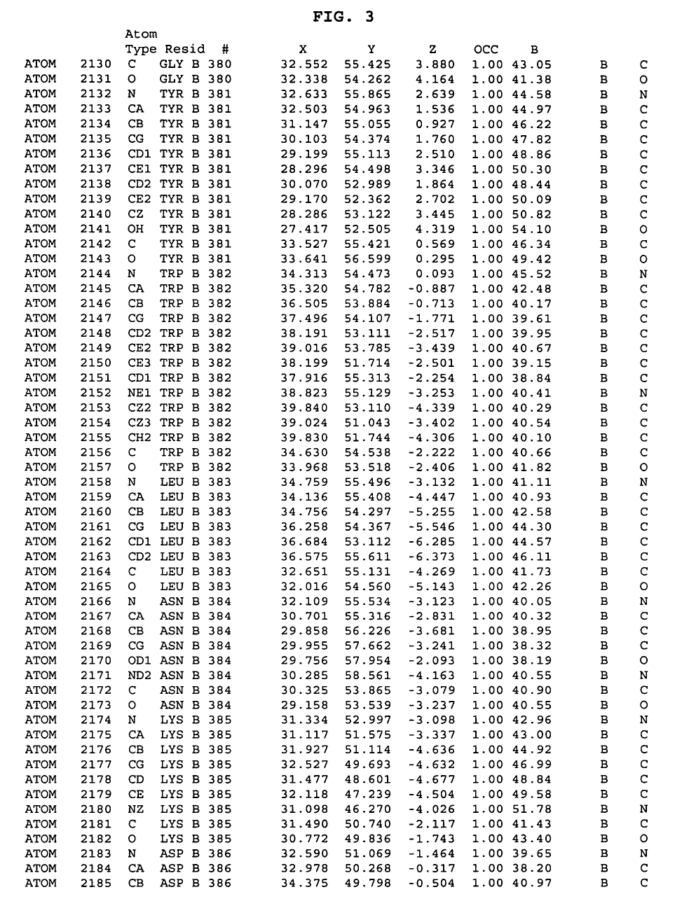

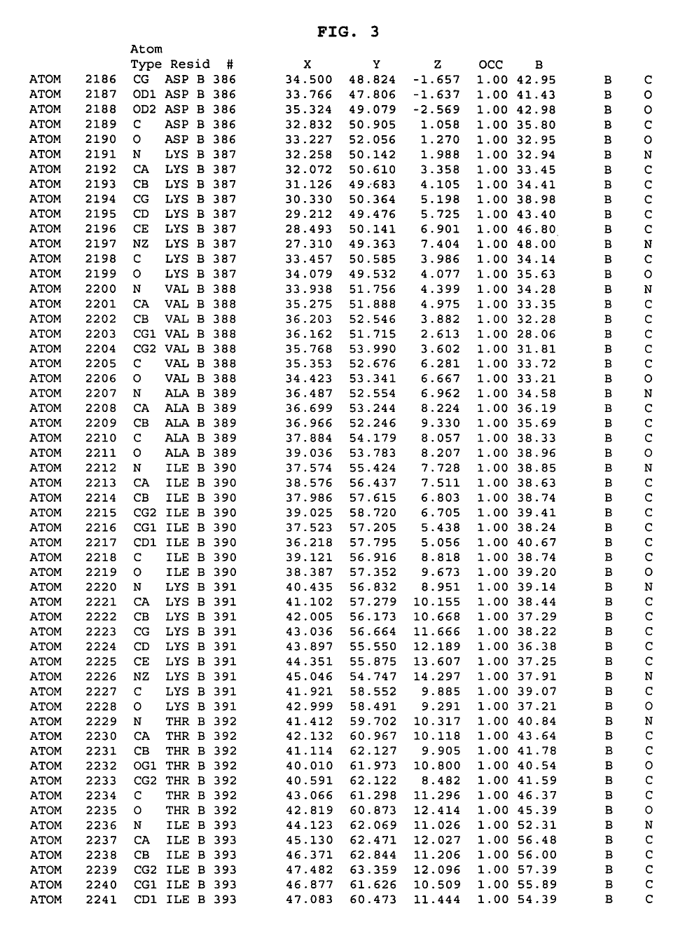

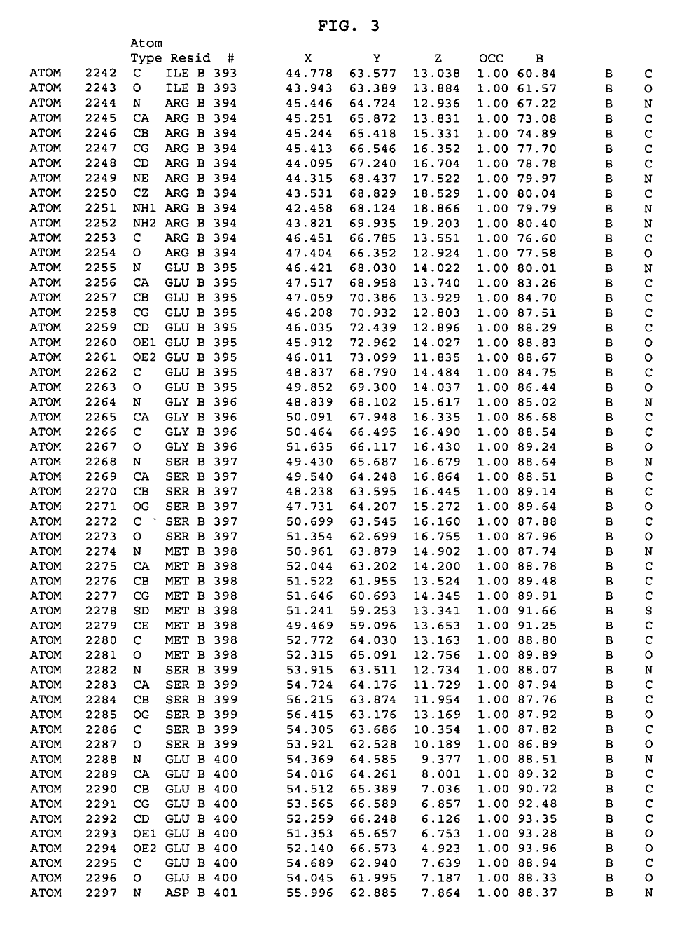

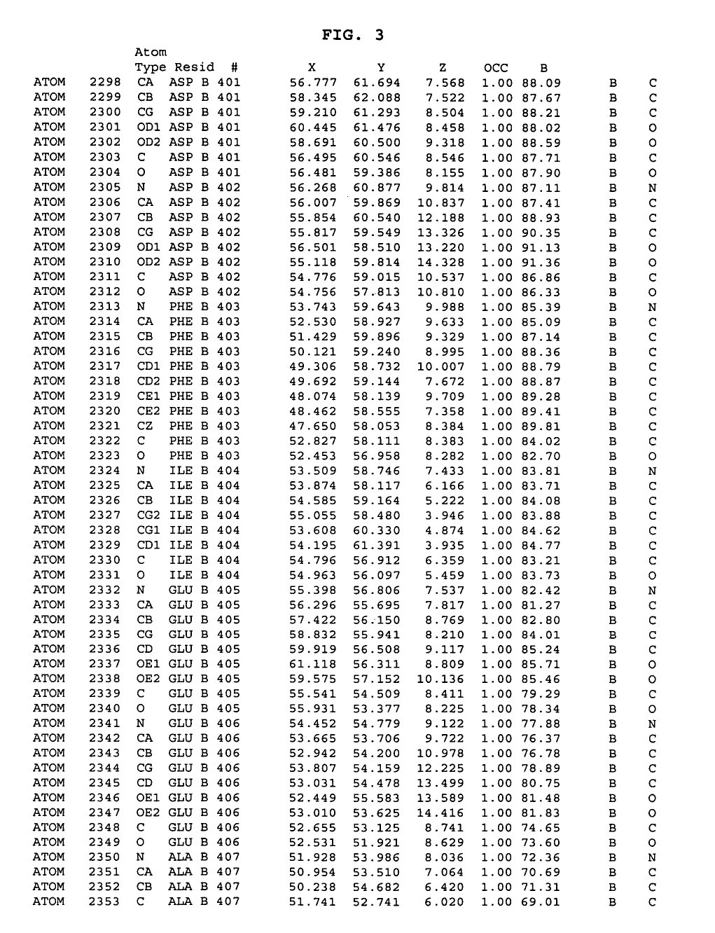

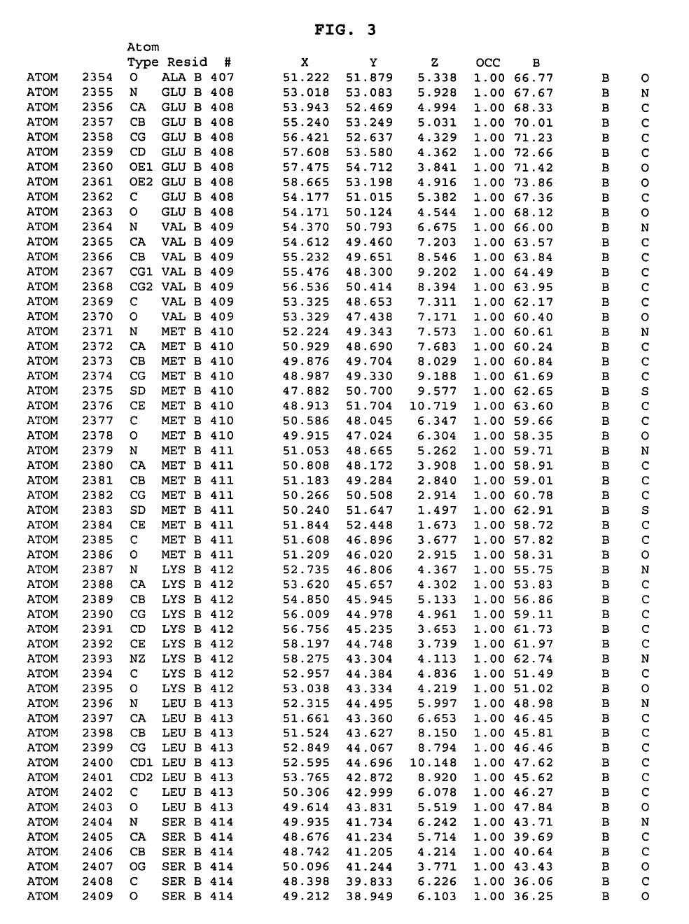

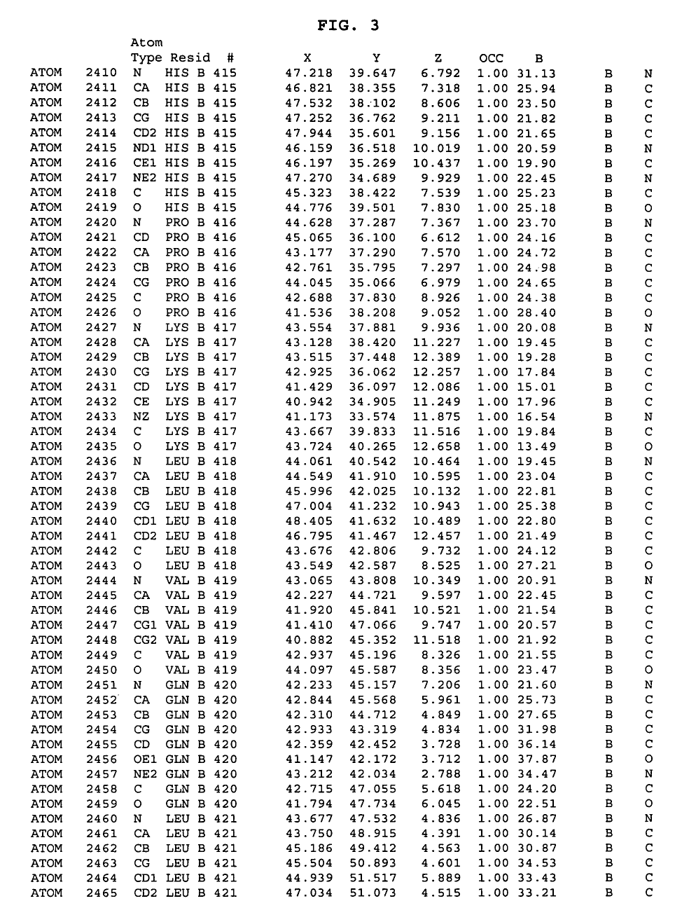

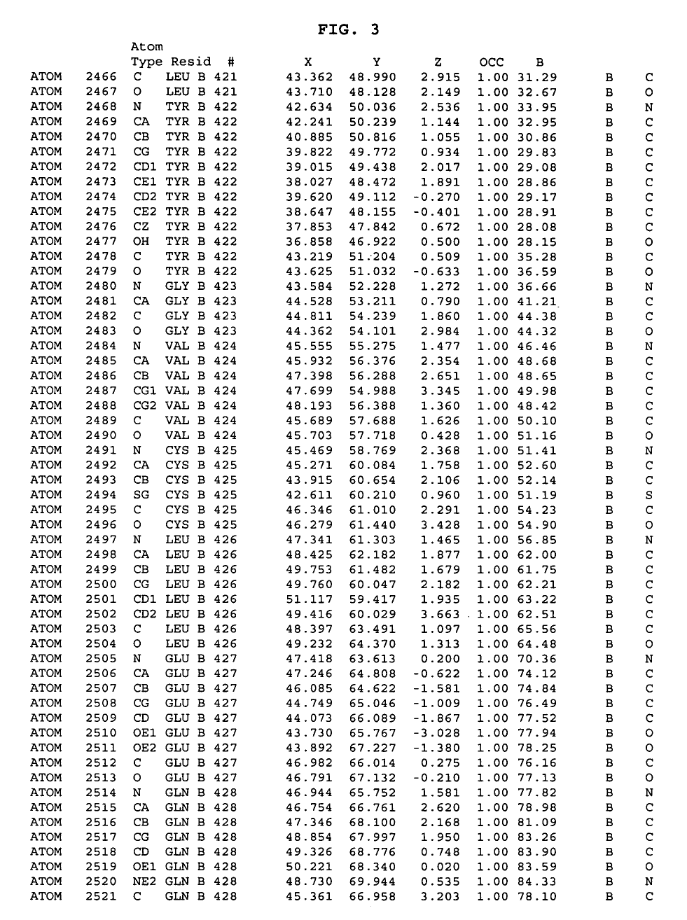

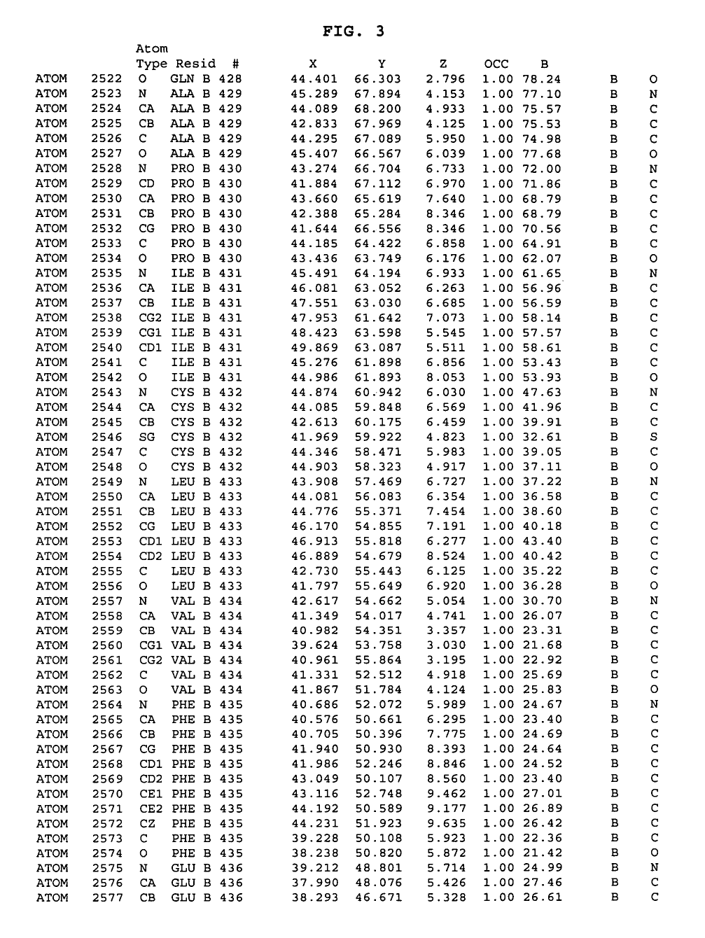

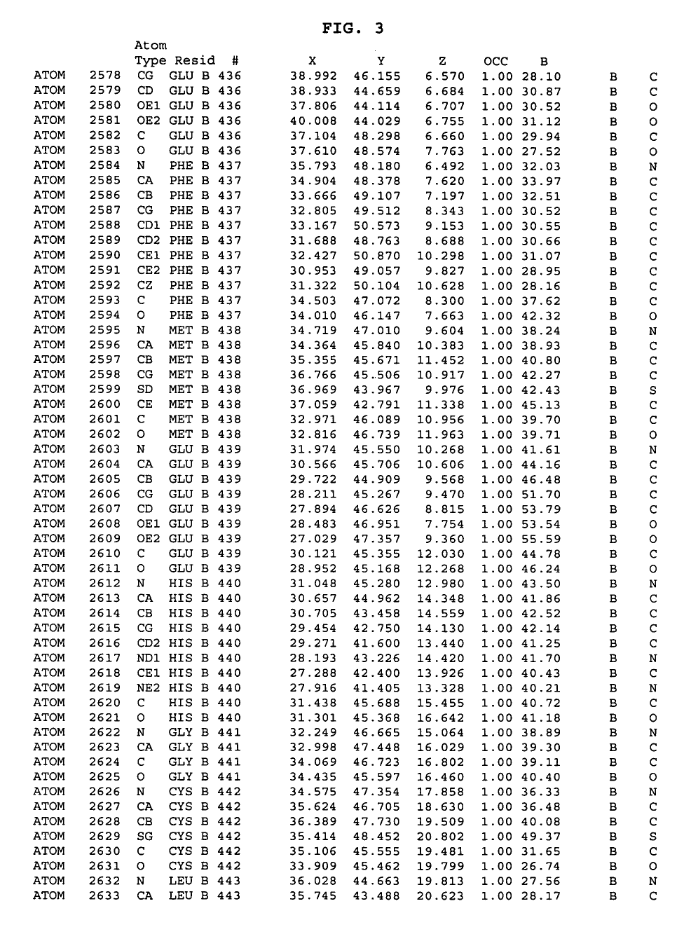

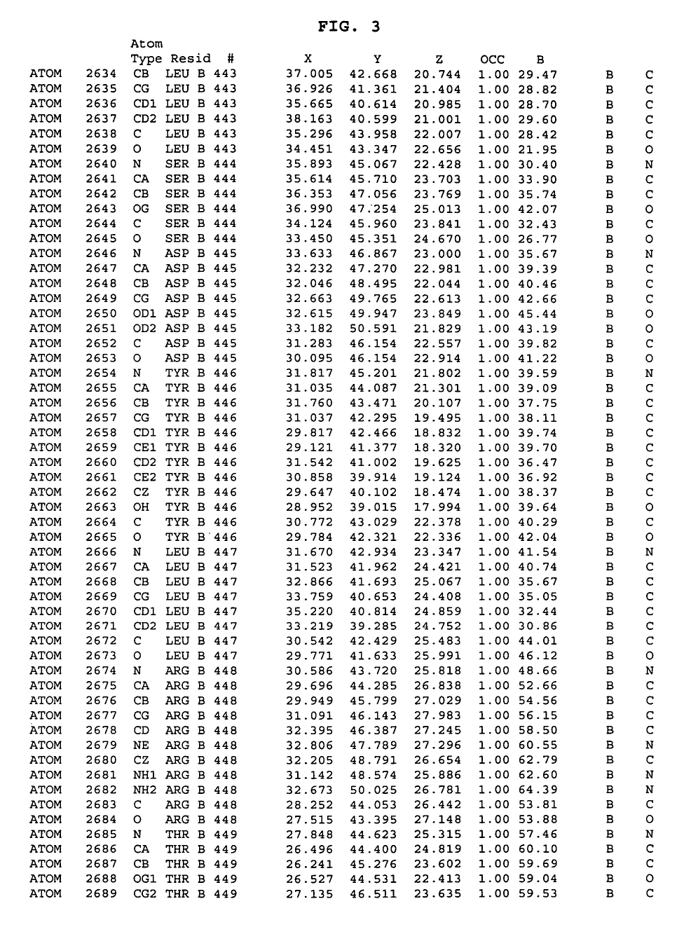

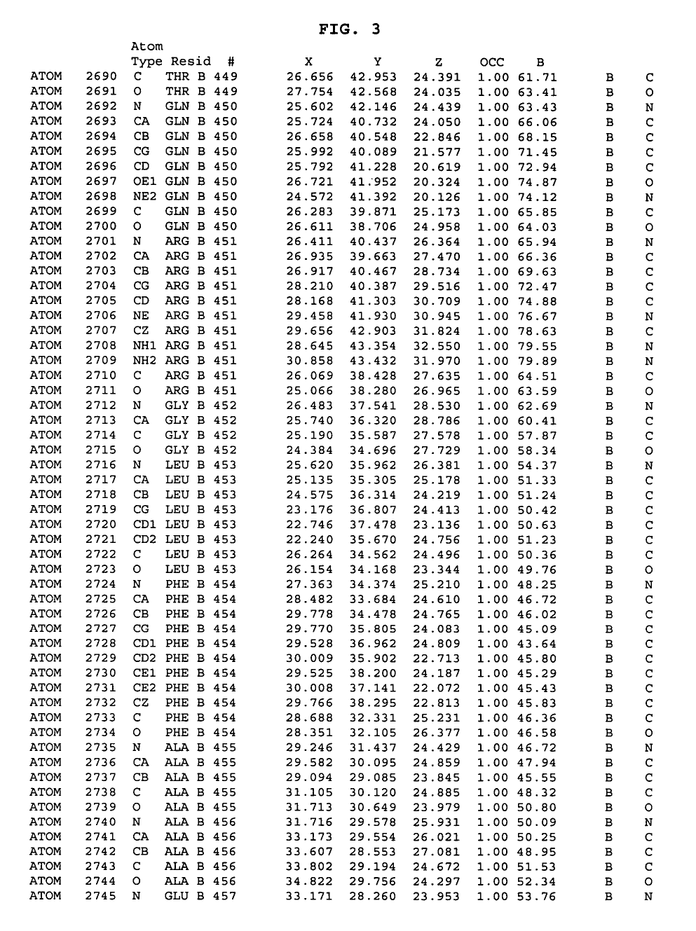

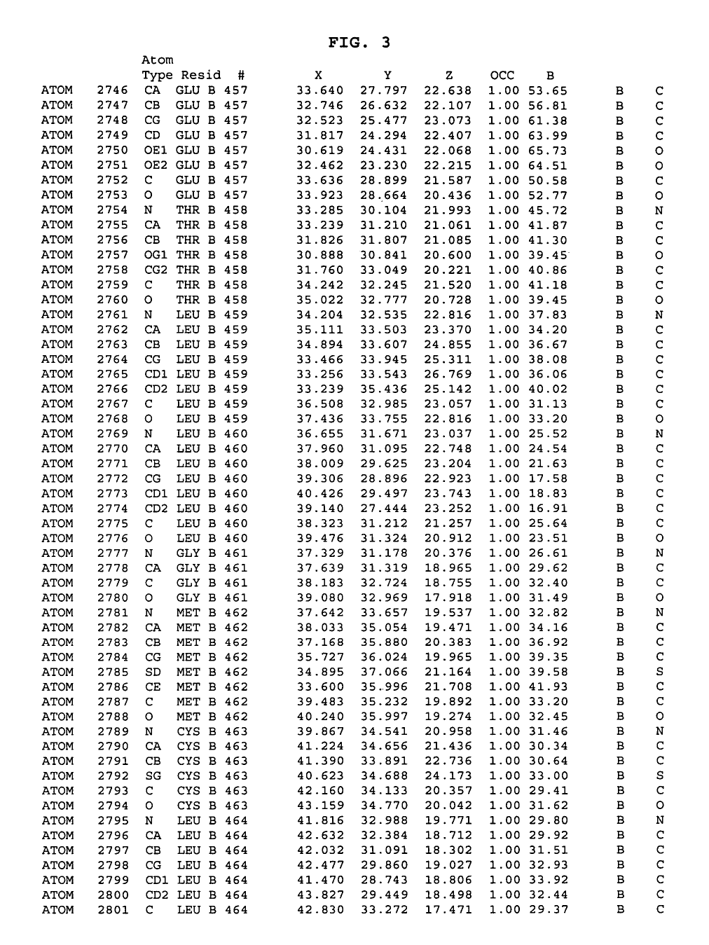

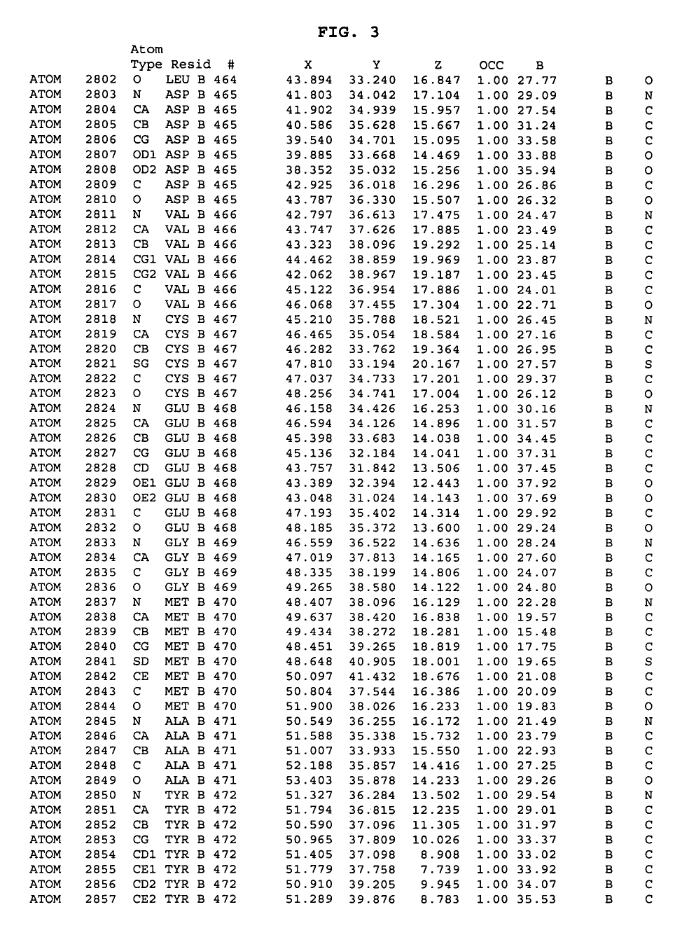

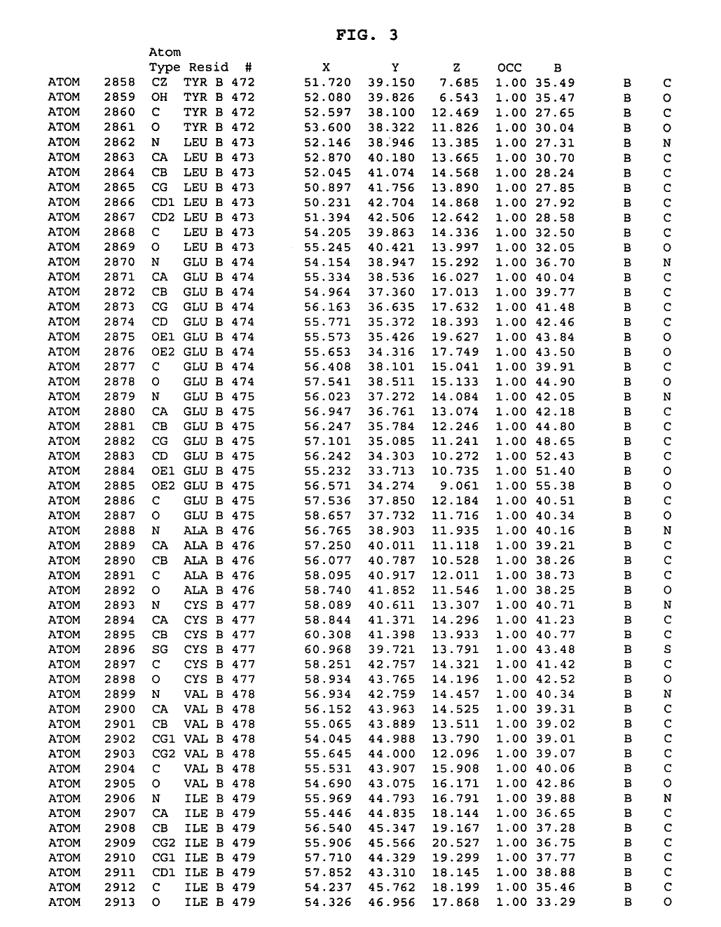

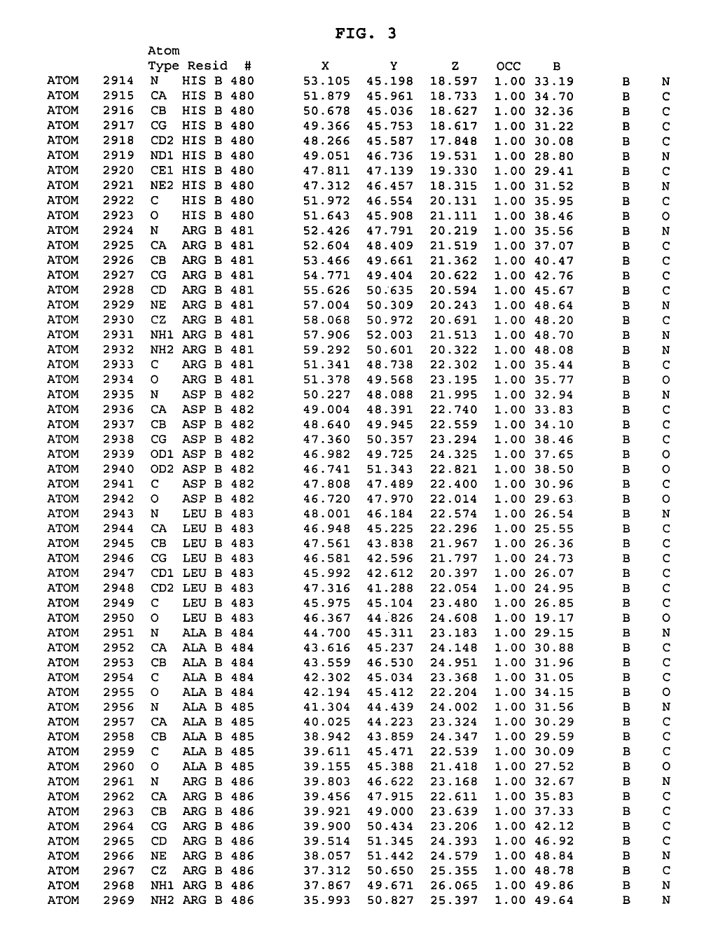

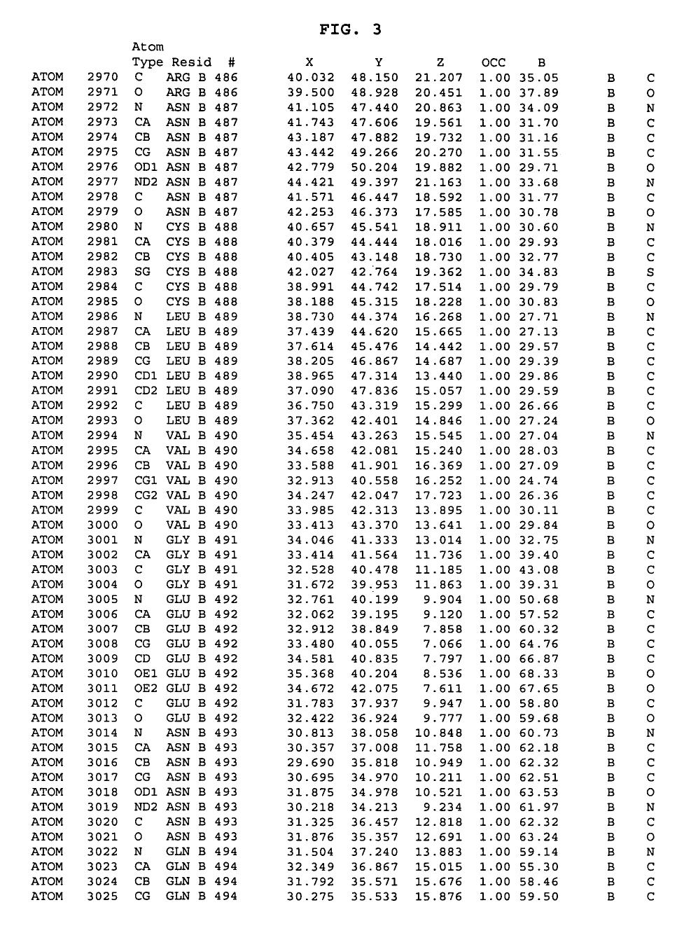

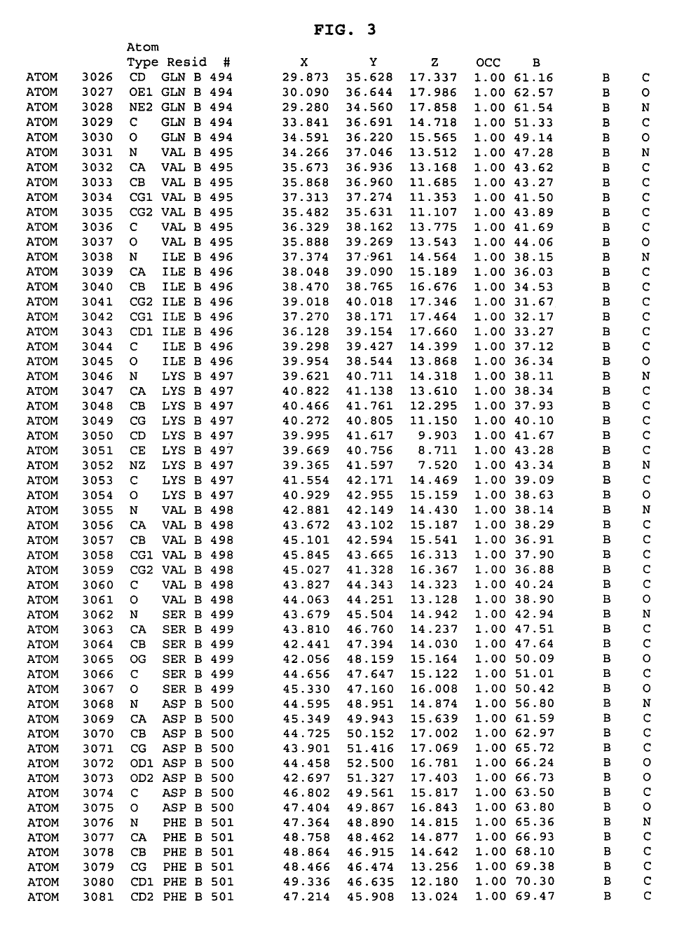

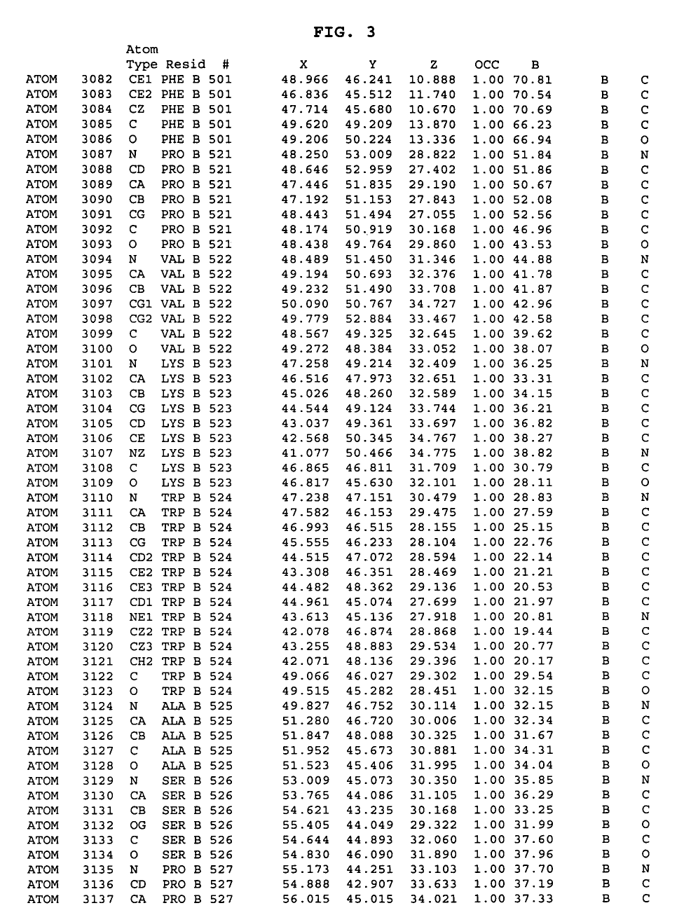

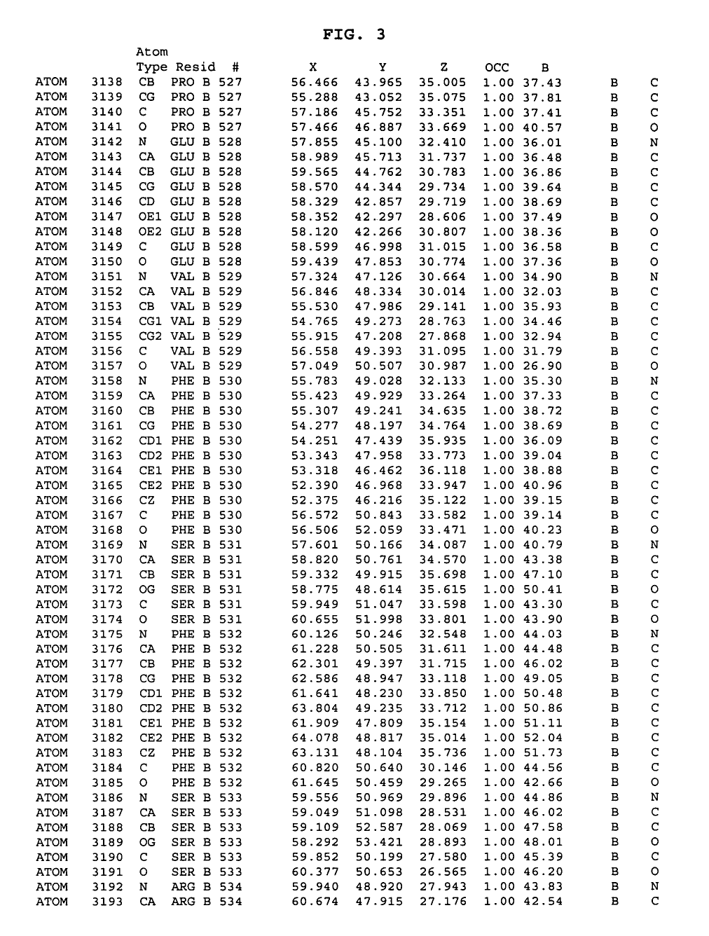

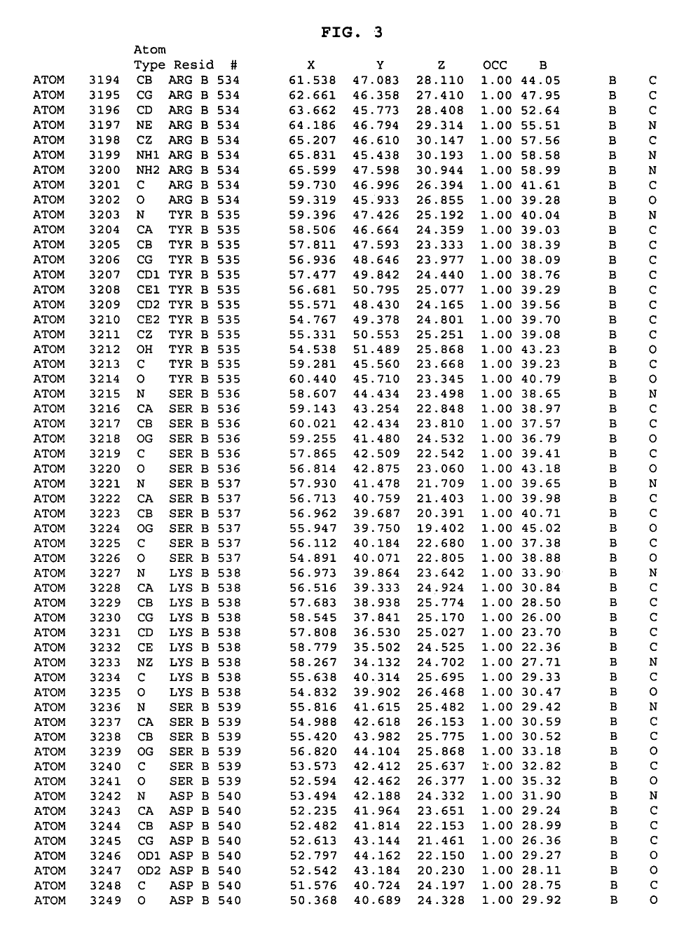

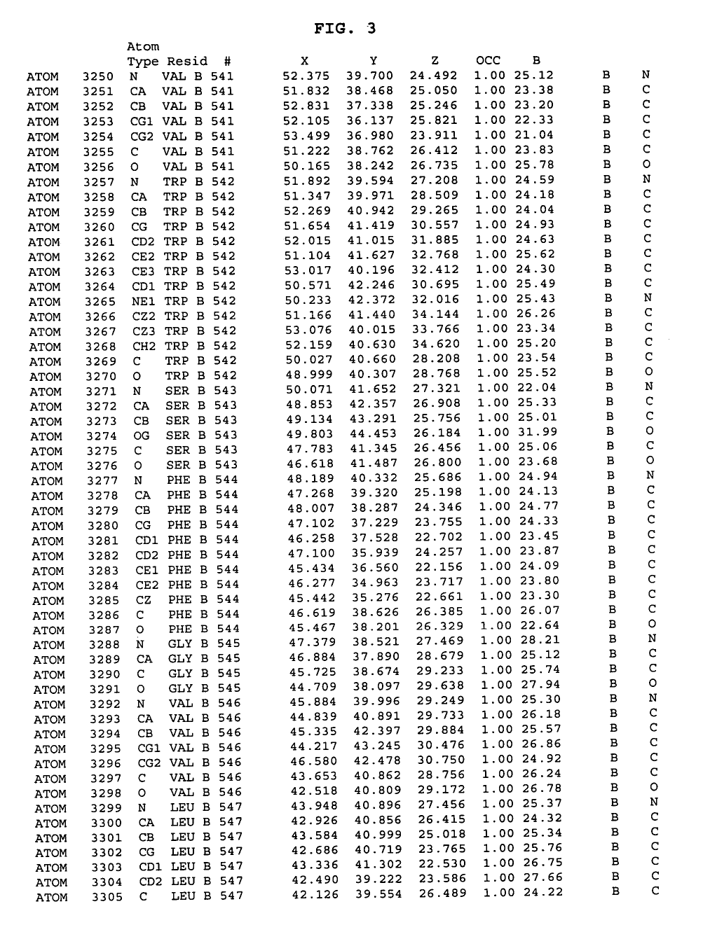

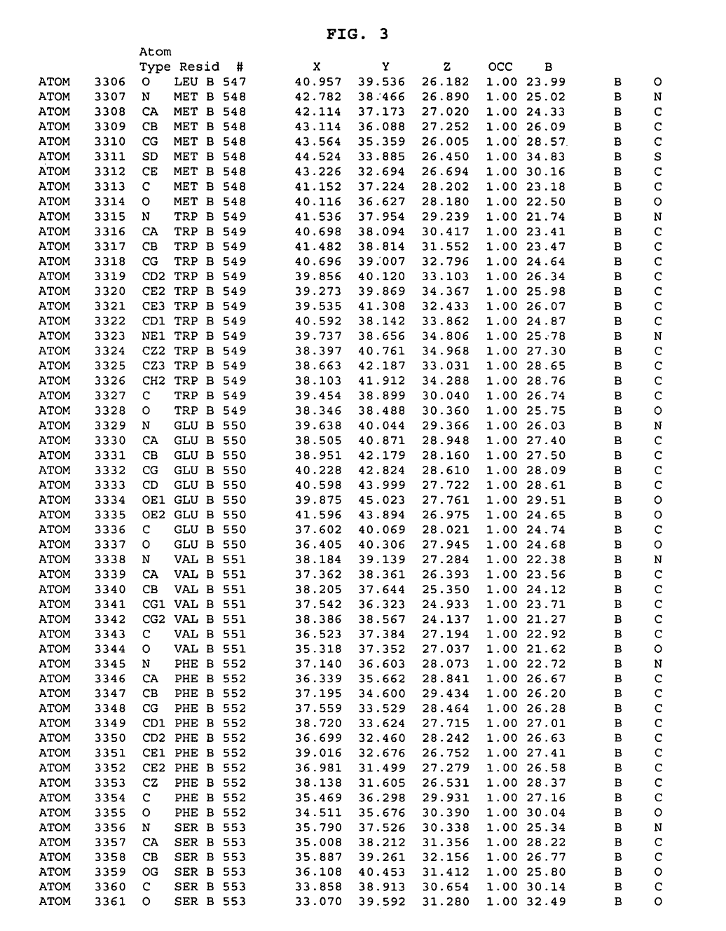

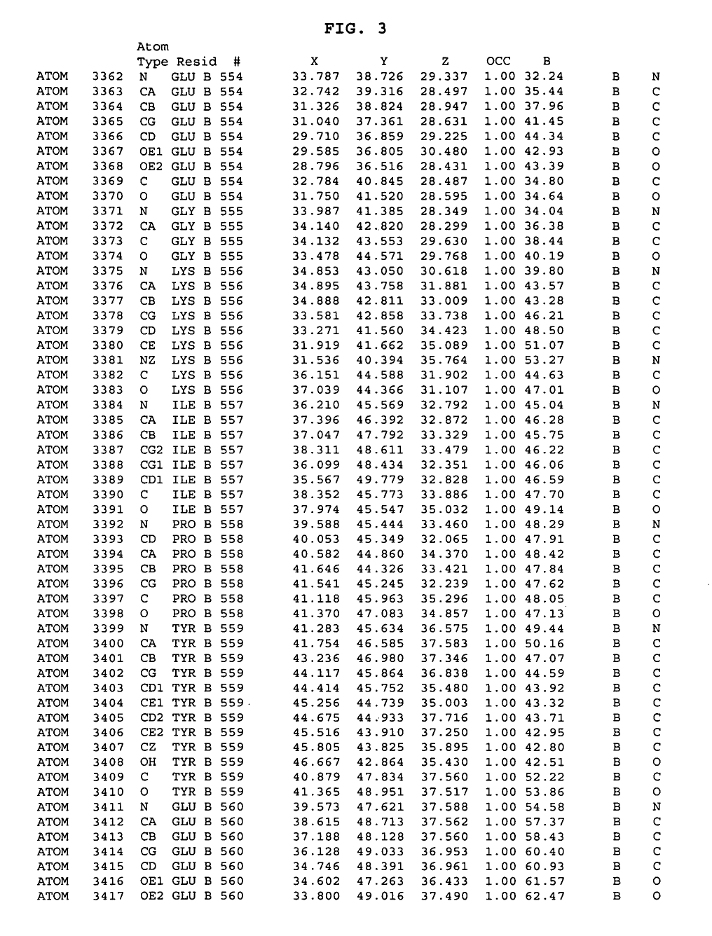

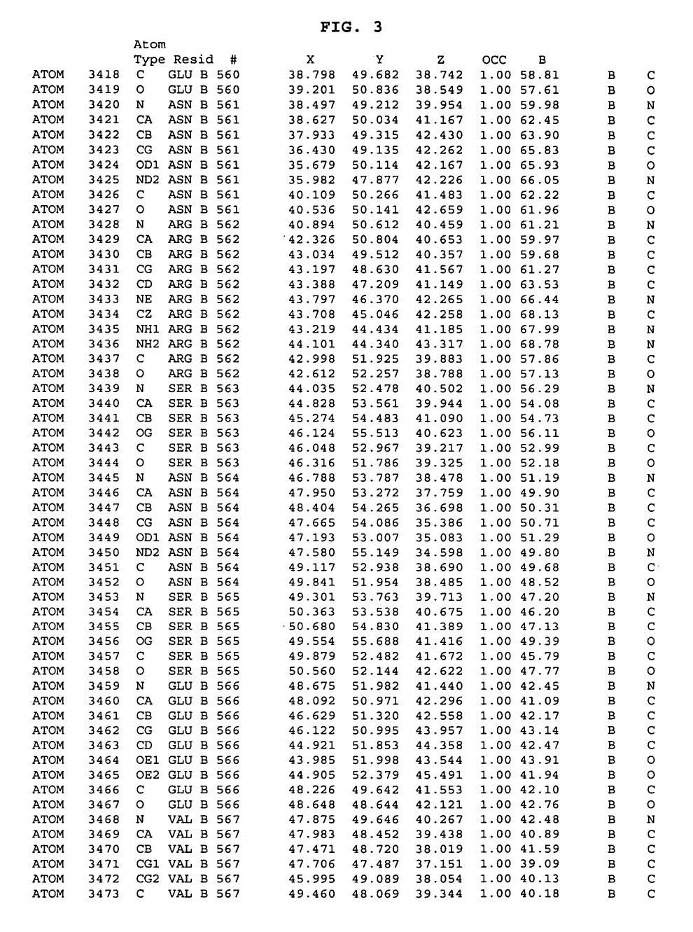

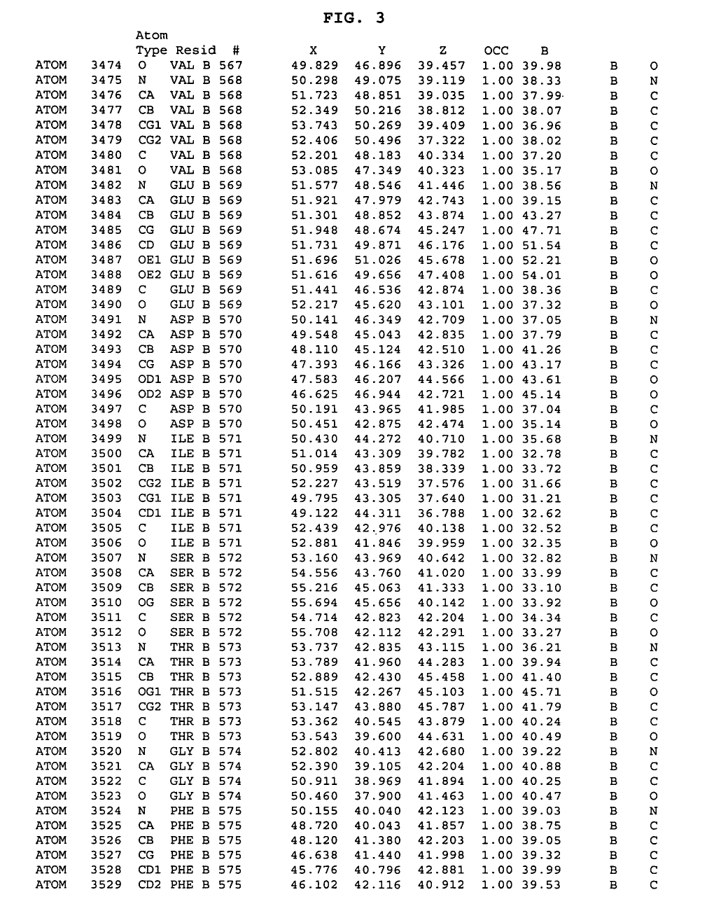

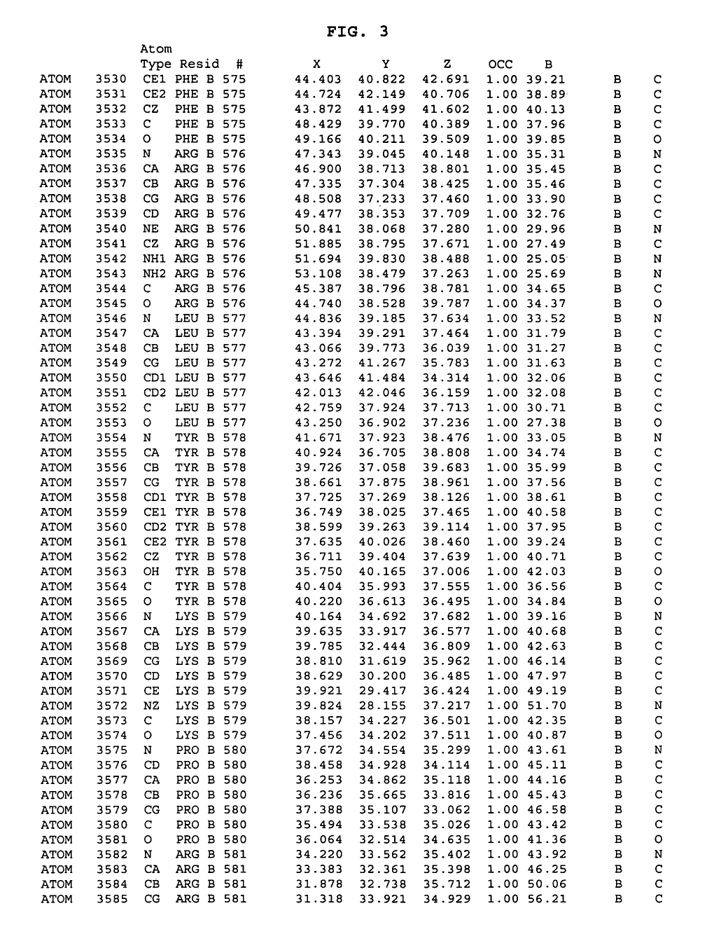

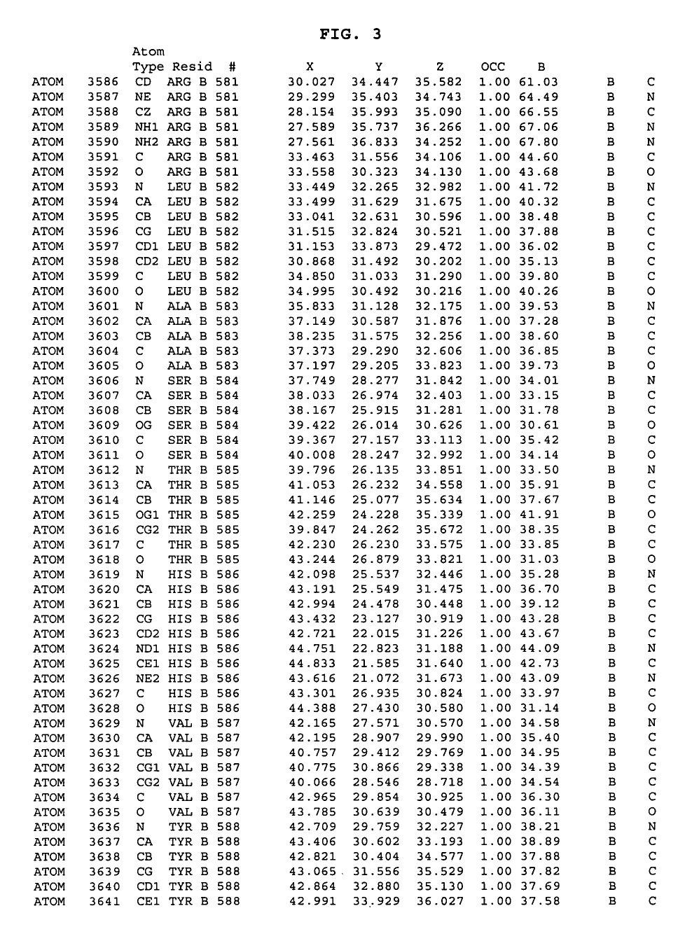

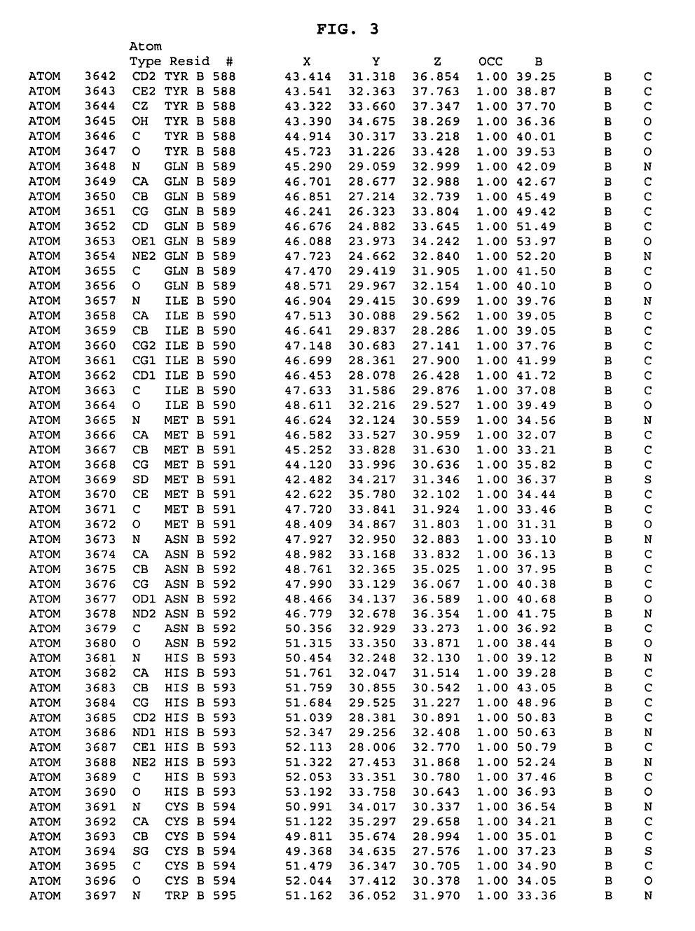

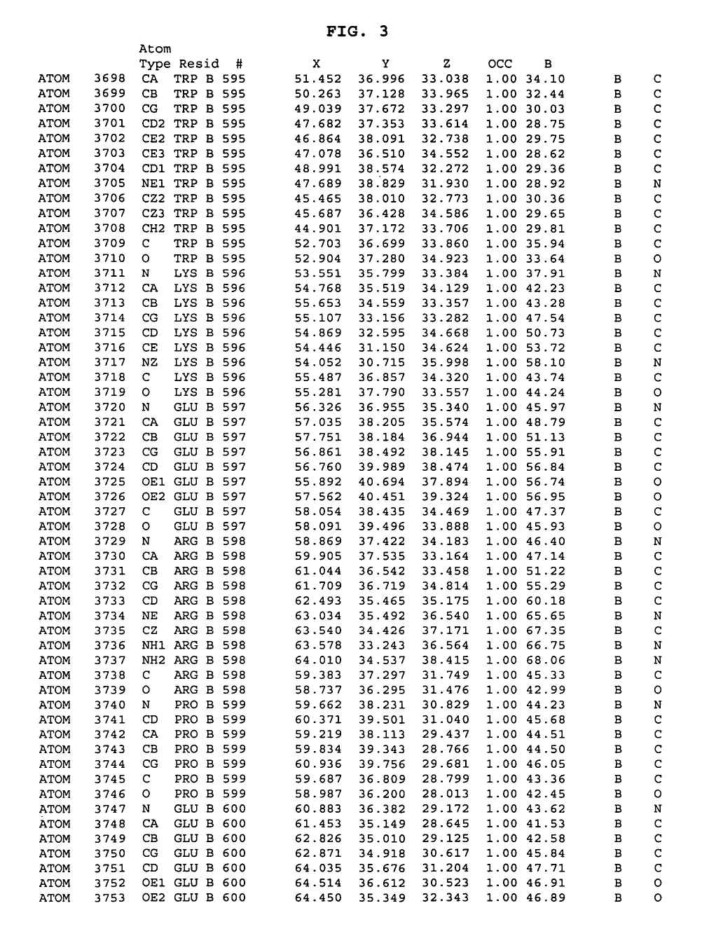

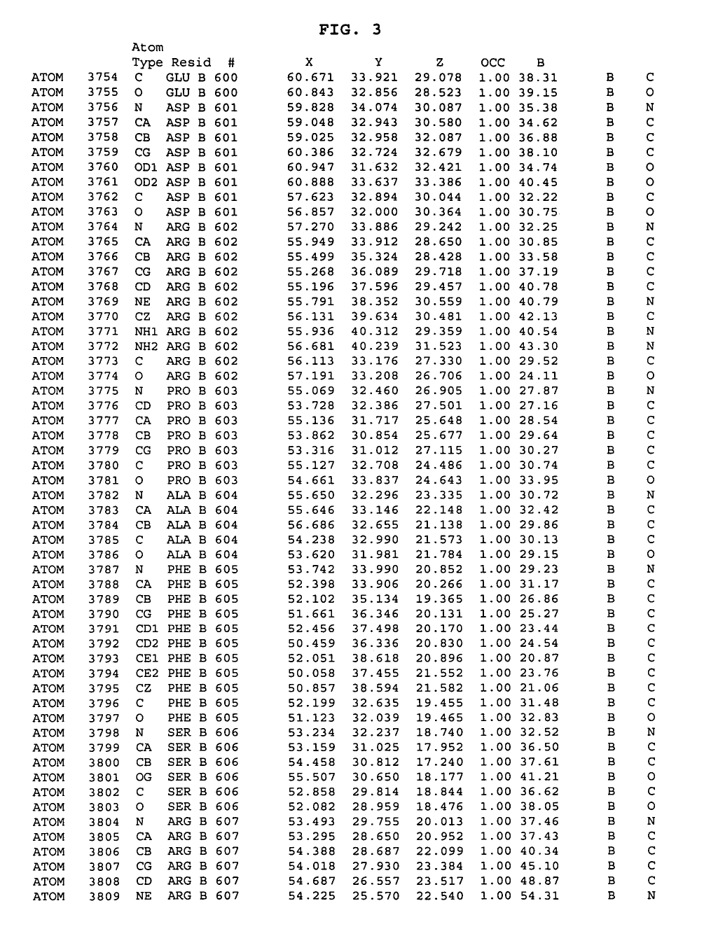

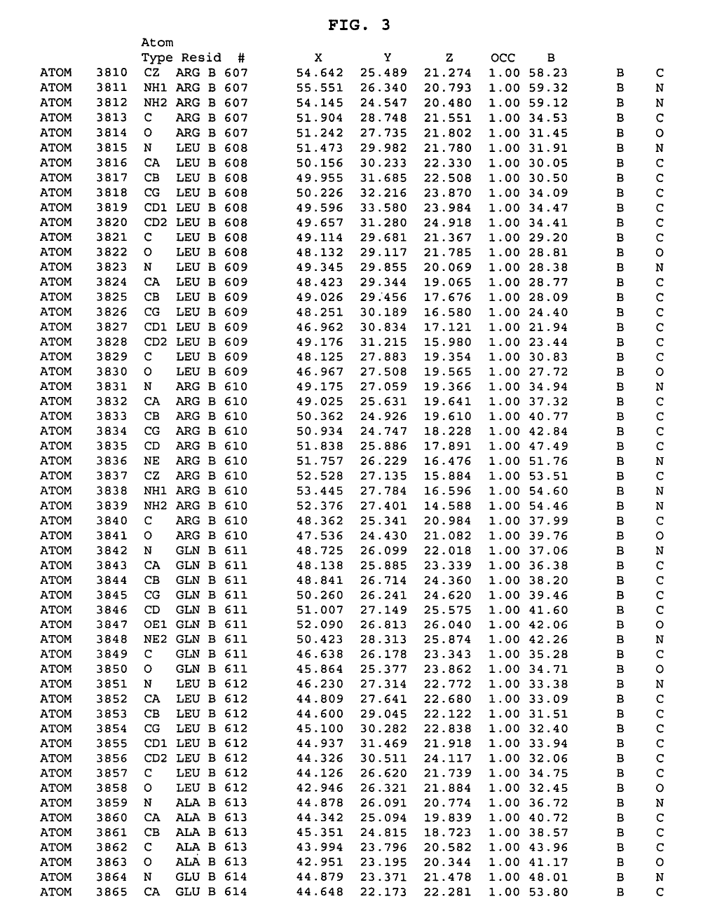

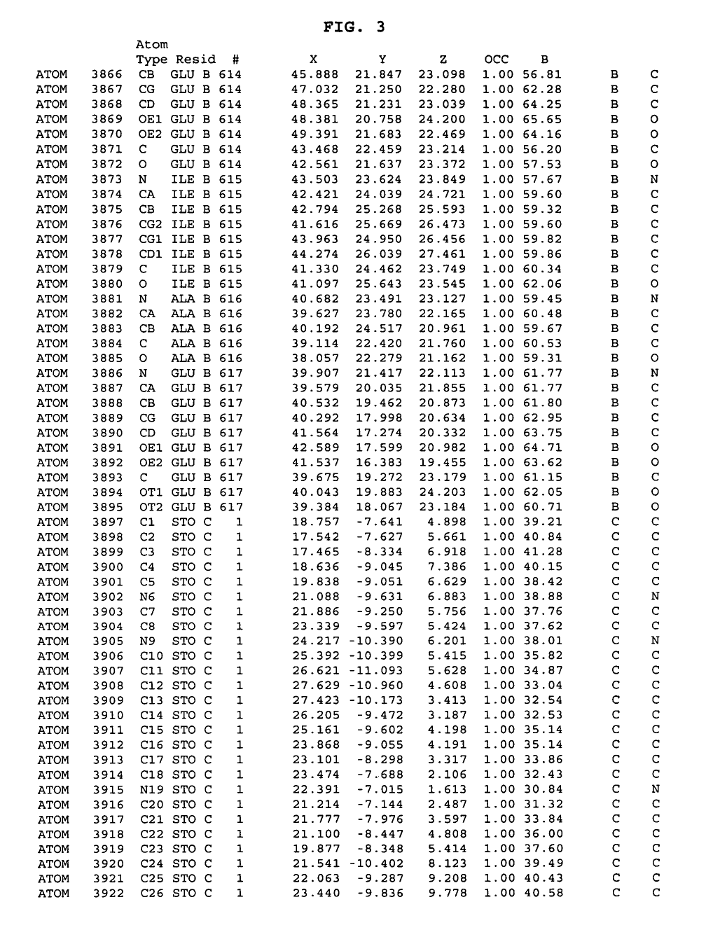

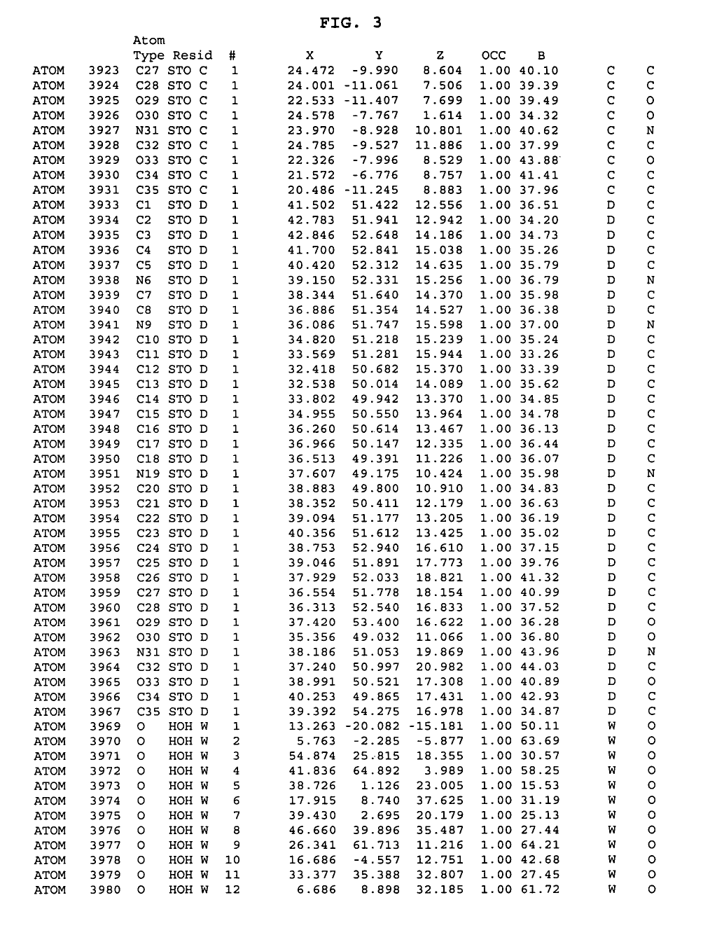

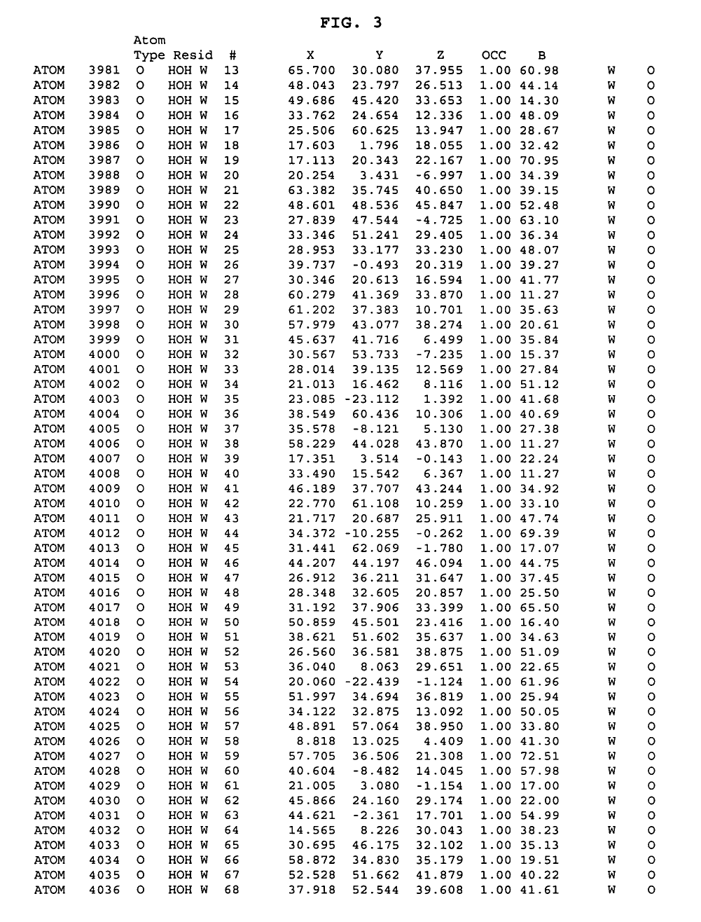

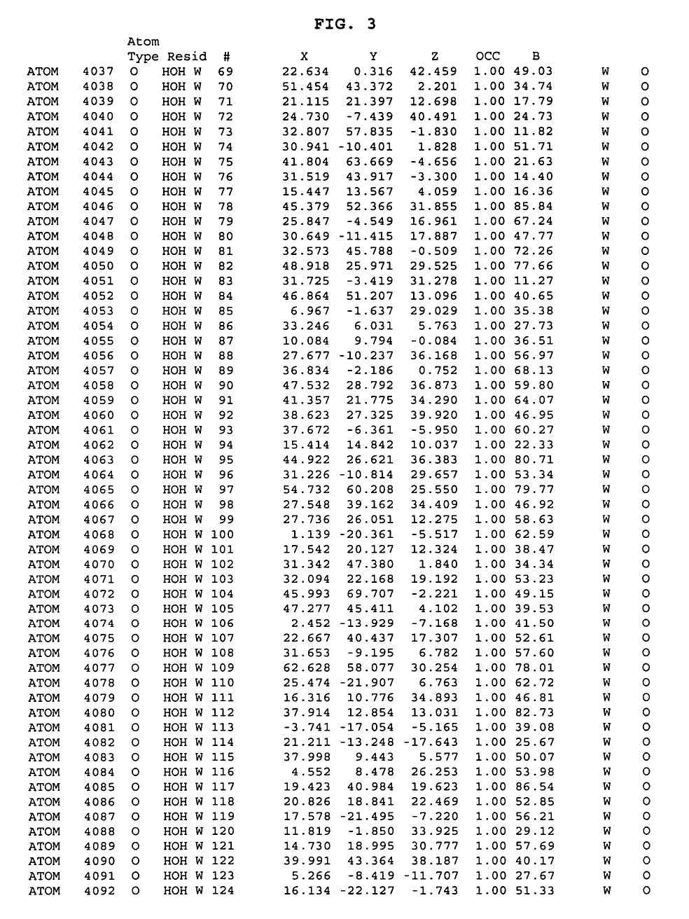

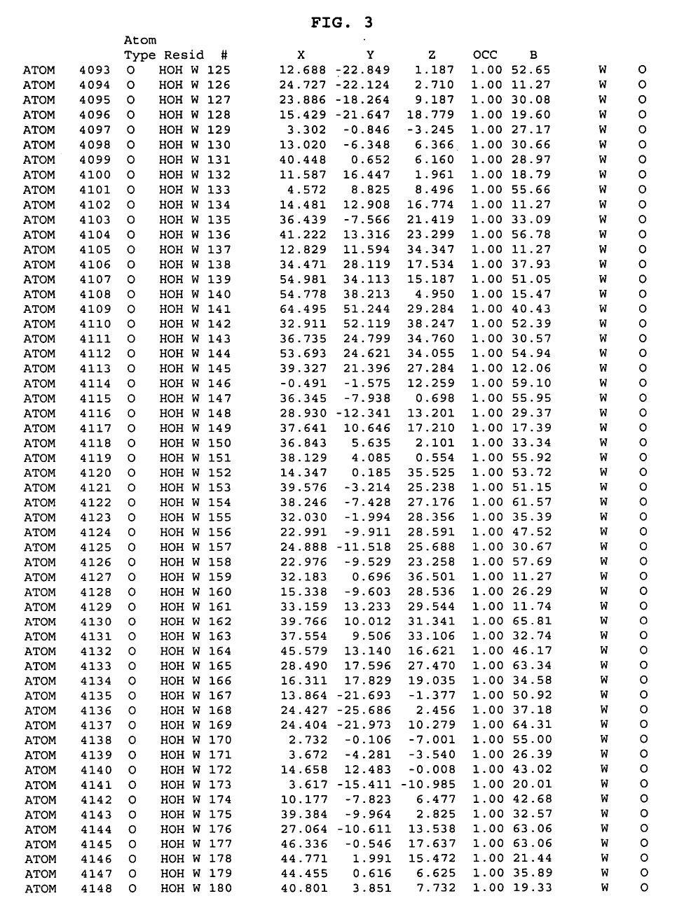

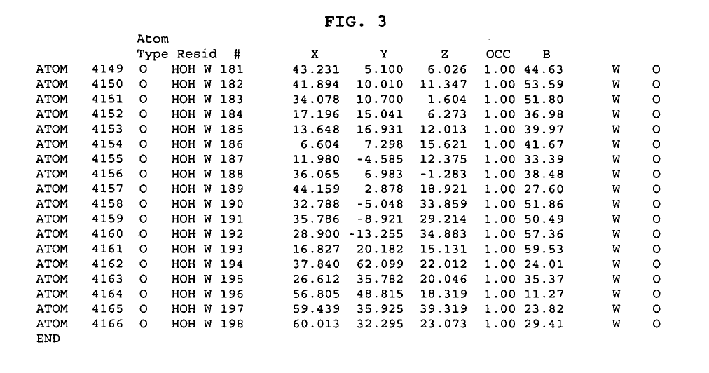

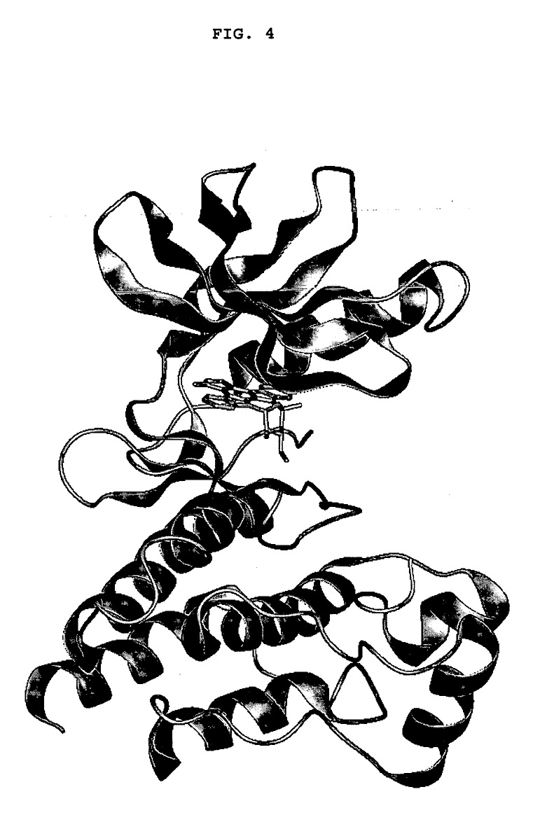

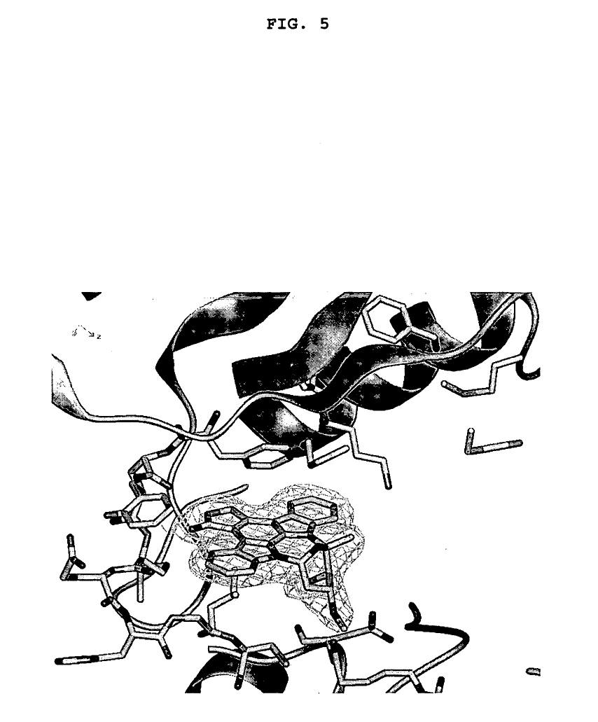

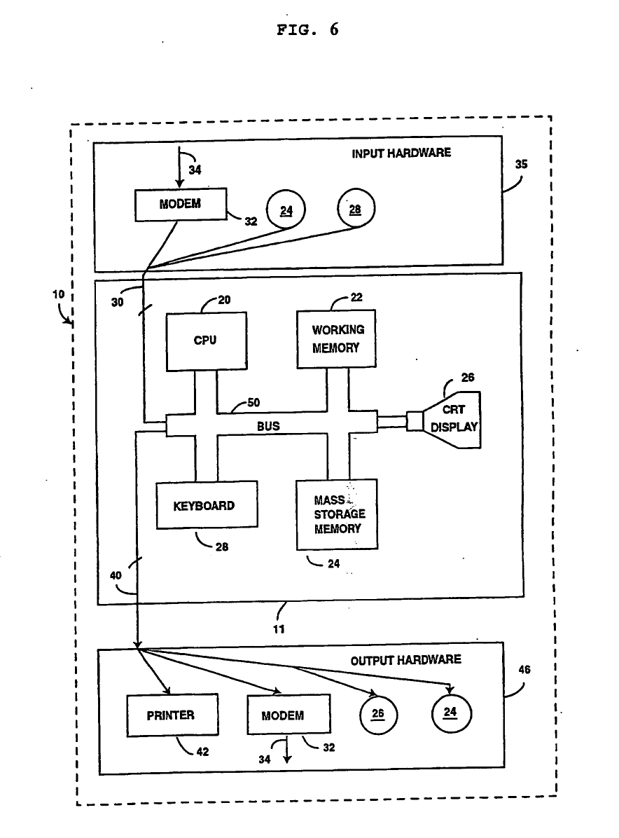

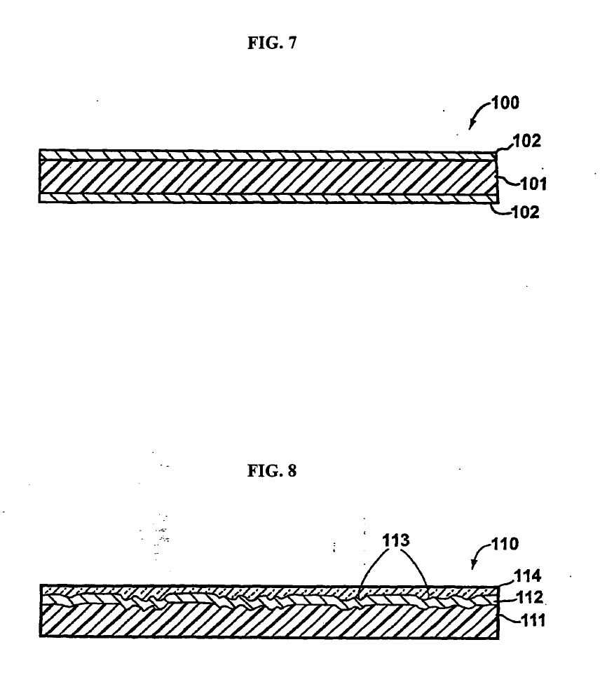

The following abbreviations are used in FIGS. 1-3:

“Atom type” refers to the element whose coordinates are measured. The first letter in the column defines the element.

“Resid” refers to the amino acid residue identity in the molecular model.

“X, Y, Z” crystallographically define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in all molecules of the crystal.

“Mol” refers to the molecule in the asymmetric unit.

FIG. 2 lists the atomic structure coordinates for the phosphorylated ITK (pITK)-staurosporine inhibitor complex as derived by X-ray diffraction from the crystal. The crystallographic asymmetric unit contains two molecular complexes. The first complex is defined as PDB chain A and C. The second is chains B and D.

FIG. 3 lists the atomic structure coordinates for the unphosphorylated ITK-staurosporine inhibitor complex as derived by X-ray diffraction from the crystal. The crystallographic asymmetric unit contains two molecular complexes. The first complex is defined as PDB chain A and C. The second is chains B and D.

FIG. 4 depicts ribbon diagrams of the overall fold of ITK-staurosporine and pITK-staurosporine complexes. The N-terminal lobe of the ITK catalytic domain corresponds to the β-strand sub-domain and encompasses residues 357 to 435. The α-helical sub-domain corresponds to residues 443 to 620. Key features of the kinase-fold such as the hinge (approximately residues 436 to 442), glycine rich loop (approximately residues 366 to 380) and activation loop or phosphorylation lip (approximately residues 500 to 521) are indicated. A number of residues in the activation loop (˜503 to 514) are disordered in each of the ITK crystal structures. They exhibited only weak electron density and could not be fitted.

FIG. 5 shows a detail representation of pockets in the catalytic active site of the pITK-staurosporine complex.

FIG. 6 shows a diagram of a system used to carry out the instructions encoded by the storage medium of FIGS. 7 and 8.

FIG. 7 shows a cross section of a magnetic storage medium.

FIG. 8 shows a cross section of an optically-readable data storage medium.

### DETAILED DESCRIPTION OF CERTAIN PREFERRED EMBODIMENTS OF THE INVENTION

In order that the invention described herein may be more fully understood, the following detailed description is set forth.

Throughout the specification, the word “comprise”, or variations such as “comprises” or “comprising” will be understood to imply the inclusion of a stated integer or groups of integers but not exclusion of any other integer or groups of integers.

The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

Additional definitions are set forth below.

The term “associating with” refers to a condition of proximity between a chemical entity or compound, or portions thereof, and a binding pocket or binding site on a protein. The association may be non-covalent—wherein the juxtaposition is energetically favored by hydrogen bonding or van der Waals or electrostatic interactions—or it may be covalent.

The term “binding pocket”, as used herein, refers to a region of a molecule or molecular complex, that, as a result of its shape and charge, favorably associates with another chemical entity or compound. The term “pocket” includes, but is not limited to, cleft, channel or site. ITK or ITK-like molecules may have binding pockets which include, but are not limited to, peptide or substrate binding, ATP-binding and antibody binding sites.

The term “chemical entity”, as used herein, refers to chemical compounds, complexes of at least two chemical compounds, and fragments of such compounds or complexes. The chemical entity may be, for example, a ligand, a substrate, a nucleotide triphosphate, a nucleotide diphosphate, phosphate, a nucleotide, an agonist, antagonist, inhibitor, antibody, drug, peptide, protein or compound.

“Conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

The term “corresponding amino acid” or “residue which corresponds to” refers to a particular amino acid or analogue thereof in an ITK homologue that corresponds to an amino acid in the ITK structure. The corresponding amino acid may be an identical, mutated, chemically modified, conserved, conservatively substituted, functionally equivalent or homologous amino acid when compared to the ITK amino acid to which it corresponds.

Methods for identifying a corresponding amino acid are known in the art and are based upon sequence, structural alignment, its functional position or a combination thereof as compared to the ITK structure. For example, corresponding amino acids may be identified by superimposing the backbone atoms of the amino acids in ITK and the ITK homologue using well known software applications, such as QUANTA [Molecular Simulations, Inc., San Diego, Calif. ©1998, 2000]. The corresponding amino acids may also be identified using sequence alignment programs such as the “bestfit” program available from the Genetics Computer Group which uses the local homology algorithm described by Smith and Waterman in Adv. Appl. Math., 2, 482 (1981), which is incorporated herein by reference.

The term “domain” refers to a portion of the ITK protein or homologue that can be separated according to its biological function, for example, catalysis. The domain is usually conserved in sequence or structure when compared to other kinases or related proteins. The domain can comprise a binding pocket, or a sequence or structural motif.

The term “sub-domain” refers to a portion of the domain as defined above in the ITK protein or homologue. The catalytic kinase domain (amino acid residues 357 to 620) of ITK is a bi-lobal structure consisting of an N-terminal, β-strand sub-domain (residues 127 to 215) and a C-terminal, α-helical sub-domain (residues 216 to 390).

The term “catalytic active site” refers to the area of the protein kinase to which nucleotide substrates bind. The catalytic active site of ITK is at the interface between the N-terminal, β-strand sub-domain and the C-terminal, α-helical sub-domain.

The “ITK ATP-binding pocket” of a molecule or molecular complex is defined by the structure coordinates of a certain set of amino acid residues present in the ITK structure, as described below. In general, the ligand for the ATP-binding pocket is a nucleotide such as ATP. This binding pocket is in the catalytic active site of the kinase domain. In the protein kinase family, the ATP-binding pocket is generally located at the interface of the α-helical and β-strand sub-domains, and is bordered by the glycine rich loop and the hinge [See, Xie et al., Structure, 6, pp. 983-991 (1998), incorporated herein by reference].

The term “ITK-like” refers to all or a portion of a molecule or molecular complex that has a commonality of shape to all or a portion of the ITK protein. In the ITK-like ATP-binding pocket, the commonality of shape is defined by a root mean square deviation of the structure coordinates of the backbone atoms between the amino acids in the ITK-like ATP-binding pocket and the amino acids in the ITK ATP-binding pocket (as set forth in FIG. 1, 2 or 3). Compared to an amino acid in the ITK ATP-binding pocket, the corresponding amino acids in the ITK-like ATP-binding pocket may or may not be identical.

The term “part of an ITK ATP-binding pocket” or “part of an ITK-like ATP-binding pocket” refers to less than all of the amino acid residues that define the ITK or ITK-like ATP-binding pocket. The structure coordinates of residues that constitute part of an ITK or ITK-like ATP-binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence. In one embodiment, part of the ITK or ITK-like ATP-binding pocket is at least two amino acid residues, preferably, E436 and M438. In another embodiment, the amino acids are selected from the group consisting of I369, V419, F435, E436, M438 and L489.

The term “ITK kinase domain” refers to the catalytic domain of ITK. The kinase domain includes, for example, the catalytic active site which comprises the catalytic residues (Table 1), the activation loop or phosphorylation lip, the DFG motif, and the glycine-rich phosphate anchor or glycine-rich loop [See, Xie et al., Structure, 6, pp. 983-991 (1998); R. Giet and C. Prigent, J. Cell Sci., 112, pp. 3591-3601 (1999), incorporated herein by reference]. The kinase domain in the ITK protein comprises residues from about 357 to 620.

The term “part of an ITK kinase domain” or “part of an ITK-like kinase domain” refers to a portion of the ITK or ITK-like catalytic domain. The structure coordinates of residues that constitute part of an ITK or ITK-like kinase domain may be specific for defining the chemical environment of the domain, or useful in designing fragments of an inhibitor that may interact with those residues. For example, the portion of residues may be key residues that play a role in ligand binding, or may be residues that are spatially related and define a three-dimensional compartment of the domain. The residues may be contiguous or non-contiguous in primary sequence. For example, part of an ITK kinase domain can be the active site, the DFG motif, the glycine-rich loop, the activation loop, or the catalytic loop [see Xie et al., supra].

The term “homologue of ITK” refers to a molecule or molecular complex that is homologous to ITK by three-dimensional structure or sequence. Examples of homologues include but are not limited to the following: human ITK with mutations, conservative substitutions, additions, deletions or a combination thereof; ITK from a species other than human; a protein comprising an ITK-like ATP-binding pocket, a kinase domain; another member of the protein kinase family, preferably the SRC kinase family or the CDK kinase family; or another member of the Tec family of protein kinases.

The term “part of an ITK protein” or “part of an ITK homologue” refers to a portion of the amino acid residues of an ITK protein or homologue. In one embodiment, part of an ITK protein or homologue defines the binding pockets, domains, sub-domains, and motifs of the protein or homologue. The structure coordinates of residues that constitute part of an ITK protein or homologue may be specific for defining the chemical environment of the protein, or useful in designing fragments of an inhibitor that may interact with those residues. The portion of residues may also be residues that are spatially related and define a three-dimensional compartment of a binding pocket, motif or domain. The residues may be contiguous or non-contiguous in primary sequence. For example, the portion of residues may be key residues that play a role in ligand or substrate binding, peptide binding, antibody binding, catalysis, structural stabilization or degradation.

The term “ITK protein complex” or “ITK homologue complex” refers to a molecular complex formed by associating the ITK protein or ITK homologue with a chemical entity, for example, a ligand, a substrate, nucleotide triphosphate, an agonist or antagonist, inhibitor, drug or compound. In one embodiment, the chemical entity is selected from the group consisting of an ATP, a nucleotide triphosphate and an inhibitor for the ATP-binding pocket. In another embodiment, the inhibitor is an ATP analog such as MgAMP-PNP (adenylyl imidodiphosphate), adenosine, staurosporine or 3-(8-Phenyl-5,6-dihydrothieno[2,3-h]quinazolin-2-ylamino)benzenesulfonamide.

The term “motif” refers to a portion of the ITK protein or homologue that defines a structural compartment or carries out a function in the protein, for example, catalysis, structural stabilization, or phosphorylation. The motif may be conserved in sequence, structure and function when compared to other kinases or related proteins. The motif can be contiguous in primary sequence or three-dimensional space. The motif can comprise α-helices and/or β-sheets. Examples of a motif include but are not limited to a binding pocket, active site, phosphorylation lip or activation loop, the glycine-rich phosphate anchor loop, the catalytic loop, the DFG loop [See, Xie et al., Structure, 6, pp. 983-991 (1998); R. Giet and C. Prigent, J. Cell Sci., 112, pp. 3591-3601 (1999)], and the degradation box.

The term “root mean square deviation” or “RMSD” means the square root of the arithmetic mean of the squares of the deviations from the mean. It is a way to express the deviation or variation from a trend or object. For purposes of this invention, the “root mean square deviation” defines the variation in the backbone of a protein from the backbone of ITK, a binding pocket, a motif, a domain, or portion thereof, as defined by the structure coordinates of ITK described herein.

The term “sufficiently homologous to ITK” refers to a protein that has a sequence homology of at least 35% compared to ITK protein. In one embodiment, the sequence homology is at least 40%, at least 60%, at least 80%, at least 90% or at least 95%.

The term “soaked” refers to a process in which the crystal is transferred to a solution containing the compound of interest. In certain embodiments, the compound is diffused into the crystal.

The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex. It would be readily apparent to those skilled in the art that all or part of the structure coordinates of FIG. 1 (either molecule A or B) may have a RMSD deviation of 0.1 Å because of standard error.

The term “about” when used in the context of RMSD values takes into consideration the standard error of the RMSD value, which is ±0.1 Å.

The term “crystallization solution” refers to a solution that promotes crystallization. The solution comprises at least one agent, and may include a buffer, one or more salts, a precipitating agent, one or more detergents, sugars or organic compounds, lanthanide ions, a poly-ionic compound and/or a stabilizer.

The term “generating a three-dimensional structure” or “generating a three-dimensional graphical representation” refers to converting the lists of structure coordinates into structural models in three-dimensional space. This can be achieved through commercially or publicly available software. The three-dimensional structure may be displayed as a graphical representation or used to perform computer modeling or fitting operations. In addition, the structure coordinates themselves may be used to perform computer modeling and fitting operations.

The term “homologue of ITK” or “ITK homologue” refers to a molecule that is homologous to ITK by three-dimensional structure or sequence and retains the kinase activity of ITK. Examples of homologues include, but are not limited to, ITK having one or more amino acid residues that are chemically modified, mutated, conservatively substituted, added, deleted or a combination thereof.

The term “homology model” refers to a structural model derived from known three-dimensional structure(s). Generation of the homology model, termed “homology modeling”, can include sequence alignment, residue replacement, residue conformation adjustment through energy minimization, or a combination thereof

The term “three-dimensional structural information” refers to information obtained from the structure coordinates. Structural information generated can include the three-dimensional structure or graphical representation of the structure. Structural information can also be generated when subtracting distances between atoms in the structure coordinates, calculating chemical energies for an ITK molecule or molecular complex or homologues thereof, calculating or minimizing energies for an association of an ITK molecule or molecular complex or homologues thereof to a chemical entity.

Crystallizable Compositions and Crystals of ITK Complexes

According to another embodiment, the invention provides a crystallizable composition comprising phosphorylated ITK protein. In another embodiment, the invention provides a crystallizable composition comprising phosphorylated ITK protein and an inhibitor. In another embodiment, the invention provides a crystallizable composition comprising phosphorylated ITK protein and a substrate analogue, such as but not limited to adenosine. In one embodiment, the aforementioned crystallizable composition further comprises a precipitant, 400-1000 nM Ammonium sulphate, 200 mM Magnesium Acetate and a buffer that maintains pH at between about 4.0 and 8.0. The composition may further comprise a reducing agent, such as dithiothreitol (DTT) at between about 1 to 20 mM. In another embodiment, the aforementioned crystallizable composition further comprises a precipitant, 1-15% Peg3350, 200 mM Ammonium Acetate and a buffer that maintains pH at between about 4.0 and 8.0. The composition may further comprise a reducing agent, such as dithiothreitol (DTT) at between about 1 to 20 mM. The phosphorylated ITK protein or complex is preferably 85-100% pure prior to forming the composition.

According to another embodiment, the invention provides a crystal composition comprising ITK protein complex. In one embodiment, the crystal has a unit cell dimension of a=125 Å, b=75 Å, c=79 Å, α=γ=90°, β=94° and belongs to space group C2. It will be readily apparent to those skilled in the art that the unit cells of the crystal compositions may deviate ±1-2 Å from the above cell dimensions depending on the deviation in the unit cell calculations.

As used herein, the ITK protein in the crystal or crystallizable compositions can be a truncated protein with amino acids 357-620 as shown in FIGS. 1-3; and the truncated protein with conservative substitutions.

The ITK protein may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products. Preferably, the protein is overexpressed from a baculovirus system. The unphosphorylated ITK protein is not phosphorylated at any of the phosphorylation sites.

The invention also relates to a method of making crystals of ITK complexes or ITK homologue complexes. Such methods comprise the steps of:
a) producing a composition comprising a crystallization solution and ITK protein or homologue thereof complexed with a chemical entity; and
b) subjecting said composition to devices or conditions which promote crystallization.

In one embodiment, the chemical entity is selected from the group consisting of an ATP analogue, nucleotide triphosphate, nucleotide diphosphate, phosphate, adenosine, or active site inhibitor. In another embodiment, the chemical entity is an ATP analogue. In certain exemplary embodiments, the chemical entity is staurosporine. In certain other exemplary embodiments, the chemical entity is 3-(8-Phenyl-5,6-dihydrothieno[2,3-h]quinazolin-2-ylamino)benzenesulfon-amide. In yet another embodiment, the crystallization solution is as described previously. In another embodiment, the composition is treated with micro-crystals of ITK or ITK complexes or homologues thereof. In another embodiment, the composition is treated with micro-crystals of ITK complexes or homologues thereof.

In certain embodiments, the invention provides a method of making ITK crystals, the method comprising steps of:
a) producing and purifying ITK protein;
b) producing a crystallizable composition; and
c) subjecting said composition to devices which promote crystallization.

In one embodiment, the crystallizable composition of step b) is made according to the conditions discussed above. In certain exemplary embodiments, the crystallization composition comprises a precipitant, ammonium sulphate, magnesium acetate, and/or a buffer that maintains pH at a desired range. In certain embodiments, the crystallizable composition comprises a a buffer that maintains pH at between about 4.0 and 8.0. In certain other embodiments, the crystallizable composition further comprises a reducing agent. In certain embodiments, the reducing agent is present at between about 1 to 20 mM. In certain exemplary embodiments, the reducing agent is dithiothreitol (DTT). In certain exemplary embodiments, the crystallizable composition comprises a precipitant, 400-1000 nM Ammonium sulphate, 200 mM Magnesium Acetate and a buffer that maintains pH at between about 4.0 and 8.0. In certain other exemplary embodiments, the crystallizable composition comprises a precipitant, 1-15% Peg3350, 200 mM Ammonium Acetate and a buffer that maintains pH at between about 4.0 and 8.0. In certain embodiments, the composition further comprises a reducing agent, such as dithiothreitol (DTT) at between about 1 to 20 mM. In certain other embodiments, the ITK protein of step a) is a phosphorylated ITK protein or complex. In certain exemplary embodiments, the phosphorylated ITK protein or complex is preferably 85-100% pure prior to forming the composition.

Devices for promoting crystallization can include but are not limited to the hanging-drop, sitting-drop, dialysis or microtube batch devices. [U.S. Pat. Nos. 4,886,646, 5,096,676, 5,130,105, 5,221,410 and 5,400,741; Pav et al., Proteins: Structure, Function, and Genetics, 20, pp. 98-102 (1994), incorporated herein by reference]. The hanging-drop or sitting-drop methods produce crystals by vapor diffusion. The hanging-drop, sitting-drop, and some adaptations of the microbatch methods [D'Arcy et al., J. Cryst. Growth, 168, pp. 175-180 (1996) and Chayen, J. Appl. Cryst., 30, pp. 198-202 (1997)] produce crystals by vapor diffusion. The hanging drop and sitting drop containing the crystallizable composition is equilibrated in a reservoir containing a higher or lower concentration of the precipitant. As the drop approaches equilibrium with the reservoir, the saturation of protein in the solution leads to the formation of crystals.

Microseeding or seeding may be used to obtain larger, or better quality (i.e., crystals with higher resolution diffraction or single crystals) crystals from initial micro-crystals. Microseeding involves the use of crystalline particles to provide nucleation under controlled crystallization conditions. Microseeding is used to increase the size and quality of crystals. In this instance, micro-crystals are crushed to yield a stock seed solution. The stock seed solution is diluted in series. Using a needle, glass rod or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art following the teachings of the specification to vary the crystallization conditions disclosed herein to identify other crystallization conditions that would produce crystals of ITK homologue, ITK homologue complex, ITK protein or other ITK protein complexes. Such variations include, but are not limited to, adjusting pH, protein concentration and/or crystallization temperature, changing the identity or concentration of salt and/or precipitant used, using a different method of crystallization, or introducing additives such as detergents (e.g., TWEEN 20 (monolaurate), LDAO, Brij 30 (4 lauryl ether)), sugars (e.g., glucose, maltose), organic compounds (e.g., dioxane, dimethylformamide), lanthanide ions or polyionic compounds that aid in crystallization. High throughput crystallization assays may also be used to assist in finding or optimizing the crystallization conditions.

Binding Pockets of ITK Protein or Homologues Thereof

As disclosed above, applicants have provided for the first time the three-dimensional X-ray crystal structures of three ITK-inhibitor complexes. The crystal structures of ITK presented here are the first reported for ITK and the first of an active kinase within the TEC-family kinases. The invention will be useful for inhibitor design to study the role of ITK in cell signaling. The atomic coordinate data is presented in FIGS. 1-3.

In order to use the structure coordinates generated for ITK, their complexes, one of their binding pockets, or an ITK-like binding pocket thereof, it is often times necessary to convert the coordinates into a three-dimensional shape. This is achieved through the use of commercially available software that is capable of generating three-dimensional graphical representations (e.g., three-dimensional structures) of molecules or portions thereof from a set of structure coordinates.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential inhibitors of the binding pockets of biologically important targets. The ATP and substrate binding pockets of this invention will be important for drug design.

In one embodiment, the ATP-binding pocket comprises amino acids I369, G370, V377, A389, K391, V419, F435, E436, F437, M438, E439, H440, C442, D445, L489 and S499 using the structure of the ITK-3-(8-Phenyl-5,6-dihydrothieno[2,3-h]quinazolin-2-ylamino)benzenesulfonamide complex according to FIG. 1. In another embodiment, the ATP-binding pocket comprises amino acids I369, G370, V377, A389, K391, V419, F435, E436, F437, M438, E439, H440, C442, D445, L489 and S499 using the structure of the pITK staurosporine complex according to FIG. 2. In another embodiment, the ATP-binding pocket comprises amino acids I369, G370, V377, A389, K391, V419, F435, E436, F437, M438, E439, H440, C442, D445, L489 and S499 using the structure of the ITK-staurosporine complex according to FIG. 3. In resolving the crystal structures of the unphosphorylated and phosphorylated ITK-inhibitor complexes, applicants have determined that the above amino acids are within 5 Å (“5 Å sphere amino acids”) of the inhibitor bound in the ATP-binding pockets. These residues were identified using the program QUANTA [Molecular Simulations, Inc., San Diego, Calif. ©1998, 2000], O [T. A. Jones et al., Acta Cryst. A, 47, pp. 110-119 (1991)] and RIBBONS [Carson, J. Appl. Cryst., 24, pp. 958-961 (1991)]. The programs allow one to display and output all residues within 5 Å from the inhibitor. Thus, a binding pocket defined by the structural coordinates of these amino acids, as set forth in FIGS. 1, 2 and 3 is considered an ITK-ATP binding pocket of this invention.

In another embodiment, the ATP-binding pocket comprises amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 using the structure of the ITK-3-(8-Phenyl-5,6-dihydrothieno[2,3-h]quinazolin-2-ylamino)benzenesulfonamide complex to FIG. 1. In another embodiment, the ATP-binding pocket comprises amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 using the structure of the pITK-staurosporine complex according to FIG. 2. In another embodiment, the ATP-binding pocket comprises amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 using the structure of the ITK-staurosporine complex according to FIG. 3. In the crystal structures of the ITK-inhibitor complexes, applicants have determined that the above amino acids are within 8 Å (“8 Å sphere amino acids”) of the inhibitor bound in the ATP-binding pockets. These residues were identified using the programs QUANTA, O and RIBBONS, supra. Thus, a binding pocket defined by the structural coordinates of these amino acids, as set forth in FIGS. 1, 2 and 3 is considered an ITK-ATP binding pocket of this invention.

In another embodiment, the ATP-binding pocket comprises amino acids L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 using the structure of the ITK-3-(8-Phenyl-5,6-dihydrothieno[2,3-h]quinazolin-2-ylamino)benzenesulfonamide complex to FIG. 1. In another embodiment, the ATP-binding pocket comprises amino acids L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 using the structure of the pITK-staurosporine complex according to FIG. 2. In another embodiment, the ATP-binding pocket comprises amino acids L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 using the structure of the ITK-staurosporine complex according to FIG. 3. Using a multiple alignment program to compare each ITK structure and structures of other members of the protein kinase family [Gerstein et al., J. Mol. Biol. 251, pp. 161-175 (1995), incorporated herein by reference], applicants have identified the above amino acids as the ATP-binding pocket. First, a sequence alignment between members of the protein kinase family including Aurora-2 [PDB Accession number 1MUO], p 38 [K. P. Wilson et al., J. Biol. Chem., 271, pp. 27696-27700 (1996); Z. Wang et al., Proc. Natl. Acad. Sci. U.S.A., 94, pp. 2327-32 (1997)], CDK2 [PDB Accession number 1B38], SRC [Xu, W., et al., Cell 3, pp. 629-638 (1999); PDB Accession number 2SRC], MK2 [U.S. Provisional application 60/337,513] and LCK [Yamaguchi H., Hendrickson W. A., Nature. 384, pp. 484-489 (1996); PDB Accession number 3LCK] is performed. Then, a putative core is constructed by superimposing a series of corresponding structures in the protein kinase family. Then, residues of high spatial variation are discarded, and the core alignment is iteratively refined. The amino acids that make up the final core structure have low structural variance and have the same local and global conformation relative to the corresponding residues in the protein family.

In one embodiment, the ATP-binding pocket comprises the amino acids of I369, V419, F435, E436, M438 and L489 according to FIGS. 1, 2 and 3. It will be readily apparent to those of skill in the art that the numbering of amino acids in other homologues of ITK may be different than that set forth for ITK. Corresponding amino acids in homologues of ITK are easily identified by visual inspection of the amino acid sequences or by using commercially available sequence homology, structural homology or structure superimposition software programs.

Those of skill in the art understand that a set of structure coordinates for a molecule or a molecular-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated because of mathematical manipulations of the ITK structure coordinates. For example, the structure coordinates set forth in FIG. 1, 2 or 3 could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that bound to the binding pocket of ITK would also be expected to bind to another binding pocket whose structure coordinates defined a shape that fell within the acceptable root mean square deviation.

Various computational analyses maybe necessary to determine whether a binding pocket, motif, domain or portion thereof of a molecule or molecular complex is sufficiently similar to the binding pocket, motif, domain or portion thereof of ITK. Such analyses may be carried out in well known software applications, such as ProFit [A. C. R. Martin, SciTech Software, ProFit version 1.8, University College London, http://www.bioinf.org.uk/software], Swiss-Pdb Viewer [Guex et al., Electrophoresis 18, pp. 2714-2723 (1997)], the Molecular Similarity application of QUANTA [Molecular Simulations Inc., San Diego, Calif. © 1998, 2000] and as described in the accompanying User's Guide, which are incorporated herein by reference.

The above programs permit comparisons between different structures, different conformations of the same structure, and different parts of the same structure. The procedure used in QUANTA [Molecular Simulations, Inc., San Diego, Calif. ©1998, 2000] and Swiss-Pdb Viewer to compare structures is divided into four steps: 1) load the structures to be compared; 2) define the atom equivalences in these structures; 3) perform a fitting operation on the structures; and 4) analyze the results. The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within the above programs is defined by user input, for the purpose of this invention we will define equivalent atoms as protein backbone atoms (N, Cα, C and O) for ITK amino acids and corresponding amino acids in the structures being compared.

The corresponding amino acids may be identified by sequence alignment programs such as the “bestfit” program available from the Genetics Computer Group which uses the local homology algorithm described by Smith and Waterman in Advances in Applied Mathematics 2, 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acids with the second protein [Hanks et al., Science, 241, 42 (1988); Hanks and Quinn, Meth. Enzymol., 200, 38 (1991)]. The identification of equivalent residues can also be assisted by secondary structure alignment, for example, aligning the a-helices, β-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. For programs that calculate an average of the individual RMSD values of the backbone atoms, an RMSD cutoff value can be used to exclude pairs of equivalent atoms with extreme individual RMSD values. In the program ProFit, the RMSD cutoff value can be specified by the user.

The RMSD values between other protein kinases the ITK protein complexes (FIGS. 1-3) and other kinases are illustrated in Tables 2-4. The RMSD values were determined by the programs ProFit from initial rigid fitting results from QUANTA. The RMSD values provided in Table 2 are averages of individual RMSD values calculated for the backbone atoms in the kinase or ATP-binding pocket. The RMSD cutoff value in ProFit was specified as 3 Å.

For the 5 Å and 8 Å sphere amino acids, the values for the RMSD values of the ATP-binding pocket between the phosphorylated pITK-staurosporine complex and the ITK-3-(8-Phenyl-5,6-dihydrothieno[2,3-h]quinazolin-2-ylamino)benzenesulfonamide inhibitor complexes are 1.31 Å and 0.98 Å, respectively. The comparison of the whole kinase domain yields RMSD values of 0.95 Å using the ITK-3-(8-Phenyl-5,6-dihydrothieno[2,3-h]quinazolin-2-ylamino)benzenesulfonamide inhibitor complex as a reference.

For the 5 Å and 8 Å sphere amino acids, the values for the RMSD values of the ATP-binding pocket between the unphosphorylated pITK-staurosporine complex and the ITK-3-(8-Phenyl-5,6-dihydrothieno[2,3-h]quinazolin-2-ylamino)benzenesulfonamide inhibitor complexes are 1.23 Å and 0.89 Å, respectively. The comparison of the whole kinase domain yields RMSD values of 0.88 Å using the ITK-3-(8-Phenyl-5,6-dihydrothieno[2,3-h]quinazolin-2-ylamino)benzenesulfonamide inhibitor complex as a reference.

For the 5 Å and 8 Å sphere amino acids, the values for the RMSD values of the ATP-binding pocket between the phosphorylated pITK-staurosporine and the unphosphorylated ITK-staurosporine complexes are 0.27 Å and 0.33 Å, respectively. The comparison of the whole kinase domain yields RMSD values of 0.27 Å using the phosphorylated pITK-staurosporine complex as a reference.

TABLE 2
RMSD values for ITK - 3-(8-Phenyl-5,6-dihydrothieno[2,3-
h]quinazolin-2-ylamino)benzenesulfonamide complex

| | RMSD value | RMSD value | |
|---|---|---|---|
| | between ATP- | between ATP- | RMSD value |
| | binding pocket (8 Å | binding pocket (5 Å | between ITK- |
| | sphere of amino | sphere of amino | complex kinase |
| | acids) and | acids) and | domain and |
| | corresponding amino | corresponding amino | kinase domain |
| Protein | acids in protein (Å) | acids in protein (Å) | in protein (Å) |
| Aur-2^{a} | 1.56 | 1.80 | 4.31 |
| P38^{b} | 1.64 | 1.79 | 12.32 |
| cdk2^{c} | 1.90 | 2.23 | 7.70 |
| SRC^{d} | 1.46 | 1.56 | 2.68 |
| MK2^{e} | 1.06 | 1.42 | 15.41 |
| LCK^{f} | 1.07 | 1.24 | 2.18 |

^{a}Aurora-2 kinase: Patent Cooperation Treaty Application No.: PCT/US03/13605.
^{b}p38: Wilson et al., J. Biol. Chem., 271, pp. 27696-27700 (1996); Z. Wang et al., Proc. Natl. Acad. Sci. U.S.A., 94, pp. 2327-2332 (1997); PDB Accession number 1WFC
^{c}Cyclin-dependent kinase 2: Brown, N. R., et al., J. Biol. Chem. 274, pp. 8746-8756 (1999); PDB Accession number 1B38.
^{d}Human kinase from Rous Sarcoma virus (SRC): Xu, W., et al., Cell 3, pp. 629-638 (1999); PDB Accession number 2SRC.
^{e}Mitogen activated protein kinase activated protein (MAPKAP) kinase 2: Patent Cooperation Treaty Application No.: PCT/US02/39070.
^{f}Lymphocyte-specific kinase (LCK): ref Yamaguchi H., Hendrickson W. A., Nature. 384, pp. 484-489 (1996); PDB Accession number 3LCK.

TABLE 3
RMSD values for pITK - staurosporine complex

| | RMSD value | RMSD value | |
|---|---|---|---|
| | between ATP- | between ATP- | RMSD value |
| | binding pocket (8 Å | binding pocket (5 Å | between ITK- |
| | sphere of amino | sphere of amino | complex kinase |
| | acids) and | acids) and | domain and |
| | corresponding amino | corresponding amino | kinase domain |
| Protein | acids in protein (Å) | acids in protein (Å) | in protein (Å) |
| Aur-2^{a} | 1.06 | 0.84 | 6.68 |
| P38^{b} | 1.41 | 1.49 | 12.42 |
| cdk2^{c} | 1.44 | 1.66 | 8.97 |
| SRC^{d} | 0.94 | 0.62 | 2.23 |
| MK2^{e} | 0.94 | 1.49 | 16.89 |
| LCK^{f} | 0.87 | 0.68 | 1.88 |

TABLE 4
RMSD values for ITK - staurosporine

| | RMSD value | RMSD value | |
|---|---|---|---|
| | between ATP- | between ATP- | RMSD value |
| | binding pocket (8 Å | binding pocket (5 Å | between ITK- |
| | sphere of amino | sphere of amino | complex kinase |
| | acids) and | acids) and | domain and |
| | corresponding amino | corresponding amino | kinase domain |
| Protein | acids in protein (Å) | acids in protein (Å) | in protein (Å) |
| Aur-2^{a} | 1.56 | 1.29 | 4.41 |
| P38^{b} | 1.34 | 1.04 | 11.96 |
| cdk2^{c} | 1.46 | 2.21 | 8.84 |
| SRC^{d} | 0.97 | 0.63 | 2.27 |
| MK2^{e} | 0.93 | 1.57 | 16.87 |
| LCK^{f} | 0.76 | 0.60 | 1.80 |

For the purpose of this invention, any molecule, molecular complex, binding pocket, motif, domain thereof or portion thereof that is within a root mean square deviation for backbone atoms (N, Cα, C, O) when superimposed on the relevant backbone atoms described by structure coordinates listed in FIG. 1, 2 or 3 are encompassed by this invention.

Therefore, one embodiment of this invention provides a molecule or molecular complex comprising all or part of an ITK ATP-binding pocket defined by structure coordinates of ITK amino acids I369, G370, V377, A389, K391, V419, F435, E436, F437, M438, E439, H440, C442, D445, L489 and S499 according to FIG. 1; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.1 Å, 0.9 Å, 0.7 Å, or 0.5 Å and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to FIG. 1; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.0 Å, 0.8 Å, or 0.6 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids L363, F365, V366, Q367, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to FIG. 1; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.0 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids 1369, V419, F435, E436, M438 and L489 according to FIG. 1; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.0 Å, and wherein at least one of said corresponding amino acids is not identical to the ITK amino acid to which it corresponds.

One embodiment of this invention provides a molecule or molecular complex comprising all or part of an ITK ATP-binding pocket defined by structure coordinates of ITK amino acids I369, G370, V377, A389, K391, V419, F435, E436, F437, M438, E439, H440, C442, D445, L489 and S499 according to FIG. 2; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.1 Å, 0.9 Å, 0.7 Å or 0.5 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to FIG. 2; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.3 Å, 1.1 Å, 0.9 Å, or 0.7 Å, or 0.5 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to FIG. 2; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.1 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids I369, V419, F435, E436, M438 and L489 according to FIG. 2; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.1 Å, and wherein at least one of said corresponding amino acids is not identical to the ITK amino acid to which it corresponds.

One embodiment of this invention provides a molecule or molecular complex comprising all or part of an ITK ATP-binding pocket defined by structure coordinates of ITK amino acids acids I369, G370, V377, A389, K391, V419, F435, E436, F437, M438, E439, H440, C442, D445, L489 and S499 according to FIG. 3; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.7 Å, 1.5 Å, 1.3 Å, 1.1 Å, 0.9 Å, or 0.7, or 0.5 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to FIG. 3; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.4 Å, 1.2 Å, 1.0 Å, 0.8 Å, or 0.6 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids L363, F365, V366, Q367, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to FIG. 3; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.3 Å, and wherein at least one of said corresponding amino acids is not identical to the ITK amino acid to which it corresponds.

Another embodiment of this invention provides a molecule or molecular complex comprising all or part of an ITK ATP-binding pocket defined by structure coordinates of ITK amino acids I369, V419, F435, E436, M438 and L489, according to FIG. 3; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.3 Å, and wherein at least one of said corresponding amino acids is not identical to the ITK amino acid to which it corresponds.

One embodiment of this invention provides a molecule or molecular complex comprising all or part of a ITK protein kinase domain defined by the structure coordinates of ITK amino acids set forth in FIG. 1; or all or part of an ITK-like protein kinase domain defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or an ITK-like protein kinase domain defined by structure coordinates of a set of corresponding amino acids, wherein the root mean square deviation of the backbone atoms between said set of corresponding amino acids and ITK amino acids is not more than about 4.5 Å, 4.0 Å, 3.5 Å, 3.0 Å, 2.5 Å, 2.0 Å, 1.5 Å, or 1.0 Å, and wherein at least one of said corresponding amino acids is not identical to the ITK amino acid to which it corresponds.

Another embodiment of this invention provides a molecule or molecular complex comprising all or part of a ITK protein kinase domain defined by the structure coordinates of ITK amino acids set forth in FIG. 2; or all or part of an ITK-like protein kinase domain defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or an ITK-like protein kinase domain defined by structure coordinates of a set of corresponding amino acids, wherein the root mean square deviation of the backbone atoms between said set of corresponding amino acids and ITK amino acids is not more than about 4.6 Å, 4.0 Å, 3.5 Å, 3.0 Å, 2.5 Å, 2.0 Å, 1.5 Å, or 1.0 Å, and wherein at least one of said corresponding amino acids is not identical to the ITK amino acid to which it corresponds.

Another embodiment of this invention provides a molecule or molecular complex comprising an ITK protein kinase domain defined by the structure coordinates of ITK amino acids set forth in FIG. 3; or all or part of an ITK-like protein kinase domain defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or an ITK-like protein kinase domain defined by structure coordinates of a set of corresponding amino acids, wherein the root mean square deviation of the backbone atoms between said set of corresponding amino acids and ITK amino acids is not more than about 3.6 Å, 3.0 Å, 2.5 Å, 2.0 Å, 1.5 Å, or 1.0 Å, and wherein at least one of said corresponding amino acids is not identical to the ITK amino acid to which it corresponds.

In one embodiment, the above molecules or molecular complexes are in crystalline form.

Computer Systems

According to another embodiment of this invention is provided a machine-readable data storage medium, comprising a data storage material encoded with machine-readable data, wherein said data comprises all or part of an ITK ATP-binding pocket defined by structure coordinates of ITK amino acids I369, G370, V377, A389, K391, V419, F435, E436, F437, M438, E439, H440, C442, D445, L489 and S499, according to FIG. 1; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.1, 0.9, 0.7 or 0.5 Å, and wherein at least one of said corresponding amino acids is not identical to the ITK amino acid to which it corresponds.

In other embodiments of this invention is provided a machine-readable data storage medium, comprising a data storage material encoded with machine-readable data, wherein said data comprises all or part of any molecule or molecular complex discussed in the above paragraphs.

In one embodiment of this invention is provided a computer comprising:
a machine-readable data storage medium, comprising a data storage material encoded with machine-readable data, wherein said data comprises all or part of an ITK ATP-binding pocket defined by structure coordinates of ITK amino acids I369, G370, V377, A389, K391, V419, F435, E436, F437, M438, E439, H440, C442, D445, L489 and S499, according to FIG. 1; or a molecule or molecular complex comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å, or 1.0 Å; or a molecule or molecular complex comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 1.1 Å, and wherein at least one of said corresponding amino acids is not identical to the ITK amino acid to which it corresponds.

In other embodiments of this invention is provided a computer comprising:
a machine-readable data storage medium, comprising a data storage material encoded with machine-readable data, wherein said data comprises all or part of any molecule or molecular complex discussed in the above paragraphs.

In one embodiment, a computer according to this invention comprises a working memory for storing instructions for processing the machine-readable data, a central-processing unit coupled to the working memory and to said machine-readable data storage medium for processing said machine-readable data into the three-dimensional structure. In one embodiment, the computer further comprises a display for displaying the three-dimensional structure as a graphical representation. In another embodiment, the computer further comprises commercially available software program to display the graphical representation. Examples of software programs include but are not limited to QUANTA [Molecular Simulations, Inc., San Diego, Calif. ©1998, 2000], O [Jones et al., Acta Cryst. A, 47, pp. 110-119 (1991)] and RIBBONS [M. Carson, J. Appl. Cryst., 24, pp. 958-961 (1991)], which are incorporated herein by reference.

This invention also provides a computer comprising:
a) a machine-readable data storage medium comprising a data storage material encoded with machine-readable data, wherein the data defines any one of the above binding pockets or protein of the molecule or molecular complex;
b) a working memory for storing instructions for processing said machine-readable data;
c) a central processing unit (CPU) coupled to the working memory and to the machine-readable data storage medium for processing said machine readable data as well as an instruction or set of instructions for generating three-dimensional structural information of said binding pocket or protein; and
d) output hardware coupled to the CPU for outputting three-dimensional structural information of the binding pocket or protein, or information produced by using the three-dimensional structural information of said binding pocket or protein. The output hardware may include monitors, touchscreens, printers, facsimile machines, modems, disk drives, CD-ROMs, etc.

Three-dimensional data generation may be provided by an instruction or set of instructions such as a computer program or commands for generating a three-dimensional structure or graphical representation from structure coordinates, or by subtracting distances between atoms, calculating chemical energies for an ITK molecule or molecular complex or homologues thereof, or calculating or minimizing energies for an association of an ITK molecule or molecular complex or homologues thereof to a chemical entity. The graphical representation can be generated or displayed by commercially available software programs. Examples of software programs include but are not limited to QUANTA [Accelrys ©2001, 2002], O [Jones et al., Acta Crystallogr. A47, pp. 110-119 (1991)] and RIBBONS [Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information about said binding pocket or information produced by using said binding pocket can be outputted through display terminals, touchscreens, printers, modems, facsimile machines, CD-ROMs or disk drives. The information can be in graphical or alphanumeric form.

FIG. 6 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bi-directional system bus 50.

Input hardware 35, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA [Molecular Simulations, Inc., San Diego, Calif. ©1998, 2000] as described herein. Output hardware might also include a printer 42, so that hard copy output may be produced, or a disk drive 24, to store system output for later use. Output hardware may also include a display terminal, a CD or DVD recorder, ZIP™ or JAZ™ drive, or other machine-readable data storage device.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

FIG. 7 shows a cross section of a magnetic data storage medium 100 which can be encoded with a machine-readable data that can be carried out by a system such as system 10 of FIG. 6. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in a manner that may be conventional, machine readable data such as that described herein, for execution by a system such as system 10 of FIG. 6.

FIG. 8 shows a cross section of an optically-readable data storage medium 110 which also can be encoded with such a machine-readable data, or set of instructions, which can be carried out by a system such as system 10 of FIG. 6. Medium 110 can be a conventional compact disk read only memory (CD-ROM) or a rewritable medium such as a magneto-optical disk that is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

In one embodiment, the data defines the above-mentioned binding pockets by comprising the structure coordinates of said amino acid residues according to FIG. 1, 2 or 3.

To use the structure coordinates generated for ITK or ITK homologue, one of its binding pockets, motifs, domains, or portion thereof, it is at times necessary to convert them into a three-dimensional shape or to generate three-dimensional structural information from them. This is achieved through the use of commercially or publicly available software that is capable of generating a three-dimensional structure of molecules or portions thereof from a set of structure coordinates. In one embodiment, the three-dimensional structure may be displayed as a graphical representation.

Therefore, according to another embodiment, this invention provides a machine-readable data storage medium comprising a data storage material encoded with machine readable data. In one embodiment, a machine programmed with instructions for using said data, is capable of generating a three-dimensional structure of any of the molecule or molecular complexes, or binding pockets thereof, that are described herein.

In certain embodiment, this invention also provides a computer for producing a three-dimensional structure of:
a) a molecule or molecular complex
   comprising all or part of an ITK ATP-binding pocket defined by structure coordinates of ITK amino acids V377, A389, V419, F435, E436, F437, M438, C442, L489 and S499, according to FIG. 1;
b) a molecule or molecular complex
   comprising all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å or 1.0 Å; or 0.5 Å; and/or
c) a molecule or molecular complex
   comprising all or part of an ITK-like ATP-binding pocket defined by structure coordinates of a set of corresponding amino acids, wherein the root mean square deviation of the backbone atoms between said set of corresponding amino acids and said ITK amino acids is not more than about 0.6 Å, 0.5 Å or 0.4 Å, and wherein at least one of said corresponding amino acids is not identical to the ITK amino acid to which it corresponds, comprising:
i) a machine-readable data storage medium, comprising a data storage material encoded with machine-readable data, wherein said data comprises all or part of an ITK ATP-binding pocket defined by structure coordinates of ITK amino acids V377, A389, V419, F435, E436, F437, M438, C442, L489 and S499, according to FIG. 1; all or part of an ITK-like ATP-binding pocket defined by structure coordinates of corresponding amino acids that are identical to said ITK amino acids, wherein the root mean square deviation of the backbone atoms between said corresponding amino acids and said ITK amino acids is not more than about 3.0 Å, 2.5 Å, 2.0 Å, 1.5 Å or 1.0 Å; or all or part of an ITK-like ATP-binding pocket defined by structure coordinates of a set of corresponding amino acids, wherein the root mean square deviation of the backbone atoms between said set of corresponding amino acids and said ITK amino acids is not more than about 0.6 Å, 0.5 Å or 0.4 Å, and wherein at least one of said corresponding amino acids is not identical to the ITK amino acid to which it corresponds; and
ii) instructions for processing said machine-readable data into said three-dimensional structure.

According to other embodiments, the computer is also for producing the three-dimensional structure of the aforementioned molecules and molecular complexes and comprises the corresponding machine-readable data storage mediums. In one embodiment, the three-dimensional structure is displayed as a graphical representation.

In one embodiment, the structure coordinates of said molecules or molecular complexes are produced by homology modeling of at least a portion of the structure coordinates of FIG. 1, 2 or 3. Homology modeling can be used to generate structural models of ITK homologues or other homologous proteins based on the known structure of ITK. This can be achieved by performing one or more of the following steps: performing sequence alignment between the amino acid sequence of an unknown molecule against the amino acid sequence of ITK; identifying conserved and variable regions by sequence or structure; generating structure co-ordinates for structurally conserved residues of the unknown structure from those of ITK; generating conformations for the structurally variable residues in the unknown structure; replacing the non-conserved residues of ITK with residues in the unknown structure; building side chain conformations; and refining and/or evaluating the unknown structure.

For example, since the protein sequence of the catalytic domains of ITK and homologues thereof can be aligned relative to each other, it is possible to construct models of the structures of ITK homologues, particularly in the regions of the active site, using the ITK structure. Software programs that are useful in homology modeling include XALIGN [Wishart, D. S. et al., Comput. Appl. Biosci., 10, pp. 687-88 (1994)] and CLUSTAL W Alignment Tool [Higgins D. G. et al., Methods Enzymol, 266, pp. 383-402 (1996)]. See also, U.S. Pat. No. 5,884,230. These references are incorporated herein by reference.

To perform the sequence alignment, programs such as the “bestfit” program available from the Genetics Computer Group [Waterman in Advances in Applied Mathematics 2, 482 (1981), which is incorporated herein by reference] and CLUSTAL W Alignment Tool [Higgins D. G. et al., Methods Enzymol, 266, pp. 383-402 (1996), which is incorporated by reference] can be used. To model the amino acid side chains of homologous ITK proteins, the amino acid residues in ITK can be replaced, using a computer graphics program such as “O” [Jones et al, (1991) Acta Cryst. Sect. A, 47: 110-119], by those of the homologous protein, where they differ. The same orientation or a different orientation of the amino acid can be used. Insertions and deletions of amino acid residues may be necessary where gaps occur in the sequence alignment.

Homology modeling can be performed using, for example, the computer programs SWISS-MODEL available through Glaxo Wellcome Experimental Research in Geneva, Switzerland; WHATIF available on EMBL servers; Schnare et al., J. Mol. Biol. 256: 701-719 (1996); Blundell et al., Nature 326: 347-352 (1987); Fetrow and Bryant, Bio/Technology 11:479-484 (1993); Greer, Methods in Enzymology 202: 239-252 (1991); and Johnson et al, Crit. Rev. Biochem. Mol. Biol. 29:1-68 (1994). An example of homology modeling can be found, for example, in Szklarz G. D., Life Sci. 61: 2507-2520 (1997). These references are incorporated herein by reference.

Thus, in accordance with the present invention, data capable of generating the three dimensional structure of the above molecules or molecular complexes (e.g., ITK, homologues and portions thereof), or binding pockets thereof, can be stored in a machine-readable storage medium, which is capable of displaying three-dimensional structural information or a graphical three-dimensional representation of the structure.

Rational Drug Design

The ITK structure coordinates or the three-dimensional graphical representation generated from these coordinates may be used in conjunction with a computer for a variety of purposes, including drug discovery. In certain embodiments, the computer is programmed with software to translate those coordinates into the three-dimensional structure of ITK.

For example, the structure encoded by the data may be computationally evaluated for its ability to associate with chemical entities. Chemical entities that associate with ITK may inhibit or activate ITK or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention provides a method for designing, selecting and/or optimizing a chemical entity that binds to the molecule or molecular complex comprising the steps of:
(a) providing the structure coordinates of said molecule or molecular complex on a computer comprising the means for generating three-dimensional structural information from said structure coordinates; and
(b) designing, selecting and/or optimizing said chemical entity by employing means for performing a fitting operation between said chemical entity and said three-dimensional structural information of said molecule or molecular complex.

Three-dimensional structural information in step (a) may be generated by instructions such as a computer program or commands that can generate a three-dimensional structure or graphical representation; subtract distances between atoms; calculate chemical energies for an ITK molecule, molecular complex or homologues thereof; or calculate or minimize energies of an association of ITK molecule, molecular complex or homologues thereof to a chemical entity. These types of computer programs are known in the art. The graphical representation can be generated or displayed by commercially available software programs. Examples of software programs include but are not limited to QUANTA [Accelrys ©2001, 2002], O [Jones et al., Acta Crystallogr. A47, pp. 110-119 (1991)] and RIBBONS [Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

Another embodiment of the invention provides a method for evaluating the potential of a chemical entity to associate with the molecule or molecular complex as described previously.

This method comprises the steps of: a) employing computational means to perform a fitting operation between the chemical entity and the molecule or molecular complex described before; b) analyzing the results of said fitting operation to quantify the association between the chemical entity and the molecule or molecular complex; and, optionally, c) outputting said quantified association to a suitable output hardware, such as a CRT display terminal, a printer, a CD or DVD recorder, ZIP™ or JAZ™ drive, a disk drive, or other machine-readable data storage device, as described previously. The method may further comprise generating a three-dimensional structure, graphical representation thereof, or both, of the molecule or molecular complex prior to step a). In one embodiment, the method is for evaluating the ability of a chemical entity to associate with the binding pocket of a molecule or molecular complex.

In another embodiment, the method comprises the steps of:
a) constructing a computer model of a binding pocket of the molecule or molecular complex;
b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of an ITK protein or homologue thereof;
c) employing computational means to perform a fitting program operation between computer models of said chemical entity to be evaluated and said binding pocket in order to provide an energy-minimized configuration of said chemical entity in the binding pocket; and
d) evaluating the results of said fitting operation to quantify the association between said chemical entity and the binding pocket model, thereby evaluating the ability of said chemical entity to associate with said binding pocket.

In another embodiment, the invention provides a method of using a computer for evaluating the ability of a chemical entity to associate with the molecule or molecular complex, wherein said computer comprises a machine-readable data storage medium comprising a data storage material encoded with said structure coordinates defining said binding pocket and means for generating a three-dimensional graphical representation of the binding pocket, and wherein said method comprises the steps of:
(a) positioning a first chemical entity within all or part of said binding pocket using a graphical three-dimensional representation of the structure of the chemical entity and the binding pocket;
(b) performing a fitting operation between said chemical entity and said binding pocket by employing computational means;
(c) analyzing the results of said fitting operation to quantitate the association between said chemical entity and all or part of the binding pocket; and
(d) outputting said quantitated association to a suitable output hardware.

The above method may further comprise the steps of:
(e) repeating steps (a) through (d) with a second chemical entity; and
(f) selecting at least one of said first or second chemical entity that associates with said all or part of said binding pocket based on said quantitated association of said first or second chemical entity.

Alternatively, the structure coordinates of the ITK binding pockets may be utilized in a method for identifying an agonist or antagonist of a molecule comprising a binding pocket of ITK. In certain embodiments, the method comprises steps of:
a) using a three-dimensional structure of the molecule or molecular complex to design, select or optimize a chemical entity;
b) contacting the chemical entity with the molecule or molecular complex;
c) monitoring the catalytic activity of the molecule or molecular complex; and
d) classifying the chemical entity as an agonist or antagonist based on the effect of the chemical entity on the catalytic activity of the molecule or molecular complex.

In one embodiment, step a) is performed using a graphical representation of the binding pocket or portion thereof of the molecule or molecular complex.

In one embodiment, the three-dimensional structure is displayed as a graphical representation.

In another embodiment, the method comprises the steps of:
a) constructing a computer model of a binding pocket of the molecule or molecular complex;
b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of an ITK protein or homologue thereof;
c) employing computational means to perform a fitting program operation between computer models of said chemical entity to be evaluated and said binding pocket in order to provide an energy-minimized configuration of said chemical entity in the binding pocket; and
d) evaluating the results of said fitting operation to quantify the association between said chemical entity and the binding pocket model, thereby evaluating the ability of said chemical entity to associate with said binding pocket;
e) synthesizing said chemical entity; and
f) contacting said chemical entity with said molecule or molecular complex to determine the ability of said compound to activate or inhibit said molecule.

For the first time, the present invention permits the use of molecular design techniques to identify, select and design chemical entities, including inhibitory compounds, capable of binding to ITK or ITK-like binding pockets, motifs and domains.

Applicants' elucidation of binding pockets on ITK provides the necessary information for designing new chemical entities and compounds that may interact with ITK or ITK-like substrate or ATP-binding pockets, in whole or in part.

Throughout this section, discussions about the ability of a chemical entity to bind to, associate with or inhibit ITK binding pockets refers to features of the entity alone. Assays to determine if a compound binds to ITK are well known in the art and are exemplified below.

The design of chemical entities that bind to or inhibit ITK binding pockets according to this invention generally involves consideration of two factors. First, the entity must be capable of physically and structurally associating with parts or all of the ITK binding pockets. Non-covalent molecular interactions important in this association include hydrogen bonding, van der Waals interactions, hydrophobic interactions and electrostatic interactions.

Second, the entity must be able to assume a conformation that allows it to associate with the ITK binding pockets directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the ITK or ITK-like binding pockets.

The potential inhibitory or binding effect of a chemical entity on ITK binding pockets may be analyzed prior to its actual synthesis and testing by the use of computer modeling techniques. If the theoretical structure of the given entity suggests insufficient interaction and association between it and the ITK binding pockets, testing of the entity is obviated. However, if computer modeling indicates a strong interaction, the compound may then be synthesized and tested for its ability to bind to an ITK binding pocket. This may be achieved by testing the ability of the molecule to inhibit ITK using the assays described in Example 7. In this manner, synthesis of inoperative compounds may be avoided.

A potential inhibitor of an ITK binding pocket may be computationally evaluated by means of a series of steps in which chemical entities or fragments are screened and selected for their ability to associate with the ITK binding pockets.

One skilled in the art may use one of several methods to screen chemical entities or fragments for their ability to associate with an ITK binding pocket. This process may begin by visual inspection of, for example, an ITK binding pocket on the computer screen based on the ITK structure coordinates in FIG. 1, 2 or 3 or other coordinates which define a similar shape generated from the machine-readable storage medium. Selected fragments or chemical entities may then be positioned in a variety of orientations, or docked, within that binding pocket as defined supra. Docking may be accomplished using software such as QUANTA and Sybyl [Tripos Associates, St. Louis, Mo.], followed by energy minimization and molecular dynamics with standard molecular mechanics force fields, such as CHARMM and AMBER.

Specialized computer programs may also assist in the process of selecting fragments or chemical entities. These include:
1. GRID [P. J. Goodford, “A Computational Procedure for Determining Energetically Favorable Binding Sites on Biologically Important Macromolecules”, J. Med. Chem. 28, pp. 849-857 (1985)]. GRID is available from Oxford University, Oxford, UK.
2. MCSS [A. Miranker et al., “Functionality Maps of Binding Sites: A Multiple Copy Simultaneous Search Method.” Proteins: Structure. Function and Genetics, 11, pp. 29-34 (1991)]. MCSS is available from Molecular Simulations, San Diego, Calif.
3. AUTODOCK [D. S. Goodsell et al., “Automated Docking of Substrates to Proteins by Simulated Annealing”, Proteins: Structure, Function, and Genetics, 8, pp. 195-202 (1990)]. AUTODOCK is available from Scripps Research Institute, La Jolla, Calif.
4. DOCK [I. D. Kuntz et al., “A Geometric Approach to Macromolecule-Ligand Interactions”, J. Mol. Biol., 161, pp. 269-288 (1982)]. DOCK is available from University of California, San Francisco, Calif.

Once suitable chemical entities or fragments have been selected, they can be assembled into a single compound or complex of compounds. Assembly may be preceded by visual inspection of the relationship of the fragments to each other on the three-dimensional image displayed on a computer screen in relation to the structure coordinates of ITK. This would be followed by manual model building using software such as QUANTA or Sybyl [Tripos Associates, St. Louis, Mo.].

Useful programs to aid one of skill in the art in connecting the individual chemical entities or fragments include:
1. CAVEAT [P. A. Bartlett et al., “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)]. CAVEAT is available from the University of California, Berkeley, Calif.
2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).
3. HOOK [M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994)]. HOOK is available from Molecular Simulations, San Diego, Calif.

Instead of proceeding to build an inhibitor of an ITK binding pocket in a step-wise fashion one fragment or chemical entity at a time as described above, inhibitory or other ITK binding compounds may be designed as a whole or “de novo” using either an empty binding pocket or optionally including some portion(s) of a known inhibitor(s). There are many de novo ligand design methods including:
1. LUDI [H.-J. Bohm, “The Computer Program LUDI: A New Method for the De Novo Design of Enzyme Inhibitors”, J. Comp. Aid. Molec. Design, 6, pp. 61-78 (1992)]. LUDI is available from Molecular Simulations Incorporated, San Diego, Calif.
2. LEGEND [Y. Nishibata et al., Tetrahedron, 47, p. 8985 (1991)]. LEGEND is available from Molecular Simulations Incorporated, San Diego, Calif.
3. LeapFrog [available from Tripos Associates, St. Louis, Mo.].
4. SPROUT [V. Gillet et al., “SPROUT: A Program for Structure Generation)”, J. Comput. Aided Mol. Design, 7, pp. 127-153 (1993)]. SPROUT is available from the University of Leeds, UK.

Other molecular modeling techniques may also be employed in accordance with this invention [see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modem Methods in Computer-Aided Drug Design”, Reviews in Computational Chemistry. Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology 4, pp. 777-781 (1994)].

Once a chemical entity has been designed or selected by the above methods, the efficiency with which that chemical entity may bind to an ITK binding pocket may be tested and optimized by computational evaluation. For example, an effective ITK binding pocket inhibitor must preferably demonstrate a relatively small difference in energy between its bound and free states (i.e., a small deformation energy of binding). Thus, the most efficient ITK binding pocket inhibitors should preferably be designed with a deformation energy of binding of not greater than about 10 kcal/mole, more preferably, not greater than 7 kcal/mole. ITK binding pocket inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to an ITK binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C [M. J. Frisch, Gaussian, Inc., Pittsburgh, Pa. ©1995]; AMBER, version 4.1 [P. A. Kollman, University of California at San Francisco, ©1995]; QUANTA/CHARMM [Accelrys, San Diego, Calif. ©2001, 2002]; Insight II/Discover [Accelrys, San Diego, Calif. ©2001, 2002]; DelPhi [Accelrys, San Diego, Calif. ©2001, 2002]; and AMSOL [Quantum Chemistry Program Exchange, Indiana University]. These programs may be implemented, for instance, using a Silicon Graphics workstation such as an Indigo2 with “IMPACT” graphics. Other hardware systems and software packages will be known to those skilled in the art.

Another approach enabled by this invention, is the computational screening of small molecule databases for chemical entities or compounds that can bind in whole, or in part, to an ITK binding pocket. In this screening, the quality of fit of such entities to the binding pocket may be judged either by shape complementarity or by estimated interaction energy [E. C. Meng et al., J. Comp. Chem., 13, pp. 505-524 (1992)].

Another particularly useful drug design technique enabled by this invention is iterative drug design. Iterative drug design is a method for optimizing associations between a protein and a compound by determining and evaluating the three-dimensional structures of successive sets of protein/compound complexes.

According to another embodiment, the invention provides compounds which associate with an ITK binding pocket produced or identified by the method set forth above.

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound complex.

Structure Determination of Other Molecules

The structure coordinates set forth in FIG. 1, 2 or 3 can also be used to aid in obtaining structural information about another crystallized molecule or molecular complex. This may be achieved by any of a number of well-known techniques, including molecular replacement.

According to an alternate embodiment, the machine-readable data storage medium comprises a data storage material encoded with a first set of machine readable data which comprises the Fourier transform of at least a portion of the structure coordinates set forth in FIG. 1, 2 or 3 or homology model thereof, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex, wherein said computer comprises:
a) a machine-readable data storage medium comprising a data storage material encoded with machine-readable data, wherein said data comprises at least a portion of the structural coordinates of ITK according to FIG. 1, 2 or 3 or homology model thereof;
b) a machine-readable data storage medium comprising a data storage material encoded with machine-readable data, wherein said data comprises X-ray diffraction data obtained from said molecule or molecular complex; and
c) instructions for performing a Fourier transform of the machine readable data of (a) and for processing said machine readable data of (b) into structure coordinates.

For example, the Fourier transform of at least a portion of the structure coordinates set forth in FIG. 1, 2 or 3 or homology model thereof may be used to determine at least a portion of the structure coordinates of ITK homologues.

Therefore, in another embodiment this invention provides a method of utilizing molecular replacement to obtain structural information about a molecule or molecular complex whose structure is unknown comprising the steps of:
a) crystallizing said molecule or molecular complex of unknown structure;
b) generating an X-ray diffraction pattern from said crystallized molecule or molecular complex;
c) applying at least a portion of the structure coordinates set forth in FIG. 1, 2 or 3 or homology model thereof to the X-ray diffraction pattern to generate a three-dimensional electron density map of the molecule or molecular complex whose structure is unknown; and
d) generating a structural model of the molecule or molecular complex from the three-dimensional electron density map.

In one embodiment, the method is performed using a computer. In another embodiment, the molecule is selected from the group consisting of ITK and ITK homologues. In another embodiment, the molecule is an ITK molecular complex or homologue thereof.

By using molecular replacement, all or part of the structure coordinates of the ITK as provided by this invention (and set forth in FIG. 1, 2 or 3) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that can not be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of the ITK according to FIG. 1, 2 or 3 or homology model thereof within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex [E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)].

The structure of any portion of any crystallized molecule or molecular complex that is sufficiently homologous to any portion of the ITK can be resolved by this method.

In a preferred embodiment, the method of molecular replacement is utilized to obtain structural information about an ITK homologue. The structure coordinates of ITK as provided by this invention are particularly useful in solving the structure of ITK complexes that are bound by ligands, substrates and inhibitors.

Furthermore, the structure coordinates of ITK as provided by this invention are useful in solving the structure of ITK proteins that have amino acid substitutions, additions and/or deletions (referred to collectively as “ITK mutants”, as compared to naturally occurring ITK). These ITK mutants may optionally be crystallized in co-complex with a chemical entity, such as a non-hydrolyzable ATP analog or a suicide substrate. The crystal structures of a series of such complexes may then be solved by molecular replacement and compared with that of wild-type ITK. Potential sites for modification within the various binding pockets of the enzyme may thus be identified. This information provides an additional tool for determining the most efficient binding interactions, for example, increased hydrophobic interactions, between ITK and a chemical entity or compound.

The structure coordinates are also particularly useful in solving the structure of crystals of ITK or ITK homologues co-complexed with a variety of chemical entities. This approach enables the determination of the optimal sites for interaction between chemical entities, including candidate ITK inhibitors. For example, high resolution X-ray diffraction data collected from crystals exposed to different types of solvent allows the determination of where each type of solvent molecule resides. Small molecules that bind tightly to those sites can then be designed and synthesized and tested for their ITK inhibition activity.

All of the complexes referred to above may be studied using well-known X-ray diffraction techniques and may be refined versus 1.5-3.4 Å resolution X-ray data to an R value of about 0.30 or less using computer software, such as X-PLOR (Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)), CNS (Brunger et al., Acta Crystallogr. D. Biol. Crystallogr., 54, pp. 905-921, (1998)) or CNX (Accelrys. ©2000, 2001). This information may thus be used to optimize known ITK inhibitors, and more importantly, to design new ITK inhibitors.

In order that this invention be more fully understood, the following examples are set forth. These examples are for the purpose of illustration only and are not to be construed as limiting the scope of the invention in any way.

### EXAMPLE 1

### Expression and Purification of ITK

The expression of ITK was carried out using standard procedures known in the art.

A truncated version of the ITK kinase domain (residues 357-620) (the same sequence as GenBank accession number L10717) incorporating an N-terminal hexa-histidine purification tag and a thrombin cleavage site was overexpressed in baculovirus expression system using Hi5 (source) insect cells.

TK was purified using Ni/NTA agarose metal affinity chromatography (Qiagen, Hilden, Germany) and the hexa-histidine tag was then removed by overnight incubation at 4° C. with 5 U mg⁻¹ thrombin (Calbiochem, La Jolla, Calif.). Thrombin was removed with benzamidine sepharose (Amersham Biotech, Uppsala, Sweden). Subsequent purification by size-exclusion on a Superdex 200 column (AmershamPharmacia Biotech, Uppsala, Sweden) yielded a homogeneous, unphosphorylated sample suitable for crystallization Activation of this purified ITK protein was performed by incubating a small protein sample with 1:100 (w/w) ITK:LCK for overnight at 4° C. in the presence of 10 mM MgCl₂ and 5 mM ATP. Residual unphosphorylated protein was removed by a further resourceQ column (Amersham Biotech, Uppsala, Sweden) purification step. Characterization of the activated sample revealed complete homogeneous phosphorylation of a single ITK residue, Y512. The unphosphorylated and phosphorylated ITK protein (pITK) samples were dialysed against 25 mM Tris, pH8.6 containing 50 mM NaCl and 2 mM DTT at 4° C. and concentrated to 10 mg ml⁻¹ for crystallization. All protein molecular weights were confirmed by electrospray mass spectrometry.

### EXAMPLE 2

### Formation of ITK-Inhibitor Complex for Crystallization

Crystals of ITK-inhibitor complex crystals were formed by co-crystallizing the protein with the inhibitors or with adenosine. The inhibitor was added to the ITK protein solution immediately after the final protein concentration step (Example 1), right before setting up the crystallization drop.

### EXAMPLE 3

### Crystallization of ITK and ITK-Inhibitor Complexes

Crystallization of ITK was carried out using the hanging drop vapor diffusion technique. The ITK formed thin plate-like crystals over a reservoir containing 800 mM Ammonium sulphate, 200 mM Magnesium acetate, 100 mM Sodium citrate pH5.7 and 10 mM DTT. The crystallization droplet contained 1 μl of 10 mg ml⁻¹ protein solution and 1 μl of reservoir solution. Crystals formed in approximately than 72 hours.

The formed crystals were transferred to a reservoir solution containing 15% glycerol. After soaking the crystals in 15% glycerol for less than 2 minutes, the crystals were scooped up with a cryo-loop, frozen in liquid nitrogen and stored for data collection.

### EXAMPLE 4

### Soaking of Preformed ITK Complex Crystals in Solutions of Other Inhibitors

An alternative method for preparing complex crystals of ITK is to remove a co-complex crystal grown by hanging drop vapour diffusion (Example 3) from the hanging drop and place it in a solution consisting of a reservoir solution containing 0.5 mM staurosporine or another inhibitor for a period of time between 1 and 24 hours.

The crystals can then be transferred to a reservoir solution containing 15% glycerol and 0.5 mM staurosporine or another inhibitor. After soaking the crystal in this solution for less than minutes, the crystals were scooped up with a cryo-loop, frozen in liquid nitrogen and stored for data collection. Subsequent data collection and structure determination (Example 5) reveals that inhibitors bound to the ATP-binding site of ITK can be exchanged for the ITK or pITK complex crystals.

### EXAMPLE 5

### X-Ray Data Collection and Structure Determination

The ITK-inhibitor complex structures and the ITK-adenosine structure were solved by molecular replacement using X-ray diffraction data collected either (i) at beam line 14.2 of the CCLRC Synchrotron Radiation Source, Daresbury, Cheshire, UK, or (ii) Vertex Pharmaceuticals (Europe) Ltd, 88 Milton Park, Abingdon, Oxfordshire OX14 4RY, UK. The diffraction images were processed with the program MOSFLM [A. G. Leslie, Acta Cryst. D, 55, pp. 1696-1702 (1999)] and the data was scaled using SCALA [Collaborative Computational Project, N., Acta Cryst. D, 50, pp. 760-763 (1994)].

The data statistics, unit cell parameters and spacegroup of the ITK-3-(8-Phenyl-5,6-dihydrothieno[2,3-h]quinazolin-2-ylamino)benzenesulfonamide crystal structure is given in Table 2. The starting phases for the ITK complexes were obtained by molecular replacement using coordinates of an ITK homology model constructed from BTK (Mao, C et al J. Biol. Chem., 276, pp. 41435-41443 (2001)) as a search model in the program AMoRe [J. Navaza, Acta. Cryst. A, 50, pp. 157-163 (1994)]. The asymmetric unit contained a single ITK complex. Multiple rounds of rebuilding with QUANTA [Molecular Simulations, Inc., San Diego, Calif. ©1998, 2000] and refinement with CNX [Accelrys Inc., San Diego, Calif. ©2000] resulted in a final model that included residues 358 to 502 and residues 515 to 619. The refined model has a crystallographic R-factor of 26.0% and R-free of 35.5%.

The data statistics, unit cell parameters and spacegroup of the pITK-staurosporine crystal structure is given in Table 3. The starting phases were obtained by molecular replacement using coordinates of the ITK-3-(8-Phenyl-5,6-dihydrothieno[2,3-h]quinazolin-2-ylamino)benzenesulfonamide complex as a search model in the program AMoRe. Multiple rounds of rebuilding with QUANTA [Molecular Simulations, Inc., San Diego, Calif. ©1998, 2000] and refinement with CNX [Accelrys Inc., San Diego, Calif. ©2000] resulted in a final model that included residues 357 to 502 and residues 521 to 619. The refined model has a crystallographic R-factor of 21.4% and R-free of 29.2%.

The data statistics, unit cell parameters and spacegroup of the ITK-staurosporine crystal structure is given in Table 4. The starting phases were obtained by molecular replacement using coordinates of the ITK-3-(8-Phenyl-5,6-dihydrothieno[2,3-h]quinazolin-2-ylamino)benzenesulfonamide complex as a search model in the program AMoRe. Multiple rounds of rebuilding with QUANTA [Molecular Simulations, Inc., San Diego, Calif. ©1998, 2000] and refinement with CNX [Accelrys Inc., San Diego, Calif. ©2000] resulted in a final model that included residues 357 to 502 and residues 521 to 619. The refined model has a crystallographic R-factor of 23.7% and R-free of 29.5%.

In the above models, disordered residues were not included in the model. Alanine or glycine residues were used in the model if the side chains of certain residues could not be located in the electron density.

### EXAMPLE 6

### Overall Structure of ITK

ITK has the typical bi-lobal catalytic kinase fold or structural domain [S. K. Hanks, et al., Science, 241, pp. 42-52 (1988); Hanks, S. K. and A. M. Quinn, Meth. Enzymol., 200, pp. 38-62 (1991)] with a β-strand sub-domain (residues 357-435) at the N-terminal end and an α-helical sub-domain at the C-terminal end (residues 443-620) (FIG. 4). The ATP-binding pocket is at the interface of the α-helical and β-strand domains, and is bordered by the glycine rich loop and the hinge. The activation loop is disorder in all three crystal structures.

Comparison with other kinases such as LCK, CDK2 and p38 revealed that the structure of ITK resembles closely the substrate-bound, activated, form of a kinase. The overall topology of the kinase domain is similar to other tyrosine kinases, particularly LCK and SRC, and distinct from the serine/threonine family (CDK-2, Aurora-2; Tables 2-4).

### EXAMPLE 7

### Catalytic Active Site of ITK-Inhibitor Complexes

The inhibitor 3-(8-Phenyl-5,6-dihydrothieno[2,3-h]quinazolin-2-ylamino)benzenesulfonamide is bound in the deep cleft of the catalytic active site in the ITK structure (FIG. 5). The inhibitor forms thee hydrogen bonds with the hinge portion of the ATP-binding pocket (dotted lines). The pyrimidine nitrogen (position 3) shares a proton with the M438 backbone amine. The adjacent pyrimidine carbon (position 4) donates its hydrogen to E436 to make an unusual hydrogen-bond. Finally the extracyclic amine of the 2-aminopyrimidine moiety shares its hydrogen with the backbone carbonyl of M438.

The side chains of D500 and K391 are positioned inside the ATP-binding pocket and make a salt-bridge interaction with each other. Like other kinases, K391 and D500 are catalytically important residue and resemble a catalytically active conformation. The sulphonamide group does not make ant direct interactions with the surrounding protein.

Perhaps the most important interaction discovered is made between the 5C and 6C atoms of the tricyclic ring system and the side chain of residue Phe 435. This is because residue Phe435 is unique to ITK within the TEC-family kinases (see Table 1). This edge-face hydrophobic interaction made between the inhibitor and Phe435 could not be made by any of the other TEC kinases, which have a Threonine at this position. The inhibitor 3-(8-Phenyl-5,6-dihydrothieno[2,3-h]quinazolin-2-ylamino)benzenesulfonamide may therefore represent a scaffold that is uniquely selective for ITK kinase.

This interaction also suggest that substitutions at the 5C and 6C positions of 3-(8-Phenyl-5,6-dihydrothieno[2,3-h]quinazolin-2-ylamino)benzenesulfonamide may favour binding to BTK, TEC, RLK and BMX, rather the ITK. Discovery of residue Phe435 as a gatekeeper of the adjacent hydrophobic pocket thus has importance for inhibitor design and tuning inhibitor selectivity within the TEC-family kinases. The crystal structures define the optimal shape and size that an inhibitor must obey in order to effectively inhibit ITK kinase.

### EXAMPLE 8

### The Use of ITK Coordinates for Inhibitor Design

The coordinates of FIG. 1, 2 or 3 are used to design compounds, including inhibitory compounds, that associate with ITK or homologues of ITK. This process may be aided by using a computer comprising a machine-readable data storage medium encoded with a set of machine-executable instructions, wherein the recorded instructions are capable of displaying a three-dimensional representation of the ITK or a portion thereof. The graphical representation is used according to the methods described herein to design compounds. Such compounds associate with the ITK at the ATP-binding pocket or substrate binding pocket.

### EXAMPLE 9

### The Use of ITK Coordinates in the Design of ITK-Specific Antibodies

The atomic coordinates in FIG. 1, 2 or 3 also define, in great detail, the external solvent-accessible, hydrophilic, and mobile surface regions of the ITK catalytic kinase domain. Anti-peptide antibodies are known to react strongly against highly mobile regions but do not react with well-ordered regions of proteins. Mobility is therefore a major factor in the recognition of proteins by anti-peptide antibodies [J. A. Tainer et al., Nature, 312, pp. 127-134 (1984)]

One skilled in the art would therefore be able to use the X-ray crystallography data to determine possible antigenic sites in the ITK kinase domain. Possible antigenic sites are exposed, small and mobile regions on the kinase surface which have atomic B-factors of greater than 80 Å² in FIGS. 1, 2 and 3. This information can be used in conjunction with data from immunological studies to design and produce specific monoclonal or polyclonal antibodies.

This process may be aided by using a computer comprising a machine-readable data storage medium encoded with a set of machine-executable instructions, wherein the recorded instructions are capable of displaying a three-dimensional representation of the ITK or a portion thereof.

TABLE 5
Summary of data collection for ITK - 3-(8-Phenyl-5,6-dihydrothieno[2,
3-h]quinazolin-2-ylamino)benzenesulfonamide complex
Space Group: C2
Unit Cell: a = 125.5 Å, b = 74.8 Å, c = 78.8 Å; α = γ = 90°, β = 94.0°

| Source | Vertex |
|---|---|
| Wavelength (Å) | 1.5418 |
| Resolution (Å) | 2.4 |
| No. of Reflections | 67,363/26,781 |
| (measured/unique) | |
| Completeness (%) | 95.0/95.0 |
| (overall/outer shell) | |
| I/σ(I) | 14.0/2.3 |
| (overall/outer shell) | |
| R_{merge}* (%) | 10.7/32.6 |
| (overall/outer shell) | |
| Molecules per asymmetric unit | 2 |

*R_{merge} = 100 × ΣhΣj<I(h)> − I(h)j/ΣhΣj<I(h)>, where <I(h)> is the mean intensity of symmetry-equivalent reflections

Structure Refinement

| | |
|---|---|
| Resolution (Å) | 20-2.4 |
| No. of reflections | 20522 |
| R factor | 26.0 |
| Free R factort† | 35.5 |
| RMSD values | 0.0156/2.1 Å/° |
| Bond lengths/angles | |

†The Free R factor was calculated with 2.4% of the data.

TABLE 6
Summary of data collection for pITK - staurosporine complex
Space Group: C2
Unit Cell: a = 125.1 Å, b = 74.5 Å, c = 78.9 Å; α = γ = 90°, β = 93.9°

| Source | Daresbury SRS 14.1 |
|---|---|
| Wavelength (Å) | 1.488 |
| Resolution (Å) | 2.3 |
| No. of Reflections | 53,151/29,885 |
| (measured/unique) | |
| Completeness (%) | 93.6/93.6 |
| (overall/outer shell) | |
| I/σ(I) | 10.1/1.5 |
| (overall/outer shell) | |
| R_{merge}* (%) | 7.2/47.0 |
| (overall/outer shell) | |
| Molecules per asymmetric unit | 2 |

*R_{merge} = 100 × ΣhΣj<I(h)> − I(h)j/ΣhΣj<I(h)>, where <I(h)> is the mean intensity of symmetry-equivalent reflections

Structure Refinement

| | |
|---|---|
| Resolution (Å) | 20-2.3 |
| No. of reflections | 20,033 |
| R factor | 21.4 |
| Free R factor†† | 29.2 |
| RMSD values | 0.016/2.1 Å/° |
| Bond lengths/angles | |

††The Free R factor was calculated with 2.3% of the data.

TABLE 7
Summary of data collection for ITK - staurosporine complex
Space Group: C2
Unit Cell: a = 124.4 Å, b = 74.2 Å, c = 78.8 Å; α = γ = 90°, β = 94.0°

| Source | Daresbury SRS 14.1 |
|---|---|
| Wavelength (Å) | 1.488 |
| Resolution (Å) | 2.5 |
| No. of Reflections | 44,498/22,705 |
| (measured/unique) | |
| Completeness (%) | 91.5/76.0 |
| (overall/outer shell) | |
| I/σ(I) | 15.3/2.3 |
| (overall/outer shell) | |
| R_{merge}* (%) | 7.9/40.0 |
| (overall/outer shell) | |
| Molecules per asymmetric unit | 2 |

*R_{merge} = 100 × ΣhΣj<I(h)> − I(h)j/ΣhΣj<I(h)>, where <I(h)> is the mean intensity of symmetry-equivalent reflections

Structure Refinement

| | |
|---|---|
| Resolution (Å) | 20-2.5 |
| No. of reflections | 17,417 |
| R factor | 23.7 |
| Free R factor††† | 29.5 |
| RMSD values | 0.017/2.23 Å/° |
| Bond lengths/angles | |

†††The Free R factor was calculated with 2.5% of the data.

What is claimed is:

1. A crystal comprising an Interleukin-2 Tyrosine kinase domain.

2. A crystal comprising an Interleukin-2 Tyrosine kinase domain homologue.

3. A crystal comprising an Interleukin-2 Tyrosine kinase domain complex.

4. A crystal comprising an Interleukin-2 Tyrosine kinase domain homologue complex.

5. The crystal according to claim 3, wherein said Interleukin-2 Tyrosine kinase domain complex is Interleukin-2 Tyrosine kinase domain bound to an active site inhibitor.

6. The crystal according to claim 3, wherein said Interleukin-2 Tyrosine kinase domain complex is Interleukin-2 Tyrosine kinase domain bound to any one of adenylyl imidodiphosphate (MgAMP-PNP), adenosine, staurosporine or 3-(8-phenyl-5,6-dihydrothieno[2,3-h]quinazolin-2-ylamino)benzenesulfonamide.

7. The crystal according to claim 3, wherein said Interleukin-2 Tyrosine kinase domain complex is Interleukin-2 Tyrosine kinase domain bound to staurosporine.

8. The crystal according to claim 3, wherein said Interleukin-2 Tyrosine kinase domain complex is Interleukin-2 Tyrosine kinase domain bound to 3-(8-phenyl-5,6-dihydrothieno[2,3-h]quinazolin-2-ylamino)benzenesulfonamide.

9. The crystal according to claim 1, 3, 5, 6, 7 or 8, wherein said Interleukin-2 Tyrosine kinase domain is phosphorylated.

10. The crystal according to claim 1, 3, 5, 6, 7 or 8, wherein said Interleukin-2 Tyrosine kinase domain is unphosphorylated.

11. The crystal according to any one of claims 1, 3, 5, 6, 7 or 8, wherein said Interleukin-2 Tyrosine kinase domain comprises Interleukin-2 Tyrosine kinase amino acid residues 357-620 according to any one of FIGS. 1, 2 or 3.

12. A crystallizable composition comprising an Interleukin-2 Tyrosine kinase domain.

13. A crystallizable composition comprising an Interleukin-2 Tyrosine kinase domain homologue.

14. A crystallizable composition comprising an Interleukin-2 Tyrosine kinase domain complex.

15. A crystallizable composition comprising an Interleukin-2 Tyrosine kinase domain homologue complex.

16. The crystallizable composition according to claim 14, wherein said Interleukin-2 Tyrosine kinase domain complex is bound to an active site inhibitor.

17. The crystallizable composition according to claim 14, wherein said Interleukin-2 Tyrosine kinase domain complex is Interleukin-2 Tyrosine kinase domain bound to any one of adenylyl imidodiphosphate (MgAMP-PNP), adenosine, staurosporine, or 3-(8-phenyl-5,6-dihydrothieno[2,3-h]quinazolin-2-ylamino)benzenesulfonamide.

18. The crystallizable composition according to claim 14, wherein said Interleukin-2 Tyrosine kinase domain complex is Interleukin-2 Tyrosine kinase domain bound to staurosporine.

19. The crystallizable composition according to claim 14, wherein said Interleukin-2 Tyrosine kinase domain complex is Interleukin-2 Tyrosine kinase domain bound to 3-(8-phenyl-5,6-dihydrothieno[2,3-h]quinazolin-2-ylamino)benzenesulfonamide.

20. The crystallizable composition according to claim 12, 14, 16, 17, 18 or 19, wherein Interleukin-2 Tyrosine kinase domain is phosphorylated.

21. The crystallizable composition according to claim 12, 14, 16, 17, 18 or 19, wherein Interleukin-2 Tyrosine kinase domain is unphosphorylated.

22. The crystallizable composition according to any one of claims 12, 14, 16, 17, 18 and 19, wherein said Interleukin-2 Tyrosine kinase domain comprises Interleukin-2 Tyrosine kinase amino acid residues 357-620 according to any one of FIGS. 1, 2 or 3.

23. A computer comprising:
(a) a machine-readable data storage medium, comprising a data storage material encoded with machine-readable data, wherein said data defines a binding pocket or domain comprising amino acid residues selected from the group consisting of:
(i) a set of amino acid residues which are identical to Interleukin-2 Tyrosine kinase amino acid residues I369, G370, V377, A389, K391, V419, F435, E436, F437, M438, E439, H440, C442, D445, L489 and S499 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(ii) a set of amino acid residues which are identical to Interleukin-2 Tyrosine kinase amino acid residues Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iii) a set of amino acid residues which are identical to Interleukin-2 Tyrosine kinase amino acid residues L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iv) a set of amino acid residues which are identical to Interleukin-2 Tyrosine kinase amino acid residues I369, V419, F435, E436, M438 and L489 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å; and/or
(v) a set of amino acid residues that are identical to Interleukin-2 Tyrosine kinase amino acid residues according to any one of FIGS. 1, 2 and 3 wherein the root mean square deviation of the backbone atoms between said set of amino acid residues and said Interleukin-2 Tyrosine kinase amino acid residues which are identical is not greater than about 3 Å;
(b) a working memory for storing instructions for processing said machine-readable data;
(c) a central processing unit coupled to said working memory and to said machine-readable data storage medium for processing said machine-readable data and a means for generating three-dimensional structural information of said binding pocket or domain; and
(d) output hardware coupled to said central processing unit for outputting three-dimensional structural information of said binding pocket or domain, or information produced using said three-dimensional structural information of said binding pocket or domain.

24. The computer according to claim 23, wherein said means for generating three-dimensional structural information is provided by means for generating a three-dimensional graphical representation of said binding pocket or domain.

25. The computer according to claim 23, wherein said output hardware is a display terminal, a printer, CD or DVD recorder, ZIP™ or JAZ™ drive, a disk drive, or other machine-readable data storage device.

26. A method of using a computer for selecting an orientation of a chemical entity that interacts favorably with a binding pocket or domain comprising amino acid residues selected from the group consisting of:
(i) a set of amino acid residues which are identical to Interleukin-2 Tyrosine kinase amino acid residues I369, G370, V377, A389, K391, V419, F435, E436, F437, M438, E439, H440, C442, D445, L489 and S499 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(ii) a set of amino acid residues which are identical to Interleukin-2 Tyrosine kinase amino acid residues Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iii) a set of amino acid residues which are identical to Interleukin-2 Tyrosine kinase amino acid residues L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å; and/or
(iv) a set of amino acid residues which are identical to Interleukin-2 Tyrosine kinase amino acid residues I369, V419, F435, E436, M438 and L489 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
said method comprising steps of:
(a) providing the structure coordinates of said binding pocket, domain or complex thereof on a computer comprising means of generating three-dimensional structural information from said structure coordinates;
(b) employing computational means to dock a first chemical entity in all or part of the binding pocket or domain;
(c) quantifying the association between said chemical entity and all or part of the binding pocket or domain for different orientations of the chemical entity; and
(d) selecting the orientation of the chemical entity with the most favorable interaction based on said quantified association.

27. The method according to claim 26, further comprising the step of generating a three-dimensional graphical representation of the binding pocket or domain prior to step (b).

28. The method according to claim 26, wherein energy minimization, molecular dynamics simulations, or rigid-body minimizations are performed simultaneously with or following step (b).

29. The method according to claim 26, further comprising the steps of:
(e) repeating steps (b) through (d) with a second chemical entity; and
(f) selecting at least one of said first or second chemical entity that interacts more favorably with said binding pocket or domain based on said quantified association of said first or second chemical entity.

30. A method of using a computer for selecting an orientation of a chemical entity with a favorable shape complementarity in a binding pocket comprising amino acid residues selected from the group consisting of:
(i) a set of amino acid residues which are identical to Interleukin-2 Tyrosine kinase amino acid residues I369, G370, V377, A389, K391, V419, F435, E436, F437, M438, E439, H440, C442, D445, L489 and S499 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(ii) a set of amino acid residues which are identical to Interleukin-2 Tyrosine kinase amino acid residues Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iii) a set of amino acid residues which are identical to Interleukin-2 Tyrosine kinase amino acid residues L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iv) a set of amino acid residues which are identical to Interleukin-2 Tyrosine kinase amino acid residues I369, V419, F435, E436, M438 and L489 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å; and/or
(v) a set of amino acid residues that are identical to Interleukin-2 Tyrosine kinase amino acid residues according to any one of FIGS. 1, 2 and 3 wherein the root mean square deviation of the backbone atoms between said set of amino acid residues and said Interleukin-2 Tyrosine kinase amino acid residues which are identical is not greater than about 3 Å;
said method comprising the steps of:
(a) providing the structure coordinates of said binding pocket and all or part of the ligand bound therein on a computer comprising the means for generating three-dimensional structural information from said structure coordinates;
(b) employing computational means to dock a first chemical entity in all or part of the binding pocket;
(c) quantitating the contact score of said chemical entity in different orientations in the binding pocket; and
(d) selecting an orientation with the highest contact score.

31. The method according to claim 30, further comprising the step of generating a three-dimensional graphical representation of all or part of the binding pocket and all or part of the ligand bound therein prior to step (b).

32. A method according to claim 30, further comprising the steps of:
(e) repeating steps (b) through (d) with a second chemical entity; and
(f) selecting at least one of said first or second chemical entity that has a higher contact score based on said quantitated contact score of said first or second chemical entity.

33. A method for designing, selecting or optimizing a chemical entity that interacts with a binding pocket or domain comprising amino acid residues selected from the group consisting of:
(i) a set of amino acid residues which are identical to Interleukin-2 Tyrosine kinase amino acid residues I369, G370, V377, A389, K391, V419, F435, E436, F437, M438, E439, H440, C442, D445, L489 and S499 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(ii) a set of amino acid residues which are identical to Interleukin-2 Tyrosine kinase amino acid residues Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iii) a set of amino acid residues which are identical to Interleukin-2 Tyrosine kinase amino acid residues L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iv) a set of amino acid residues which are identical to Interleukin-2 Tyrosine kinase amino acid residues I369, V419, F435, E436, M438 and L489 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å; and/or
(v) a set of amino acid residues that are identical to Interleukin-2 Tyrosine kinase amino acid residues according to any one of FIGS. 1, 2 and 3 wherein the root mean square deviation of the backbone atoms between said set of amino acid residues and said Interleukin-2 Tyrosine kinase amino acid residues which are identical is not greater than about 3 Å;
said method comprising the step of using all or part of the binding pocket or domain to design, select or optimize a chemical entity that interacts with said binding pocket or domain.

34. A method for designing a compound or complex that interacts with a binding pocket or domain comprising amino acid residues selected from the group consisting of:
(i) a set of amino acid residues which are identical to Interleukin-2 Tyrosine kinase amino acid residues I369, G370, V377, A389, K391, V419, F435, E436, F437, M438, E439, H440, C442, D445, L489 and S499 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(ii) a set of amino acid residues which are identical to Interleukin-2 Tyrosine kinase amino acid residues Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iii) a set of amino acid residues which are identical to Interleukin-2 Tyrosine kinase amino acid residues L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iv) a set of amino acid residues which are identical to Interleukin-2 Tyrosine kinase amino acid residues I369, V419, F435, E436, M438 and L489 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å; and/or
(v) a set of amino acid residues that are identical to Interleukin-2 Tyrosine kinase amino acid residues according to any one of FIGS. 1, 2 and 3 wherein the root mean square deviation of the backbone atoms between said set of amino acid residues and said Interleukin-2 Tyrosine kinase amino acid residues which are identical is not greater than about 3 Å;
said method comprising the steps of:
(a) providing the structure coordinates of said binding pocket or domain on a computer comprising the means for generating three-dimensional structural information from said structure coordinates;
(b) using the computer to dock a first chemical entity in part of the binding pocket or domain;
(c) docking at least a second chemical entity in another part of the binding pocket or domain;
(d) quantifying the association between the first or second chemical entity and part of the binding pocket or domain;
(e) repeating steps (b) through (d) with another first and second chemical entity;
(f) selecting a first and a second chemical entity based on said quantified association of both of said first and second chemical entity;
(g) optionally, visually inspecting the relationship of the selected first and second chemical entity to each other in relation to the binding pocket or domain on a computer screen using the three-dimensional graphical representation of the binding pocket or domain and said first and second chemical entity; and
(h) assembling the selected first and second chemical entity into a compound or complex that interacts with said binding pocket or domain by model building.

35. A method of utilizing molecular replacement to obtain structural information about a molecule or a molecular complex of unknown structure, wherein the molecule is sufficiently homologous to an Interleukin-2 Tyrosine kinase domain, comprising the steps of:
(a) crystallizing said molecule or molecular complex;
(b) generating an X-ray diffraction pattern from said crystallized molecule or molecule complex; and
(c) applying at least a portion of the structure coordinates set forth in any of FIG. 1, 2 or 3 or a homology model thereof to the X-ray diffraction pattern to generate a three-dimensional electron density map of at least a portion of the molecule or molecular complex of unknown structure; and
(d) generating a structural model of the molecule or molecular complex from the three-dimensional electron density map.

36. The method according to claim 35, wherein the molecule is selected from the group consisting of an Interleukin-2 Tyrosine kinase domain, a homologue of Interleukin-2 Tyrosine kinase domain, an Interleukin-2 Tyrosine kinase protein, and a homologue of Interleukin-2 Tyrosine kinase protein.

37. The method according to claim 35, wherein the molecular complex is selected from the group consisting of an Interleukin-2 Tyrosine kinase domain complex, a homologue of Interleukin-2 Tyrosine kinase domain complex, an Interleukin-2 Tyrosine kinase protein complex, and a homologue of Interleukin-2 Tyrosine kinase protein complex.

38. A method for identifying a candidate inhibitor that interacts with a binding site of a Interleukin-2 Tyrosine kinase domain or a homologue thereof, comprising the steps of:
(a) obtaining a crystal comprising an Interleukin-2 Tyrosine kinase domain or homologue thereof;
(b) obtaining the structure coordinates of amino acids of the crystal obtained in step (a);
(c) generating a three-dimensional structure of the Interleukin-2 Tyrosine kinase domain or homologue thereof using the structure coordinates of the amino acids obtained in step (b) with a root mean square deviation from the backbone atoms of said amino acids of not more than ±3.0 Å;
(d) determining a binding site of the Interleukin-2 Tyrosine kinase domain or homologue thereof from said three-dimensional structure; and
(e) performing docking to identify the candidate inhibitor which interacts with said binding site.

39. The method according to claim 38, further comprising the step of:
(f) contacting the identified candidate inhibitor with the Interleukin-2 Tyrosine kinase domain or homologue thereof in order to determine the effect of the inhibitor on catalytic activity.

40. The method according to claim 38, wherein the binding site of the Interleukin-2 Tyrosine kinase domain or homologue thereof determined in step (d) comprises the structure coordinates of Interleukin-2 Tyrosine kinase amino acids I369, G370, V377, A389, K391, V419, F435, E436, F437, M438, E439, H440, C442, D445, L489 and S499 according to any one of FIGS. 1, 2 and 3, wherein the root mean square deviation from the backbone atoms of said amino acids is not more than ±1.5 Å.

41. The method according to claim 38, wherein the binding site of the Interleukin-2 Tyrosine kinase domain or homologue thereof determined in step (d) comprises the structure coordinates of Interleukin-2 Tyrosine kinase amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3, wherein the root mean square deviation from the backbone atoms of said amino acids is not more than ±1.5 Å.

42. The method according to claim 38, wherein the binding site of the Interleukin-2 Tyrosine kinase domain or homologue thereof determined in step (d) comprises the structure coordinates of Interleukin-2 Tyrosine kinase amino acids L363, F365, V366, Q367, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3, wherein the root mean square deviation from the backbone atoms of said amino acids is not more than ±1.5 Å.

43. The method according to claim 38, wherein the binding site of the Interleukin-2 Tyrosine kinase domain or homologue thereof determined in step (d) comprises the structure coordinates of Interleukin-2 Tyrosine kinase amino acids I369, V419, F435, E436, M438 and L489 according to any one of FIGS. 1, 2 and 3, wherein the root mean square deviation from the backbone atoms of said amino acids is not more than ±1.5 Å.

44. The method according to any one of claims 38 to 43, wherein the crystal is an Interleukin-2 Tyrosine kinase domain bound to an active site inhibitor.

45. The method according to any one of claims 38 to 43, wherein the crystal belong to space group C2, and has unit cell parameters of a=125 Å, b=75 Å, c=79 Å, α=γ=90°, and β=94°.

46. The method according to any one of claims 38 to 43, wherein the structure coordinates of the amino acids are according to any one of FIGS. 1, 2 and 3±a root mean sqaure deviation from the backbone atoms of said amino acids of not more than 3.0 Å.

47. A method for identifying a candidate inhibitor that interacts with a binding site of an Interleukin-2 Tyrosine kinase domain or a homologue thereof, comprising the steps of determining a binding site from a three-dimensional structure to the Interleukin-2 Tyrosine kinase domain or homologue thereof to design or identify the candidate inhibitor which interacts with said binding site.

48. The method according to claim 47, wherein the binding site of the Interleukin-2 Tyrosine kinase domain or homologue thereof comprises the structure coordinates of Interleukin-2 Tyrosine kinase amino acids I369, G370, V377, A389, K391, V419, F435, E436, F437, M438, E439, H440, C442, D445, L489 and S499 according to any one of FIGS. 1, 2 and 3, wherein the root mean square deviation from the backbone atoms of said amino acids is not more than ±1.5 Å.

49. The method according to claim 47, wherein the binding site of the Interleukin-2 Tyrosine kinase domain or homologue thereof comprises the structure coordinates of Interleukin-2 Tyrosine kinase amino acids Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3, wherein the root mean square deviation from the backbone atoms of said amino acids is not more than ±1.5 Å.

50. The method according to claim 47, wherein the binding site of the Interleukin-2 Tyrosine kinase domain or homologue thereof comprises the structure coordinates of Interleukin-2 Tyrosine kinase amino acids L363, F365, V366, Q367, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3, wherein the root mean square deviation from the backbone atoms of said amino acids is not more than ±1.5 Å.

51. The method according to claim 47, wherein the binding site of the Interleukin-2 Tyrosine kinase domain or homologue thereof comprises the structure coordinates of Interleukin-2 Tyrosine kinase amino acids I369, V419, F435, E436, M438 and L489 according to any one of FIGS. 1, 2 and 3, wherein the root mean square deviation from the backbone atoms of said amino acids is not more than ±1.5 Å.

52. A method for identifying a candidate inhibitor of a molecule or molecular complex comprising a binding pocket or domain comprising amino acid residues selected from the group consisting of.
(i) a set of amino acid residues which are identical to Interleukin-2 Tyrosine kinase amino acid residues I369, G370, V377, A389, K391, V419, F435, E436, F437, M438, E439, H440, C442, D445, L489 and S499 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(ii) a set of amino acid residues which are identical to Interleukin-2 Tyrosine kinase amino acid residues Q367, I369, G370, G375, V377, H378, L379, K387, V388, A389, I390, K391, V419, L426, L433, V434, F435, E436, F437, M438, E439, H440, C442, L443, S444, D445, R486, N487, L488, L489, V490, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iii) a set of amino acid residues which are identical to Interleukin-2 Tyrosine kinase amino acid residues L363, F365, V366, Q367, Q373, G375, V377, H378, L379, G380, Y381, W382, K387, V388, A389, I390, K391, T392, A407, E408, V409, H415, K417, L418, V419, L426, L421, Y422, G423, V424, C425, I431, C432, L433, V434, F435, E436, F437, M438, E439, H440, C442, L443, S444, D445, Y446, T458, L459, L460, G461, M462, C463, L464, D465, V466, C467, E468, G469, M470, A471, Y472, L473, E474, E475, A476, C477, V478, I479, H480, R481, D482, L483, A484, A485, R486, N487, L488, L489, V490, G491, E492, Q494, V495, I496, K497, V498, S499 and D500 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å;
(iv) a set of amino acid residues which are identical to Interleukin-2 Tyrosine kinase amino acid residues I369, V419, F435, E436, M438 and L489 according to any one of FIGS. 1, 2 and 3 wherein the root mean square deviation of the backbone atoms between said set of amino acid residues and said Interleukin-2 Tyrosine kinase amino acid residues which are identical is not greater than about 1.5 Å; and/or
(v) a set of amino acid residues that are identical to Interleukin-2 Tyrosine kinase amino acid residues according to any one of FIGS. 1, 2 and 3 wherein the root mean square deviation of the backbone atoms between said set of amino acid residues and said Interleukin-2 Tyrosine kinase amino acid residues which are identical is not greater than about 3 Å;
said method comprising the steps of:
(a) using a three-dimensional structure of all or part of the binding pocket or domain to design, select or optimize a plurality of chemical entities; and
(b) selecting said candidate inhibitor based on the inhibitory effect of said chemical entities on the catalytic activity of the molecule or molecular complex.

53. A method of using the crystal according to any one of claims 1 to 8 in an inhibitory assay comprising steps of.
(a) selecting a potential inhibitor by performing rational drug design with a three-dimensional structure determined for the crystal, wherein said selecting is performed in conjunction with computer modeling;
(b) contacting the potential inhibitor with a kinase; and
(c) detecting the ability of the potential inhibitor to inhibit the kinase.

54. A method of making a crystal comprising an Interleukin-2 Tyrosine kinase domain or homologue thereof, said method comprising steps of:
(a) producing and purifying Interleukin-2 Tyrosine kinase protein;
(b) producing a crystallizable composition comprising purified Interleukin-2 Tyrosine kinase protein; and
(c) subjecting said composition to devices or conditions which promote crystallization.

55. The method according to claim 54, wherein Interleukin-2 Tyrosine kinase protein comprises Interleukin-2 Tyrosine kinase amino acid residues 357-620 according to any one of FIGS. 1, 2 or 3.

56. The method according to claim 54, wherein Interleukin-2 Tyrosine kinase protein is between 85% and 100% pure.

57. The method according to claim 54, wherein the crystallizable composition further comprises a crystallization solution.

58. The method according to claim 57, wherein the crystallization solution comprises a precipitant, ammonium sulphate, magnesium acetate, and a buffer that maintains pH at between about 4.0 and 8.0.

59. The method according to claim 58, wherein the crystallization solution further comprises a reducing agent.

60. The method according to claim 59, wherein the reducing agent is dithiothreitol.

61. The method according to claim 57, wherein the crystallization solution comprises a precipitant, Peg3350, ammonium acetate, and a buffer that maintains pH at between about 4.0 and 8.0.

62. The method according to claim 61, wherein the crystallization solution further comprises a reducing agent.

63. The method according to claim 62, wherein the reducing agent is dithiothreitol.

64. The method according to claim 54, wherein the crystallizable composition is treated with at least one micro-crystal comprising an Interleukin-2 Tyrosine kinase domain or homologue thereof.

65. A method of making a crystal comprising an Interleukin-2 Tyrosine kinase domain complex or an Interleukin-2 Tyrosine kinase domain homologue complex, said method comprising steps of:
(a) producing a crystallizable composition comprising a crystallization solution and Interleukin-2 Tyrosine kinase protein complexed with a chemical entity; and
(b) subjecting said crystallizable composition to devices or conditions which promote crystallization.

66. The method according to claim 65, wherein Interleukin-2 Tyrosine kinase protein comprises Interleukin-2 Tyrosine kinase amino acid residues 357-620 according to any one of FIGS. 1, 2 or 3.

67. The method according to claim 65, wherein the chemical entity is selected from the group consisting of an ATP analogue, a nucleotide triphosphate, a nucleotide diphosphate, adenosine, and an active site inhibitor.

68. The method according to claim 65, wherein the chemical entity is an ATP analogue.

69. The method according to claim 65, wherein the chemical entity is staurosporine.

70. The method according to claim 65, wherein the crystallization solution comprises a precipitant, ammonium sulphate, magnesium acetate, and a buffer that maintains pH at between about 4.0 and 8.0.

71. The method according to claim 70, wherein the crystallization solution further comprises a reducing agent.

72. The method according to claim 71, wherein the reducing agent is dithiothreitol.

73. The method according to claim 65, wherein the crystallization solution comprises a precipitant, Peg3350, ammonium acetate, and a buffer that maintains pH at between about 4.0 and 8.0.

74. The method according to claim 73, wherein the crystallization solution further comprises a reducing agent.

75. The method according to claim 74, wherein the reducing agent is dithiothreitol.

76. The method according to claim 65, wherein the crystallizable composition is treated with at least one micro-crystal comprising an Interleukin-2 Tyrosine kinase domain complex or an Interleukin-2 Tyrosine domain homologue complex.

77. A crystal comprising an Interleukin-2 Tyrosine kinase domain or homologue thereof produced by a method according to claim 54.

78. A crystal comprising an Interleukin-2 Tyrosine kinase domain complex or Interleukin-2 Tyrosine domain complex homologue produced by a method according to claim 65.

79. The crystal according to claim 78, wherein said Interleukin-2 Tyrosine kinase domain complex is Interleukin-2 Tyrosine kinase domain bound to an active site inhibitor.

80. The crystal according to claim 78, wherein said Interleukin-2 Tyrosine kinase domain complex is Interleukin-2 Tyrosine kinase domain bound to staurosporine.

81. The crystal according to claim 80, wherein said Interleukin-2 Tyrosine kinase domain is phosphorylated.

82. The crystal according to claim 80, wherein said Interleukin-2 Tyrosine kinase domain is unphosphorylated.

83. The crystal according to claim 78, wherein said Interleukin-2 Tyrosine kinase domain complex is Interleukin-2 Tyrosine kinase domain bound to 3-(8-phenyl-5,6-dihydrothieno[2,3-h]quinazolin-2-ylamino)benzenesulfonamide.

84. The crystal according to claim 83, wherein said Interleukin-2 Tyrosine kinase domain is phosphorylated.

85. The crystal according to claim 83, wherein said Interleukin-2 Tyrosine kinase domain is unphosphorylated.
